SEQUENCE LISTING (53 sequences)

SEQ ID NO: 1 - DNA, 3114 bp, Rattus norvegicus
  misc_feature (314)..(1051): coding region (ORF)
ccagagtgaaggataaatcatggaggtcaacaaggaacagtaggacctatgagtaaggagacctgcacagggcactgagaagcatcagttgggttggtagcctgtctctgaaagccttcatcctaaccgacgccaacgagtcctggctgtgcatgctggtgcaagcctggaatgctagaactcaggaggtggaggctggagaatcaagagtttgaggccaacttggactacgtaagagtctgcctttaaacgcaacaaaaacgaatggagagagatcagaaattgaataacttctgccctgctcgttcagggcatggagaagtggacggcctgggagccgcagggcgccgatgcgctgcggcgctttcaagggttgctgctggaccgccgcggccggctgcactgccaagtgttgcgcctgcgcgaagtggcccggaggctcgagcgtctacggaggcgctccttggcagccaacgtagctggcagctctctgagcgctgctggcgccctagcagccatcgtggggttatcactcagcccggtcaccctgggagcctcgctcgtggcgtccgccgtgggcttaggggtggccaccgccggaggggcagtcaccatcacgtccgacctctctctgatcttctgcaattcccgggaggtacggagggtgcaagagatcgccgccacctgccaggaccagatgcgcgaactcctgagctgccttgagttcttctgtcagtggcaggggcgcggggaccgccagctgctgcagagcgggagggacgcctccatggctctttacaactctgtctacttcatcgtcttcttcggctcgcgtggcttcctcatccccaggcgtgcggagggggccaccaaagtcagccaggccgtgctgaaggccaagattcagaaactgtctgagagcctggagtcctgcactggtgccctggatgaacttagtgagcagctggaatcccgggtccagctctgtaccaaggccggccgtggtcacaacctcaggaactcccctgatctggatgcagcgttgtttttctaagagcatcctctagctgtgtggaatgttctagattcgcagcatccacaaggaagtgctacatgggcggagtgcaaaggatttcagaagctcttcttgcagggcatcagtccgtagctccttgtgtgtgcgaaagacttttcacttgtgtaatcccaactgagtatgtgaccctaaacagtcactttggggactccccaaatcctttttagctgcacacagcttgtcagactgtccttcaattagagttattggggtgggggggcttgatggcttgagtaatagaggtctggcgaggtgtctccctcttggacctcttatgtgttgttactagaatcctgagattctcaaatgttggtgagaggagacttttacttttcaactttgcttcagcagtttccgatacacaggactccagaatccagaacaagaaagaagaaccttgtgtttgtagggtgtgcagacccagacggggccgaggagctgacttgctcagctctcacacacagccagtttatccactcacagaccaaacctggctactgcatagactgttccagtgtggcttcaaatccacacctctaggtaccctgagaaggaaagccacctgaagagtcactctaatcccaacacgctcacccccttcacgtccataaaggagctgggcaaggggtgagatgaagaccctgacaattttaaatgactgtagcatagagagccatggcctttgagtttaagagtcttgatcccaggttctgtcccccactgtcctgtgacttagccaccttgtcttgctacagatggtggtaggaggccaccctgttgcgaagccctgagataatgacaaacacagaggctagctcacaaaaatgtacttcctggcctggcttctgaagggttaactgttgggctccatcccagatttctgagatcaggaactccaaatatgaggcccgcctctggctgattctgatgccccataaatgtttgaaaatgacacagcaaaggttcatctccagccaggtgtggtgggacacacctgtaaggccagcgcttggagatggagacagggggaccagtagttcagggtcattcttggctacatagcaaactcaaggccaccctggtctcaaaaaccaaaacaaaaagccatcttctgactcccttcaattgttcaaagcctttccagggccttcagaatcacgctcagagtgttctgggaagattagcccagaagccagagaaagagtacgctgtgtgcttgtaaagccagttactctgtcccctgtgaactaggagacagagcacttccgaccctatagagggcagtagtggccattccttgtaggggactggtatagaagtaatgtgaacttaccaggaaaaaacaaacaaacacaacagcaaaatccctttggtctctgaaaactccagacaacctatctttatttatttaaaaatagttatttaattgctgcctgttatttacatttgattttatttaaccttcacattatttagaaaataataagagtagtaagtgtctgaataggaagggagtctcttaaggctctttccaagagctcaggtttggatttctagagtccccccgaccccagagaggactctttagtgtttgacacggtctttgtaagtaagatggggagtcctggagagagagaccaagctgatttttaaactaggaaatggagtcttgaactgtggaagatttgaaaagttaagcctatgtgtcttgaaggtacttggccagaaaagcacttggcttgaaaaagaaaacctgtttaattcaggggtggaggaatagagacagacgaagaaagcatttagacctcggaaacctgatgtcctatgaaattctgtttttataaaattgtgttatggtggagatctgttgcatttcaactttgtggctgtaagaaacctgttatctatgtttaagaaagtacttctaatttattcaatgtcttcctaaattatcctttaaaaaaaaaagttggaaagtctatgagaccgtacccaagaaaaaaaaaaaaaaaaaaaaa

SEQ ID NO: 2 - DNA, 2924 bp, Rattus norvegicus
  CDS (247)..(984)
ctgcgtttggaggggaaagcgaacacacaatgttcatttcctaaatacgggacgtgctttgccagcgtctctttttccaacatgtcatatcctggccaaaggcagcaggggtcagggcaggaaactgcagcttctcagaatgagacaaggctttcccagagccgtcattggttcctgggaactataaagcacgcttatccagaaacagtctcccactttgcttcctggaggccagagtgaaggggcatggagaagtggacggcctgggagccgcagggcgccgatgcgctgcggcgctttcaagggttgctgctggaccgccgcggccggctgcactgccaagtgttgcgcctgcgcgaagtggcccggaggctcgagcgtctacggaggcgctccttggcagccaacgtagctggcagctctctgagcgctgctggcgccctagcagccatcgtggggttatcactcagcccggtcaccctgggagcctcgctcgtggcgtccgccgtgggcttaggggtggccaccgccggaggggcagtcaccatcacgtccgacctctctctgatcttctgcaattcccgggaggtacggagggtgcaagagatcgccgccacctgccaggaccagatgcgcgaactcctgagctgccttgagttcttctgtcagtggcaggggcgcggggaccgccagctgctgcagagcgggagggacgcctccatggctctttacaactctgtctacttcatcgtcttcttcggctcgcgtggcttcctcatccccaggcgtgcggagggggccaccaaagtcagccaggccgtgctgaaggccaagattcagaaactgtctgagagcctggagtcctgcactggtgccctggatgaacttagtgagcagctggaatcccgggtccagctctgtaccaaggccggccgtggtcacaacctcaggaactcccctgatctggatgcagcgttgtttttctaagagcatcctctagctgtgtggaatgttctagattcgcagcatccacaaggaagtgctacatgggcggagtgcaaaggatttcagaagctcttcttgcagggcatcagtccgtagctccttgtgtgtgcgaaagacttttcacttgtgtaatcccaactgagtatgtgaccctaaacagtcactttggggactccccaaatcctttttagctgcacacagcttgtcagactgtccttcaattagagttattggggtgggggggcttgatggcttgagtaatagaggtctggcgaggtgtctccctcttggacctcttatgtgttgttactagaatcctgagattctcaaatgttggtgagaggagacttttacttttcaactttgcttcggcagtttccgatacacaggactccagaatccagaacaagaaagaagaaccttgtgtttgtagggtgtgcagacccagacggggccgaggagctgacttgctcagctctcacacgcagccagtttatccactcacagaccaaacctggctactgcatagactgttccagtgtggcttcaaatccacacctctaggtaccctgagaaggaaagccacctgaagagtcactctaatcccaacacgctcacccccttcacgtccataaaggagctgggcaaggggtgagatgaagaccctgacaattttaaatgactgtagcatagagagccatggcctttgagtttaagagtcttgatcccaggttctgtcccccactgtcctgtgacttagccaccttgtcttgctacagatggtggtaggaggccaccctgttgcgaagtcctgagataatgacaaacacagaggctagctcacaaaaatgtacttcctggcctggcttctgaagggttaactgttgggctccatcccagatttctgagatcaggaactccaaatatgaggcccgcctctggctgattctgatgccccataaatgtttgaaaatgacacagcaaaggttcatctccagccaggtgtggtgggacacacctgtaaggccagcgcttggagatggagacagggggaccagtagttcagggtcattcttggctacatagcaaactcaaggccaccctggtctcaaaaaccaaaacaaaaagccatcttctgactcccttcaattgttcaaagcctttccagggccttcagaatcacgctcagagtgttctgggaagattagcccagaagccagagaaagagtacgctgtgtgcttgtaaagccagttactctgtcccctgtgaactaggagacagagcacttccgaccctatagagggcagtagtggccattccttgtaggggactggtatagaagtaatgtgaactatttaaaaatagttatttaattgctgccttcacatttgattttatttaaccttcacattatttagaaaataataagagtagtaagtgtctgaataggaagggagtctcttaaggctctttccaagagctcaggtttggatttctagagtccccccgaccccagagaggactctttagtgtttgacacggtctttgtaagtaagatggggagtcctggagagagagaccaagctgatttttaaactaggaaatggagtcttgaactgtggaagatttgaaaagttaagcctatgtgtcttgaaggtacttggccagaaaagcacttggcttgaaaaagaaaacctgtttaattcaggggtggaggaatagagacagatgaagaaagcatttagacctcggaaacctgatgtcctatgaaattctgtttttataaaattgtgttatggtggagatctgttgcatttcgactttgtggctgtaagaaacctgttatctatgtttaagaaagtacttctaatttattcaatgtcttcctaaattatcctttaaaaaaaaaaaaaaaaaa

SEQ ID NO: 3 - protein, 246 aa, Rattus norvegicus
MEKWTAWEPQGADALRRFQGLLLDRRGRLHCQVLRLREVARRLERLRRRSLAANVAGSSLSAAGALAAIVGLSLSPVTLGASLVASAVGLGVATAGGAVTITSDLSLIFCNSREVRRVQEIAATCQDQMRELLSCLEFFCQWQGRGDRQLLQSGRDASMALYNSVYFIVFFGSRGFLIPRRAEGATKVSQAVLKAKIQKLSESLESCTGALDELSEQLESRVQLCTKAGRGHNLRNSPDLDAALFF

SEQ ID NO: 4 - DNA, 16613 bp, Mus musculus
  gene (1)..(16611): L119 genomic DNA
gaattctaaacttagctttttagagataaaattaatcttgaagtgtattcagagggcagatagagtcttgagtcctggcaccattgactgatatattcgctcttttggtttgtcttgtaaccccttggcatagattctctgagatccatctggttcttatctctcagttctgtttaatctctcaggactcaccatgtaaaggagaactaccttgaactctgatcctcctgcctctgcctctagacttttgattgacactgtgcagcaccatgcccacattatgcagggatggggcggaacccagggtttgccaggcatctgaaactaaaaccaaaccctctagaaatgaaacaccaaaccctttagagcagtttggattgctcagaagtgaaggagaagaggcagattttcgccgagaagagagagtttcgtggtatggggaggaagcctgcttcccaagtgtgaagttagttggaccttaaataaagctatttattcttcccaggttgcaccagttcttaatgtccctcaggccctccacagcagtagggccaacccctctccttccgctgggctgcagaaggtaactgattagctgcctcccagtaacgttgggggcgttgcccttgcagagagtgtggaggttttcttagctctgacctaaaactgtctgcattaatttactcttaattatttttacagattctattcctggtccattgcttaataaaactctaggggtttctgtctctctctctctctctctctctctctctctctctctctctctcttcaatataataaagtcagctgcatcagacatggatggaatcatctgctaagctgaggttctgaacggtgccccctgcacccctaagtgtctgtgtgtgttccccattctctttcagaaaaagaatgtgtgtataaaattgtcagctatggtcagaaaccaggttaaaagtgaatctacaatcttccatcaggaaggaaggaaggaaggaaggaaggaaggaaggaaggaaggaagtcattctgctcagagaaggatagagggttctagcctgtaccagaacctcctactctgccttaatatggctaaagagagtagggcctcctggatttttggctaaagatgactgttctgtctttggaaaagcaactctctatgactcggaggggggaaaatacttcctaggtagccatggagccaggaagtgatccatccaagaataaaaatacctttgggggtatttcagaataaatgtttgtaggaaccaggctgaaagggatgacacgggactctgtgtgatcctgagatggggctgcatatccacagagatattctcttagcaaggtggccctggttcaacctcttccagcatgtgcctatgttaagctaactaatagaacagattgcctccccagagtgggcagactcatgtgcatctaatctctggagtttctcccaggttccatggaccccaagataagcaagcactcatatgatgaagtaggctgaatctccattgctgagaaggacgtcaccaagatagataggacagacctaggaaattccaatgttagagtaggcagtttgtcaggaggcctagctaatgaggaatgtggctggctcttctgataggaaccacagagcttaagagaggaaattaaacaacccagtgtgtatccctcccctcccccaataatgagagctccaagacatgaactgcctggatctgtgttattccaccccgcatcccattttgcaaggtctgctgttgctaaaaatgacaccattctcctcaactgcgggcaaagcatgatgaggtggatcagcataactggagcaccaggctgcaggatgtacttgacccaggcagcgtgaagcatttgagccctgattagtaaatcaagagagacagaagcacggttgtgctggagatgccttttaagcacggaagagctgtcacccacatcataactgtggtggcaaataagtgatacctttgatcatgcgaatgaatagcagacgttgtaaatacaggaaggggagaggaggagtttcacgtgtgatttcagttactttttctctcctgggtttccacatgaagttctgagaaagcaaaaagaaacaaagatgggcagttgccctggagaaggagaaaataggaccgtgcaaagggacaggaaacagcaaaggagaggcattgggcgtccagatagaggtctaggaacactgccaggaaccagggcttaagggtagaggaacagttggataggcatgaagtttgtgtgcatggagggacagtattcccaggtatagtacatatgtatatctatagcaactggacagggaggagatatggggttggagaaaagaattgggattcagcctgggtccatctttgtactagaaccaacatctaacagaacgaagaggaaggattaattttggctcctggttttatcagtccatcaactagagctttcagggtgtgtacagcccctcttatcacaacaggttggaagtgggaagagagagtacaggcaagataagatatggctcccaggaaccccagtgacctacttcctctgagctctgcctcccacctttcaccgcctccccataaggccactgtgtcatgaatctatcaatgaacaaatccacgtcttaagtcagagctttcattctaatttcacttctgtttgtatgataaaaagaaaaaacaaaaacaaacaaacaaaaaaccctgaaggtaaaaggaggtcattttagctcacaaatccaggccactgatcaccattgtgggaaggtcaagaacacctactcacatcatatccatggtcaagaacacagagaaataaatgcatgcctacctaatactcggctcatgctctttcattttatacagttcagaaccccatcatagggaatggtgctgcccacagtggactgagtctccccaagtcaattaagatattcaaggcaatccccatgcccacaggccaactttatctggacaattcctcattgagatctcttccagggtatttctaggttatgttgagttgacaattgaaactaatccttgcactactaaaatccccctgcggctgtgtcttccccacctccatgcatgactgtaagggggttttaggtaggttactcaggtagaaatatccattctaaatgccagcaatcatttttgtctgctttcaatgcaatctctggcgttgatcatctgtctcctgattctcccaccatgccttaccgatcatgatggcatataccagcaaagtgtgattgaaataaaccctttattcttaggttgccttttgttcgatatttcggtacagcagtgaagtgacttacacaccagggaaaggctaacagatagagggaaaaccagagcagtctaggaactggagatgtaaagggtcctggggagcaaggagaggaaactggtaaggaaggacactctaatagttagagctggaagtatcagaatacagcatttctagcattgtagagcaggccagcaaaggccaggggaatgtggagatgattgtacttcagcaccagtattcctaataaggtagttctgagtattcctcagggtgagtcagagactttgaggctctgacaggaaggaaggatcaaagtgatgatgtttcataaggtgagacacttcaatcagtgcaatcctgtctcccaaagtaaagggcagcatgttaggcatggctccccagaggaaggtggacaagagggagtgtgtgtgtgtgtgtgtgagagagaggggggaggggtttgtatatatgacacacagagtggaattgtgatgtgtgtgtccaaatgtgtgtgtggtatgtggtatgcatatgtgtatgtgtgtgatgaaaaagagaagggaaaagagaaagatgtagagagagatggagaaaaagagggagggaaattaagatagatggagggatacacgaataagaaaaagatgataaatggaaagacagatggagaaagagacagagggatattgaaagataagagatggggagagagaaagaaatggcaggagcagggaatagagagatggggaaaacatgcagggagatagagatgagagatggaaagacaggtggatagagagaaaaggagagaacaagagatcagagatggagagaaataaagaggaggggggggggagaaaggtagagagacagaggtggaaagacaaagagggagagatggagagagctgtgagcaagcttattcccttttctttatttttggctttttaagacaggatctcaccatgtagctctggctgaatttaaatttactatgtagcccagagtggccttaaacttgcagcaatcctcctgtctctttcttcagagtacaggaattgcaggcatgtgccagcacacccaccttgatttagctttttccttagcacacccaccttgatttagttttttccttctggttaaatggtagtttccatcttactcatatttaagcacacatccttacattgtgaagcaagactaccatttgcctatagagcttctgtctcgtgataaactcaaactccatctggcatctctgtttttttccactgcccctgtggttctctggagaactcattggctggtccatctctaagtctccatccatgaggtaactgtctgggcatctttggagtcaaatgaacccagtccatctggttgtgaagagggggaggggatgatgttggttacccagagtttgatggtcacatcttgggaccacagacaggacagacagccgcaatgagagctcagtgcttcagaagcctgagccacaaagtctttcattggtactgttgtgtgataattccatatacacgcataacgtactttgttcgagtcactccattaccatcttttatctcactctcattcccattgatccccttctttccaaaaacaaacaaaaaagcccatctcctatttttatgtctttttttgtttattttgcttttttaaaaagtgatccagtcagttttaattagtcagctccatgtgcctgaataattatactcacttaattaacaaatattaattaatgagctactgtgtatccttaatcaattaatgggttattaatagggccaaaagaaacctaccagtgccttgactactgaaggaaatgtttctcccagcaaccattgactgactgtagatccccagggaggagtagagtcttgggagccccttccaagcagggcacagctgctatgagtttatgaccagaatggccatttcctgttgccagaagaggacagaatttcattgcactcctgtacattttcagtctcttagactctttttgtgacctttttaaaaaatttttagtttcttttcttcataggacaacttcattgagaatctattttgtccagcagacctcttttttttttttttttaaatagaaatctagtgtctaatttctctagatcctcctttaggtcttgggcattgattaaaattgaccctgacttaagattctctgatgatttgaggttgatgtctcccacaattctctggcatttgaatatctgatctccagttggtagctatttacgggaggctcaggagatatggcatgctagaagcagtatgtcactggggacaggctttgagttttcaaaaggctcatgccatttccagccctctctgcttactattttttttttttttttggttccagatgtgagctctctccacttgcccctccaactaccttccacctgctacctctgatgtgccatcatggactctaatcatctacacccataagtttaaaataaactctctttgttttcttgatcatgaagttttaccacagcaacaaaaaagtaaccaatgcagatcctgtccctgccaacaatctctgtttgcataaaagactatttattagttactgactagttatccaaaggaaaggcattgttttggggaccagatctgagaatgtgttggtagaaggggtacctacaacactccatgatcaatggtgaggtgtttgatgtttgtgcgatacattttctactcctgatacgagggtcttagctctaacaccaattcttttatcctcctaactttctatgcaccaactatggaagaactttctaacaaatagtgcattatccttacatggaccttctgtcttgtataattttacaggcagaagggtcatgagttcaaggccagcctgagctacccaacaaattccaacctagtttaggctgcatagtgaggccacatattctagtttcctttctgctgctgtgataaaacatgctggccaaagggttcatttacacttccacgttacagtccattgctttagggaaatcaaggctggaacctaaggcatcacattcacagtcaagagtaattagagtaaacacatgaatctttgcttgcttacttgcttatatctgttggcattctcctctcaaatcatttgggaatccctatctagggaatggtaccacccacaatggaatgggtcttccttcattgactaacaataaagacaatctcccacagattcaccttcaagcctgtatgatttagataattcctcattgagacccttcccaggtgattctagtttgtatcaggttgccagttaaaactagtcaacacaccatgcctcaggaaaacaaaagattgggggtacgcgtatagctcgtaagctacatagttggctttgtagtttgggaggctgacaagacaggaaatgtgtggtcatgcatgctttttttatgaagagagggtaagctctctcccaagatgcctgccccagagtaccgtttagcttggattcatctctcagggaaaggagctacagttgaaaaccctttcatgggggtcacttaggactataagaaaacacagatatttgcactaaagttcataacagcagcaaagttacagttatgaagtagcaacgaaaaataattttacgatcaggggagtgagctcaacatgaggaactgtattaaagtgtcacagaattaggaaggctgagaaccactgctctaggagaacagaactgatagagtgaatatatattataaaatggtgtctgtccattagcttacaggatacagactaggtggttcaacaatggctgtcttcatactgaagaggctaggaacatggtagctgctcagtctatgacactggatgcctcagtagcctgaatgaatggtaaaggcttgggtagtccctggagagccactttagaaggctgaagaagctgacattagtgaaagaggctaaagcatcagaatcccagcataaatgcaccaccatctagaagtgaatgaaggcaagcaatgatatttttccatctgggttttttttttttttttagttaggctgttgtagaaggtggatcccactctgagggaagagtctatccagcagaatgtctcttagttgactccaaatccgaccaagttgacacccaagattgagcacaaacaccatcctggagaacaaccagactccactgctcatgggtagagatggcattgagtaaagagagaactctagctttccttcttgcctacaaaaactagaggctgttaaataacagaccagcactgggaaagtggctcagttggaagagtgcttgccaagcatgcacaaggtctggggtcccgttcccagcatcactcaaatcgggtgtggtggtacacacctctaatcggatggagacagcaagatctacttaatgagttctaggctagccagaactccatagtgagaccgcactgaacagatgcaagagactccaaatgaataagggggacttttgtttcgttttgggaacagggtctcacgtagcccaggctggattccaactagctatgaatccgaagctgaccttgaatgtctaaccctcaagcttttacttcctggtgctggaattacaggggtgggccacagtgatgggtttctgggtgccagtgttagaatccagggcctccgcatgctagtctactgcacctccagctggagacatgaatcatttaggtgaaaaccagtttggcctttgacgatggtgtcaggggtcctttggtgagactgggtttcccaactaagcttcagcttctcccctccccccacccacaccctgactgcttcagggatagctatggggtgcatcgccatctggtctgggtaaaggcacagaaagaagctacattcatttccccgcctgcacgctctgtaatagataactgctcttcagaccctgctggggaacctgtagctagaatccactcatttaaccatgatgtccacgcttcatatgaaaaccccggaagccatcatctccccgctgccatggaaagtgtataattagctcggtgtgcagcttgaccccagtgatttttctgacgcacttggccccgcagtgtgccctttctgagacctcctgtgtcttctccacagctggagatgccctaaactgcacgcagcacttcctgtgggcgtggtcttgcattttaggcgctccttggtgctggctgccctcccttgatgggtcacatgcttcagctacttacatccccacaaagctctttgaaaaggaccatgagtggctgtatcgatcataattaagttttccggtccctcctatttctttttaaaaatgattttctgatggagtcctctcaaagaaacactataattgggcagcctggggcatgtgggaaagcctcccccgatggcgtcagtagctattctcaggagaggaaaggcagggtatccccactgggagatgacagcacttgtttcaagttggggaagagcctgtggtttctcttcctgcgtttggaggggaaagcgaacacacaatattcatttcctaaatacgggacgtgctttgccagcgtctctttttccaacatgtcatatcctggccgaaggcagcaggggtcagggcaggaaacagcagcttctcagaatgagacaaggctttcccagagccgtcattggctcctgggagctataaagtatgctcgtccagaaacggtctcccacttttcttcctggaggccagagtgaagggtaagtggggagtccgagggatgcgtctgcaatgggattggtgatatcggggtcaactctcgaggcgtcatgtatttggagtgactttttcccaacggttcttgttacctgaaaacttcttactggtcagctagatcatggaggtcaaccaggagctgtaggacctatgagtcaggagaccagtgttcttctgggggcactgagaaacatcagctgttggtagccggtctctaaaggccttcattttaaccgaggacaatgagttctggctgtgcatgccgatgcaagcctggaattctagaactcaggaggtggaggccggagaatcgggggtttgaggtcaacttggactatgaaacagcctgtctttaaaaacaacaaaaacaaatggagaaagataaaaaattaaataacttctgccctgctcgttcaggtccgacatgctctttcagtgacttatttggtgcctttaagaagttctgactggtggagagtacagtgtgtatgaaatggggctccctaattaccaggacagatggccttttacagatgaacctggtacattagctttctcccttagtcaccttttattggcttcagtacatctgggcccaaggaagcctccagtgagctgctagctagcaccttttcttttttaccttgagacaagttcttgttatgtagccttgaatggcctagaactcagtatttagaccaggttggccttgaactctcaatgatcctgcctctgcctcccaagtgctaggattagagagagggacagggacattgagagagggagaaacagagggagatggagggaggtagagagaggaagaggggtgagaaagaatgagagagagaaagagagagagagagagagagagagagagagagagagaatataatgagaagggggggcaagtaccttgagtccctaggacacaagtctccacataatgaaaagtttttgttacattacatgcacggtttgctacttttaaggtgttctcttctttttattggtccttgtttgtcaggggttggggaatagaatagggtgtcacaccagacaatggctctacccctgagctgcatcctcagcccaagcgtcattttgaaaacagagcaaagtctctgttttcagagcattaacctggagcagctttctaatagtctccaccttcccgtttaaacctttatttaaatctgtggatggtgtccatagattctgttccctggaaggaaagcagaagacagcataatctctgtaggaacgcacagttaacgggttctgggaacttgtgtgacttgtcccaggctccagagcctttggaaacttgacccataacccagtagtaacctacctccctgttcccagtcctgagtggtttatttattcgtgtattcatttcggcagccttgtggattgagtctgagggttgaacgctaggtaagtgatctaccattaaggcaacacgcccagcataaagtctattaattatttgaggactctctagagagtgcaccgggcagctctagcctagggcagcaacgggtgcggaaactcgggctaactgtgcatctgtgtccctcacagggcatggagaagcgggcggcctgggagccgcagggcgccgatgcgctgcggcgcttccaaggattgctgctggaccgccgtggccggctgcacagccaagtgctgcgcctgcgcgaagtggcccggaggctagagcggctacgcaggcgctccctggcggccaacgtggcaggcagctctctgagcgccgctggtgccctggcagccatcgtggggttgtcactcagcccggtcaccctgggagcctcgctcgtggcgtcggccgtgggcttaggggtggccaccgccggaggggcagtcaccatcacgtccgacctctctctgatcttctgcaattcccgggaggtgcggagggtgcaggagatcgccgccacctgccaggaccagatgcgcgagctcctgagctgtctggagttcttctgtcagtggcagggacgcggggaccgccagctgctgcagagcgggagggacgcctccatggccctttacaactctgtctacttcatcgtcttcttcggctcacgtggcttcctcatccccagacgtgccgagggggccaccaaagtgagccaggccgtcctgaaggccaagattcagaaactgtctgagagcctggagtcgtgcactggcgccctggatgaacttagtgagcagctggagtcccgggtccagctctgcaccaaggccggccgtggccacaacctcaggatctccactgatctggacgcagcgttgtttttctaagagcatcctctacctatatggaatgttctagagtcgtagcatccacagggaagtgctacatgggtggagtgcaaaggatgttaggaactcttcttgcagggcttccgtcagtccttagctccttgtgtgtgtgaaggacttttcgcttgtgtaatcccaactgagtttttgtctttgtagggattgtagacccagcctgggccgcggagctgacttgctctgctcttcccaccccagtttatccactcatagaccaaatctgactattgcatactttcccagcatggcttcaaattcacacctctaggtaccctgagagggaaagccaccggaagagtcactttaatcgcaacatactcacccgccttcacttctgtaaagaagctgggggaggggcgagatgcagtccctgacaattttaaataactgaccacaaagaggagagcaacggcctttgagttaaacagtcttggactgatcacatgctctgtcccccaccgtcatgtgacttagccaccttctcttgctgagacttggcttgttcagtgacacagatggtggtaagaacacaccctgttgtgaagccctgagataagaaaggaccttcagagtcactctcagaaagtccaggaaagactggcccagcagcccaagaaagactatgctgtgtgcttgtaaagccagttactctgtcccctgtaaactagaagacagagcagcacagaccctttagagggcagtgttgcccattccatgtagagactggtacagaggtaatgtgaagttaccagcaaaaacaaacaaaatcaaaaacaaaaaataaaaacaaaacaaacaaacaaataaacccccaaacctctttatcctctgaaaactccagacaccatatctttatttatttaaaattagttatttaatttctgtgtattatttacatttgattttatttaactatagcatttagaaaataataagaatagtaagggtctgaataggaagggagtctgttaaggctctttccaggaggtcaggtttggatttctagagtctttttccctcagagaggactctctagtgtttggcacgatccctgtaagtaagatgggggagtcctgaggagagagagagagactaagctgatttttaaactggtaaatggagtcttgaattgtggaatatatgaatgattaagcctgtgtgtcctgaaggtactttgccagaaagcacttgacttgaaaaagaaaacctatttaattcaggagtggaggaatagagactcatgaaggaaacatttagaccttggaaacctgatgtcctatgaaattctgtttttataaaattgtgttatggtagagatctgtcacattttgactttggggctgtaagaaacctgctatctatgcttaagaaagtacttctaatttattcaatatcttcctaaattatcctttagaaagagttgcaaagtctttgggaccatacccaggaaaccttggctgtatatttaaattatttaatgctatacatttgcgcagcccctatgattcccagtgatggccacgtgtctgaggaaatgttttggcatgaggggaaggggtgctctttctatatttatttttgtttcctataaactgtagttggggtatatcctgatttaatttgacattgatgtggctttttttttttttgacactgtgtctttctctaggtagccctggatgtcccagaactcactctgtagaccaggctgacctcaaactcacagagctctgcctgcttctgcctcctgagtgctgggattaaaggaatacacagcatgcccagcttggttttttttttttttgtttttgtttttgttttgttttgtttttaactgctgtcacttttagactgcctgtgtgggcactggagtatacctcaggctctccttccatcatccggctgaagccattccctgatgccgtcatgcattggtgctttgtctcgggttatactcctccattaccgacctgttccatataacccaaaatgggaatgttttgagtatatagcaaagtactaagcttctgaaggttttatcagtggtctgctgtgtgcgcatctgagagtgtctactttccatccacagatggcagaagggtctagaggttgagttccagggcacccgagctacacaatgagacctcatctcaaaaatacaatgaaacaacaaaaactgaaagcacatttgaataattcgctgtttgtctgtattatgagggattagcttggtcattctctttcttatgtgtaggcatgttgtgaaaattggaggatttgccaggggttagcaaagttcagtacaattgatcctagagacaaattgtttatttctttcatgggacagtggagtgggttttccctttcgaccgtcattctataagcaagcaggtcgaaagggtttgtagcgcaaatgaaaagagagaaataaatgttagggtttggaaaatacatgtttgttctaaatgctggggtgttcttgtgttccttttttggttttacagtcacctggattgttttgtttcacagtaaagggaccacatttggtttgcatcttaaaactctgataaagtttgggggctggagagatggctcagcaatgaagagcatctgttgctctcacagagttctgggattccattcccagcacgcacatgacagctcacaattatctgtgaatccagttccaggggataccctcttctgatctccacagacatcagccatgcactcggtacgaatacatatatgtgggcaaaacattcatatatacaatttaaaaaaaatcataaaaaggaccaaccctctccaagaaagaaagaagaacccttagtgagcactttgaccctaactccttttgtgtatttgccctagacacaggtcatgtccaatatgatgaatttcaaaggtaaaagtatttgctgggctgggggcaggggagagaggagtgctgcagagaccattctagattaggtcatggtcagtcaactttctgaatctccccttcagcaacttggtatgaataataaggaaaaatagacaaagaggttgtatgggggctgaagtgatggctcacccgctaagagtctttcttctttcggagaagcacagtttggttccaacattgatttcagttggctcacaactgtgtggagttctggcacctcaggggatttaatgcccttgtctggcctctgtgggcacctgcataccctcagacatacagacgtagattaaaactaagttgaagaaaggaggctacatagtggtaagtggagattagcgtagctgagggtgggagcttcctaattcactcttccattacagtaaatccatccttcctgttctactagagtaaatggctcaatactaaaagttttcttcttttcataattactggtcccattttgtctttgaatgcttttaagctgtgtgagtcaattgtttaatcaaatgctatacaggaattcccatgtgatctagaaagcaggctttttagtcatcttacaaatttgaaagtgagttggtcctagtgatcccagaactcgaaaagctgaggcaggaagaattttatgttctacctgagcccaggctacatagtgagaccctacctttgagaagaggagggggaggaagaggaggaagagggagaagaggaagaggaggaggaggaggaacagcagcagcagcaacaatgacgacgagatgacaacaacaacaacaacaaaataataccaactaaaaagaaaccctcaaaacaaaaataaaaaaccacacccaaccctggtttggtggtgcttgcttgtaaccctagcattcaggaggtaagaggcaggaagattaggaagtcaaggtcagctttgcctatggaagccagcctgggttataagagactgtctttcaaaatacagaagcattcaaaatgatcagctgatgcccaagtttgatctctattcctgtttttccaggcctagggaactctagacaggaggatgaagaattcaaggcgagcctggtcttcagagacctatcttgaaaacaaaacaaagcaaaagcttatgtggtggctgccgtaggcagttgggaacttacattctggagtcactctaaccgctggtctgtgcacctgcgccttggaaagcttccggggtctgtgtcagcctcagtctgtcacagcggacctgaagctggagtcctatctttgtcaaagcatacagaattcctgtgataacaagcatccttcccgttaatagcaacgggtctcttgttgataggaacttaaaagctgcttttgtttctgacaaaactggaagaccgtaagaaggaataaaaccctgctgcacactggagctggacaaagagggccctaagttccaggaatgttctgttagcaaccagagttgtcagaagggaaactgtgctgatagagtgggtcccatctggggaccaggaatgtacacctggaatcctcagcagcccctgtctcactggcaatgacaatgggctgtggagagtggtggtgttgtggtcttactgtttaatgaggttttgtacctccgttataaccaaccctccctcaccgtaccacccactcaaagtgttgtactagaaggcctgctgtttgcttgatcccagaagtatagccaggggccactgccagtgtctcctcacccacagtgagacactaactggttcccatggccaagtgtatgaagagagttgaaaccgagataggctgtttctgaaggtggagctgggaaggagtcatgcctcaaatctttctcgaaggcaaaagctgctcttccaggtcaacaaacaacactgccctcatcttatacgggagattagatggcggctctataggcgaaggccttctctacggctccccatggcggatcccgatgaaaagagacagttcatggttttaaggcattcattgtcatggcgtcaagtggatgagtaaaactgtaccccacttttcaggacggtcctgaggttaaatatcttttgcagggaggagtgtctgggaaggggagtttattggtaagcctccaggcctttaggtacctcattaaaatggagatgtcttgaggctagc

SEQ ID NO: 5 - DNA, 894 bp, Homo sapiens
  CDS (1)..(891)
  CDS (154)..(891)
atgatccactggaaacagacccgttcccctagcgtggcagtggctgctccgctgaactcgtgccaagttcccgctggcgtccgggcagcagggcgggagcggcggctggcacggagactccaggctgaccgtgtgtctatgtccccgcagggaatggagaggccggcggcccgggagccgcatgggcccgacgcgctgcggcgcttccagggactgctgctggaccgccgaggccggctgcacggccaggtgctgcgcctgcgcgaggtggcccggcgcctggagcgcctgcgcaggcgctccctcgtagccaacgtggccggcagctcgctgagcgcaacgggcgccctcgccgccatcgtggggctctcgctcagcccggtcaccctggggacctcgctgctggtgtcggccgtggggctgggggtggccacagccggaggggccgtcaccatcacgtccgatctctcgctgatcttctgcaactcccgggagctgcggagggtgcaggagatcgcggccacctgccaggaccagatgcgagagatcctgagctgcctcgagtttttctgccgctggcagggctgcggggaccgccagctgctgcagtgcgggaggaacgcctccatcgccctgtacaattctgtctacttcatcgtcttctttggctcacgtggcttcctcatccccaggcgggcggagggggacaccaaggttagccaggccgtgctgaaggccaagattcagaaactggccgagagcctggagtcctgcaccggggctctggacgaactcagcgagcagctggagtctcgggttcagctctgcaccaagtccagtcgtggccacgacctcaagatctctgctgaccagcgtgcagggctgtttttctga

SEQ ID NO: 6 - protein, 297 aa, Homo sapiens [sequence omitted]

SEQ ID NO: 7 - protein, 246 aa, Homo sapiens [sequence omitted]

SEQ ID NO: 8 - DNA, 24 bp, Artificial Sequence
  Description of Artificial Sequence oligonucleotide primer
tatcactcagcccggtcaccctgg

SEQ ID NO: 9 - DNA, 24 bp, Artificial Sequence
  Description of Artificial Sequence oligonucleotide primer
acgcctggggatgaggaagccacg

SEQ ID NO: 10 - DNA, 32 bp, Artificial Sequence
  Description of Artificial Sequence oligonucleotide primer
ctatgaattcaccatgatccactggaaacaga

SEQ ID NO: 11 - DNA, 31 bp, Artificial Sequence
  Description of Artificial Sequence oligonucleotide primer
cactagtctagagaaaaacagccctgcacgc

SEQ ID NO: 12 - DNA, 24 bp, Artificial Sequence
  Description of Artificial Sequence oligonucleotide primer
agttatgtcttctgggtgacagac

SEQ ID NO: 13 - DNA, 24 bp, Artificial Sequence
  Description of Artificial Sequence oligonucleotide primer
ttgcaagcctgatgtcctatcaag

SEQ ID NO: 14 - DNA, 20 bp, Artificial Sequence
  Description of Artificial Sequence oligonucleotide primer
atcgtggggctctcgctcag

SEQ ID NO: 15 - DNA, 22 bp, Artificial Sequence
  Description of Artificial Sequence oligonucleotide primer
cgtcaccatcacgtccgatctc

SEQ ID NO: 16 - DNA, 22 bp, Artificial Sequence
  Description of Artificial Sequence oligonucleotide primer
cagtctaggagatgacaccagc

SEQ ID NO: 17 - DNA, 22 bp, Artificial Sequence
  Description of Artificial Sequence oligonucleotide primer
agggtgcggacagattgggtac

SEQ ID NO: 18 - DNA, 22 bp, Artificial Sequence
  Description of Artificial Sequence oligonucleotide primer
gctctcggccagtttctgaatc

SEQ ID NO: 19 - DNA, 22 bp, Artificial Sequence
  Description of Artificial Sequence oligonucleotide primer
gctcgctgagttcgtccagagc

SEQ ID NO: 20 - DNA, 24 bp, Artificial Sequence
  Description of Artificial Sequence oligonucleotide primer
gaccgctatcaggacatagcgttg

SEQ ID NO: 21 - DNA, 24 bp, Artificial Sequence
  Description of Artificial Sequence oligonucleotide primer
actatgtagcctgggctcaggtag

SEQ ID NO: 22 - DNA, 13104 bp, Homo sapiens
taagttcatcaacttactatatgaaattcacatgcttctattttgagtgtagctggaagctgctgtgtattatactcattatttcactgtggccatggtatcaggattgccaaggaactttataaatacctccatttctagtaatttttttttaattgtgtatacatttaaggctgggtatgatggctcatgcctgtaatcctagcactttgcgaggctgaggcaggcaaatcacttgaggtcaggagtttgagaccagtctggccaacgatgtgaaaccccatctatactaaagatacaaaaattagctgggcatggtggtgcgtgcctgtaatcccagctacttgggaggctgacgcatgagaatcgtttgaacctggatagcggaggctgcagtgagccgatatcgtaccactgcagtccagcctgggcaacagagcacgactccgtctcaaatcgtcacacaaacatgcaatcaaacaaacaaacactaccagttcctagagcagtggttcacaaacctggctgcaggtaggaatcacctgtattctcccagatcccactccagacctcttgaattagactctccagggctggggcctgaagattcctatgcttaaaaagctctttctcgtccttcctacactgaatttcctcatggttcgaggactcagaattcaaactagtacagctaatgccgcggttcagctgcatcaagagtttaacacagtgacaacttgtctctacaaaaagtcaaaaaataaggcaggaggattgcttgatctcaggaggtcaaggctgcggtgaggcgtgatggtgccattgcactcccacttgagtgacagagtgagaccctgcctcaacaaaacaaaacaaaacaaaaaaagagttgaacagatctgaaggctagtttgaaggccaactaccattttcagtgtagtctctctagggtgatgtgttggaagtgcccaacaaacaatttaaaagagaagtctgaggatgtggtccacagatttttttttgtggaaactgaccacacagaacttttgaacaaaatgccttcatttgttcctaaaatttgttattgtgtctttactatatgaaattaaacaaatgctggctgggcatggtggctcatgcctgtaatcccagcactttgggaggctggggcaggtggatcacccggggtcaggagttcgagaccagcctgtccaacatggcgaaaccccgtctctactaaaaatacaaaaattagccaggtgtagtggtgcacgcctgtaatcccagcttctcgggaggctgaggcaggagaattgcttgaatctgggaggcggagattgcagtgagctgagatcatgccactgtactctagcctgggcgacagactgagacgccgtctcaaaaataaataaataaataaaagtaaacaaatgctgaattgaatcgctaagttcaagtgtcagttcttataaacaaggtgaagtaccagttgggttttttgtttgtttgtttgtttgtttttggagacagagttttactccgttacccaggctggagtgcagcggtgtgatctcggctcactgcaactgctgcctcccaggttcaagtgatcctcctgcctcagtctcctgagtagctgggactacaggagcacgccaccacacacagctgttttttttgtatttttagtagagacggggtcttaccatgttggtcaggctggtctcgaactcctgacctcaggtgatccaccagccttagcctcccaaagtgctgggattacaggggtgagccactgcacccggccccagttggtttttttttgttttttttttttttgctttttttttgagacaaggtctctatcatccaggctgtgttgcagtggtaccattatagctcactgcagccttgacctcctgggtgcaagtgatcctcctgcctcagcgtcctgaatagctaggactacaggtgtgagtcactatgcccggccaatttttaaattttttgtaaagatggggtctcattatgtcaaccaggctggtctcgaactcctgacttcaagcgaccctccctcctctgcctcccaaagtgctgggattataggcatgggccaccatgactggccaaagtaccaggtttttatgggcagtaaaactttagcccaaactgtcaattaactcaattttttttgcctccataaagaggcaaatacaaagaaaatgggctttcaacacagagaaaaacaaattccaagatatgggctgaggtcagtatacacgctgtctattttcctctaccttctggttcttcatcccacctctgtactatgatattgatgctctgaggaagaccctgtatcccagcaatattcctcatctataaaataaagatttaattgtgttcctctaaacccaaaacacacaggatgatgtccagggaagttagaggaacatgtgtcagctgaagaatcagtggtatgttccctcttgtctacctccccaattcgcgctggcccacatcccatttggagtgaccatgtctatggagatagaggaggatattcttccacttctaacctcaaaaggacacactggaggcatctagattgaggttacagctatcctttgaggagctgtaacaagaatatttggaagtggaccaaatctagaataatcaagtttgaaaggactatggttgtcctttctgttgctactgttgttatactgtgttgataaatacaaagtatagtcataaaggtgggggatttaatcatgttccagaatgatacggctgttaaggctttccaggtaagccagagagttgagataatccttattcaagtgagaaacgaatttggccctccttatctggaggagagggttagaaggacgatggatggatttggggacatgtagtaggactgaattcctaggctgagctccaaccacctctgaatgccccagagatatctatgttccatctggtctggtctgcagcaagtccacagaagctacattcacttttctgtgctgaattatcaaaataattgcccttcagcccatgcctcatgaccctgtagacagaatccactcatttccctatcacatgggctacaattgctacttcaaatgaaaacctgggaagccaatgccctattgtggttgaaagtgtacaattagctcctcatgcagcctgaccccactgatttttctgatgcatgtgacctggagtgtgccctttctgagaccgccaatgtcttctccacagctggagatgcccaaaactgcacgcagcacttcctgtgggtgtggccttgcattcttaggactgcagggtacccgctgcatgccaaatgatcccaggctccagccactcacagattcacaacgcttttaaaaatacagcagtggagtatgagcggctatgtcagatatgagggttttccaccccctcccttttcttttaaaatatgattttgtaatggtggcctcaagaaacactataattggtcagcctggggtgcgtttgaaggccttctctatcgcgtcattagctattctcaggaaaggcagggtatcccactgggagaatgacaacacacttgtttcaagttagggaagagcctgtggttctcttcctgcgttcaggggaaagcgaacacacaatgttcgtttcctaaatacgggatgtgctgtgctggcaggtcattttccaccatgtcacgtccttctagatgaaggcagcaggggtcatgacaggaaatggcaaattctcagaatgagacaaggctttcccagggcagccattggttctctggaactataaagcacactcatccagaaacagcctcagattttactttcctggaggcagacagaagtgaatggtaagtggggaaccctgaggcatatattcgggatgactttttctcattttctcttttcacctggaaaattatccctggtgggttggattatggaagcatggaagtaaaattaagtcctgtgaataaggagatgaggattctagactgggctctgtgaagaatcagctgcttctagaatctgatgtctgatgcaggcaattcttccagatgagaatgatttctgccctgatagctcagttttgaaatgctctatttctatggctcatttgccacttttaagaagttcctaataatgaagagtacagtggacatgaaggtgggaatctgcaactaggatgaatatacttttcccccaagatgtacaacttgttcgtatctttgtcagtcatcttcagttggcttcaacgtggtagaagttgttgaacaaaaattttggccactttattttattcaaccattactcatttttaaaagaaaactttcataacaaagagacataatggtattaatttaatgtgacttagtttaaatcaagcacccagcaaagactgatgtttctgaagatccaaggagccctagttagaataaacagtgcaaatctcattttaggcttgattctaagaaaagagcctggaagaatcacaagcagaagagttgcttcaggcaagcatctaacttagatgtttcttctgcttggtagaaaatacgatttttgaggttttggtgaagtctttttcattcattagagagagagaaaagtgtgttgcaaaactcctaggatccaattttcacatgatgaaagggctttgtaaactgtacaacccaccacttacaaagtgttatcttaagtagagcaaaggcctcagtactggagaagccttctaataccacgcccctcccagtttaaatcttatttaacacatagctgatgaagcccatcgttaacgttccctgggagagaagcagaagatagcattattcctatttccatatgcagagataaggtcctgagaacttgtgtggctccccccggattccagaacaggtctttgagaactcgtctcatgatccactggaaacagacccgttcccctagcgtggcagtggctgctccgctgaactcgtgccaagttcccgctggcgtccgggcagcagggcgggagcggcggctggcacggagactccaggctgaccgcgtgtctatgtccccgcagggaatggagaggccggcggcccgggagccgcatgggcccgacgcgctgcggcgcttccagggactgctgctggaccgccgaggccggctgcacggccaggtgctgcgcctgcgcgaggtggcccggcgcctggagcgcctgcgcaggcgctccctcgtagccaacgtggccggcagctcgctgagcgcaacgggcgccctcgccgccatcgtggggctctcgctcagcccggtcaccctggggacctcgctgctggtgtcggccgtggggctgggggtggccacagccggaggggccgtcaccatcacgtccgatctctcgctgatcttctgcaactcccgggagctgcggagggtgcaggagatcgcggccacctgccaggaccagatgcgagagatcctgagctgcctcgagtttttctgccgctggcagggctgcggggaccgccagctgctgcagtgcgggaggaacgcctccatcgccctgtacaattctgtctacttcatcgtcttctttggctcacgtggcttcctcatccccaggcgggcggagggggacaccaaggttagccaggccgtgctgaaggccaagattcagaaactggccgagagcctggagtcctgcaccggggctctggacgaactcagcgagcagctggagtctcgggttcagctctgcaccaagtccagtcgtggccacgacctcaagatctctgctgaccagcgtgcagggctgtttttctgagaacatcctttccccctaatgaccgaggccagcaaatcatcctcatgggatgctccagaatttgtagctcccttaggaaaacaccaagctgggttaggagccgaaggcaaaggatgagaaaaactgtttttgaagtgggcaggtccccaaagcccttcttttcccatcactgtgacatctgcctgggcttgagtgctacggacttttcagtcttcctagtggaaaaatgtgacccaaaaactctttttcctttatcaaaaactttctgtctaaacacagctgggcaggcactcctgttttaaagttatttcggggtccctgaccctgccctggtggcttggcctgagactggagagagtgccatcctctgggtcctctccaagtcctactagtctttgaagtcctcaaaatgtgcgtgaggaaggcgtttgcctctattccagaatttctgatacaaagaactccagaatccagagcaaatcagcccttctctgaacgttgtaggatggttcagaacccagagaggaccctggtgctgatatctcctcctcttccctttcccctcagcttacttactcccagatgcggcctgggtatgaagtaggcctttcctgagtggctcccaatccagtcctccaagtactcagaggggaagcccgtgaagccgtcatctaagtcctgctccctcacatgaagctgagggccagatagatggagcgactgccaacttcatttcccgacatcattgtgttcagaagagagtgatgggttttgagttagacagtcctgggcttgagacaggctttgtcactactgtgtgagtgtagccacctaatctctctgagactgtgtaaaacaaagatgataaaatctcaccctgttgtgagatattaaatgagccaaagtgcctagcatgatggtgctggctcatatagtgtagtccctggaatggcaaattaacatcacccaggaacttgttagaaaggcaaattcttggacacaaccctcctgatttatggaatcagaaactctggctgtggggcccagcaacctgagtttaaacaatttctctgggtggttctgcggcacactaaggtttgaaaatcactgcaacaaatgctaacttctaatccccttgatgagctttcacgaagtctcacggcttctctagggactccatggtcttcagagtcgttcacagatgaccaaggacagactgtgtcccagaagccaaaatgagagagagagagagagagcacgcgtacgtgcaccctggggcagtgtctcaccgtatgaacaagggatgtaacactaaaagcccattagggggcagtgtttcccgcctgttgtagaaactggtacagaaaggaatatgaagttcctgaaactgacctttgtctattattaccttctctgaaaagtgccagtccatgtattttttatttattttaagtttgtaatttaatttttaattattgtttagtgtttgcatttaattttatttaatcaccacatttagaaaataataagagcaagtttctaaatgggagactgctgaggctctttgcaagagatgagattaagtttgagtttctaaggcagggcatgagctggaaatagcattgctttccttgattgtctctctccttcagggagattctttttctctagtgttttaagtgatcctttgaagtaagtgtggagagtcttgaatggcaagaccaggagctgagtttaagcttgtaatggaagcttgcattgtgggatatataactgaggaagcatatttatcctgaaggtattttgccagaaggtatcacttgacctggaaaaggaatctatttagttcaggaaagataaaaagtttagaggtatgtgaaggaagcacttagaacttgcaagcctgatgtcctatcaagttatgtcttctgggtgacagacaaaatcgcttgtcttatggtggtgatgtgttgcattttcactttggggtctgtaagaaactgtcagtgaaaatatgtacaattccttcaatttccattcttaacaactgtaatgttgaaaaataagttgaaaagtctttgggaccatacatgcaaaaacggtgcctctgttacttaattatttaatattctataaatgtacccaatctgtccgcacccttcccagtgatggggcagtatgtctgaggaagtataatttcagtactggggtcggggagaggaggtgatgtttctacatttttattttttctataaattgcaattggtctgtatgctggtttattttgaaatttatattggtttcttttcaagctggtgtcatctcctagactgtttcacccagatgctagcattttttttttttttttgagacagagtctcactctgtcacctaggctggagttgcagtggtttgatctcggctcactgcaacctccgactcctgggttcaagcaattcttctgcctcagcctcctgagtagctgggattacagatgtgcaccagcacacccggctaattttttgtatttttagtagagacagggtttcgccatgttggccaggctggtcttgaactcctggccttatgtgatccgcccaccttggcttcccaaagtgctgggattacaggcatgagccacctcgcctggccagatgctagcattttagatcaaacaattcattttagatgaattgttttgtttcacaatcattttaaatcattttagaatgtacttcacattattagttgtgttatggcataaaggtacaaccattccctaactccatcttttattaatgcttaagtttaaattatattcttccaatgcctaagctattccctagaattaaactgggcacttttggaagcagcaacagtaacagcagcagcaaacttttcctctcatattttgggtgtatcaaaagttctagacttttgaagttatgatttcagtggcccactttatttctaaggaagagtgtctactttggaacgatactttgcacatagtaggaactcaagaaatacatttgaataattataattaactgtttagctatcttaatgagaatttgttgacaacaaaagatcatccatcgccttatgtgtgagtaagattggagcctctatcaagatttagtcaagttcagttagattgattctagaaacaaatatttatttctttcttttacggggatgtgaataaggcttttccttaaggccttcattctttaaacaaacaggttgaaatggtatgttgtaaaagagaagacgggagagaggtatttagatgataagtgtacttcacaaaaatgccaaagtttgaaaaataggtatgtttgttctaaatgtttaagtgcttctctgttaggttctggggcttgcaatcatttgaattgttctgtttcacaataaaggagattcactgggttctgcattttcaggattcaatagaactgctccattaaaaaaataatccttagcaagcattcgaatcctaactgctttgatgcacttgccctcgggcacctgtcatttccaatatggtaggtgtcaaagtcaaaagtatttactgggagaaaaaagagaggagtggttgtagaagtctccctaaatcagacatgtcaagcaatcagccaacgtggtgtatttctcattcaatattttagtgtgaattgagacactgagataaagacatcgtgcagagataaatggggatacagttaaatgtagcaactcttgagttcattttttcccactgtagcaaaattaatgctttctctttattgaaataaattgctcattcctcaaatttttttatctttctgtaattaatttcctagttctatgtttccttgtgtatgaattagtaactttgaaaaagggcagatgtttaatcattttcacttagaatattcagatcaaactatatgaatcatttttttttaataatcaatgcatacacaggaattcccttttgcctagaaatcagacttccagcagtctcaaaacctttgaacataattacaacctgggaaggaattgaaaggaaacctagaatcagccaaattagccttcacaacatccaaacactaaagagaggtgggcagggagggtgaagggcagcatgtatatcaggaaagaaaactttcccgtaaatctctgagggacttcaccttgcaagccaccgatcttgaccatgcaaggcaggctgggaaatgtagtatttttagctgagaacaacgctgcccctaatatacctaagattctcttggtaagaaagaaaaggagggtgatcattaagtagggggtccagcaatttaaacaaaaagcctattgtggttgcggcagccagttggggacgactactctggagacgacctcactatctcagtgccagctgcaccttggaaacattcctgggtctggaggctgcactgttaataactgtacaaccaaaaaaggctgtgttggcaggcccctgactgtttgcagaggccagccgcaagctgggatcctatctttgctgaagcatatggtattcctgtagtaacaagtgtccttttagttaattgcaataaatctcttgttgagttgacaggaggttgggatgttactttttgctcctggcaaactcataagagggaaagtaaaccctgctgtggtctggaataggacaaagagtgagcccttagatctgaaaacaatctcttagcaaccagagagctgttaggagaggggaaatcgtgctgatgaagcgagacccatcgtccaggaatgcacattaggggtcctcagcacccccaaacctgactagcattagcaccgggttgtggagagaaggccctctaatgtggtctttgcctgcacgcatagagattatctatatgaagctacatctgcagaaaatgatgtttaatccaattttgtatttacactaaaaccccagaggtgggaggacaggcaggcctgtgacctgattccccctgagctttagccatgggtcacggccaaggcatctgcctcccttacacacaggagcagccagatcctggcctcctccagcatttgtgtgaatacggttgccaaattcaccaaaataattataactgggagaagctacacaaaaagggtaggggtgatgtctaaagtctttcttttaggcgaaacttctttttctacgctccccggttgataagcagacatttctttctgccctcactttttattaaaactgcactctcaaaatatcatttaaaaacaatatttttttcagtgcttaaataaaatgacatttcaataatattgaacatacatgtgggatatttaacaataacaatagctaacatttattgagaactcactagatactcttctaagtacgatttgacctgtatcatttcatttaccttacaaaaatcttatggtgtgcacactataattaccctatattattagataaagtaaggaatttgagaggttgcattgccagcatgaggtgccccagaatttgaattctggcagcctgactccagagttatgctcttagctattcttttccttttttttttttggagtcttgctctgtcacccaggctggagtccagtggcatgatctcagctcactgcaacctccgcttcctgggttcaagcgattctcctgcttcagcctcctgagtagctgggattacaggcgcccaccaccatgcccagctaatttttgtgtgtgtgtgtatttttagtagagatggggtttcaccatgttggccaggctagtcttgaactcctgacctcaagtgatccacacaccttggcctcccaaagtgctgggattacaggcatgagccgctgtgcccatccaatgctcttggcttttctgattttttttttttagacggagttttgctcttttcgcccaggctggagtacagtggcatgacctcagctcactgcaacctgtgcctctcgggttcaggcgattctcccacctcagcatcctgagtagctgggattacaggcgcccaccaccatgcctggctaatttttgtatttttggtagagacagggttttgccatgttggccagggtggttttgaactcctggcctcaggtgatctgcccatctcagcctcccaaagttctgggattacaggtgtgagccactgcgcctagccaatcctagcaccttgggaggccgaggcaggtgggttgcttgagtccaggagttagagaccagcctgagcaacatggtgagacctcatctgtacaaataatttttaaatgagctgagtgtggtgttgcacacctatagtcccagcttctcaggaggttgaggtgggaggatcacttaagcccaggaggtcgaggttgcagtgaaccgtgattgggccactgcactccagccagcctggggaacagagcaagaacctgtctcaaaaaaaataaaaacatctcctggcagtaacagtctcatctaggatgagtaatcttatgccagtaggctagtcaataagaaaaaaataaaatatagctttgcaagtcactagtatttataataaacagtgtgggtgtgatttgaaagttataaaacttttgctctgatcataaaaacctatgtatgggtttttatggtggctgggcacagtggctcacacctgtaatcccagcactttgggaggctgaggtgggaggatcaactgaggtcaggagttcaagactagcctggccaacatggtgaaaccccgtctctactaaaaatacaaaaattagctgggcatggtggcaggcacctgtaatctcagctactcgggagactgaggcaggagaatcgcttgagcccagaagacagaggttgcagtgagctgagatcacgccactgcactccagcctaggcgacagagcaaaactctgtctcaaaaacaaacacaacctctcttcacttactctaggacaagttcagaaaaccatgtatggcttacaaagtcattcgtgatctggcccctgtttaccattccagaatgatatctctctctccattcaccctcttttttttttttttttttttttttttttttttgagactgagtcttgctctatcgcccgggttggtggtgcatggtcaccgaaagactgcacattttggacatgaagtggggtcagtgcaccattaaataccctcacagacccgaaccactgatcaccatgcaggaagagtaacaaaaagaaaaaatgcaacacaccatcgatgaactaaca

SEQ ID NO: 23 - DNA, 741 bp, Mus musculus
  CDS (1)..(738)
atggagaagcgggcggcctgggagccgcagggcgccgatgcgctgcggcgcttccaaggattgctgctggaccgccgtggccggctgcacagccaagtgctgcgcctgcgcgaagtggcccggaggctagagcggctacgcaggcgctccctggcggccaacgtggcaggcagctctctgagcgccgctggtgccctggcagccatcgtggggttgtcactcagcccggtcaccctgggagcctcgctcgtggcgtcggccgtgggcttaggggtggccaccgccggaggggcagtcaccatcacgtccgacctctctctgatcttctgcaattcccgggaggtgcggagggtgcaggagatcgccgccacctgccaggaccagatgcgcgagctcctgagctgtctggagttcttctgtcagtggcagggacgcggggaccgccagctgctgcagagcgggagggacgcctccatggccctttacaactctgtctacttcatcgtcttcttcggctcacgtggcttcctcatccccagacgtgccgagggggccaccaaagtgagccaggccgtcctgaaggccaagattcagaaactgtctgagagcctggagtcgtgcactggcgccctggatgaacttagtgagcagctggagtcccgggtccagctctgcaccaaggccggccgtggccacaacctcaggatctccactgatctggacgcagcgttgtttttctaa

SEQ ID NO: 24 - protein, 246 aa, Mus musculus
MEKRAAWEPQGADALRRFQGLLLDRRGRLHSQVLRLREVARRLERLRRRSLAANVAGSSLSAAGALAAIVGLSLSPVTLGASLVASAVGLGVATAGGAVTITSDLSLIFCNSREVRRVQEIAATCQDQMRELLSCLEFFCQWQGRGDRQLLQSGRDASMALYNSVYFIVFFGSRGFLIPRRAEGATKVSQAVLKAKIQKLSESLESCTGALDELSEQLESRVQLCTKAGRGHNLRISTDLDAALFF

SEQ ID NO: 25 - DNA, 32 bp, Artificial Sequence
  Description of Artificial Sequence oligonucleotide primer
acaccggaattcagcatggagaagtggacggc

SEQ ID NO: 26 - DNA, 34 bp, Artificial Sequence
  Description of Artificial Sequence oligonucleotide primer
ccctagtctagagaaaaacaacgctgcatccaga

SEQ ID NO: 27 - DNA, 30 bp, Artificial Sequence
  Description of Artificial Sequence oligonucleotide primer
ccctagtctagagagaagtggacggcctgg

SEQ ID NO: 28 - DNA, 34 bp, Artificial Sequence
  Description of Artificial Sequence oligonucleotide primer
acaccggaattcttagaaaaacaacgctgcatcc

SEQ ID NO: 29 - DNA, 27 bp, Artificial Sequence
  Description of Artificial Sequence oligonucleotide primer
tggtgggtcgacatggagaggtggacg

SEQ ID NO: 30 - DNA, 36 bp, Artificial Sequence
  Description of Artificial Sequence oligonucleotide primer
agaagaagaggcggccgcttagaaaaacaacgctgc

SEQ ID NO: 31 - DNA, 34 bp, Artificial Sequence
  Description of Artificial Sequence oligonucleotide primer
acaccggaattctgagaagtggacggcctgggag

SEQ ID NO: 32 - DNA, 35 bp, Artificial Sequence
  Description of Artificial Sequence oligonucleotide primer
cacgcggatccttagaaaaacaacgctgcatccag

SEQ ID NO: 33 - DNA, 35 bp, Artificial Sequence
  Description of Artificial Sequence oligonucleotide primer
tcactggaattctgatggagaagtggacggcctgg

SEQ ID NO: 34 - DNA, 35 bp, Artificial Sequence
  Description of Artificial Sequence oligonucleotide primer
cacgcggatccgagaaaaacaacgctgcatccaga

SEQ ID NO: 35 - DNA, 30 bp, Artificial Sequence
  Description of Artificial Sequence oligonucleotide primer
ggtcgacggagaagtggacggcctgggagc

SEQ ID NO: 36 - DNA, 30 bp, Artificial Sequence
  Description of Artificial Sequence oligonucleotide primer
agcggccgcttagaaaaacaacgctgcatc

SEQ ID NO: 37 - DNA, 31 bp, Artificial Sequence
  Description of Artificial Sequence oligonucleotide primer
cacgcggatccaggcgtgcggagggggccac

SEQ ID NO: 38 - DNA, 32 bp, Artificial Sequence
  Description of Artificial Sequence oligonucleotide primer
ccgacgtcgacttagaaaaacaacgctgcatc

SEQ ID NO: 39 - DNA, 24 bp, Artificial Sequence
  Description of Artificial Sequence oligonucleotide primer
ctacatggtctacatgttccagta

SEQ ID NO: 40 - DNA, 22 bp, Artificial Sequence
  Description of Artificial Sequence oligonucleotide primer
tgatggcatggactgtggtcat

SEQ ID NO: 41 - DNA, 23 bp, Artificial Sequence
  Description of Artificial Sequence oligonucleotide primer
aagtttgtcattcggaacattgt

SEQ ID NO: 42 - DNA, 21 bp, Artificial Sequence
  Description of Artificial Sequence oligonucleotide primer
cacctctttacatgggctttg

SEQ ID NO: 43 - DNA, 34 bp, Artificial Sequence
  Description of Artificial Sequence oligonucleotide primer
aaatatgcggccgcagtgtgccctttctgagacc

SEQ ID NO: 44 - DNA, 24 bp, Artificial Sequence
  Description of Artificial Sequence oligonucleotide primer
ctccatgccctgtgagggacacag

SEQ ID NO: 45 - DNA, 24 bp, Artificial Sequence
  Description of Artificial Sequence oligonucleotide primer
gggtctgaataggaagggagtctg

SEQ ID NO: 46 - DNA, 24 bp, Artificial Sequence
  Description of Artificial Sequence oligonucleotide primer
ataggacatcaggtttccaaggtc

SEQ ID NO: 47 - DNA, 20 bp, Artificial Sequence
  Description of Artificial Sequence oligonucleotide primer
accccaccgtgttcttcgac

SEQ ID NO: 48 - DNA, 20 bp, Artificial Sequence
  Description of Artificial Sequence oligonucleotide primer
catttgccatggacaagatg

SEQ ID NO: 49 - DNA, 24 bp, Artificial Sequence
  Description of Artificial Sequence oligonucleotide primer
gtgaccatgtcgtttactttgacc

SEQ ID NO: 50 - DNA, 24 bp, Artificial Sequence
  Description of Artificial Sequence oligonucleotide primer
ggttaacgcctcgaatcagcaacg

SEQ ID NO: 51 - DNA, 20 bp, Artificial Sequence
  Description of Artificial Sequence oligonucleotide primer
ctctagcctagggcagcaac

SEQ ID NO: 52 - DNA, 20 bp, Artificial Sequence
  Description of Artificial Sequence oligonucleotide primer
gagagaggtcggacgtgatg

SEQ ID NO: 53 - DNA, 20 bp, Artificial Sequence
  Description of Artificial Sequence oligonucleotide primer
ggcgattaagttgggtaacg

(19) United States
(12) Patent Application Publication
(10) Pub. No.: US 2004/0260058 A1
(43) Pub. Date: Dec. 23, 2004

(54) Novel endothelially expressed dnas and proteins, and their use

(76) Inventors: Sigrid Scheek, Dossenheim (DE); Holger Hiemisch, Heidelberg (DE); Anthony Lanahan, Bridgewater, VT (US); Jean B. Regard, Palm Beach, FL (US); Paul F. Worley, Baltimore, MD (US); Eckart Krupp, Dossenheim (DE); Markus Schwaninger, Mannheim (DE); Nauder Faraday, Baltimore, MD (US)

Correspondence Address: FOLEY AND LARDNER, SUITE 500, 3000 K STREET NW, WASHINGTON, DC, 20007, US

(21) Appl. No.: 10/482,065

(22) PCT Filed: Jun. 19, 2002
(86) PCT No.: PCT/EP02/06770

(51) Int. Cl. A61K 38/17; C07K 14/47; C07K 7/08

(52) U.S. Cl. 530/324; 530/326; 530/327; 536/23.5

(57) ABSTRACT

The present invention relates to novel, specifically expressed proteins and to nucleic acid sequences or transgenic nucleic acid constructs which encode the proteins.

The invention also relates to transgenic organisms or animals which harbor the nucleic acid sequences or recombinant nucleic acid constructs and also to monoclonal or polyclonal antibodies and binding factors which are directed against the isolated proteins.

The invention furthermore relates to a process for finding substances which possess specific binding affinity with the proteins according to the invention, and to a process for qualitatively or quantitatively detecting the nucleic acid sequences according to the invention or the proteins according to the invention. The invention furthermore relates to the use of nucleic acid sequences and proteins.

The invention also encompasses processes for finding substances which modulate the function of the proteins according to the invention. It also relates to the use of these proteins for producing drugs.

Because of its anatomical location, the vascular endothelium constitutes an important biological boundary. It defines intravascular and extravascular compartments, serves as a selectively permeable boundary layer and forms a non-thrombogenic boundary to the cardiovascular-system. The vascular endothelium possesses the ability to monitor, integrate and transmit signals which have been generated in the lumen. This applies not only to stimuli of soluble factors (e.g. hormones and cytokines) but also to the perception of different types of mechanical forces which act, via the blood stream, on the endothelium (e.g. shearing forces, wall shearing stress and pulsatory stretching of the vessel wall). Consequently, the endothelium constitutes a sensory organ.

The endothelium is, for example, involved in the regulation of arterial and arteriolar vessel tonus by means of the synthesis and release of vasoactive local hormones (e.g nitric oxide and prostacyclin) and by means of the uptake and/or breakdown of vasoactive substances which circulate with the blood (for a review, see Hierholzer K and Schmidt R F (1994) Pathophysiologie des Menschen (Human Pathophysiology), Chapman & Hall, Weinheim). Disturbances in the vasomotor and hemostatic functions of the endothelium are involved in the impairment of tissue perfusion which occurs in association with various acute and chronic cardiovascular disturbances and disturbances of metabolism. These disturbances in the function of the endothelium consequently constitute an important pathogenic factor in diseases such as septic shock, hypertension, arteriosclerosis, cardiac insufficiency, diabetes mellitus, h-vperlipidemia and homocysteinuria.

Stimuli which act on the endothelium and which have an effect-on vessel tonus include, inter alia, hemostatic factors (e.g. ADP, ATP, adenosine, serotonin, platelet-activating factor and thrombin), neurotransmitters and peptides (acetylcholine, bradykinin, substance P, vasoactive intestinal peptide and calcitonin gene-related peptide) and also hormones (angiotensin II, vasopressin, noradrenaline, adrenaline and histamine) and physical stimuli (wall shearing stress and pulsatility). When the endothelium has been damaged, these stimuli have a directly vasoconstrictory effect on the blood vessels and lose their dilatory influence, which is mediated by way of intact endothelial cells, on these vessels.

In response to various stimuli (see above), endothelial cells can form and release endothelial autacoids (e.g. NO and PGI₂). However, they also have the potential to produce vasoconstrictive substances (e.g. endothelin). Disturbed endothelial functions are involved in vascular spasms, as occur, for example, in association with arteriosclerosis, various immunological processes and following thrombotic events. These vascular spasms are incorrectly regulated, excessive local constrictions which lead to ischemia in the distal organ regions concerned. Arteriosclerotic changes in the vessel wall are associated with augmented constrictions which are caused, inter alia, by impaired endothelial vasodilatory mechanisms. Endothelial cells are also involved in the control of blood coagulation, with the anticoagulatory effects predominating under physiological conditions. Disturbances to the integrity of the endothelial cells lead to the rapid adhesion and aggregation of platelets and to activation of the plasma coagulation cascade.

Lipid mediators (metabolites of arachidonic acid) are also involved in blood supply disturbances which develop as a result of arteriosclerosis, thrombosis or vascular spasms in combination with inflammations. In this connection, the vascular system is both the site of formation and the site of action of these metabolites (see, for example, Hierholzer K and Schmidt R F (1994) Pathophysiologie des Menschen (Human Pathophysiology), Chapman & Hall, Weinheim).

The brain, in particular, reacts very sensitively to disturbances in blood supply. Anoxia and ischemic states which only last for a few seconds can lead to symptoms of neurological failure. If the blood supply remains interrupted for a matter of minutes, this may result in irreversible neuronal damage. The blood flow must ensure efficient provision of the brain with oxygen, glucose and other nutrients and also dispose, in turn, of CO₂, lactate and other metabolic products. Although the human brain only constitutes approximately 2% of the total body weight, it nevertheless receives about 15% of the blood ejected by the heart and is responsible for approximately 20% of the total oxygen requirement. These values underline the high level of metabolic turnover in the brain. The cerebral blood vessels, which have to cope with these high demands, have developed mechanisms of autoregulation for the purpose of maintaining optimal cerebral blood flow. These autoregulation mechanisms may be very local and coupled to neuronal activity, as can be visualized, for example, using MRI/PET techniques. Similar mechanisms can, inter alia, be responsible for regulating blood flow in other organs (for a review, see Schmidt R F and Thews G (1987) Physiologie des Menschen (Human Physiology), Springer Verlag, Heidelberg).

During and after epileptic seizures, the local cerebral blood flow and metabolism are altered in the brain areas concerned. In this connection, there are marked local increases in blood flow during the seizure, with these increases subsequently changing into a hypoperfusion. There are conflicting opinions with regard to the extent to which blood flow is regulated in a manner which is appropriate for the increased metabolic demands of the brain areas (ictally and/or post-ictally) and whether a relative hypoperfusion develops as a consequence (see, for example, Ingvar M and Siesjo B K (1983) Acta Neurol Scand 68:129-144; Duncan R (1992) Cerebrovasc Brain Metab Rev 4:105-121).

Blood pressure is influenced, at least in part, by a multiplicity of genetic factors. Because of the nature of their influence, the underlying gene allele variants are termed “quantitative trait loci” (QTLs). The identification of such QTLs is an important step toward identifying genes which are involved in regulating the blood pressure. A difficulty with the identification is the lack of suitable populations of individuals who, while differing in the phenotype to be investigated (in this present case, for example, high blood pressure, systolic or diastolic pressure, or the like) otherwise exhibit a very similar genotype. Such populations can be found in regions where there is a very low rate of migration and very little mixing with external population groups (e.g. Mormons, Amish people and Icelanders). Another possibility is, for example, that of examining monozygotic and dizygotic twins. Monozygotic twins have the same genotype and are at least partially exposed to the same environmental factors. By contrast, only 50% of the genotype of dizygotic twins is identical while these twins are subject to environmental influences in the same way as are monozygotic twins. Comparison of monozygotic and dizygotic twins with regard to phenotype and genotype makes it possible to investigate the contribution made by genetic factors to a particular phenotype. The analysis of the genotype is customarily carried out using suitable genomic markers (e.g. what are termed microsatellite markers). Another possibility of identifying genetic factors using the means of population genetics consists in investigating the correlation between genotype and phenotype in families without any restriction to twins.

In the OMIM database (Online Mendelian Inheritance of Man; Internet address www.ncbi.nlm.nih.gov/htbin-post/Omim), the syndrome hypertension together with brachydactyly (HTNB), having the locus 12p12.2-p11.2 is given as the high blood pressure disease classified under OMIM number 112410. Furthermore, a locus for essential high blood pressure (essential hypertension; EHT, OMIM number 145500) is present in the 12p13 region. A further reference to the presence of a QTL on chromosome 12, which was said to be connected with a genetic predisposition to high blood pressure, comes from a study carried out by Frossard and Lestringant, 1995 (see OMIM 172410; Frossard P M and Lestringant G G (1995) Clin Genet 48:284-287) and also from Nagy et al. (Nagy Z et al. (1999) J Am Soc Nephrol 10:1709-1716).

The HTNB syndrome was described, as an autosomally dominant disease characterized by brachydactyly and severe hypertension, for the first time in a Turkish family in 1973 (Bilginturan N et al. (1973) J Med Genet 10:253-259). The two symptoms were characterized as being completely cosegregating, such that it could be assumed that they were due to a defect in one single pleiotropic gene or two very closely adjacent genes. In a molecular biological study (Schuster H et al. (1996) Hypertension 28:1085-1092; Schuster H et al. (1996) Nat Genet 13:98-100), the syndrome was mapped to between markers D12S364 and D12S87 on chromosome 12. From the position of these markers, it can be concluded that the chromosomal region concerned is 12p12.2-p11.2 (cf. OMIM entry). The syndrome is characterized by high blood 25 pressure, with the difference between affected and unaffected family members being at least 50 mm Hg. Subsequent studies showed that the affected patients were not salt-sensitive and that their humoral reactions (renin, aldosterone and catecholamines) to volume expansion or reduction were normal, indicating that the renin-angiotensin-aldosterone system and the sympathic nervous system are not responsible for the increased hypertension. The HTNB syndrome thus resembles essential hypertension (Schuster H et al. (1996) Hypertension 28:1085-1092; Schuster H et al. (1996) Nat Genet 13:98-100).

Blood vessels are formed by way of two different processes: angiogenesis and vasculogenesis. During embryogenesis, what are, termed angioblasts (i.e. vascular endothelial cells which have not yet formed any lumen) are formed from mesodermal precursor cells. The angioblasts then differentiate, leading to the formation of a first vascular plexus from which primitive blood vessels are then formed. This process of the de novo formation of blood vessels is termed vasculogenesis (Risau W and Flamme I (1995) Annu Rev Cell Dev Biol 11:73-91).

After the primary vascular plexus has developed, further endothelial cells are then formed from the vessels which already exist (angiogenesis). In this process, the new capillaries can be formed either by budding from the vessels or by the vessels being divided along their length. The type of angiogenesis which predominates varies from organ to organ. While, for example, lung vessels develop by non-budding growth, the brain vessels are formed by budding, due to an absence of angioblasts in the brain anlage (Risau W (1997) Nature 386:671-674). A mature vascular system, possessing smaller and larger blood vessels, is formed from the vascular plexus by means of a process of “trimming” and remodeling. In this process, “surplus” blood vessels are lost; the endothelial cells can either integrate into other vessels or dedifferentiate.

The molecular mechanisms underlying this process (e.g. whether and to what extent apoptosis is involved) are still not understood. Both extraluminal and intraluminal factors are involved in the further maturation of the blood vessels. The blood vessels grow or retrogress depending on the development of the organ which they supply. Improved perfusion leads to hyperoxygenation, resulting in the involution of blood vessels. Unperfused blood vessels become atrophied. The direction of flow can change; arterioles can become venules or vice versa. While being originally independent, the vascular system becomes more and more dependent on the blood supply (in addition to circulating signal molecules) and the forces which are caused thereby (e.g. shearing forces).

Angiogenesis also takes place in the adult body, for example in the female reproductive system, and in association with hair growth and wound healing. Endothelial cells are not postmitotic but, instead, can be stimulated (in the main locally and transiently) to form new blood vesssels. In association with pathological changes and wound healing, there is a close connection between angiogenesis and inflammatory processes. The balance between local inhibitory controls and angiogenic inducers is disturbed, resulting in altered vessel growth. These disturbances are causatively involved in many human diseases, including, for example, diseases of the cardiovascular system, rheumatoid arthritis, diabetic retinopathy and tumor growth.

The transition from resting to activated vascularization of a tumor is probably triggered by a hypoxia stimulus, which occurs when the tumor has reached a size at which simple diffusion no longer suffices for providing all the tumor cells with nutrients (for a literature review, see Brower V (1999) Nat Biotechnol 17:963-968; Zetter B R (1998) Annu Rev Med 49:407-424 and references contained therein). This results in the expression of hypoxia-induced genes, such as vascular endothelial growth factor A (VEGF-A) and placental growth factor (PIGF), both of which specifically stimulate the growth of endothelial cells by means of binding to their receptors. Endothelial cells, for their part, produce many nonspecific angiogenic stimulators (including OFGF, αXFGF, TGFα, TGFβ) which also contribute to the invasive growth. Tumor cells and endothelial cells produce proteolytic enzymes (matrix metalloproteinases, and serine proteases such as tissue plasminogen activator) which degrade the extracellular membrane. However, the proteolytic medium also activates cryptic angiogenesis inhibitors (the best-known representatives are angiostatin and endostatin) and various protease inhibitors. Endothelial cells express particular adhesion molecules on their surface (integrin αvβ3 and αvβ5) which interact with the extracellular membrane. The expression of some growth factor receptor tyrosine kinases (including VEGFR-1 and VEGFR-2) within the endothelial cells is upregulated. The activation of Tie-1 and Tie-2 receptors appears to play a role in the mediation of cytokine- and angiopoietin-mediated capillary-organizing signals. Cytokines and chemokines are also responsible for attracting monocytes and leukocytes, in turn contributing to the development of a local inflammatory reaction. Naturally occurring inhibitors of angiogenesis are able to trigger apoptosis in cultured vascular endothelial cells (Jimenez B et al. (2000) Nat Med 6:41-48, and references contained therein). This indicates that apoptosis could be an important regulator of angiogenesis.

The “immediate early genes” (subsequently termed IEGs) have an important function in intracellular regulation. A gene is termed an IEG when it fulfills three conditions:

(1) its mRNA must be expressed at a low level in resting cells or in unstimulated cells,

(2) its mRNA can also be expressed in the absence of de novo protein synthesis,

(3) it is transcriptionally active after suitable stimulation (Nathans (1991) in Origins of Cancer: A Comprehensive Review, Brugge J, ed., pp. 353-364.).

Based on the kinetics of the accumulation of their mRNA, IEGs are frequently subdivided into three classes:

I. IEGs belonging to class I are frequently not detectable in resting/unstimulated cells and the maximum mRNA concentration is reached about 30 to 60 minutes after stimulation. After about 1.5 to 2 hours, this concentration returns once again to basal values. Examples are c-fos, c-jun and zif268.

II. IEGs belonging to class II achieve maximum mRNA concentrations 2 hours after stimulation and reach basal values after about 8 hours. Examples of these IEGs are Narp, c-myc and GLUT1.

III. Genes belonging to class III are very rapidly induced but, even so, their mRNAs accumulate over many hours and have a long half-life (e.g. fibronectin) (Lau L and Nathans D (1991) in The hormonal control of gene transcription, Cohen P and Foulkes J G, eds., pp. 257-293).

Since IEGS can be transcriptionally activated in the absence of de novo protein synthesis, the regulatory proteins required for inducing IEGs must already be present in the unstimulated cell and ready for an activation. It has been observed that stimulating cells in the presence of cycloheximide, a potent inhibitor of protein synthesis, leads to IEGs being superinduced. This observation has been attributed to two effects, namely an extended period of transcription and an increase in mRNA stability. AT-rich sequences in the 3′-untranslated region appear to play an important role for the rapid degradation of mRNAs which encode IEGs and cytokines. An AUUUA motif has been identified in almost all IEG mRNAs which have short half-lives (Lau L and Nathans D (1991) in The hormonal control of gene transcription, Cohen P and Foulkes J G, eds., pp. 257-293). The observation that inhibitors of protein synthesis stabilize IEG mRNAs can be explained by a variety of hypotheses. Newly synthesized or labile RNases are required for degradation, or else degradation of the mRNA is directly coupled to translation. Experimental evidence exists to support both theories. In the case of the gene c-myc, a cytosolic factor has been described which, following stimulation, binds c-myc mRNA and destabilizes it, and which cannot be detected during treatment with cycloheximide. Numerous studies have shown that translation is a prerequisite for mRNA degradation (e.g. Yen T J et al. (1988) Nature 334:580-585 in the case of the tubulin gene).

The functional significance of the neuronal IEGs was initially completely unclear; it was only after further investigations had been carried out that it was found that these genes constitute important intracellular points of regulation, inter alia. For example, in the case of the Homer 1A IEG, it was shown that this IEG is a truncated variant of a member of a larger gene family and that the induction of this variant leads to the (dominant-negative) interruption of the signal transmission which is mediated, between extracellular receptors and internal calcium stores, by the other members of the gene family (Tu J C et al. (1998) Neuron 21:717-726; Xiao B et al. (1998) Neuron 21:707-716). Consequently, an external stimulus (e.g. a convulsive seizure) leads to direct changes in important second messenger systems.

Further evidence of the important role played by neuronally expressed IEGs in the hippocampus was provided using Arc as an example. After a seizure has been triggered, expression of the mRNA of this gene is also induced in pyramidal cells of the hippocampal subregions CA1 and CA3. It was shown that expression of Arc mRNA is induced in the CA1 area simply by bringing the rat into a new environment. Since the pattern of the neurons which were induced was specific for the particular environment in which the rat was located, it was possible to demonstrate that induction of Arc mRNA expression correlates with neuronal information storage processes in the hippocampus (Guzowski J F et al. (1999) Nat Neurosci 2:1120-1124).

The gene L119 has hitherto only been described as IEG cDNA in the rat (WO 99/40225). This cDNA was cloned on the basis of stimulating the expression of L119 mRNA in the rat hippocampus following a repeated maximum electroconvulsive seizure. In this study, it was assumed that the stimulus leads to the induction of neuronal immediate early genes (IEGs). All the previously described genes which had been cloned in this way are expressed neuronally (see, for example, Yamagata K et al., (1994) J Biol Chem 269:16333-16339, 1994; Lyford G L et al. (1995) Neuron 14:433-445; Brakeman P R et al. (1997) Nature 386:284-288).

Taking as a starting point the significance of the endothelium in a multiplicity of diseases, such as of the brain, of the immune system and of the cardiovascular system, or in association with cancer, and a need, which is still great, for methods for treating these diseases, it was an object of the present invention to develop new approaches for treating said diseases efficiently.

We have found that this object is achieved by preparing the L119 proteins and the nucleic acid sequences encoding them, by using the same for the diagnosis, prophylaxis and therapy of vascular diseases, especially including endothelial, coagulation and platelet diseases, and also by means of novel methods for modulating or standardizing L119 activity for the purpose of treating said vascular diseases while involving these nucleic acids and/or proteins.

When L119 cDNA was discovered in the rat (WO 99/40225), it was assumed that another neuronal IEG had been identified. However, subsequent analyses showed, completely surprisingly, that L119 mRNA is expressed neither in neurons nor in glia cells, even after stimulation by means of a repeated maximum electroconvulsive seizure. By contrast, analyses of in situ hybridization showed that signals are only obtained in the endothelial cells of capillaries and larger blood vessels (see Example 5). In experiments in which a blocker of protein synthesis (cycloheximide) was administered systemically, it was demonstrated that it is possible to induce L119 mRNA expression not only in the blood vessels of the brain but also in all the other tissues and organs investigated (see Example 5). Consequently, L119 is not a neuronal IEG but rather a gene the expression of whose mRNA is induced in the endothelial cells of blood vessels in response to a variety of stimuli, which are described below in detail. L119 is thus the only endothelium-specific gene which is so far known to be induced in the endothelial cells of blood vessels following acute seizures.

L119 is expressed in the endothelial cells of capillaries and larger blood vessels in the brain and other organs. The mRNA corresponding to rat cDNA encoding L119 has 8 AUUUA motifs (compare SEQ ID NO: 1 and SEQ ID NO: 2, respectively), which is typical for IEG mRNAs having short half-lives (see above; Lau L and Nathans D (1991) in The hormonal control of gene transcription, Cohen P and Foulkes J G, eds., pp. 257-293). Based on the abovementioned criteria for IEGs, L119 can be classified as a class I IEG. The rapid regulation of the degradation of L119 mRNA, which is observed experimentally, can be explained, inter alia, by the above-described mechanisms.

L119 has demonstrated to be a key player in several disease models, including but not limited to the following:

a) Epilepsy

By demonstrating that the expression of L119 during and following epileptic seizures was correlated with blood flow and metabolism, L119 was shown to play an important role in regulating these processes (see Example 5). These data were further strengthened by results from a model of excitotxicity (kainate induced; Example 12), demonstrating a strong upregulation of L119 under these conditions.

b) Cancer

In addition, L119 was demonstrated to have an important function in tumor development. Basally, L119 mRNA is either only expressed at a very low level or cannot be detected at all. By contrast, L119 mRNA is expressed at a high level in the blood vessels of a variety of tumors (see Example 6). Biochemical studies provide documentary evidence of an interaction of L119 protein with membrane receptors, including the VEGF receptor neuropilin (Example 9). These data, and the fact that expression of the L119 gene is induced by stimuli which generate a global or local hypoxia (animal model, see Example 5; in vitro cell culture model, see Example 7), indicate that there is a connection between the expression of L119 and the processes of angiogenesis. These latter can be either physiological processes (e.g. neoangiogenesis during the development of an organism) or pathological mechanisms, as occur, for example, in association with tumor growth.

c) Inflammatory Diseases

L119 is upregulated in a model of inflammation and septic shock after induction with lipopolysaccharide (LPS) (Example 13) indicating a function in acute and/or chronic inflammatory diseases.

d) Ischemia

L119 is upregulated under ischemic conditions in vitro (see b above; Example 7). In addition, the infarct volume in L119 ko mice is significantly increased when compared with wild-type mice (Example 17).

e) Thrombotic Diseases

Most astonishing, L119 ko mice showed significantly decreased bleeding times compared to wild-type littermates (Example 18). Blood derived from L119 ko mice aggregated more vigorously than blood from wild-type mice (Example 19) suggesting that the L119 gene product might have anti-thrombotic effects. The experiments reveal that L119 ko mice exert a stronger, more intense pro-thrombotic response to injuries, supporting the hypothesis that the L119 null phenotype is related to a hyper-activation of platelet function (Example 20).

The results from the above mentioned disease models strongly indicate that L119 is a key player in vascular functions and/or vascular homeostasis, especially in endothelial, platelet and/or coagulation functions.

Homology searches carried out with the genomic sequence of mouse L119 in the EMBL nucleotide databases using the Blast program (Altschul S F et al. (1997) Nucleic Acids Res 25, 3389-3402) showed a high degree of similarity with an entry consisting of sequences derived from human genomic data. The entry AC007215 (Release 62, last updated, Version 21; dated 24 JAN 2000) consists of 131 unordered sequence segments of the BAC clone RPCI11-59H1, which derives from chromosome 12 (region 12p12). On the basis of the high degree of sequence homology between mouse L119 sequences and sequences from the entry AC007215, it can be assumed that this BAC at least contains parts of the genomic sequence of L119 (see Examples 3 and 4). In summary, therefore, the human genomic locus of L119 can be assigned to chromosome 12, region 12p12.

The OMIM database (see above) was examined to determine whether there are syndromes in the region of the L119 locus whose possible cause could be mutations in the L119 gene. In doing this, consideration was also given to the specific expression of L119 in blood vessels, to its inducibility by a variety of stimuli and to its interaction with important receptors in the blood vessel system. Surprisingly, it was possible to identify two syndromes in the region of the L119 locus (12p12) for which L119 constitutes a bona fide candidate gene. Surprisingly, a locus for essential hypertension (see above) was found on chromosome 12 in the immediate vicinity of the L119 locus.

The locations of the two QTLs for hypertension which were found to be present in the region of the HTNB locus on chromosome 12 (Nagy Z et al. (1999) J Am Soc Nephrol 10:1709-1716; Frossard P M and Lestringant G G (1995) Clin Genet 48:284-287) were only defined in an extremely approximate manner. Based on the information provided in the OMIM database, it was not possible to draw any conclusion with regard to the causative gene, either directly or indirectly.

The data showing that L119 is specifically expressed in blood vessels and the fact that the expression can be regulated by a variety of stimuli, make L119 a bona fide candidate gene, the mutation of which could be the cause of the abovementioned disease. In addition to families which possess this monogenetic defect, various L119 allele variants could also contribute, as

QTLs, to polygenically inherited diseases of the cardiovascular system.

Once a connection has been established between L119 and the abovementioned syndromes, it is then readily possible, using methods with which a skilled person is familiar, to identify and characterize the mutation. In this connection, genomic DNA will normally be isolated from the patients being investigated. The DNA of affected individuals is then examined for the presence of mutations in the L119 locus which do not occur in samples obtained from healthy control persons (or, in the case of QTLs, not at the same frequency). For this, the genomic region to be investigated is either cloned into suitable vectors, isolated and subsequently analyzed, or else directly amplified by means of PCR and then analyzed. Examples of current analytical methods are detection of single-stranded conformation polymorphism (SSCP) or the direct sequencing of amplified PCR products. Other processes and methods are mentioned below.

Because L119 is specifically expressed in vascular endothelial cells and the expression of L119 is augmented by a variety of stimuli, it is possible to deduce that L119 is importantly involved, directly or indirectly, in the abovementioned regulatory functions of the endothelium. Depending on the nature of the disease, an increase or decrease in an L119 protein, or in one of its essential properties or in its activity, could be advantageous. Thus, for example, treatment of a tumor may require a different approach from that used when treating stroke or cardiac infarction.

The present invention relates to novel, specifically expressed proteins and nucleic acid sequences, preferably isolated proteins and nucleic acid sequences, to nucleic acid constructs which encode the proteins, and to functional equivalents or functionally equivalent parts thereof.

The invention also relates to transgenic organisms which harbor the nucleic acid sequences or nucleic acid constructs in functional or nonfunctional form, and to transgenic animals in whose germ cells and/or in the totality or a part of the somatic cells of which a nucleic acid sequence according to the invention has been altered transgenically by means of genetic manipulation methods or has been interrupted by inserting DNA elements.

The invention furthermore relates to methods for finding compounds which have specific binding affinity for one of the proteins or nucleic acids according to the invention, and to methods for finding compounds which modulate or normalize at least one of the essential properties, or the expression, of one of the proteins according to the invention.

The invention furthermore relates to compounds which can be obtained using the methods according to the invention, for example monoclonal or polyclonal antibodies or low molecular weight compounds, such as agonists and antagonists, for the proteins according to the invention.

The invention also relates to the use of the proteins and nucleic acid sequences according to the invention, and of the compounds which bind to, or modulate or normalize, the proteins and nucleic acid sequences according to the invention, for finding specifically binding proteins, for finding substances having specific binding affinity-or for finding genomic sequences, and also in analytical, diagnostic, prognostic or therapeutic methods and for producing drugs.

An “isolated” protein means a protein which is essentially free of other cellular material or other contaminating proteins from the cell, the tissue or an expression system from which the protein has been isolated, or which is essentially free from chemical starting compounds or other chemicals if it has been prepared synthetically using chemicals.

“Essentially free from other cellular material” means preparations of an L119 protein which contain less than 30% (based on dry weight) of a non-L119 protein, preferably less than 20% of a non-L119 protein, particularly preferably less than 10% of a non-L119 protein, very particularly preferably less than 5% of a non-L119 protein.

An “isolated” nucleic acid means a nucleic acid which is essentially free from other cellular material or other contaminating nucleic acids from the cell, the tissue or an expression system from which the nucleic acid has been isolated, or which is essentially free of chemical starting compounds or other chemicals if it has been prepared synthetically using chemicals.

“Essentially free from other cellular material” means preparations of an L119 nucleic acid which contains less than 30% (based on the dry weight) of a non-L119 nucleic acid, preferably less than 20% of a non-L119 nucleic acid, particularly preferably less than 10% of a non-L119 nucleic acid, very particularly preferably less than 5% of a non-L119 nucleic acid.

“Essentially free from chemical starting compounds or other chemicals” encompasses preparations of an L119 protein or L119 nucleic acid which contain less than 30% (based on dry weight) of chemical starting compounds or other chemicals, preferably less than 20% of chemical starting compounds or other chemicals, particularly preferably less than 10% of chemical starting compounds or other chemicals, very particularly preferably less than 5% of chemical starting compounds or other chemicals.

Isolated proteins which are particularly preferred in accordance with the invention are understood as being proteins which contain one of the amino acid sequences depicted in SEQ ID NO: 3, 6, 7 or 24.

A functional equivalent is understood as meaning, in particular, natural or artificial mutations of an L119 nucleic acid sequence as depicted in SEQ ID NO:1, 2, 4, 5, 22 or 23 or of an L119 protein sequence as depicted in SEQ ID NO: 3, 6, 7 or 24 and also their homologs from other animal or plant genera and species which in addition, where appropriate after transcription and translation, still exhibit at least one of the essential biological properties of the protein depicted in SEQ ID NO: 3, 6, 7 or 24.

The isolated protein and its functional equivalents can advantageously be isolated from the vascular endothelium of mammalia such as Homo sapiens, Mus musculus or Rattus norvegicus. Functional equivalents are also to be understood as being homologs from other mammalia. Preference is given to homologs from other mammalian species. Particular preference is given to those homologs which can be obtained from the genera and species humans, monkey species such as chimpanzees and gorilla, mouse, rat, bovine, pig, horse or sheep. Very particular preference is given to homologs from humans. Other examples of L119 nucleic acid sequences or protein sequences in different organisms whose genomic sequences are known can readily be identified, for example, from databases by carrying out homology comparisons using the nucleic acid sequences as depicted in SEQ ID NO: 1, 2, 4, 5, 22 or 23 or the protein sequences as depicted in SEQ ID NO: 3, 6, 7 or 24.

Natural or artificial mutations encompass substitutions, additions, deletions, inversions or insertions of one or more nucleotide or amino acid residues. Consequently, the present invention also encompasses, for example, those nucleotides and/or amino acid sequences which are obtained by modifying an L119 nucleic acid sequence as described by SEQ ID NO: 1, 2, 4, 5, 22 or 23 or a protein sequence as depicted in SEQ ID NO: 3, 6, 7 or 24. The aim of such a modification can, for example, be the insertion of additional restriction enzyme cleavage sites, the removal surplus DNA or amino acid sequences or the addition of additional sequences, for example of sequences encoding transit or signal peptides. However, it is also possible for the sequences of one or more amino acids or nucleotides to be switched or for one or more amino acids or nucleotides to be added or removed, or for several of these procedures to be combined with each other.

When it is a matter of carrying out insertions, deletions or substitutions, such as transitions or transversions, it is possible to use techniques which are known pet se, such as in vitro mutagenesis, primer repair, restriction or ligation. The ends of the fragments to be used for the ligation can be made complementary by means of manipulations, such as restriction, or the “chewing-back” or filling-in of protruding ends when making blunt ends. Analogous results can also be achieved with the aid of the polymerase chain reaction (PCR) and using specific oligonucleotide primers.

Substitution in relation to proteins is understood as meaning the replacement of one or more amino acids or nucleotides with one or more amino acids or nucleotides. Preference is given to performing what are termed conservative replacements, in which the amino acid which is used for the replacements, or the amino acid which the substituted nucleotides encode, has a similar physicochemical property (space-filling, basicity, hydrophobicity, etc., for example hydrophobic, acidic or basic property) to that of the original amino acid, for example replacement of Glu with Asp, Gln with Asn, Val with Ile, Leu with Ile and Ser with Thr.

Deletion is the replacement of an amino acid or nucleotide with a direct linkage. Preferred positions for deletions are the termini of the polypeptides and the linkages between the individual protein domains.

Insertions are insertions of amino acids or nucleotides into the polypeptide or polynucleotide chain, respectively, with formally, a direct linkage being replaced by one or more amino acids or nucleotides, respectively.

The proteins which have been altered in this way, as compared with SEQ ID NO: 3, 6, 7 or 24, possess at least 60%, preferably at least 70%, and particularly preferably at least 90%, identity of sequence with the sequences in SEQ ID NO: 3, 6, 7 or 24 as calculated in accordance with the algorithm of Altschul et al. (Altschul S F (1990) J Mol Biol 215, 403-410).

The nucleic acid sequences which have been altered in this way as compared with SEQ ID NO: 1, 2, 4, 5, 22 or 23 possess at least 60%, preferably at least 70%, and, particularly preferably at least 90% identity of sequence with the sequences in SEQ ID NO: 1, 2, 4, 5, 22 or 23 as calculated in accordance with the algorithm of Altschul et al. (Altschul S F (1990) J Mol Biol 215, 403-410).

An “essential biological property” of the proteins according to the invention is to be understood as being at least one of the following properties:

a) the putative transmembrane region(s), the amino-terminal region or the carboxy-terminal region, the coiled-coil region (see Example 2), or

b) the presence of at least one of the following amino acid sequences:

| | |
|---|---|
| 1. | DALRRFQGLLLDRRGRLH |
| 2. | QVLRLREVARRLERLRRRSL |
| 3. | GALAAIVGLSLSPVTLG |
| 4. | SAVGLGVATAGGAVTITSDLSLIFCNSRE |
| 5. | RRVQEIAATCQDQMRE |
| 6. | ALYNSVYFIVFFGSRGFLIPRRAEG |
| 7. | TKVSQAVLKAKIQKL |
| 8. | ESLESCTGALDELSEQLESRVQLCTK |

c) interaction with at least one of the following proteins

1. Nel

2. Notch 4

3. Notch 3

4. Notch 2

5. matrilin-2

6. TIED

7. laminin alpha-4 chain

8. Ten-m3

d) a molecular weight of from 20 to 35 kD, preferably of from 25 to 30 kD, very particularly preferably of 27 kD

e) expression in an endothelial cell or tissue

f) expression inducible by hypoxia, cycloheximide, pentylenetetrazole, kainate, focal or global ischemia and/or MECS stimulation.

These protein regions enable the proteins according to the invention to exert their specific biological effect. These essential biological properties additionally comprise the binding of specific synthetic or natural agonists and antagonists to the proteins according to the invention having the amino acid sequences depicted in SEQ ID NO: 3, 6, 7 or 24.

The invention furthermore relates to nucleic acid sequences which encode the above-described proteins, in particular to those which have the primary structures depicted in SEQ ID NO: 3, 6, 7 or 24. The nucleic acid sequence from Rattus norvegicus is depicted in SEQ ID NO: 1 or SEQ ID NO: 2, that from Mus musculus in SEQ ID NO: 4 or SEQ ID NO: 23 and that from Homo sapiens in SEQ ID NO: 5 or SEQ ID NO: 22. The invention also encompasses functional equivalents of these nucleic acid sequences.

The nucleotide sequences according to the invention SEQ ID NO: 1, 2, 4, 5, 22 or 23, or their functional equivalents, such as allele variants, can be obtained following isolation and sequencing. Allele variants are understood as being variants of SEQ ID NO: 1, 2, 4, 5, 22 or 23 which exhibit from 60% to 100% identity at the amino acid level, preferably from 70% to 100% identity, and very particularly preferably from 90% to 100% identity. Allele variants encompass, in particular, those functional variants which can be obtained by deleting, inserting or substituting nucleotides from, into or within, respectively, the sequences depicted in SEQ ID NO: 1, 2, 4, 5, 22 or 23, with at least one of the essential biological properties still being retained in the protein obtained after transcription and translation.

In addition, the invention encompasses sequences which are complementary to the nucleic acid sequences depicted in SEQ ID NO: 1, 2, 4, 5, 22 or 23 and also functional equivalents or functionally equivalent parts thereof. With regard to complementary sequences, “functionally equivalent” or “functional equivalent” generally means those nucleic acid sequences which possess a identity of at least 60%, preferably at least 70%, particularly preferably at least 90%, with a nucleic acid sequence as depicted in SEQ ID NO: 1, 2, 4, 5, 22 or 23, or a part thereof, and have a length of at least 15 nucleotides, preferably at least 25 nucleotides, particularly preferably at least 50 nucleotides, very particularly preferably at least 100 nucleotides, and which are able to fulfill a specific function which is intended for them, for example a decrease in expression of an L119 protein.

Homologs or nucleic acid sequences whose sequences are related to those of the nucleic acid sequences depicted in SEQ ID NO: 1, 2, 4, 5, 22 or 23 can be isolated from any mammalian species, including humans, using customary methods, for example by screening homology by hybridizing with a sample of the nucleic acid sequences according to the invention or parts thereof.

Functional equivalents are also understood as meaning homologs of SEQ ID NO: 1, 2, 4, 5, 22 or 23, for example their homologs of other mammalia, truncated sequences, single-stranded DNA or RNA corresponding to the coding, non-coding or complementary DNA sequences.

Such functional equivalents can be isolated from other vertebrates, such as mammalia, using the DNA sequences described in SEQ ID NO: 1, 2, 4, 5, 22 or 23, or parts of these sequences, as the starting material and employing, for example, customary hybridization methods or the PCR technique. These DNA sequences hybridize with the sequences according to the invention under standard conditions. For the hybridization, use is advantageously made of short oligonucleotides which encode the abovementioned amino acid sequences 1 to 8. However, it is also possible to use longer fragments of the nucleic acids according to the invention, or the complete sequences, for the hybridization. These standard conditions vary depending on the nucleic acid, oligonucleotide, longer fragment or complete sequence employed or depending on which nucleic acid type, i.e. DNA or RNA, is used for the hybridization. Thus, the melting temperatures for DNA:DNA hybrids are approx. 10° C. lower than those for DNA:RNA hybrids of the same length.

The expression “standard hybridization conditions” is to be understood broadly and means both stringent and less stringent hybridization conditions. Such hybridization conditions are described, inter alia, in Sambrook J, Fritsch E F, Maniatis T et al., in Molecular Cloning (A Laboratory Manual), 2nd edition, Cold Spring Harbor Laboratory Press, 1989, pages 9.31-9.57) or in Current Protocols in Molecular Biology, John Wiley & Sons, N.Y. (1989), pp. 6.3.1-6.3.6.

Standard conditions are to be understood as meaning, for example, depending on the nucleic acid, temperatures of between 42° C. and 58° C. in an aqueous buffer solution having a concentration of between 0.1 and 5×SSC (1×SSC=0.15 M NaCl, 15 mM sodium citrate, pH 7.2) or, additionally, in the presence of 50% formamide. Hybridization conditions which may be mentioned by way of example are:

a) 45° C. in 6×SSC, or

b) 42° C. in 5×SSC, 50% formamide.

The hybridization conditions for DNA:DNA hybrids are advantageously 0.1×SSC and temperatures of between about 20° C. and 45° C., preferably of between about 30° C. and 45° C. For DNA:RNA hybrids, the hybridization conditions are advantageously 0.1×SSC and temperatures of between about 30° C. and 55° C., preferably of between about 45° C. and 55° C. These temperatures which are specified for the hybridization are melting temperature values, which are calculated by way of example, for a nucleic acid having a length of approx. 100 nucleotides and a G+C content of 50% in the absence of formamide. Where appropriate, SDS can also be added for the purpose of increasing the stringency.

The experimental conditions for the DNA hybridization are described in relevant textbooks of genetics, such as Sambrook et al., 1989, and can be calculated using formulae known to the skilled person, for example in dependence on the length of the nucleic acids, the nature of the hybrids or the G+C content. The skilled person can obtain additional information with regard to the hybridization from the following textbooks: Ausubel F M et al., (1998) Current Protocols in Molecular Biology (New York: John Wiley & Sons); Hames B D and Higgins S J (1985) Nucleic Acids Hybridization: A Practical Approach, IRL Press at Oxford University Press, Oxford; Brown T A (1991) Essential Molecular Biology: A Practical Approach, IRL Press at Oxford University Press, Oxford.

In addition, homologs of the sequences SEQ ID NO: 1, 2, 4, 5, 22 or 23 are also understood as being derivatives such as promoter variants. The promoters, which are located upstream of the given nucleotide sequences, jointly or individually, may be altered by one or more nucleotide exchanges, or by (an) insertion(s) and/or (a) deletion(s), without, however, the essential property or activity of the promoters being impaired. Furthermore, the activity of the promoters can be increased or decreased by changing their sequences, or else the promoters can be completely replaced with other promoters, even from organisms of a different species.

Derivatives are also advantageously to be understood as meaning variants whose nucleotide sequences have been altered in the region -1 to -10000 upstream of the start codon, or in other regulatory cis-flanking elements, such that gene expression and/or protein expression is altered, preferably increased. Furthermore, derivatives are also to be understood as being variants which have been altered at the 3′ end.

The invention furthermore relates to nucleic acid constructs, preferably transgenic nucleic acid constructs, which contain the nucleic acid sequences according to the invention. In these nucleic acid constructs, an L119 nucleic acid sequence which is to be expressed transgenically, or its functional equivalent, can, for example, be functionally linked to other genetic regulatory elements. Moreover, the nucleic acid constructs can contain additional functional elements. These nucleic acid constructs can preferably constitute vectors or expression vectors which contain the nucleic acid sequences according to the invention. These vectors or expression vectors are covered by the term nucleic acid construct below.

The term “vector” means a nucleic acid molelcule which is suitable for transporting another nucleic acid which has been linked to the vector. Apart from plasmids, vectors are also to be understood as meaning any other vectors known to the skilled person, such as phages, viruses, such as SV40, CMV, baculovirus, adenovirus, transposons, IS elements, phasmids, phagemids, cosmids, BACs, YACs, mammalian (mini)chromosome vectors, or linear or circular DNA. Advantageously, the nucleic acids according to the invention are inserted into a host-specific vector which enables the genes to be express optimally in the chosen host. Vectors are well known to the skilled person and are listed, for example, in Pouwels P H (1985) Cloning Vectors, Elsevier, Amsterdam-New York-Oxford. Vectors can either be replicated autonomously in the host organism or can integrate into the host genome and be replicated chromosomally. Linear DNA is advantageously used for chromosomal integration in mammalia. A preferred form of a vector is a “plasmid”, with this term covering a double-stranded, circular DNA molecule.

“Nucleic acid construct” or “nucleic acid sequence” is understood, according to the invention, as meaning, for example, a genomic sequence or a complementary DNA sequence or an RNA sequence and also semisynthetic or completely synthetic analogs thereof. These sequences can be present in linear or circular form and be present extrachromosomally or integrated into the genome. The L119 nucleic acid sequences may be prepared synthetically or isolated naturally or contain a mixture consisting of synthetic and natural DNA constituents, and also consist of different heterologous L119 gene segments obtained from different organisms.

Preference is given to nucleic acid constructs which transgenically contain the nucleic acid sequences according to the invention as depicted in SEQ ID NO: 1, 2, 4, 5, 22 or 23 or their functional equivalents or functionally equivalent parts thereof.

In addition, artificial L119 nucleic acid sequences are suitable as long as, as described above, they mediate at least one essential property of one of the L119 proteins according to the invention in a cell or an organism or, when a complementary (“antisense”) sequence is used, are still able to fulfill the function which is intended for them, for example that of reducing the expression of an L119 protein.

For example, it is possible to produce synthetic nucleotide sequences which contain codons which are preferred by the organisms to be transformed. For heterologous genes to be expressed optimally in organisms, it is advantageous for the nucleic acid sequences to be altered in accordance with the specific codon usage which is employed in the organism. These preferred codons can be determined, in a customary manner, from the codons which are most frequently used for encoding the proteins. The codon usage which is specific for a particular organism can readily be ascertained with the aid of computer evaluations of other known genes in the organism concerned. Such artificial nucleotide sequences can be determined, for example, by back-translating L119 proteins which have been constructed by molecular modeling or by means of in vitro selection. Coding nucleotide sequences which have been obtained by back-translating a polypeptide sequence in accordance with the codon usage which is specific for the host organism are particularly suitable

All the abovementioned nucleotide sequences can be prepared, in a manner known per se, by chemical synthesis from the nucleotide building blocks, for example by fragment condensation of individual overlapping, complementary nucleic acid building blocks of the double helix. Oligonucleotides can be synthesized chemically, for example, in a known manner in accordance with the phosphoamidite method (Voet, Voet, Biochemistry, 2nd edition, Wiley Press New York, pages 896-897).

When an expression cassette is being prepared, different DNA fragments can be manipulated such that a nucleotide sequence having a correct direction of reading and a correct reading frame is obtained. In order to bond the nucleic acid fragments to each other, it is possible to attach adapters or linkers to the same. The adding-on of synthetic oligonucleotides, and the filling-in of gaps with the aid of the DNA polymerase Klenow fragment, and ligation reactions and general cloning methods, are described in Sambrook et al. (1989), Molecular cloning: A laboratory manual, Cold Spring Harbor Laboratory Press.

With regard to the example of a nucleic acid sequence (for example L119 nucleic acids as depicted in SEQ ID NO: 1, 2, 4, 5, 22 or 23), a nucleic acid construct which contains said nucleic acid sequence or an organism which is transformed with said nucleic acid sequence or said nucleic acid construct, “transgene” means all those constructs which have been brought about by genetic manipulation methods and in which either

a) the nucleic acid sequence (for example an L119 nucleic acid sequence as depicted in SEQ ID NO: 1, 2, 4, 5, 22 or 23 or a functional equivalent or functionally equivalent part thereof), or

b) a genetic regulatory element, for example a promoter, which is functionally linked to the nucleic acid sequence (for example an L119 nucleic acid sequence as depicted in SEQ ID NO: 1, 2, 4, 5, 22 or 23 or a functional equivalent or functionally equivalent part thereof), or

c) (a) and (b)

is/are not present in its/their natural genetic environment or has/have been modified by means of genetic manipulation methods, it being possible for the modification to be, by way of example, a substitution, addition, deletion, inversion or insertion of one or more nucleotide radicals. “Natural genetic environment” means the natural chromosomal locus in the organism of origin or the presence in a genomic library. In the case of a genomic library, the natural, genetic environment of the nucleic acid sequence is preferably at least partially still preserved. The environment flanks the nucleic acid sequence at least on one side and has a sequence length of at least 50 bp, preferably at least 500 bp, particularly preferably at least 1000 bp, very particularly preferably at least 5000 bp.

Preference is given to the L119 sequences which are contained in the transgenic nucleic acid constructs being functionally linked to at least one genetic regulatory element, such as transcription and translation signals. Depending on the desired application, this linkage can lead to an increase or a decrease in the expression of an L119 gene. Host organisms are subsequently transformed with the recombinant transgenic nucleic acid constructs which have been prepared in this way.

The term “genetic regulatory element” is to be understood broadly and means all those sequences which have an influence on the genesis or the function of the nucleic acid constructs according to the invention. For example, genetic regulatory elements ensure transcription and, where appropriate, translation in prokaryotic or eukaryotic organisms. The nucleic acid constructs according to the invention preferably include, as additional genetic regulatory elements, a promoter and a transcription termination signal, which are located 5′-upstream and 3′-downstream, respectively, of the particular nucleic acid sequence which is to be expressed transgenically, and also, where appropriate, additional customary regulatory elements such as polyadenylation signals or enhancers, in each case functionally linked to the nucleic acid sequence which is to be expressed transgenically.

In this connection, the regulatory sequences or factors can preferably influence the expression positively and thereby increase it. Thus, the regulatory elements can advantageously be augmented at the transcription level by using strong transcription signals such as promoters and/or enhancers. In addition to this, however, it is also possible to augment translation by, for example, improving the stability of the mRNA.

“Functionally linked” is to be understood broadly and means that the nucleic acid sequence has been linked to the genetic regulatory elements such that the genetic regulatory sequence can in each case exert the function which is intended for it on the nucleic acid sequence, as desired, optionally following introduction into a host cell. Thus, the regulatory sequence can, for example, modulate or normalize expression of the nucleic acid sequence, i.e. ensure transcription and/or translation.

A functional linkage is understood as meaning, for example, the sequential arrangement of a promoter, an L119 nucleic acid sequence which is to be expressed transgenically, and, where appropriate, further regulatory elements, such as a terminator, such that each of the regulatory elements is able to fulfill its function in the transgenic expression of the nucleic acid sequence. A direct linkage in the chemical sense is not necessarily required for this. Genetic regulatory elements such as enhancer sequences can also exert their function on the target sequence from more distant positions or even from other DNA molecules. Preference is given to arrangements in which the L119 nucleic acid sequence to be expressed transgenically is located downstream of the sequence functioning as a promoter such that both sequences are linked to each other covalently. In this connection, preference is given to the distance between the promoter sequence and the nucleic acid sequence to be expressed transgenically being less than 200 base pairs, particularly preferably less than 100 base pairs, and very particularly preferably less than 50 base pairs. However, additional sequences, which have, for example, the function of a linker, possessing particular restriction enzyme cleavage sites, or of a signal peptide, can be located between the two sequences. The insertion of sequences can also lead to the expression of fusion proteins.

Examples are sequences to which inducers or repressors bind and in this way regulate the expression of the nucleic acid. In addition to these new regulatory elements, or instead of these sequences, the natural regulation of these sequences can still be present upstream of the actual structural gene and, where appropriate, have been genetically altered such that the natural regulation has been switched off and the expression of the genes has been increased. However, the nucleic acid construst can also be assembled in a simpler manner, i.e. no additional regulatory signals are inserted upstream of the abovementioned genes and the natural promoter, together with its regulation, is not removed. Instead, the natural regulatory element is mutated such that there is no longer any regulation and gene expression is increased. These altered promoters can also be placed on their own upstream of the natural genes for the purpose of increasing activity.

Genetic regulatory signals which are suitable in accordance with the invention have been described and are known to the skilled person (see Goeddel; Gene Expression Technology: Methods in Enzymology 185, Academic Press, San Diego, Calif. (1990)).

A genetic control sequence, or a combination of different genetic control sequences, can enable expression to take place in one or more eukaryotic and/or prokaryotic host organisms or in cells which are derived therefrom. Suitable host organisms can be bacteria, such as E.coli, insect cells (when using a Baculovirus expression system, for example), yeast cells or mammalian cells. Suitable host organisms are known to the skilled person (Goeddel, Gene Expression Technology: Methods in Enzymology 185, Academic Press, San Diego, Calif. (1990)).

Alternatively, it is also possible to effect transcription and/or translation in vitro, for example using a T7 promoter and a T7 polymerase.

The invention also encompasses L119 fusion proteins or chimeric proteins, with these terms being understood to mean proteins in which the L119 polypeptide is functionally linked to a non-L119 polypeptide. “L119 polypeptide” means L119 proteins as depicted in SEQ ID NO: 3, 6, 7 or 24 or their functional equivalents in accordance with the abovementioned definition. “Non-L119” polypeptide means all those polypeptides which diverge significantly from the sequence of an L119 protein and do not satisfy the abovementioned criteria with regard to homology and function.

An L119 protein can also be expressed in the form of a fusion protein. In this case, the nucleic acid construct adds a number of amino acids N-terminally or C-terminally to the protein which is to be expressed. These additional amino acids can, for example, have the function of increasing the expression of the recombinant protein, raising its solubility, enabling it to be detected, or facilitating its purification. In the case of the last-mentioned property, for example, the amino acids which are added on then have the function of a ligand within the context of an affinity purification. Furthermore, amino acid sequences can be added onto the L119 polypeptide, which sequences permit or augment expression and/or secretion in particular host cells (e.g. mammalian cells). Furthermore, fusion proteins can advantageously be used as antigens when preparing anti-L119 antibodies.

In addition to this, the L119 proteins according to the invention can also be expressed in the form of therapeutically or diagnostically suitable fragments. In order to generate the recombinant protein, it is possible to use vector systems or oligonucleotides which extend the nucleic acids or the nucleic acid construct by particular nucleotide sequences and thereby encode altered polypeptides which simplify purification. “Tags” of this nature are either known in the literature, e.g. hexahistidine anchor, or are epitopes which can be recognized as being antigens of various antibodies (Studier F W et al. (1990) Methods Enzymol 185, 60-89 and Ausubel F M et al., (1998) Current Protocols in Molecular Biology (New York: John Wiley & Sons)).

In a preferred embodiment, the amino acids which have been added on can be eliminated proteolytically once they have fulfilled their purpose. To do this, it is possible to insert additional amino acid sequences, which function as recognition sequences for sequence-specific proteases,,at the connection point between the protein which is to be expressed and the amino acids which are added on additionally. Examples of suitable proteases are factor Xa, thrombin and enterokinase. Suitable vectors for preparing the nucleic acid constructs according to the invention for expressing fusion proteins include, for example, fusion expression vectors such as PGEX (Pharmacia Biotech Inc; Smith D B and Johnson K S (1988) Gene 67:31-40), PMAL (New England Biolabs, Beverly, Mass.) and pRIT5 (Pharmacia, Piscataway, N.J.), which add on glutathione S-transferase (GST), maltose E-binding protein and protein A, respectively, to the protein which is to be expressed transgenically.

Purified L119 fusion proteins can be used in test systems for identifying L119-modulating or -normalizing compounds or else for preparing antibodies.

Inducible E.coli expression vectors include, for example, pTrc (Amann et al., (1988) Gene 69:301-315) and pET 11d (Studier et al., Gene Expression Technology: Methods in Enzymology 185, Academic Press, San Diego, Calif. (1990) 60-89). The techniques for obtaining expression are known to the skilled person as are the methods for optimizing expression, with regard to level, and other parameters, for example by selecting the suitable E.coli strain or adapting the codons to those which are customary in E.coli (Gottesman S, Gene Expression Technology: Methods in Enzymology 185, Academic Press, San Diego, Calif. (1990) 119-128; Wada et al., (1992) Nucleic Acids Res. 20:2111-2118).

Various expression vectors are available to the skilled person for expression in yeast cells, for example pYepSec1 (Baldari, et al., (1987) Embo J. 6:229-234), pMFa (Kurjan and Herskowitz, (1982) Cell 30:933-943), pJRY88 (Schultz et al. (1987) Gene 54:113-123), pYES2 (Invitrogen Corporation, San Diego, Calif.) and picZ (InVitrogen Corp, San Diego, Calif.).

Alternatively, an L119 protein can also be expressed in insect cells (e.g. Sf9 or “High 5” cells) using Baculovirus expression vectors. The pAc series (Smith et al. (1983) Mol Cell Biol 3:2156-2165) and the pVL series (Lucklow and Summers (1989) Virology 170:31-39) may be mentioned by way of example.

The L119 proteins are preferably expressed in mammalian cells. Examples of vectors which are suitable for expression in mammalian cells include pCDM8 (Seed B (1987) Nature 329:840), pMT2PC (Kaufman et al. (1987) EMBO J -6:187-195) and vectors of the pCDNA3 series (invitrogen).

Other vectors which are suitable for expression in prokaryotic and eukaryotic cells have been described (see Chapters 16 and 17 in Sambrook J, Fritsh E F and Maniatis T “Molecular Cloning: A Laboratory Manual” 2nd ed., Cold Spring Harbor Laboratory, Cold Spring Harbor Laboratory Press, Cold Spring Harbor, N.Y., 1989).

Various regulatory elements are suitable depending on the host organism or the starting organism which is converted, by introducing the nucleic acid constructs, into a genetically altered or transgenic organism.

Advantageous regulatory sequences for the process according to the invention are contained, for example, in promoters such as the cos, tac, trp, tet, lpp, 1ac, lacIq, T7, T5, T3, gal, trc, ara, SP6, 1-PR or 1-PL promoters, which are advantageously used in Gram-negative bacteria. Further advantageous regulatory sequences are contained, for example, in the Gram-positive promoters such as amy and SPO2, in the yeast promoters such as ADC1, MFa, AC, P-60, CYC1 or GAPDH, or in mammalian promoters such as those of the von Willebrand factor gene, preproendothelin-1, angiotensin-converting enzyme, vascular endothelial growth factor (VEGF) receptor-2 (Flk-1), Tie-2/Tek, vascular endothelial cadherin, eNOS, intercellular adhesion molecule-2 and ICAM-2.

In principle, it is possible to use all natural promoters together with their regulatory sequences such as those mentioned above. In addition to this, it is also possible advantageously to use synthetic promoters.

The regulatory sequences should enable the nucleic acid sequences to be expressed (i.e. transcribed and/or, where appropriate, optionally translated) in a specific manner. Depending on the host organism this can, for example, mean that the gene is only expressed or overexpressed after induction or that it is expressed and/or overexpressed immediately.

In a preferred embodiment, the L119 proteins according to the, invention, or their functional equivalents, are expressed in a cell-specific or tissue-specific manner. Such a specific expression can be achieved by functionally linking the L119 nucleic acid sequences, or their functional equivalents, to cell-specific or tissue-specific transcriptional regulatory elements (e.g. promoters or enhancers). Numerous sequences of this nature are known to the skilled person; others can be derived from genes whose cell-specific or tissue-specific expression is known (WO 96/06111, in particular pp. 36-37). The following may be mentioned by way of example but not in a limiting manner:

Lens: g2-Crystallin (Breitman M L et al. (1987) Science 238: 30 1563-1565); aA-Crystallin (Landel C P et al. (1988) Genes Dev. 2: 1168-1178, Kaur S et al. (1989) Development 105: 613-619)

Pituitary gland: growth hormone (Behringer R R et al. (1988) Genes Dev. 2: 453-461)

Pancreas: insulin (Ornitz D M., Palmiter, R. D., Hammer, R. E., Brinster, R. L.), elastase (Swift G H and MacDonald R J (1985) Nature 131: 600-603; Palmiter R D et al. (1987) Cell 50: 435-443)

T cells: lck promoter (Chaffin K E et al. (1990) EMBO Journal 40 9: 3821-3829)

B cells: immunoglobulin (Borelli E et al. (1988) Proc. Natl. Acad. Sci. USA 85: 7572-7576; Heyman R A et al. (1989) Proc. Natl. Acad. Sci. USA 86: 2698-2702)

Schwann cells: P0 promoter (Messing A et al. (1992) Neuron 8: 45 507-520), myelin basic protein (Miskimins R et al. (1992) Brain Res Dev Brain Res Vol 65: 217-221)

Spermatids: protamine (Breitman M L et al. (1990) Mol. Cell. Biol. 10: 474-479)

Lung: surfactant gene (Ornitz D M et al. (1985) Nature 131: 600-603)

Adipocytes: P2 (Ross S R et al. (1993) Genes and Dev 7: 1318-24

Muscle: myosin light chain (Lee K J et al. (1992 Aug. 5) J. Biol. Chem. 267: 15875-85), alpha actin (Muscat G E et al. (1992) Gene Expression 2, 111-126)

Neurons: neurofilament (Reeben M et al. (1993) BBRC 192: 465-70)

Liver: tyrosine aminotransferase, albumin and apolipoproteins.

Preferred embodiments include the albumin promoter (liver-specific; Pinkert et al. (1987) Genes Dev. 1:268-277), lymphoid-specific promoters (Calame and Eaton (1988) Adv Immunol 43:235-275), promoters of the T cell receptors (Winoto and Baltimore (1989) EMBO J. 8:729-733) and immunglobulins (Benerji et al. (1983) Cell 33:729-740; Queen and Baltimore (1983) Cell 33:741-748), neuron-specific promoters (e.g. the neurofilament promoter; Byrne and Ruddle (1989) Proc Natl Acad Sci USA 86:5473-5477), pancreas-specific promoters (Edlund et al. (1985) Science 230:912-916) and mammary gland-specific promoter (U.S. Pat. No. 4,873,316, EP 0 264 166). Promoters which are regulated in a development-dependent manner, such as the murine hox promoter (Kessel and Gruss (1990) Science 249:374-379) and the α-fetoprotein promoter (Campes and Tilghman (1989) Genes Dev. 3:537-546) are also included. Very particular preference is given to promoters which ensure endothelial expression, such as the Tie-2 promoter (Fadel B. M. et al. (1998) Biochem. J. 330:335-343).

Additional, advantageous sequences, such as further regulatory elements or terminators, can also be inserted at the 3′ end of the nucleic acid sequences which are to be expressed transgenically. The nucleic acid sequences which are to be expressed transgenically can be present in one or more copies in the nucleic acid construct or in the vector.

The nucleic acid construct can advantageously contain one or more enhancer sequences which is/are functionally linked to the promoter and which enable(s) the nucleic acid sequence to be expressed transgenically at an elevated level. “Enhancers” are to be understood as meaning, for example, DNA sequences which bring about an increased expression by means of improving the interaction between the RNA polymerase and the DNA.

Genetic regulatory elements furthermore also include the 5′-untranslated region, introns and the non-coding 3′ region of genes.

Other regulatory sequences which may be mentioned by way of example are the locus control regions and silencers, or particular part sequences thereof. These sequences can advantageously be used for tissue-specific expression.

The skilled person is familiar with different ways for arriving at a nucleic acid construct according to the invention. For example, a nucleic acid construct according to the invention is preferably prepared by directly fusing a nucleic acid sequence, which functions as the promoter, to a nucleotide sequence which encodes an L119 protein and to a terminator signal or polyadenylation signal. To do this, use is made of customary recombination and cloning techniques as described, for example, in T. Maniatis, E. F. Fritsch and J. Sambrook, Molecular Cloning: A Laboratory Manual, Cold Spring Harbor Laboratory, Cold Spring Harbor, N.Y. (1989) and in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current-Protocols in Molecular Biology, Greene Publishing Assoc. and Wiley Interscience (1987). The nucleic acid construct, consisting of a link association of the promoter and the L119 nucleic acid sequence, can preferably be present in integrated form in a vector and be inserted into a eukaryotic genome, for example by means of transformation.

However, a nucleic acid construct is also to be understood as meaning those constructs in which a regulatory element, for example a promoter, without previously having been linked functionally to the L119 nucleic acid sequence, is introduced, for example by way of a specific homologous recombination or a random insertion, into a host genome, where it assumes regulatory control over an endogenous L119 nucleic acid sequence, which is then linked to it functionally, and controls the transgenic expression of this nucleic acid sequence. Inserting the promoter, for example by means of homologous recombination, upstream of a nucleic acid sequence encoding an L119 polypeptide results in a nucleic acid construct according to the invention which controls expression of the L119 polypeptide.

In an analogous manner, an L119 nucleic acid sequence can, for example, also be placed, by means of homologous recombination, downstream of an endogenous promoter, thereby resulting in a nucleic acid construct according to the invention which controls expression of the L119 nucleic acid sequence.

In this connection, regulatory elements are furthermore to be understood as meaning those which make possible homologous recombination or insertion into the genome of a host organism or which enable removal from the genome to take place. During the homologous recombination, the natural promoter of a particular L119 gene can, for example, be replaced with a constitutive promoter or a promoter having an altered specificity. Methods such as the cre/lox technology enable the nucleic acid construct to be removed from the genome or the host organism in a manner which is tissue-specific and possibly inducible (Sauer B. Methods. 1998; 14(4):381-92). In this case, particular flanking sequences (lox sequences) are added onto the target gene, which sequences subsequently enable removal to take place using the cre recombinase.

.OMEGA. or O vectors can, for example, be used for the purpose of homologous recombination (Thomas and Capecchi (1987) Cell 51:503-512; Mansour et al. (1988) Nature 336:348-352; Joyner, et al. (1989) Nature 338:153-156).

The nucleic acid constructs according to the invention and the vectors which are derived from them can contain additional functional elements. The term functional element is to be understood broadly and means all those elements which have an influence on the preparation, replication or function of the novel nucleic acid constructs, vectors or transgenic organisms which are transformed with these constructs or vectors. The following may be mentioned by way of example but not in a limiting manner:

a) Selection markers, which confer resistance to antibiotics or biocides. For example the npt gene, which confers resistance to the aminoglycoside antiobiotics neomycin (G 418), kanamycin and paromycin (Deshayes A et al., EMBO J. 1985; 4(11):2731-2737). The hygro gene, which confers resistance to hygromycin (Marsh J L et al., Gene. 1984; 32(3):481-485). The sul gene, which confers resistance to sulfadiazine (Guerineau F et al., Plant Mol Biol. 1990; 15(l):127-136). Other suitable selection marker genes are those which confer resistance to bleomycin, etc. Other suitable selection markers are those which confer an antimetabolite resistance, for example the dhfr gene as resistance to methotrexate (Reiss, Plant Physiol (Life Sci Adv) 1994, 13:142-149). Other suitable genes are those such as trpB, which enables cells to use indole instead of tryptophan, or hisD, which enables cells to use histinol instead of histidine (Hartman S C and Mulligan R C, Proc Natl Acad Sci USA. 1988; 85(21): 8047-8051). Also suitable is the gene for mannose phosphate isomerase, which enables cells to make use of mannose (WO 94/20627), or the ODC (ornithine decarboxylase) gene, which confers resistance to the ODC inhibitor DFMO (2-difluoromethyl-DL-ornithine) (McConlogue, 1987 in: Current Communications in Molecular Biology, Cold Spring Harbor Laboratory, publishers), or Aspergillus terreus deaminase, which mediates resistance to blasticidin S (Tamura K et al., Biosci Biotechnol Biochem. 1995; 59(12): 2336-2338). hprt and thymidine kinase are also suitable.

b) Suitable markers without selection pressure are, furthermore, various cell surface markers such as Tac, CD8, CD3, Thy1 and the NGF receptor.

c) Reporter genes which encode readily quantifiable proteins and ensure assessment of transformation efficiency or the site or time of expression by way of an inherent color or an enzyme activity. In this connection, very particular preference is given to reporter proteins (Schenborn E, Groskreutz D. Mol Biotechnol. 1999; 13(l):29-44) such as the green fluorescence protein (GFP) (Gerdes H H and Kaether C, FEBS Lett. 1996; 389(1):44-47; Chui W L et al., Curr Biol 1996, 6:325-330; Leffel S M et al., Biotechniques. 23(5):912-8, 1997), chloramphenicol transferase, a luciferase (Giacomin, Plant Sci 1996, 116:59-72; Scikantha, J Bact 1996, 178:121; Millar et al., Plant Mol Biol Rep 1992 10:324-414), the β-galactosidase or β-glucuronidase (Jefferson et al., EMBO J. 1987, 6, 3901-3907).

d) Origins of replication which ensure replication of the novel nucleic acid constructs or vectors in E.coli, for example. Those which may be mentioned by way of example are ORI (origin of DNA replication), the pBR322 ori or the P15A ori (Sambrook et al.: Molecular Cloning. A Laboratory Manual, 2nd ed. Cold Spring Harbor Laboratory Press, Cold Spring Harbor, N.Y., 1989).

The skilled person is familiar with the fact that the functional elements also do not necessarily have to be combined with the other nucleic acid sequences on one molecule. The invention furthermore also encompasses functional analogs, i.e. those combinations in which a functional element and the other nucleic acids come together as a result of

1. combination on one polynucleotide (multiple constructs)

2. combination as a result of cotransforming several polynucleotides into a cell

3. combination as a result of crossing different transgenic organisms which in each case contain at least one of the nucleic acid sequences.

Cotransformation suggests itself in particular in cases in which the physical coupling of, for example, a marker gene and the other nucleic acid sequences is unwanted. This can be advantageous since, in this way, after a primary transgenic organism has been selected, the marker gene and the other nucleic acid sequences can then segregate once again in subsequent crosses. Another method for subsequently removing the marker gene once again is that of using flanking DNA sequences and sequence-specific recombinases. Appropriate methods can, by way of example, be carried out using the cre/lox system or the FLP/FRT system, as also described below.

In order to select cells which have been successfully homologously recombined or else transformed, it is as a rule necessary additionally to insert a selectable marker which confers on the successfully recombined cells a resistance to an antibiotic or a metabolism inhibitor (see above). The selection marker enables the transformed cells to be selected from untransformed cells.

The expression of the nucleic acid sequences according to the invention or of the recombinant nucleic acid construct can advantageously be increased by increasing the gene copy number and/or by strengthening regulatory factors which exert a positive effect on gene expression. Thus, regulatory elements can preferably be strengthened at the transcription level by using stronger transcription signals such as promoters and enhancers. However, in addition to this, it is also possible to strengthen translation by, for example, improving the stability of the mRNA or increasing the efficiency with which this mRNA is read on the ribosomes.

In order to increase the gene copy number, the nucleic acid sequences or homologous genes can, for example, be incorporated into a nucleic acid fragment or into a vector which preferably contains the regulatory gene sequences, or promoter activity which acts in an analogous manner, which are assigned to the genes. Use is in particular made of those regulatory sequences which augment gene expression.

In a preferred embodiment, the nucleic acid construct contains one of the novel nucleic acid sequences as depicted in SEQ ID NO: 1, 2, 4, 5, 22 or 23, or a functional equivalent or functionally equivalent part thereof, in the antisense orientation to a promoter which is controlling its expression. “Antisense” means constructs in which the counterstrand which is complementary to one of the novel nucleic acid sequences as depicted in SEQ ID NO: 1, 2, 4, 5, 22 or 23, or a functional equivalent or a functionally equivalent part thereof, is transcribed. In regard to complementary sequences, “functionally equivalent” or “functional equivalent” means, in a general manner, those nucleic acid sequences which possess a homology of at least 60%, preferably at least 70%, particularly preferably at least 90%, with a nucleic acid sequence as depicted in SEQ ID NO: 1, 2, 4, 5, 22 or 23, or a part thereof, and have a length of at least 15 nucleotides, preferably at least 25 nucleotides, particularly preferably at least 50 nucleotides, and very particularly preferably at least 100 nucleotides, and which are able to fulfill a specific function which is intended for them, for example that of decreasing the expression of an L119 protein. In this connection, the decrease in the expression in a transgenic cell or organism which is transformed with the novel nucleic acid construct which enables an antisense nucleic acid to be expressed preferably amounts to at least 20%, particularly preferably at least 50%, very particularly preferably at least 80%, most preferably at least 90%, as compared with the untransformed but otherwise identical cell or organism. The appropriate methods for using antisense nucleic acids to achieve gene regulation are known to the skilled person (Weintraub H et al. Antisense RNA as a molecular tool for genetic analysis, Reviews-Trends in Genetics, Vol. 1(1) 1986) and are described below im detail.

The invention also relates to transgenic organisms which are transformed with at least one of the novel nucleic acid sequences or transgenic nucleic acid constructs and also to cells, cell cultures, progeny, organs, tissues or parts which are derived from such organisms. The term organism encompasses both multicellular organisms (e.g. whole animals) and unicellular organisms and cells which are derived from multicellular organisms.

Suitable starting organisms or host organisms for preparing the transgenic organisms are, in principle, all those organisms which enable the novel nucleic acids, their allelic variants, or their functional equivalents or derivatives, or the transgenic nucleic acid construct, to be expressed. Any prokaryotic or eukaryotic cell can be a host organism. Host organisms are to be understood as being, for example, bacteria, fungi, yeasts or plant or animal cells. Preferred organisms are bacteria, such as Escherichia coli, Streptomyces, Bacillus or Pseudomonas, eukaryotic microorganisms, such as Saccharomyces cerevisiae or Aspergillus, and higher eukaryotic cells derived from humans or animals, such as insect cells or mammalian cells (e.g. Chinese hamster ovary (CHO) or COS cells). Very particular preference is given to endothelial cells, such as HUVEC, HUAEC, HCAEC, HAEC, HMVEC, UtMVEC, HPAEC, ECV-304 and YPEN-1 cells.

The novel nucleic acid sequences and nucleic acid constructs can be introduced into the abovementioned host organisms, for the purpose of preparing a transgenic organism, using conventional transfection or transformation methods. Transfection or transformation means any type of method which can be used for introducing a nucleic acid sequence into an organism. A large number of methods are available for carrying out this procedure (see also Keown et al. 1990 Methods in Enzymology 185:527-537; Sambrook, et al. (Molecular Cloning: A Laboratory Manual. 2nd ed., Cold Spring Harbor Laboratory, Cold Spring Harbor Laboratory Press, Cold Spring Harbor, N.Y., 1989). Thus, the DNA can, by way of example, be inserted directly by means of microinjection or by means of bombardment with DNA-coated microparticles (biolistic method). The cell can also be permeabilized chemically, for example with polyethylene glycol, such that the DNA can penetrate into the cell by means of diffusion. The DNA can also be inserted by means of fusion with other DNA-containing units, such as minicells, cells, lysosomes or liposomes. Electroporation, in which the cells are permeabilized reversibly by means of an electrical impulse, is another suitable method for inserting DNA. Calcium phosphate or calcium chloride coprecipitation, DEAE dextran-mediated transfection, lipofection and electroporation are preferred methods. For the purpose of ensuring stable transfection, a gene encoding a selection marker is as a rule introduced into the cell which is to be transformed stably. The correspondingly stably transfected cells can be selected under the appropriate selection pressure. Suitable selection markers have been described above. Transgenic organisms which have been produced in this way, and which are transformed stably or transiently, can be used, for example, for preparing one of the novel L119 proteins recombinantly.

The transgenic organisms can be used for preparing nonhuman transgenic animals. In a preferred embodiment, the transgenic organism is a fertilized oocyte or an embryonic stem cell into which one of the novel nucleic acid sequences or nucleic acid constructs has been introduced. Organisms of this nature can be used in order to generate nonhuman transgenic animals into which an exogenous L119 sequence has been introduced or in which an endogenous L119 sequence has been altered, for example by means of homologous recombination: Such animals can advantageously be used for investigating the function of an L119 protein or the consequences of modulating or normalizing this protein.

The transgenic organisms can contain one of the novel nucleic acid sequences or nucleic acid constructs-in functional or non-functional form. Functional forms include, for example, the transgenic overexpression of an L119 protein or of an L119 antisense nucleic acid, whereas nonfunctional forms include, for example, the knocking-out of an L119 gene by means of homologous recombination or the insertion of null mutations.

The invention encompasses transgenic or knock-out or conditional or region-specific knock-out animals or specific mutations in recombinantly altered animals (Ausubel F M et al., (1998) Current Protocols in Molecular Biology, John Wiley & Sons, New York; and Torres R M et al. (1997) Laboratory protocols for conditional gene targeting, Oxford University Press, Oxford). By way of transgenic overexpression or genetic mutation (null mutation or specific deletions, insertions or modifications), all of which are effected by means of homologous recombination in embryonic stem cells, it is possible to produce animal models which supply valuable additional information about the (patho)physiology of the sequences according to the invention. A preferred embodiment consists in introducing into the germ line of transgenic animals the mutations in the L119 gene which are found in human hereditary diseases or polygenically inherited diseases. Animal models which have been prepared in this way can constitute essential test systems for evaluating novel therapeutic agents which exert an effect on the function of L119.

“Transgenic animal” means a nonhuman animal, preferably a mammal, particularly preferably a rodent such as a rat or a mouse. The term also includes nonhuman primates, sheep, dogs, cows, goats, chickens, amphibia and the like. The skilled person is familiar with methods for preparing transgenic animals (U.S. Pat. No. 4,736,866, U.S. Pat. No. 4,870,009, U.S. Pat. No. 4,873,191, Hogan B Manipulating the Mouse Embryo, Cold Spring Harbor Laboratory Press, Cold Spring Harbor, N.Y., 1986; Thomas K R and Capecchi M R (1987) Cell 51:503, Li E et al. (1992) Cell 69:915, Bradley A in Teratocarcinomas and Embryonic Stem Cells: A Practical Approach, Robertson E J ed. (IRL, Oxford, 1987) pp. 113-152; Bradley A (1991) Current Opinion in Biotechnology 2:823-829; WO 90/11354; WO 91/01140; WO 92/0968; WO 93/04169).

Advantageously, the abovementioned approaches can be combined with recombination systems, such as the bacteriophage P1 cre/loxP recombinase system, in order to achieve inducibility (Lakso et al. (1992) Proc Natl Acad Sci USA 89:6232-6236). Alternatively, it is also possible to use the Saccharomyces cerevisiae FLP recombinase system (O'Gorman et al. (1991) Science 251:1351-1355). The corresponding methods for generating suitable transgenic animals are known to the skilled person. Clones of the abovementioned nonhuman animals can be obtained using methods which are known to skilled persons (Wilmut, I. et al. (1997) Nature 385:810-813, WO 97/07668, WO 97/07669).

In an advantageous embodiment, the introduction of the nucleic acid sequences or nucleic acid constructs is effected using plasmid vectors. Preference is given to those vectors which enable the nucleic acid construct to be integrated stably into the host genome.

For the purpose of a biochemical analysis, it can be desirable, for example, for the cloning to take place in vectors which are suitable for transgenically expressing L119 proteins in E.coli or reticulocyte lysate.

In order to express L119 proteins in mammalian cells, the nucleic acid sequence encoding an L119 is introduced into a corresponding expression vector which is suitable for expressing proteins in mammalian cells. Appropriate vectors are known to the skilled person (see above) and commercially available in a very wide variety of embodiments.

If desired, the gene product can also be expressed in transgenic organisms such as transgenic animals, e.g. mice, rats, sheep, cattle or pigs. It is also possible to conceive, in principle, of transgenic plants. The transgenic organism can also be what are termed knock-out animals.

In this context, the transgenic animals can harbor a functionsl or nonfunctional nucleic acid sequence according to the invention or a functional or nonfunctional nucleic acid construct.

Another embodiment, according to the invention, of the above-described transgenic animals is constituted by transgenic animals in whose germ cells, or the entirety or a part of the somatic cells, the novel nucleotide sequence has been altered by recombinant methods or interrupted by inserting DNA elements.

The combination of the host organisms and the vectors, such as plasmids, viruses or phages, for example plasmids containing the RNA polymerase/promoter system and the Band Mu phages, or other temperate phages, or transposons and/or further advantageous regulatory sequences, which are suitable for the organisms forms an expression system. The term “expression systems” is preferably to be understood as meaning, for example, the combination of mammalian cells, such as cells of endothelial origin, and vectors, such as pcDNA3 vectors or CMV vectors, which are suitable for mammalian cells.

The invention also relates to processes for finding compounds which have a specific binding affinity for one of the proteins according to the invention or nucleic acids according to the invention. The invention furthermore encompasses processes for finding compounds which directly or indirectly modulate or normalize at least one essential property, or the expression, of one of the proteins according to the invention.

A process for finding compounds having specific binding affinity for the proteins according to the invention or protein heteromers according to the invention can comprise the following steps:

a) incubating the protein(s) according to the invention with the compound to be tested, and

b) detecting the binding of the compound to be tested to the protein.

A particularly preferred embodiment encompasses a process for finding substances which bind specifically to an L119 protein having an amino acid sequence as depicted in SEQ ID NO: 3, 6, 7 or 24, or a functional equivalent thereof, which process contains one or more of the following steps:

a) expressing the protein in eukaryotic or prokaryotic cells,

b) incubating the protein with the substances to be tested,

c) detecting the binding of a substance to the protein, or detecting an effect on the function of the protein.

A process for finding compounds having specific binding affinity for one of the nucleic acid sequence according -to the invention can comprise the following steps:

a) incubating at least one of the nucleic acids according to the invention with the compound to be tested,

b) detecting the binding of the compound to be tested to the nucleic acid.

A process for finding compounds which modulate or normalize at least one essential property, or the expression, of one of the novel proteins can comprise the following steps:

a) incubating one of the novel proteins or nucleic acid sequences, one of the novel nucleic acid constructs, one of the novel transgenic organisms or one of the novel transgenic animals with the compound to be tested,

b) determining the modulation or normalization of an essential property, or of the expression, of one of the novel proteins.

In relation to the abovementioned compounds having binding affinity for one of the novel nucleic acid sequences or proteins, “specific binding affinity” means a bond under in vitro or in vivo conditions, preferably under in vivo conditions. “In vivo conditions” comprise a presence in prokaryotic or eukaryotic cells, preferably in eukaryotic cells, particularly preferably in the form, with regard, for example, to location, shape, folding, modification and quantity, which corresponds to the natural state. In this connection, the binding of the compound to the novel nucleic acid sequence or protein is stronger than that to at least one other non-L119 nucleic acid sequence or non-L119 protein. Preferably, the binding is stronger by at least 100%, particularly preferably stronger by at least 500%, very particularly preferably stronger by at least 1000%, most preferably stronger by at least 10000%.

Within the context of one of the abovementioned processes, the term “compound” is to be understood broadly and means, in a general manner, all the material means which directly or indirectly bring about the desired effect. The term also encompasses, for example, nucleic acids or proteins, natural or artificial binding or interaction partners of an L119 protein or an L119 nucleic acid sequence, natural or artificial transcription factors, anti-L119 antibodies, L119-agonists or antagonists, a peptidomimetic of an L119 agonist or antagonist, or low molecular weight compounds.

Preferred low molecular weight compounds are those which

a) possess a molecular weight of less than 2000 g/mol, preferably less than 1000 g/mol, particularly preferably less than 750 g/mol, most preferably less than 500 g/mol, and

b) bind to one of the L119 proteins according to the invention with a binding constant of less than 10 μM, preferably less than 1 μM, particularly prefeerably less than 100 nM, most preferably less than 10 nM.

Binding or modulation or normalization is generally detected by measuring the interaction with one of the L119 proteins or nucleic acids according to the invention, by measuring the increase or decrease of at least one essential property, or the expression of one of the L119 proteins according to the invention, or the L119 activity, or by measuring a physiological effect of L119.

For this purpose, it is possible to use direct or indirect detection methods, as are familiar to the skilled person, for finding interaction partners and/or signal transduction pathways. These methods comprise, for example,

a) a number of methods which are summarized under the term “yeast N-hybrid” system

b) antibody selection techniques

c) phage display systems

d) immunoprecipitations

e) immunoassays such as ELISA or Western blotting

e) reporter test systems

f) the screening of libraries of low molecular weight compounds,

g) molecular modeling using structural information relating to an L119 protein or nucleic acid.

The proteins, nucleic acid sequences, nucleic acid constructs or transgenic organisms according to the invention can be used for finding compounds, for example proteins, which exhibit specific binding affinities for the protein according to the invention, or for identifying nucleic acids which encode proteins which possess specific binding affinities for a protein according to the invention.

Yeast-N-hybrid systems, such as the yeast-2-hybrid system, or other biochemical methods, alone or in combination, are advantageously used for this purpose. In this way, it is possible to determine interaction domains of the protein according to the invention and thus points of pharmacotherapeutic intervention. The invention therefore also relates to the use of a yeast-N-hybrid system, or of biochemical methods, for identifying interaction domains of L119, and also to their use for pharmacotherapeutic intervention.

Substances which possess a specific binding affinity can also be found, in a specific manner, by analyzing the structure of the protein according to the invention. Substances of this nature can also be used as pro-L119 or anti-L119 compounds in accordance with the definition given below.

The processes according to the invention encompass processes (screening assays) for finding compounds which bind to L119 proteins or nucleic acids or which modulate or normalize at least one essential property, or the expression, of one of the L119 proteins according to the invention or of L119 activity.

The compounds which are to be tested for the desired property can be produced, for example, using one of the numerous methods for generating combinatorial libraries. These libraries can comprise biological and/or synthetic libraries. The skilled person is familiar with the methods for preparing these libraries (Lam K S (1997) Anticancer Drug Des. 12:145; DeWitt et al. (1993) Proc Natl Acad Sci USA 90: 6909; Erb et al. (1994) Proc Natl Acad Sci USA 91:11422; Zuckermann et al. (1994) J Med Chem 37:2678; Cho et al. (1993) Science 261:1303; Carrell et al. (1994) Angew Chem Int Ed Engl. 33:2059; Carell et al. (1994) Angew Chem Int Ed Engl. 33:2061; Gallop et al. (1994) J Med Chem 37:1233). The libraries can be present in solution (Houghten (1992) Biotechniques 13:412-421) or coupled to solid phases such as spheres (Lam (1991) Nature 354:82-84), chips (Fodor (1993) Nature 364:555-556), bacteria (Ladner U.S. Pat. No. 5,223,409), plasmids (Cull et al. (1992) Proc Natl Acad Sci USA 89:1865-1869) or be present on phages (for example within the context of a phage display system; Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310).

Furthermore, the process (screening assay) can be effected using cells and comprise, for example, incubating a cell, which is expressing an L119 protein, with a compound to be tested and subsequently determining the modulation or normalization of at least one esssential property of the L119 protein.

Determining the ability to modulate or normalize at least one essential property of an L119 protein comprises, for example, modulating or normalizing the ability of an L119 protein to interact with one of its binding partners, determined, for example, using the yeast two-hybrid approach (see Example 9). The ability of a compound to augment or diminish such an interaction can be detected, for example, by means of an immunoprecipitation, where appropriate in combination with a labeling (for example a radioactive labeling) of at least one of two interaction partners. The skilled person can use customary methods, such as gel electrophoresis and immunoblotting, in this connection.

Furthermore, the binding or modulation or normalization can also be determined using other methods, such as using a microphysiometer (McConnell H M et al. (1992) Science 257:1906-1912). In addition to this, it is possible to use cell-free methods (e.g. “real-time biomolecular interaction analysis (BIA)”; Sjolander S and Urbaniczky C (1991) Anal Chem 63:2338-2345; Szabo et al. (1995) Curr Opin Struct Biol 5:699-705). The skilled person is familiar with appropriate methods. The instruments which are required for the determination are commercially available (e.g. BIAcore).

Furthermore, binding partners can also be obtained from biological samples using techniques such as SELDI (surface-enhanced laser desorption ionization; CIPHERGEN Inc., Fremont, Calif., USA).

Cell-free test systems can contain both soluble and membrane-bound L119 proteins. In the case of membrane-bound proteins, it can be desirable to add a solubilizing agent in order to keep the protein in solution. Solubilizing agents comprise, for example, nonionic detergents such as N-octyl glucoside, N-dodecyl glucoside, N-dodecyl maltoside, octanoyl-N-methyl glucamide, decanoyl-N-methyl glucamide, Triton® X-100, Triton® X-114, Thesit®, isotridecylpoly(ethylene glycol ether), 3-[(3-cholamidopropyl)dimethylammonio]-1-propanesulfonate (CHAPS), 3-[(3-cholamidopropyl)dimethylammonio]-2-hydroxy-1-propanesulfonate (CHAPSO) and N-dodecyl-N,N-dimethyl-3-ammonio-1-propanesulfonate.

In one of the abovementioned methods, it may be advantageous to immobilize one of the L119 proteins according to the invention, or one of its interaction partners, in order, for example, to enable the bound form, or the non-bound form, to be separated off. The immobilization can be effected in many different ways which are known to the skilled worker. It can, for example, be effected on the walls of, for example, microtiter plates or microreaction tubes. However, it can also be effected on a matrix, for example using a GST/L119 fusion protein or a biotin-labeled L119 protein.

In a process according to the invention, an L119 protein can be used as the “bait protein in a two-hybrid assay or three-hybrid assay (U.S. Pat. No. 5,283,317; Zervos et al. (1993) Cell 72:223-232; Madura et al. (1993) J Biol Chem 268:12046-12054; Bartel et al. (1993) Biotechniques 14:920-924; Iwabuchi et al. (1993) Oncogene 8:1693-1696; WO 94/10300) in order to identify interaction partners for the L119 protein. Within the context of this invention, these systems are defined generally as “N-hybrid systems”. The way in which these systems work, and the implementation of these systems, have been described in detail and are known to the skilled person. While N-hybrid systems are preferably implemented in yeast, they can also be implemented in other eukaryotic cells such as mammalian cells. Appropriate systems are either commercially available or can readily be derived from commercially available systems.

In order to identify binding partners or compounds which modulate or normalize at least one essential property or the expression of an L119 protein (for example anti-L119 or pro-L119 compounds), it is possible, in particular, to use methods such as the “yeast-3-hybrid” system (Griffith E C et al. (2000) Methods Enzymol 328:89-103. Licitra E J and Liu J O (1996) Proc Natl Acad Sci USA 93(23):12817-21; Topcu Z and Borden K L (2000) Pharm Res 17(9):1049-55; Kraemer B et al. (2000) Methods Enzymol 328:297-321; Zhang J (2000) Methods Enzymol 328:103-10). The systems which are described in these publications can be used to identify compounds (low molecular weight compounds, proteins and nucleic acids) which interact with a particular protein, preferably an L119 protein, or which augment or diminish the interaction of this protein with other interaction partners.

One part of the subject matter of the invention relates to antibodies which recognize one of the L119 proteins according to the invention. In the first place, such antibodies themselves constitute compounds which possess a specific binding affinity for one of the proteins according to the invention and/or are able to modulate or normalize at least one essential property of an L119 proteins. Such antibodies can be identified using one of the abovementioned processes. In-the second place, these antibodies can be used in one of the abovementioned processes for finding compounds which bind specifically to one of the proteins according to the invention or modulate or normalize at least one property, or the expression, of the same. Thus, using antibodies, it ispossible to determine the activity or the quantity of the proteins having the sequences SEQ ID NO: 3, 6, 7 or 24. For this reason, the invention also relates to a process for quantifying the activity or quantity of a protein having the sequences SEQ ID NO: 3, 6, 7 or 24.

Proceeding from the amino acid sequences SEQ ID NO: 3, 6, 7 or 24, it is possible to generate synthetic peptides or recombinant proteins which are then used as antigens for producing antibodies. It is also possible to employ the isolated protein itself, or fragments thereof, for generating antibodies.

Antibodies are understood as meaning polyclonal, monoclonal, human or humanized, or recombinant antibodies, or fragments thereof, single-chain antibodies or synthetic antibodies. Antibodies according to the invention, or their fragments, are in principle to be understood as meaning all the immunoglobulin classes, such as IgM, IgG, IgD, IgE and IgA, or their subclasses, such as the IgG subclasses, or their mixtures. Preference is given to IgG and its subclasses, such as IgG₁, IgG₂, IgG₂ₐ, IgG_{2b},

IgG₃ and IgG_{M}. Particular preference is given to the IgG subtypes IgG₁/κ or IgG_{2b}/κ. Fragments which may be mentioned are all the truncated or modified antibody fragments which have one or two binding sites which are complementary to the antigen, such as antibody moieties having a binding site which corresponds to the antibody and which is formed from a light chain and a heavy chain, such as Fv, Fab or F(ab′)₂ fragments or single-strand fragments. Preference is given to truncated double-strand fragments, such as Fv, Fab and F(ab′)₂. These fragments can be obtained, for example, either enzymically, by cleaving off the Fc moiety of the antibodies using enzymes such as papain or pepsin, or by means of chemical oxidation or by means of recombinantly manipulating the antibody genes. Genetically manipulated non-truncated fragments can also be advantageously used.

Monoclonal antibodies can be obtained, in the manner with which the skilled person is familiar, for example, by means of the hybridoma technique (Kohler and Milstein (1975) Nature 256:495-497; Brown et al. (1981) J Immunol 127:539-46; Brown et al. (1980) J Biol Chem 255:4980-83; Yeh et al. (1976) Proc Natl Acad Sci USA 76:2927-31; Yeh et al. (1982) Int J Cancer 29:269-75). Variants which are suitable in accordance with the invention are the human B cell hybridoma technique (Kozbor et al. (1983) Immunol Today 4:72), the EBV hybridoma technique (Cole et al. (1985), Monoclonal Antibodies and Cancer Therapy, Alan R. Liss, Inc., pp. 77-96) or the trioma techniques. The methods for preparing appropriate hybridomas are also known to the skilled person (Kenneth R H in Monoclonal Antibodies: A New Dimension In Biological Analyses, Plenum Publishing Corp., New York, N.Y. (1980); Lerner E A (1981) Yale J Biol Med 54:387-402; Gefter M L et al. (1977) Somatic Cell Genet. 3:231-36; Galfre G et al. (1977) Nature 266:55052). A large number of suitable myeloma cell lines are known to the skilled person (e.g. P3-NS1/1-Ag4-1, P3-x63-Ag8.653 or Sp2/b-Ag14 myeloma cell lines) and can be obtained, for example, from the ATCC (American Type Culture Collection). Positive hybridoma cell lines can be selected in the manner with which the skilled person is familiar, for example using an ELISA technique or using the protein (for example an L119 protein) which is employed for the immunization.

Alternatively, it is also possible to identify monoclonal anti-L119 antibodies by screening a combinatorial immunoglobulin library (e.g. a phage-display library of antibodies) using the relevant L119 protein. Kits for preparing and screening phate-display libraries are commercially available (Pharmacia Recombinant Phage Antibody System; Stratagene SurfZAP™ Phage Display Kit). Other methods which are preferred in this context are known to the skilled person (U.S. Pat. No. 5,223,409; WO 92/18619; WO 91/17271; WO 92/20791; WO 92/15679; WO 93/01288; WO 92/01047; WO 92/09690; WO 90/02809; Fuchs et al. (1991) Bio/Technology 9:1370-1372; Hay et al. (1992) Hum Antibod Hybridomas 3:81-85; Huse et al. (1989) Science 246: 1275-1281; Griffiths et al. (1993) EMBO J 12:725-734; Hawkins et al. (1992) J Mol Biol 226:889-896; Clarkson et al. (1991) Nature 352:624-628; Gram et al. (1992) Proc Natl Acad Sci USA 89:3576-3580; Garrad et al. (1991) Bio/Technology 9:1373-1377; Hoogenboom et al. (1991) Nuc Acid Res 19:4133-4137; Barbas et al. (1991) Proc Natl Acad Sci USA 88:7978-7982; and McCafferty et al. Nature (1990) 348:552-554).

It is furthermore possible to use standard methods to obtain recombinant anti-L119 antibodies, for example chimeric or humanized monoclonal antibodies, which contain both human and nonhuman moieties, within the context of this invention (WO 87/02671; EP 0 184 187; EP 0 171 496; EP 0 173 494; WO 86/01533; U.S. Pat. No. 4,816,567; EP 0 125 023; Better et al. (1988) Science 240:1041-1043; Liu et al. (1987) Proc Natl Acad Sci USA 84:3439-3443; Liu et al. (1987) J Immunol 139:3521-3526; Sun et al. (1987) Proc Natl Acad Sci USA 84:214-218; Nishimura et al. (1987) Canc Res 47:999-1005; Wood et al. (1985) Nature 314: 446-449; Shaw et al. (1988) J Natl Cancer Inst 80:1553-1559; Morrison S L (1985) Science 229:1202-1207; Oi et al. (1986) BioTechniques 4:214; U.S. Pat. No. 5,225,539; Jones et al. (1986) Nature 321:552-525; Verhoeyan et al. (1988) Science 239:1534; Beidler et al. (1988) J Immunol 141:4053-4060).

The antibody genes which are required for the recombinant manipulation can be isolated in a manner known to the skilled person, for example from the hybridoma cells (Harlow E and Lane D (1988) Antibodies: A Laboratory Manual, Cold Spring Harbor Press, New York; Ausubel et al., 1998). For this, antibody-producing cells are grown and, when the cells have reached an adequate optical density, the mRNA is then isolated from the cells, in a known manner, by means of lysing the cells with guanidinium thiocyanate, then acidifying with sodium acetate, extracting with phenol and chloroform/isoamyl alcohol, precipitating with isopropanol and washing with ethanol. After that, cDNA is synthesized from mRNA using reverse transcriptase. The synthesized cDNA can be inserted into suitable animal, fungal, bacterial or viral vectors, either directly or after genetic manipulation, for example by means of site-directed mutagenesis or the introduction of insertions, inversions, deletions or base exchanges, and then expressed in the appropriate host organisms. Preference is given to bacterial or yeast vectors such as pBR322, pUC18/19, pACYC184, lambda or yeast mu vectors for cloning the genes and to expression in bacteria, such as E. coli, or in yeast, such as Saccharomyces cerevisiae.

An anti-L119 antibody can be used, for example, to isolate a natural or recombinant L119 protein from biological material, such as cells, by means of standard methods such as affinity chromatography or immunoprecipitation. In addition to this, such an antibody can be used for detecting an L119 protein (for example in a cell lysate or cell supernatant). Anti-L119 antibodies can be used in diagnostic methods in order, for example, to determine the tissue level of an L119 protein. In this way it is possible to determine, for example, the necessity and/or the efficiency of an L119-modulating or -normalizing therapy. For the purpose of the detection, an anti-L119 antibody is preferably labeled with a detectable compound.

The skilled person is familiar with the methods for preparing these antibodies or protein-binding or DNA-binding factors (Famulok M and Jenne A; Curr Opin Chem Biol 1998, 2(3):320-7; Current Protocols in Protein Science. Volume 1. Coligon, J E, Dunn, B M, Plough, H L, Speicher, D W, Wingfield, P T eds. John Wiley & Sons, Inc. (1995) Chapter 9: Purification of DNA-Binding Proteins, Chapter 19: Identification of Protein Interactions, Antibody Production: Essential Techniques. Delves P (1997) John Wiley & Sons, Inc. New York; Antibody Technology: A Comprehensive Overview; Liddell J E and Weeks I (1995) Bios Scientific Publishers, Ltd., United Kingdom; Owen M et al., Biotechnology (N Y). 1992; 10(7):790-794; Franken E et al., Curr Opin Biotechnol. 1997; 8(4):411-416; Whitelam Trend Plant Sci 1996, 1, 286-272).

The antibodies or fragments can be used either on their own or in mixtures.

Specific antibodies directed against the proteins according to the invention can be suitable for use both as diagnostic reagents and as therapeutic agents in association with syndromes which are characterized, inter alia, by changes in endothelial cells.

Other embodiments of the invention are represented by processes for finding compounds which decrease or increase the interaction of ligands with the protein heteromer according to the invention or the proteins according to the invention having amino acid sequences as depicted in SEQ ID NO: 3, 6, 7 or 24, or a process for finding substances which decrease or increase the interaction of proteins having amino acid sequences such as SEQ ID NO: 3, 6, 7 or 24 with the proteins described in Table 1 or other signal transduction molecules. The interaction of proteins containing the amino acids in accordance with the invention can be detected using the two-hybrid system. Substances of this nature can likewise be used as pro-L119 or anti-L119 compounds in accordance with the definition given below.

In addition, the processes can be carried out by expressing the proteins in eukaryotic cells and linking to a reporter assay for the activation of the L119 protein.

The invention furthermore relates to a process for qualitatively and quantitatively determining proteins having amino acid sequences such as SEQ ID NO: 3, 6, 7 or 24 using specific agonists or antagonists. In this connection advantage is taken of the L119 ligand binding for the detection.

“Modulation” or “modulate” means the increase or decrease of at least one essential property, or the expression, of an L119 protein.

“Normalize” means that at least one essential property, or the expression, of one of the L119 proteins according to the invention in the recombinantly treated organism corresponds by at least 20%, preferably by at least 50%, particularly preferably by at least 90%, to a normal value which is obtained from a healthy individual or to a mean value which is obtained from several healthy individuals, or exceeds this value by not more than 500%, preferably by not more that 200%, particularly preferably by not more than 100%, very particularly preferably by not more than 50%.

In this connection, “pro-L119 compound” means, in a general manner, those compounds which bring about an increase of at least one essential property or of the expression, of an L119 protein, preferably of an L119 protein as depicted in SEQ ID NO: 3, 6, 7 or 24, or of a functionl equivalent thereof, in a cell or an organism.

“Anti-L119 compound” means, in a general manner, those compounds which bring about a decrease in at least one essential property, or in the expression, of an L119 protein, preferably of an L119 protein as depicted in SEQ ID NO: 3, 6, 7 or 24, or of a functional equivalent thereof, in a cell or an organism.

In relation to the pro-L119 or anti-L119 compound, the term “compound” is to be understood broadly and means, in a general manner, all the material means which directly or indirectly bring about the desired effect. By way of example, but not in a limiting manner, pro-L119 or anti-L119 compounds can be nucleic acids or proteins, natural or artificial binding or interaction partners of an L119 protein, antibodies, L119 agonists or antagonists, a peptidomimetic of an L119 agonist or antagonist, antisense nucleic acids, apatamers, natural or artificial transcription factors, nucleic acid constructs, vectors or low molecular weight compounds.

Pro-L119 or anti-L119 compounds may be identical to compounds which can be obtained using one of the processes according to the invention and which bind to one of the novel nucleic acid molecules or proteins or modulate or normalize at least one property, or the expression, of an L119 protein. The given definitions and term clarifications are mutually inclusive.

Preferred low molecular weight “pro-L119” or “anti-L119” compounds are such that they

a) have a molecular weight of less than 2000 g/mol, preferably less than 1000 g/mmol, particularly preferably less than 750 g/mol, most preferably less than 500 g/mol, and

b) bind to one of the L119 proteins according to the invention with a binding constant of less than 10 μM, preferably less than 1 μM, particularly preferably less than 100 nM, most preferably less than 10 nM.

In connection with the modulation or normalization of at least one important property, or of the expression, of one of the L119 proteins according to the invention, the term “increase” is to be interpreted widely and covers the increase in at least one function of an L119 protein, when using a pro-L119 compound, in an organism or a part derived therefrom, or in cells or tissue. The invention encompasses different strategies for increasing a function of an L119 protein. The skilled person will recognize that a number of different methods are available for influencing a function of the L119 protein in a desired manner. In this respect, the methods which are described below are to be understood as being by way of example and not limiting.

The strategy which is preferred in accordance with the invention comprises using, as the pro-L119 compound, a nucleic acid sequence which can be transgenically transcribed and, where appropriate, translated into a polypeptide which increases at least one function of the L119 protein. The above-described L119 nucleic acid sequences as depicted in SEQ ID NO: 1, 2, 4, 5, 22 or 23, or their functional equivalents, are particularly preferred for nucleic acid sequences of this nature.

In addition, it is also possible to increase a function of an L119 protein by, for example, mutagenizing endogenous genes, preferably L119 genes, or the factors which regulate their expression. Furthermore, an elevated transcription and translation of the endogenous L119 genes can be achieved, for example, by using artificial transcription factors, for example of the zinc finger protein type. These factors bind to the regulatory regions of the endogenous genes and, depending on the configuration of the factor, cause the endogenous gene to be expressed or repressed. The use of such a method makes it possible to repress or overexpress a particular endogenous gene without having to recombinantly manipulate its sequence. Appropriate methods for preparing the corresponding factors have been described and are known to the skilled person (Beerli R R et al., Proc Natl Acad Sci USA. 2000; 97 (4):1495-1500; Beerli R R, et al., J Biol Chem 2000; 275(42):32617-32627; Segal D J and Barbas C F 3rd., Curr Opin Chem Biol 2000; 4(1):34-39; Kang J S and Kim J S, J Biol Chem 2000; 275(12):8742-8748; Beerli R R et al., Proc Natl Acad Sci USA 1998; 95(25):14628-14633; Kim J S et al., Proc Natl Acad Sci USA 1997; 94(8):3616-3620; Klug A, J Mol Biol 1999; 293(2):215-218; Tsai S Y et al., Adv Drug Deliv Rev 1998; 30(1-3):23-31; Mapp A K et al., Proc Natl Acad Sci USA 2000; 97(8):3930-3935; Sharrocks A D et al., Int J Biochem Cell Biol 1997; 29(12):1371-1387; Zhang L et al., J Biol Chem 2000; 275(43):33850-33860). The factors can be selected using the promoter region of the gene for an L119 protein. The skilled person can obtain the corresponding segments from Genbank by means of database interrogation or else with the aid of the L119 nucleic acid sequences according to SEQ ID NO: 1, 2, 4, 5, 22 or 23, which were prepared within the context of this invention, or else proceeding from an L119 cDNA, whose gene is not present in Genbank, by means of screening a genomic library for corresponding genomic clones. The skilled person is familiar with the methods which are required for doing this. Factors can, for example, be isolated by using a reporter system in which the promoter region of an L119 gene is linked to a label, for example Luciferase or GFP (green fluorescence protein), and controls the expression of this label instead of that of an L119 protein. Using such nucleic acid constructs according to the invention, it is possible, following introduction into a suitable expression system, to assess compounds with regard to their effect on the expression activity of the L119 promoter.

Compounds which bring about one of the above-described methods for increasing an essential L119 property must be understood as being pro-L119 compounds. In this connection, the quantity of an L119 protein, or at least one of its essential properties, is increased, in a cell or an organism, by at least 50%, preferably at least 100%, particularly preferably at least 500%, very particularly preferably at least 1000%.

In connection with modulating or normalizing at least one essential property, or the expression, of one of the L119 proteins according to the invention, the term “decrease” is to be interpreted widely and comprises the partial, or essentially complete, suppression or blocking, based on different cell-biological mechanisms, of at least one essential property, or of the expression, of an L119 protein, when using an anti-L119 compound, in an organism, or a part derived therefrom, or in cells or tissue. The organism is preferably a mammal. A decrease within the meaning of the invention also encompasses a quantitative decrease in an L119 protein through to an essentially complete absence of the L119 protein (i.e. the inability to detect an essential L119 property or the inability to detect an L119 protein immunologically). In this connection, the expression of a given L119 protein, or at least one of its essential properties, is decreased in a cell or an organism by preferably more than 50%, particularly preferably by more than 80%, very particularly preferably by more than 90%.

The invention encompasses various strategies for decreasing the essential L119 property. The skilled person will recognize that a number of different methods are available for influencing the essential L119 property in the desired manner.

The strategy which is preferred in accordance with the invention comprises using an L119 nucleic acid sequence as an anti-L119 compound which can be transcribed into an antisense nucleic acid sequence which is capable of decreasing the expression of an L119 protein, for example by decreasing the expression of the corresponding endogenous L119 protein. In accordance with a preferred embodiment, the anti-L119 nucleic acid sequences can contain the nucleic acid sequence encoding an L119 protein, or functional equivalents or functionally equivalent fragments thereof, inserted in the antisense orientation.

An “antisense” nucleic acid means, first of all, a nucleic acid sequence which is entirely or partially complementary to a part of the “sense” strand of an L119 nucleic acid sequence (i.e. of the strand which encodes a corresponding L119 protein). L119 nucleic acid sequences which are preferred in this connection are those which encode proteins which are described by SEQ ID NO: 3, 6, 7 or 24, or their functional equivalents or functionally equivalent parts thereof. Particular preference is given to L119 nucleic acids which are described by SEQ ID NO: 1, 2, 4, 5, 22 or 23, or their functional equivalents or functionally equivalent parts thereof. The abovementioned nucleic acid sequences as depicted in SEQ ID NO: 1, 2, 5 or 23 describe L119 cDNA sequences. The sequences depicted in SEQ ID NO: 4 or-22 describe L119 genes which still contain introns. The skilled person is aware of the fact that he is able alternatively to use cDNA or the corresponding gene as the starting template for appropriate antisense constructs.

The “antisense” nucleic acid is preferably complementary to the coding region of an L119 nucleic acid sequence or a part thereof. However, the “antisense” nucleic acid can also be complementary to the non-coding region or a part thereof. Proceeding from the sequence information for an L119 nucleic acid sequence as depicted in SEQ ID NO: 1, 2, 4, 5, 22 or 23, or a functional equivalent thereof, it is possible to design an antisense nucleic acid while observing the Watson and Crick base pairing rules in the manner with which the skilled person is familiar. An antisense nucleic acid can be complementary to all or part of an L119 nucleic acid sequence. In a preferred embodiment, the antisense nucleic acid is an oligonucleotide having a length of, for example, 15, 20, 25, 30, 35, 40, 45 or 50 nucleotides.

An antisense nucleic acid can be prepared chemically and/or enzymically using methods with which the skilled person is familiar. In this connection, it is possible to use natural or non-natural nucleotide building blocks. Non-natural nucleotide building blocks comprise modified nucleotides whose incorporation increases the biological stability of the antisense nucleic acid or the physical stability of the duplex which is formed between the antisense nucleic acid and the sense nucleic acid. Phosphorothioate derivatives and acridine-substituted nucleotides may be mentioned by way of example. The following may be mentioned by way of example: 5-fluorouracil, 5-bromouracil, 5-chlorouracil, 5-iodouracil, hypoxanthine, xanthine, 4-acetylcytosine, 5-(carboxyhydroxymethyl)uracil, 5-(carboxymethylaminomethyl)-2-thiouridine, 5-(carboxymethylaminomethyl)uracil, dihydrouracil, β-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, pseudouracil, queosine, 2-thiocytosine, 5-methyl-2-thiouracil, 2-thiouracil, 4-thiouracil, 5-methyluracil, methyl uracil-5-oxyacetate, uracil-5-acetic acid, 5-methyl-2-thiouracil, 3-(3-amino-3-N-2-carboxypropyl)uracil and 2,6-diaminopurine.

Alternatively, an antisense nucleic acid can also be produced biologically using an expression vector into which the corresponding nucleic acid has been inserted, in the antisense orientation, downstream of a suitable promoter. In order to achieve appropriate intracellular concentrations, the antisense nucleic acid which is to be expressed can be placed under the control of strong promoters such as the pol II promoter or the pol III promoter. This method is preferably employed in combination with the methods which are suitable for a recombinant approach.

In a preferred embodiment, the antisense nucleic acid encompasses α-anomeric nucleic acid molecules. α-Anomeric nucleic acid molecules form special double-stranded hybrids with complementary RNA, in which hybrids the strands run parallel to each other, in contrast to the normal β units (Gaultier et al. (1987) Nucleic Acids Res. 15:6625-6641).

The antisense nucleic acid furthermore comprises 2′-o-methylribonucleotides (Inoue et al. (1987) Nucleic Acids Res. 15:6131-6148) or chimeric RNA-DNA analogs (Inoue et al. (1987) FEBS Lett. 215:327-330).

The invention also encompasses the use of the above-described sequences in the sense orientation which, as the skilled person is aware,, can lead to cosuppression, and also to the use of the sequences within the context of methods such as gene regulation using double-stranded RNA (“double-stranded RNA interference”). Appropriate methods are known to the skilled person and have been described in detail (e.g. Matzke M A et al. (2000) Plant Mol Biol 43:401-415; Fire A. et al (1998) Nature 391:806-811; WO 99/32619; WO 99/53050; WO 00/68374; WO 00/44914; WO 00/44895; WO 00/49035; WO 00/63364). The processes and methods which are described in the abovementioned reference citations are hereby expressly incorporated by reference.

The antisense strategy can advantageously be coupled to a ribozyme method. Ribozymes are catalytically active RNA sequences which, when coupled to the antisense sequences, catalytically cleave the target sequences (Tanner N K. FEMS Microbiol Rev. 1999; 23 (3):257-75). This can increase the efficiency of an antisense strategy. The expression of ribozymes for the purpose of decreasing particular proteins is known to the skilled person and is described, for example, in EP-A1 0 291 533, EP-A1 0 321 201 and EP-A1 0 360 257. Suitable target sequences and ribozymes can be determined, for example as described in Steinecke (Ribozymes, Methods in Cell Biology 50, Galbraith et al., eds., Academic Press, Inc. (1995), 449-460), by means of secondary structure calculations of ribozyme and target RNA and also by means of their interaction (Bayley C C et al., Plant Mol Biol. 1992; 18(2):353-361; Lloyd A M and Davis R W et al., Mol Gen Genet. 1994 Mar; 242(6):653-657). “Hammerhead” ribozymes may be mentioned by way of example (Haselhoff and Gerlach (1988) Nature 334:585-591). Preferred ribozymes are based on derivatives of Tetrahymena L-19 IVS RNA (U.S. Pat. No. 4,987,071; U.S. Pat. No. 5,116,742). It is possible to select additional ribozymes which have selectivity for an L119 mRNA (Bartel D und Szostak J W (1993) Science 261:1411-1418).

In another embodiment, an L119 can be expressed using nucleic acid sequences which are complementary to regulatory elements of the endogenous L119 genes and form a triple-helical structure with these genes and thereby prevent gene transcription (Helene C (1991) Anticancer Drug Des. 6(6):569-84; Helene C et al. (1992) Ann NY Acad Sci 660:27-36; Maher L J (1992) Bioassays 14(12):807-815).

In another embodiment, L119 nucleic acids, or antisense nucleic acids which are complementary to them, can be modified on the base subunit, the sugar subunit or the phosphate subunit in order, for example, to improve the stability, the hybridization or the solubility. For example, it is possible to use peptide nucleic acids (PNAs) (Hyrup B et al. (1996) Bioorganic & Medicinal Chemistry 4(l):5-23). In these nucleic acids, the deoxyribose phosphate backbone chain is replaced with a pseudopeptide backbone chain. Only the four natural nucleobases are retained. The skilled person is familiar with the synthesis of such compounds (Hyrup B et al. (1996) see above; Perry-O'Keefe et al. Proc Natl Acad Sci USA 93: 14670-675). Such PNAs can be used in diagnostic and therapeutic methods.

In another embodiment, it is possible to add additional groups, such as peptides, to one of the nucleic acid sequences according to the invention (e.g. in order to achieve transport through the cell membrane (Letsinger et al. (1989) Proc Natl Acad Sci USA 86:6553-6556; Lemaitre et al. (1987) Proc Natl Acad Sci. USA 84:648-652; WO 88/09810), or through the blood brain barrier (WO 89/10134), or to target particular cell types by way of particular receptors).

Other methods are the introduction of nonsense mutations, or mutations which decrease an essential L119 property, into endogenous L119 genes using, for example, recombinant approaches, for example using RNA/DNA oligonucleotides.

It is furthermore also possible to decrease the expression of an L119 gene using specific DNA-binding factors, for example using factors of the zinc finger transcription factor type. These factors preferentially bind to the regulatory regions of the genomic sequence of the endogenous target gene and, depending on the configuration of the factor, bring about expression or repression of the endogenous gene. The use of such a method makes it possible to decrease the expression of an endogenous L119 gene without having to manipulate its sequence recombinantly. Appropriate methods for preparing corresponding factors have been described and are known to the skilled person (Beerli R R et al., Proc Natl Acad Sci USA 2000; 97 (4):1495-1500; Beerli R R, et al., J Biol Chem 2000; 275(42):32617-32627; Segal D J and Barbas C F 3rd., Curr Opin Chem Biol 2000; 4(1):34-39; Kang J S and Kim J S, J Biol Chem 2000; 275(12):8742-8748; Beerli R R et al., Proc Natl Acad Sci USA 1998; 95(25):14628-14633; Kim J S et al., Proc Natl Acad Sci USA 1997; 94(8):3616-3620; Klug A, J Mol Biol 1999; 293(2):215-218; Tsai S Y et al., Adv Drug Deliv Rev 1998; 30(1-3):23-31; Mapp A K et al., Proc Natl Acad Sci USA 2000; 97(8):3930-3935; Sharrocks A D et al., Int J Biochem Cell Biol 1997; 29(12):1371-1387; Zhang L et al., J Biol Chem 2000; 275(43):33850-33860). These factors can be selected using any arbitrary segment of the gene of an L119 protein. This segment is preferably located in the promoter region. However, when a gene is to be suppressed, it can also be located, in contrast to when a gene is to be activated, in the region of the coding exons or introns. The skilled person can obtain the corresponding segments from Genbank by means of database interrogation or using the L119 nucleic acid sequences which are depicted in SEQ ID NO: 1, 2, 4, 5, 22 or 23, and which were prepared within the context of the invention, or else proceeding from L119 cDNA, whose gene is not in Genbank, by means of screening a genomic library for corresponding genomic clones. The skilled person is familiar with the methods which are required to do this. Factors can be isolated, for example, by using a reporter system in which the promoter region of an L119 gene is linked to a label, for example Luciferase or GFP (green fluorescence protein), and controls the expression of this marker instead of that of an L119 protein. Following introduction into a suitable expression system, such nucleic acid constructs according to the invention can be used to assess compounds with regard to their effect on the expression activity of the L119 promoter.

The regulatory sequences of the L119 nucleic acids according to the invention, in particular the promoter, the enhancers, the locus control regions and silencers, or given part sequences thereof, can be used for the tissue-specific expression of this gene and other genes. This results in the possibility of expressing genes in nucleic acid constructs in an endothelium-specific manner. The preferred application of this possibility is its use as a point of attack for preparing or selecting novel anti-L119 or pro-L119 compounds.

In order to isolate a DNA fragment which contains the regions which regulate the transcription of the sequences SEQ ID NO: 1, 2, 4, 5, 22 or 23, the region upstream of the transcription start is first of all linked to a reporter gene, such as β-galactosidase or GFP (=green fluorescent protein), and then tested in cells or in transgenic animals, for example in mice, to see whether it leads to the expression pattern which is specific for sequence SEQ ID NO: 1, 2, 4, 5, 22 or 23 (Ausubel F M et al., (1998) Current Protocols in Molecular Biology, John Wiley & Sons, New York). Since cis-regulatory sequences can, inter alia, also be located at a very great distance from the transcription start site, it is advantageous if very large genomic regions are included in the analysis. For the cloning, it can be advantageous to use vector systems which have a very high cloning capacity, such as BACs or YACs (bacterial artificial chromosome and yeast artificial chromosome), respectively. In this connection, the reporter gene can be inserted into the vector by way of homologous recombination and then investigated with regard to its expression (see, for example, Hiemisch H et al. (1997) EMBO J. 16, 3995-4006). By making suitable deletions in the construct and then examining the effects of these deletions on the expression of the reporter gene, it is possible to identify important regulatory elements (see, for example, Montoliu L et al. (1996) EMBO J 15, 6026-6034).

The regulatory sequences of the nucleic acids according to the invention identified in this way, in particular the promoter, the enhancer, the locus control regions and the silencers, or relevant part sequences thereof, can be used for finding specific pro-L119 or anti-L119 compounds. Furthermore, these sequences can be used for the tissue-specific expression of sequences SEQ ID NO: 1, 2, 4, 5, 22 or 23 and other genes. This thereby results in the possibility of expressing genes in nucleic acid constructs in an endothelium-specific manner. The construct containing the regulatory sequences can be linked to other cDNAs in order to construct animal models in which the respective cDNA is expressed in a region-specific manner (see, for example, Oberdick J et al. (1990) Science 248, 223-226). In this connection, it can be a matter of the expression of sequence-specific DNA recombinases, such as CRE recombinase or FLP recombinase, or their derivatives.

Control regions which have been identified in this way are preferential points of attack for pro-L119 or anti-L119 compounds in accordance with one of the above definitions.

In addition, factors which inhibit an L119 target protein itself or which specifically decrease an essential property can be introduced into a cell or an organism. The protein-binding factors or binding factors can, for example, be aptamers (Famulok M, und Mayer G. Curr Top Microbiol Immunol. 1999; 243:123-36) or antibodies or antibody fragments or single-chain antibodies. The isolation of these factors has been described and is known to the skilled person. For example, a cytoplasmic scFv antibody has been used to modulate the activity of the phytochrome A protein in recombinantly modified tobacco plants (Owen M et al., Biotechnology (N Y). 1992; 10(7):790-794; Franken E et al., Curr Qpin Biotechnol. 1997; 8(4):411-416; Whitelam Trend Plant Sci 1996, 1, 286-272). Corresponding methods can be implemented in any cells. The above-described documents, and the methods disclosed therein for regulating gene expression, are hereby expressly incorporated by reference.

The corresponding factors (as well as their expression systems or vehicle systems for introducing them into an organism), which directly or indirectly decrease at least one essential property of an L119 protein, are to be understood as being anti-L119 compounds within the meaning of the invention.

An anti-L119 compound within the meaning of the present invention is consequently selected, in particular, from:

a) antisense nucleic acid sequences, preferably antisense L119 nucleic acid sequences;

b) antisense nucleic acid sequences combined with a ribozyme method

c) nucleic acid sequences, preferably L119 nucleic acid sequences, which bring about gene regulation by means of double-stranded RNA,

d) nonsense mutants of endogenous L119-encoding nucleic acid sequences;

e) nucleic acid sequences encoding knockout mutants;

f) nucleic acid sequence which are suitable for homologous recombination;

g) nucleic acid sequences which encode specific DNA-binding or protein-binding factors having anti-L119 activity,

with the transgenic expression of each single one of these anti-L119 sequences being able to bring about a decrease in at least one essential property of an L119 protein within the meaning of the invention. It is also possible to conceive of a combined use. Other methods are known to the skilled person and can comprise the obstruction or suppression of the processing of an L119 nucleic acid or protein, of the transport of an L119 protein or its mRNA, the inhibition of binding to ribosomes, the inhibition of RNA splicing, the induction of an RNA-degrading enzyme and/or the inhibition of translation elongation or termination.

In a preferred embodiment, pro-L119 or anti-L119 compounds, or else binding factors against the novel nucleic acids or proteins, can be identified by means of screening combinatorial libraries which encode low molecular weight compounds, peptides or nucleic acid sequences (e.g. aptamers). The preparation of such libraries for nucleic acid sequences or peptides is based, for example, on using degenerate nucleotide sequences or degenerate oligonucleotides which are expressed, where appropriate, in the case of peptide libraries, in the form of phage-display libraries. Methods for preparing such degenerate oligonucleotides are known to the skilled person (see, for example, Narang S A (1983) Tetrahedron 39:3; Itakura et al. (1984) Annu Rev Biochem 53:323; Itakura et al. (1984) Science 198:1056; Ike et al. (1983) Nucleic Acid Res 11:477). Peptide libraries can also be obtained by cloning said libraries of nucleic acid sequences into suitable expression vectors, transforming the expression vectors into a suitable host and expressing the peptide under the conditions which are in each case suitable and adjusted to the expression vector and the host.

“Recursive ensemble mutagenesis” (REM) is another method for generating nucleic acid or peptide libraries (Arkin and Yourvan (1992) Proc. Natl. Acad. Sci. USA 89:7811-7815; Delgrave et al. (1993) Protein Engineering 6(3):327-331).

In accordance with the differing nature of the above-described approaches, while the anti-L119 sequence can exert its function directly (for example by inserting into an endogenous L119 gene), the function can also be exerted indirectly following transcription into an RNA (for example in the case of antisense approaches) or following transcription and translation into a protein (for example in the case of binding factors). Both anti-L119 which act directly and those which act indirectly are encompassed by the invention.

The invention furthermore relates to the use, for producing drugs, of the compounds which bind to one of the novel nucleic acids or proteins-or which are suitable for modulating or normalizing at least one essential property, or the expression, of an L119 protein. These compounds can be obtained using one of the abovementioned processes.

The compounds are preferably employed for the treatment and prophylaxis of human and animal diseases, in particular for the treatment and prophylaxis of “vascular and endothelial diseases”. Depending on the nature of a disease, either an increase or a decrease in an essential property, or in the expression, of one of the L119 proteins according to the invention may be advantageous.

“Vascular and endothelial diseases” includes but is not limited to diseases comprising vascular homeostasis diseases, endothelial diseases, coagulation diseases, thrombotic diseases and/or platelet diseases.

In the context of this invention, “vascular and endothelial diseases” firstly means, in a general manner, all those diseases in which an increase or decrease in an essential property, or the expression, of one of the L119 proteins according to the invention is advantageous.

In the context of this invention, “endothelial diseases” firstly means, in a general manner, all those diseases in which an increase or decrease in an essential property, or the expression, of one of the L119 proteins according to the invention is advantageous. The endothelium may be directly or indirectly involved in these diseases.

“Endothelial diseases” comprises, for example, tumor diseases, diseases in which angiogenesis or vasculogenesis is altered, diseases of the cardiovascular system, vascular diseases, diseases involving inflammatory processes, diseases involving hypoxic or ischemic cells or tissues, and diseases in which the status of blood vessels or lymph vessels has been altered. These diseases include, inter alia, various solid tumors, hemangiomas, hemangiosarcomas, Kaposi's sarcoma, prostate cancer, glioblastoma, metastasis and growth of mesenchymal tumors, various retinopathies, cardiac infarction, cardiac insufficiency, coronary heart diseases, cardiomyopathies, hypertension, angina pectoris, arrythmia, acute or chronic kidney failure, chronic cardiac insufficiency, renal insufficiency, subarachnoidal hemorrhages, migraine, pulmonary hypertension, Raynaud's syndrome, cerebral vasospasms, benign prostate hyperplasia, erection disturbances, glaucoma, ischemic kidney failure or kidney failure caused by intoxication, pancreatitis, gastrointestinal ulcers, asthma, arteriosclerosis, septic or endotoxic shock, endotoxin-induced organ failure, intravascular coagulation, restenosis following angioplasty and by-pass operations, hyperlipidemias, homocysteinuria, disturbances of hair growth or wound healing, menstruation disturbances, ischemia, stroke, acute myocardial infarction, CADASIL, epilepsies, gangrene, rheumatoid arthritis, psoriasis, diabetes, diabetic retinopathy, lung diseases, kidney diseases and chronic ulcers, amputations, wounds and vascular changes. The term also encompasses deficient supply of the placenta and other disturbances during pregnancy. In a general manner, preference is given to those diseases in which the L119 mRNA is upregulated or in which upregulation brings about a positive effect.

“Vascular homeostasis diseases”, “thrombotic diseases”, “coagulation diseases” and/or platelet diseases include but are not limited to diseases with abnormally increased blood clotting like thrombosis (deep-vein clot) or pulmonary embolism and diseases with abnormally decreased blood clotting like hemophilia, and platelet aggregation disorders like, e.g. von Willebrand-disease, and other pathological conditions of blood coagulation like, e.g. Disseminated Intravascular Coagulation (DIC).

In addition, “platelet disease” includes but is not limited to acquired platelet dysfunction, an acquired abnormality of platelet function, common because use of aspirin, which predictably affects platelet function. Many other drugs may also induce platelet dysfunction. Many clinical disorders (eg, myeloproliferative and myelodysplastic disorders, uremia, macroglobulinemia and multiple myeloma, cirrhosis, SLE) can affect platelet function as well. Patients with uremia caused by chronic renal failure may have a long bleeding time for unknown reasons. The bleeding time may shorten transiently after vigorous dialysis, administration of cryoprecipitate, or desmopressin infusion. Raising the RBC count by transfusion or by giving erythropoietin also causes the bleeding time to shorten.

“Platelet disease” also include hereditary intrinsic platelet disorders. The most common hereditary intrinsic platelet disorders are a group of mild bleeding disorders that may be considered disorders of amplification of platelet activation. They may result from decreased adenosine diphosphate (ADP) in the platelet-dense granules (storage pool deficiency), from an inability to generate thromboxane A2 from arachidonic acid released from the membrane phospholipids of stimulated platelets, or from an inability of platelets to respond normally to thromboxane A2. They present with a common pattern of platelet aggregation test results: (1) impaired-to-absent aggregation after exposure to collagen, epinephrine, and a low concentration of ADP and (2) normal aggregation after exposure to a high concentration of ADP. Aspirin and other NSAIDs may produce the same pattern of platelet aggregation test results in healthy persons. Because aspirin's effect can persist for several days, it must be confirmed that a patient has not taken aspirin for several days before testing to avoid confusion with a hereditary platelet defect.

Thrombasthenia is a rare hereditary platelet defect that affects platelet surface membrane glycoproteins. It is an autosomal recessive disorder. Consanguinity is common in affected families. Thrombasthenia patients may have severe mucosal bleeding (eg, nosebleeds that stop only after nasal packing and transfusions of platelet concentrates). Their platelets, lacking the membrane glycoprotein GP IIb-IIIa, fail to bind fibrinogen during platelet activation and thus fail to aggregate. Typical laboratory findings are failure of platelets to aggregate with any physiologic aggregating agent, including a high concentration of exogenous ADP; absence of clot retraction; and single platelets without aggregates on a peripheral blood smear of capillary blood obtained from a finger stick. Bernard-Soulier syndrome is another rare autosomal recessive disorder that affects surface membrane glycoproteins. Unusually large platelets are present that do not agglutinate with ristocetin but aggregate normally with the physiologic aggregating agents ADP, collagen, and epinephrine. A surface membrane glycoprotein (GP Ib-IX) that contains a receptor for VWF is missing from the platelet surface membrane in this disorder. Therefore, the platelets do not adhere normally to subendothelium despite normal VWF levels in plasma. Large platelets associated with functional abnormalities also may be found in the May-Hegglin anomaly, a thrombocytopenic disorder with abnormal WBCs, and in the Ch6diak-Higashi syndrome. Serious bleeding in a patient with an intrinsic platelet disorder may require platelet transfusion.

Depending on the disease increase or decrease in an essential property, or the expression, of one of the L119 proteins according to the invention might be advantageous.

Diseases where an increase in an essential property, or the expression, of one of the L119 proteins according to the invention is preferred include but are not limited to ischemic diseases like stroke or myocardial infarction and thrombotic diseases like, e.g. thrombosis (deep-vein clot) or pulmonary embolism.

Diseases where an decrease in an essential property, or the expression, of one of the L119 proteins according to the invention is preferred include but are not limited to diseases with abnormally descreased blood clotting like hemophilia, and platelet aggregation disorders like, e.g. von Willebrand-disease.

The invention relates to the use of the nucleic acids according to the invention, or parts thereof, of the nucleic acid constructs according to the invention, and of the pro-L119 or anti-L119 compounds according to the invention for gene therapy.

Preference is given to applying the above to mammals, particularly preferably humans. Preference is given to using the nucleic acid sequences which are described by SEQ ID NO: 1, 2, 4, 5, 22 or 23 and the transgenic nucleic acid constructs which are derived therefrom. Sequences which are complementary to the nucleic acids according to the invention or to parts thereof can also preferably be used for gene therapy.

“Gene therapy” encompasses, in a general manner, all the methods which are suitable for modulating or normalizing at least one essential property, or the expression, of one of the L119 proteins according to the invention.

“Normalizing” means that at least one essential property, or the expression, of one of the L119 proteins according to the invention in the organism which has been treated by gene therapy corresponds by at least 20%, preferably by at least 50%, particularly preferably by at least 90%, to a normal value which is obtained from a healthy individual or from a mean value for several healthy individuals, or does not exceed this normal value by more than 500%, preferably by not more than 200%, particularly preferably by not more than 100%, very particularly preferably by not more than 50%.

The invention also encompasses all the forms of therapy which either introduce sequences in accordance with SEQ ID NO: 1, 2, 4, 5, 22 or 23, or their functional equivalents or parts thereof, into an organism, for example into a human body, or modulate or normalize an essential property, or the expression, of a protein according to the invention as depicted in SEQ ID NO: 3, 6, 7 or 24, or of a nucleic acid sequence as depicted in SEQ ID NO: 1, 2, 4, 5, 22 or 23, or of their functional equivalents.

Preference is furthermore given to using pro-L119 or anti-L119 compounds insofar as they are nucleic acid sequences or can be obtained from nucleic acid sequences by transcription and, where appropriate, translation as well. These can include, for example: oligonucleotides, e.g. antisense or hybrid RNA-DNA oligonucleotides or double-stranded RNA molecules which possess any arbitrary modifications and which contain, for example parts of the sequences as depicted in SEQ ID NO: 1, 2, 4, 5, 22 or 23 or of their functional equivalents. It is likewise possible to use viral constructs which contain, for example a sequence as depicted in SEQ ID NO: 1, 2, 4, 5, 22 or 23 or their functional equivalents or parts thereof. It is likewise possible to use naked DNA which contains, for example, a sequence as depicted in SEQ ID NO: 1, 2, 4, 5, 22 or 23, or their functional equivalents or parts thereof. It is likewise possible to use nucleic acid fragments which possess enzymic activity (e.g. ribozymes, see above) for the purposes of gene therapy. Further possible pro-L119 or anti-L119 compounds which are preferred within the context of gene therapy are mentioned above.

Two generalized approaches for gene therapy comprise

(a) administering “naked” DNA which is complexed with lipid, which is formulated in liposomes or which is formulated in another manner, or

(b) administering the heterologous nucleic acid sequences using viral vectors.

One of the nucleic acid constructs according to the invention may have to be adapted for these approaches so as to achieve optimal expression (e.g. incorporation of an intron into the 5′-untranslated region, or elimination of unnecessary or inhibitory sequences (Felgner, et al. (1995) Ann NY Acad Sci 126-139). Formulations of the DNA which make use of different lipids or liposomes can then be used for the administration and are known to the skilled person (see above).

Various viral vectors can be used in connection with the second type of administration. Preference is given to retroviral, AAV and adenoviral systems (Dubensky et al. (1984) Proc. Natl. Acad. Sci. USA 81, 7529-7533; Kaneda et al., (1989) Science 243,375-378; Hiebert et al.(1989) Proc. Natl. Acad. Sci. USA 86, 3594-3598; Hatzoglu et al. (1990) J. Biol. Chem. 265, 17285-17293; Ferry, et al. (1991) Proc. Natl. Acad. Sci. USA 88, 8377-8381). Systems based on herpes simplex virus (HSV) are likewise preferred.

Retroviruses are a class of RNA viruses in which the RNA is reverse-transcribed into DNA in the infected cell. The retroviral genome is able to integrate into the genome of the host cell. The three viral genes gag, pol and env, and also the viral long terminal repeats (LTRs), are essential for the function of a retrovirus. LTRs can also function as enhancers and promoters of viral or heterologous genes. For the purpose of expressing heterologous nucleic acid sequences, the viral genes can be partially replaced with the sequences which are to be expressed. Following transfection into what is termed a packaging cell line, which contains the packaging components for forming infectious virus particles, a packaged virus is generated and released into the cell culture medium. Since retroviruses are only able to infect dividing cells, they are for the most part used in ex vivo gene therapy.

Adeno-associated viruses (AAV) are particularly preferred. They are particularly suitable use as vehicles for gene therapy which is carried out on a large number of tissues, such as lung or muscle tissue, and, in particular, for the therapy of vascular and endothelial diseases. AAV vectors infect cells and integrate into their genome with high efficiency. AAVs are also able to integrate into cells whose growth has been stopped (such as the lung epithelium) and are, moreover, not pathogenic. AAV-based expression vectors generally contain the AAV inverted terminal repeats (ITRs), which consist of 145 nucleotides and which flank, inter alia, a restriction cleavage site for the uptake of heterologous nucleic acid sequences (such as nucleic acid constructs which are suitable for expressing L119 proteins or pro-L119 or anti-L119 compounds). The capacity is approximately 4.4 kb. AAV vectors have been successfully employed in gene therapy for expressing a variety of proteins (Kotin R M (1994) Human Gene Therapy 5:793-801, Table I). Suitable promoters are those mentioned above or else an AAV promoter (ITR itself or AAV p5; Flotte et al. (1993) J Biol Chem 268:3781-3790). A vector of this nature can be packaged into AAV virions in the manner known to the skilled person (Carter B J (1992) Current Opinion in Biotechnology 3:533-539; Kotin R M (1994) Human Gene Therapy 5:793-801). Various methods are known for increasing virus titers. Furthermore, it is possible to increase the efficiency of the virus transduction (WO 96/39530). Various methods for concentrating and purifying viruses are known to the skilled person and encompass density gradient centrifugation (Flotte et al. (1993) J Biol Chem 268:3781-3790) and chromatographic purification (WO 97/08298). The skilled person is familiar with detailed methods of-AAV technology which can be used within the context of one of the processes according to the invention and which relate to the incorporation of a transgenic nucleic acid sequence and to the replication and purification of the AAV vector and its use for transfecting cells and mammals (e.g. U.S. Pat. No. 4,797,368; U.S. Pat. No. 5,139,941; U.S. Pat. No. 5,173,414; U.S. Pat. No. 5,252,479; U.S. Pat. No. 5,354,678; U.S. Pat. No. 5,436,146; U.S. Pat. No. 5,454,935; WO 93/24641; U.S. Pat. No. 5,658,776).

A variety of adenoviral vectors have proved their value in the gene therapy of mammals (including humans). For example, replication-deficient adenoviral vectors have been used for expressing CFTR in the pulmonary epithelium. The first generation of E1a-deleted adenoviral vectors has been improved such that the second generation now contains a temperature-sensitive E2a viral protein which is used for decreasing the expression of viral proteins and which makes the infected cell less of a target for an immune response (Goldman et al., Human Gene Therapy 6:839-851, 1995). Furthermore, viral vectors have been reported which do not contain any viral open reading frames (ORF) (Fisher et al. (1996) Virology 217:11-22). In addition to this, it has been demonstrated that the expression of viral IL-10 suppresses an immune response to an adenoviral antigen (Qin et al. (1997) Human Gene Therapy 8:1365-1374).

DNA sequences for a large number of adenoviruses can be obtained from Genbank. Several strains are available from the American Type Culture Collection (ATCC), Rockville, Md., USA or from a large number of commercial and academic sources. An adenoviral vector is constructed in a similar manner to any other vector as described above. It is likewise possible to use hybrid adenovirus-AAV vectors, which consist of an adenovirus capsid which contains selected constituent parts of adenoviral sequences, 5′ and 3′ AAV ITR sequences, which flank the transgene, and, where appropriate, additional regulatory elements (WO 96/13598).

The skilled person is familiar with the detailed information with regard to the adenovirus technology which can be used within the context of one of the processes according to the invention and which relates to the incorporation of a transgenic nucleic acid sequence and the replication and purification of the adenoviral vector and its use for transfecting cells and mammals (WO 94/28938, WO 96/13597 and WO 96/26285, and also the reference citations which are mentioned therein).

In general, DNA or virus particles are transferred into a biologically compatible solution or a pharmaceutically acceptable solvent, such as a sterile salt solution or a sterile aqueous or non-aqueous, isotonic injection solution or suspension. The skilled person is familiar with numerous examples, such as Ringer's solution, PBS (phosphate-buffered saline), etc. For the purpose of gene therapy, the DNA or the recombinant virus is preferably administered in a quantity which is sufficient for achieving a therapeutic effect without at the same time giving rise to unwanted side effects. This optimal dose depends on a variety of factors and can vary from patient to patient. Therapeutically effective doses can, for example, be in a range from 1 to 50 ml of a salt solution containing a virus gconcentration of from approximately 1×10⁷ to approximately 1×10¹⁰ pfu of virus/ml, preferably of from 1×10⁸ to approximately 1×10⁹ pfu of virus/ml.

The use of gene therapy methods, preferably of those based on AAV or adenoviral systems, is a preferred method for treating vascular and endothelial diseases. As a site of therapy, the endothelium is particularly readily accessible to the abovementioned methods.

The invention furthermore encompasses processes which are suitable for use in preventative medicine, for example as diagnostic tests and prognostic tests and for monitoring and assessing series of clinical experiments. The aim of these processes is to treat an individual prophylactically or therapeutically in a targeted manner.

The invention encompasses a process for qualitatively or quantitatively detecting the presence, the absence, the incorrectly regulated expression or an incorrect function of an L119 protein according to the invention or of an L119 nucleic acid sequence according to the invention in a biological sample, which process comprises one or more of the following steps:

a) isolating a biological sample from a test subject

b) incubating the biological sample with a reagent which is suitable for detecting an L119 protein according to the invention or an L119 nucleic acid sequence according to the invention in a manner such that the presence, the absence, the incorrectly regulated expression or an incorrect function of an L119 protein according to the invention or of an L119 nucleic acid sequence according to the invention can be detected.

The invention furtheremore relates to a process for qualitatively and quantitatively detecting a nucleic acid according to the invention in a biological sample, which process comprises the following steps:

a) incubating a biological sample with a known quantity of nucleic acid according to the invention or a known quantity of oligonucleotides which are suitable for use as primers for amplifying the nucleic acid according to the invention,

b) detecting the nucleic acid according to the invention by means of specific hybridization or PCR amplification,

c) comparing the quantity of hybridized nucleic acid or of nucleic acid obtained by PCR amplification with a quantity standard.

In addition, the invention relates to a process for qualitatively and quantitatively detecting a protein heteromer according to the invention or a protein according to the invention in a biological sample, which process comprises the following steps:

a) incubating a biological sample with an antibody which is specifically directed against the protein heteromer or against the protein according to the invention,

b) detecting the antibody/antigen complex,

c) comparing the quantities of the antibody/antigen complex with a quantity standard.

The term “biological sample” comprises tissues, cells or biological fluids which are obtained from or are present in a test subject. The abovementioned processes according to the invention for qualitative or quantitative detection can be carried out in vitro or in vivo. In vitro techniques for detecting an L119 mRNA comprise, for example, Northern hybridizations and in situ hybridizations. In vitro methods for detecting an L119 protein comprise ELISAs (enzyme-linked immunosorbent assays), Western blots (immunoblots), immunoprecipitations and immunofluorescence. In vitro methods for detecting an L119 genomic DNA comprise Southern hybridizations. In vivo methods for detecting an L119 protein comprise, for example, introducing a labeled anti-L119 antibody into a test subject. The labeling can, for example, be effected radioactively and the location and quantity of the antigen can be detected by means of imaging methods which are known to the skilled person.

A preferred reagent for detecting a nucleic acid (mRNA or genomic DNA) is a labeled nucleic acid which is able, as a probe, to hybridize with the L119 nucleic acid to be detected. This nucleic acid probe can, for example, comprise an L119 nucleic acid sequence, preferably a nucleic acid sequence as depicted in SEQ ID NO: 1, 2, 4, 5, 22 or 23, very preferably a human L119 nucleic acid sequence, most preferably a nucleic acid sequence as depicted in SEQ ID NO: 5 or 22. The invention also encompasses parts of the abovementioned probe, such as oligonucleotides which have a length of at least 15, 30, 50, 100, 250 or 500 nucleotides and which are able, under sufficiently stringent conditions, to hybridize with an L119 nucleic acid sequence or which can be used in the form of oligonucleotide primers for a detection method which is based on the polymerase chain reaction technique.

A preferred reagent for detecting an L119 protein is an antibody which is able to bind an L119 protein, preferably a labeled antibody. These antibodies may be polyclonal or, preferably, monoclonal. The invention encompasses both complete antibodies and fragments of these antibodies (e.g. Fab or F(ab′)2 fragments). Methods for preparing said antibodies are described above and are known to the skilled person.

The term “labeled” means the direct or indirect linking, for example of a probe, an dligonucleotide primer or an antibody, to detectable substances. Such detectable substances comprise various enzymes, prosthetic groups and fluorescent or luminescent or bioluminescent or radioactive materials. Examples of enzymes comprise horseradish peroxidase, alkaline phosphatase, β-galactosidase and acetylcholinesterase. Suitable prosthetic groups comprise, for example, streptavidin/biotin and avidin/biotin. Examples of suitable fluorescent materials comprise umbelliferone, fluorescein, fluorescein isothiocyanate, rhodamine, dichlorotriazinylaminofluorescein, dansyl chloride and phycoerythrin. An example of a luminescent material which may be mentioned is luminol, examples of bioluminescent materials are luciferase, luciferin and aequorin, and examples of radioactive materials are the isotopes ¹²⁵I, ¹³¹I, ³²P, ³³P, ³⁵S and ³H. The invention encompasses both direct methods for labeling, for example by means of a physicochemical bond, and indirect methods using substances which can in turn be directly labeled. Indirect methods can comprise, for example, detection of an antibody using a labeled secondary antibody or end-labeling a probe with biotin and detecting it using labeled streptavidin.

Normally, the abovementioned methods are carried out in parallel to a method using a biological sample obtained from a control individual. A biological sample removed from a healthy body is normally used as the standard.

The novel processes of this nature and/or the reagents and auxiliary substances which are required for implementing them, can be made available in the form of previously prepared tests together with directions for carrying them out.

The aim of such an investigation may be to establish whether an individual is affected by a disease, or is running the risk of developing a disease, which is connected, directly or indirectly, with a disruption of at least one essential property of an L119 protein.

A preferred embodiment of the process according to the invention for qualitatively or quantitatively detecting the presence, the absence, the incorrectly regulated expression or an incorrect function of an L119 protein according to the invention or of an L119 nucleic acid sequence according to the invention encompasses, for example, detecting genetic changes (“mutations”) in an L119 gene in a biological sample. These processes can be used, for example, to predict the risk to a person of contracting, for example, an L119-mediated vascular or endothelial disease.

Mutations in L119 genes can be of varying nature. They can be either mutations of relatively large regions or else relatively small changes in the nucleic acid sequence. The skilled person is familiar with example of both possibilities, which comprise, inter alia, deletions, insertions and rearrangements which affect the L119 nucleic acid sequence and also base exchanges/point mutations. The mutations may alter the protein sequence-encoding region of L119. This-can lead to changes in the protein sequence (substitution, inversion, insertion or deletion of one or more amino acid residues) and consequently to the altered protein having new properties (gain-of-function mutation). Alternatively, mutations may be present which alter the regulation of the expression of the L119 gene (transcription, translation and posttranslational modifications). These mutations may affect the 5′-untranslated or 3′-untranslated region of the L119 gene and may change/delete (flanking) regulatory sequences (e.g. promoters, intron sequences, spliced sequences, enhancers, silencers, locus control regions, matrix attachment regions, inter alia). Dysregulation of the expression can also lead to hypomorphic L119 alleles. Deletions, insertions or rearrangements of the L119 locus can occur, leading to the gene losing function (loss-of-function). It is furthermore possible for mutations to occur which result in the use of open reading frames which are not used in the wild-type allele. Chromosomal mutations can also lead to the recombination of new functional units. In this connection, a fusion transcript can be formed from parts of L119 which are recombined with a second gene. This fusion gene may encode a protein which possesses new properties. Alternatively, the coding sequence of one of the fusion partners may come (e.g. without its own sequence having been altered) under the control of regulatory elements belonging to the second partner.

In a particularly preferred embodiment, the invention relates to processes for detecting a genetic change in a cell sample taken from an individual. In this connection, the term “genetic change” means”

a) the deletion of one or more nucleotides in an L119 gene, or

b) the addition of one or more nucleotides to an L119 gene, or

c) the substitution of one or more nucleotides in an L119 gene, or

d) a chromosomal rearrangement within an L119 gene

e) a change in the quantity of the expressed mRNA of an L119 gene

f) a divergent modification of an L119 gene, such as a change in the methylation pattern of the genomic L119 DNA, or

g) the appearance of a splicing pattern in the mRNA of an L119 gene, which pattern differs from that of the wild type, or

h) a change in the quantity of an L119 protein which is expressed

i) the allelic loss of an L119 gene, or

j) a posttranslational modification of an L119 protein, which modification differs from that of the wild type.

The skilled person is familiar with a large number of methods for determining and analyzing such changes. These methods can comprise specific probes or primers in a polymerase chain reaction (PCR) (see U.S. Pat. No. 4,683,195 and U.S. Pat. No. 4,683,202). Modified forms of the PCR are also possible, such as “anchor PCR”, “RACE PCR”, and “ligation chain reaction (LCR)” (Landegran et al. (1988) Science 241:1077-1080; Nakazawa et al. (1994) Proc Natl Acad Sci USA 91:360-364), with it being possible to employ the last-mentioned particularly advantageously, when detecting point mutations in an L119 gene (Abravaya et al. (1995) Nucleic Acids Res. 23:675-682). Alternative amplification methods include: “self sustained sequence replication” (Guatelli J C et al. (1990) Proc Natl Acad Sci USA 87:1874-1878), transcription/amplification systems (Kwoh D Y et al., (1989) Proc Natl Acad Sci USA 86:1173-1177), Q-beta replicase (Lizardi P M et al. (1988) Bio-Technology 6:1197) and other amplification methods, after which the amplified nucleic acid molecules are detected using the method known to the skilled person.

Furthermore, changes in an L119 gene can be detected by means of changes in restriction enzyme cleavage patterns. In this connection, control or sample DNA can, for example, be isolated, (optionally) amplified and treated with one or more restriction endonucleases, with the resulting fragment lengths subsequently being determined and compared by, for example, gel electrophoresis. Differences in fragment length point to a change in the L119 gene.

In addition, sequence-specific ribozymes can be used for detecting particular mutations on the basis of the appearance and/or removal of a specific cleavage site (see U.S. Pat. No. 5,498,531).

In another preferred embodiment, mutations in an L119 gene can be detected by hybridizing sample and control nucleic acid molecules (for example DNA or RNA molecules) to high density arrays of hundreds or thousands of different oligonucleotides (Cronin M T et al. (1996) Human Mutation 7:244-255; Kozal M J et al. (1996) Nature Medicine 2:753-759).

In another preferred embodiment, mutations can be determined by using one of the numerous sequencing methods, with which the skilled person is familiar, by ascertaining the nucleic acid sequence and then comparing the latter with a wild-type control.

Methods which may be mentioned by way of example are those of Maxam and Gilbert (Proc Natl Acad Sci USA (1977) 74:560) or Sanger (Proc Natl Acad Sci USA (1977) 74:5463), or methods based on mass spectroscopy (see WO 94/16101; Cohen et al. (1996) Adv Chromatogr 36:127-162; Griffin et al. (1993) Appl Biochem Biotechnol 38:147-159).

Methods for detecting mutations in L119 genes comprise methods in which pairing errors in RNA/RNA, DNA/DNA or RNA/DNA duplexes are detected on the basis of the lack of protection against cleaving reagents (“mismatch cleavage”; Myers et al. (1985) Science 230:1242). For example, RNA/DNA duplexes can be treated with RNase and DNA/DNA hybrids can be treated with S1 nuclease, with the cleavage in each case taking place in the region of the mispairing. The fragment sizes of the treated material can subsequently be analyzed, for example by means of gel electrophoresis, thereby making it possible to determine the location of the mutation (see Cotton et al. (1988) Proc Natl Acad Sci USA 85:4397; Saleeba et al. (1992) Methods Enzymol. 217:286-295). For this, the control RNA or DNA can, in a preferred embodiment, be labeled, for example radioactively or using fluorescent dyes. Furthermore it is possible, in such a reaction, to use enzymes which recognize and cleave the specific base mispairings (for example E.coli mutY enzyme; thymidine DNA glycosylase (Hsu et al. (1994) Carcinogenesis 15:1657-1662); U.S. Pat. No. 5,459,039).

In another preferred embodiment, mutations in L119 genes can be detected on the basis of changes in electrophoretic mobility. What are termed single-strand conformation polymorphisms (SSCPs) can be used for detecting differences in electrophoretic mobility between the mutated sample and the control sample. Different embodiments are known to the skilled person (Orita et al. (1989) Proc Natl Acad Sci USA: 86:2766, Cotton (1993) Mutat Res 285:125-144; Hayashi (1992) Genet Anal Tech Appl 9:73-79; Keen et al. (1991) Trends Genet 7:5; Myers et al. (1985) Nature 313:495; Rosenbaum and Reissner (1987) Biophys Chem 265:12753).

Other methods for finding point mutations comprise, by way of example and not in a limiting manner: selective oligonucleotide hybridization, selective amplification and selective primer extension. Selective hybridization comprises using oligonucleotide primers which only hybridize with a sample when there is perfect complementarity and not when the sample contains a point mutation (Saiki et al. (1986) Nature 324:163; Saiki et al. (1989) Proc Natl Acad Sci USA 86:6230). The methods of selective amplification (Gibbs et al. (1989) Nucleic Acids Res 17:2437-2448) and selective primer extension (Prossner et al. (1993) Tibtech 11:238) function in an analogous manner.

In addition, the processes according to the invention can be used for detecting the activity of a compound which modulates at least one essential property, or the expression, of an L119 protein. These processes can be used within the context of clinical investigations of the activity of said compounds.

The nucleic acid sequences or nucleic acid constructs, or parts thereof, which are made available within the context of the invention can have many different uses:

a) for finding the corresponding L119 genes on a chromosome (chromosome mapping) and thereby, where appropriate, locating a region which is linked to a genetically determined disease. The techniques of chromosome mapping are known to the skilled person (D'Eustachio P et al. (1983) Science 220:919-924, Fan Y et al. (1990) Proc Natl Acad Sci USA, 87:6223-27, Verma et al. (1988) Human Chromosomes: A Manual of Basic Techniques, Pergamon Press, New York). As soon as a sequence has been located on a particular chromosome using known techniques (e.g. FISH; fluorescence in situ hybridization), this position can be compared with data on a gene map. These data can be obtained, for example, from the OMIM database (see above). The relationship between the gene and the disease can, for example, be established by means of what is termed linkage analysis (Egeland J et al. (1987) Nature, 325:783-787). It is furthermore possible to analyze differences in the sequence between affected and unaffected individuals.

b) in a method for qualitatively or quantitatively detecting one of the nucleic acids according to the invention in a biological sample.

The diagnostic processes which are made available within the context of the invention can furthermore be used for predicting the risk of an individual contracting one of the abovementioned vascular or endothelial diseases which can be attributed to an L119 protein, nucleic acid expression or activity. Preference is given to carrying out such a test using a protein or nucleic acid sample (mRNA or genomic DNA) which has been isolated from a test subject. Such a sample can be isolated from a biological fluid (e.g. serum), cells or tissue, for example within the context of a biopsy.

In another preferred embodiment, the diagnostic methods are used for predicting the probability of success when treating, or the possibility of treating, a patient who is suffering from an vascular or endothelial disease with L119-modulating or -normalizing substances (e.g. pro-L119 or anti-L119 compounds).

The processes which are encompassed by the invention can, for example, be used in the form of previously prepared diagnostic kits. Furthermore, the diagnostic processes can also be used during clinical investigations into the activity of L119-modulating or -normalizing compounds in order, for example, to examine the level at which an L119 protein is expressed or to select a suitable patient cohort for a particular L119-modulating or -normalizing approach.

Furthermore, the cDNA, the genomic DNA, the regulatory elements of the nucleic acid sequences according to the invention and also the polypeptide, and fragments thereof, can be used in recombinant or nonrecombinant form for developoing a test system. This test system is suitable for measuring the activity of the promoter or of the protein in the presence of the test substance. Preferably, the test systems are simple measurement methods (calorimetric, luminometric, fluorescence-based or radioactive methods) which enable a large number of test substances to be measured rapidly (Bohm, Klebe, and Kubinyi. (1996). Wirkstoffdesign (Active compound design) (Heidelberg: Spektrum-Verlag). The above-described test systems enable chemical or biological libraries to be screened for substances which have agonistic and antagonistic effects on SEQ ID NO: 3, 6, 7 or 24 or the complex which consists of a protein mentioned in Table 1 and the protein described in SEQ ID NO: 3, 6, 7 or 24.

An alternative route for developing active compounds which attack L119 consists in rational drug design (Bohm et al., 1996). In this case, the structure or a part structure of the protein depicted in SEQ ID NO: 3, 6, 7 or 24, insofar as it is available, or a structural model generated by computers, is used in order to find, with the support of molecular modeling programs, structures which can be predicted to have a high affinity for L119. These substances are then synthesized and tested. Selected substances having high affinity are then tested for their use as drugs for treating vascular or endothelial diseases as defined above.

The determination of the quantity, activity and distribution of the protein depicted in SEQ ID NO: 3, 6, 7 or 24, or of its underlying mRNA, in the human body can be used for diagnosis, determining predisposition, and monitoring, in association with particular diseases. In the same way, the sequence of the cDNA, or of the sequences SEQ ID NO: 3, 6, 7 or 24, and also of the genomic DNA, can be used for drawing conclusions with regard to the genetic causes of, and predispositions to, particular diseases. It is possible to use a very wide variety of both DNA/RNA samples and antibodies for this purpose. In this connection, the above-described nucleotide sequence SEQ ID NO: 1, 2, 4, 5, 22 or 23, or parts thereof, is used in the form of suitable samples for unearthing point mutations or deletions/insertions/rearrangements.

The present nucleic acid sequence SEQ ID NO: 1, 2, 4, 5, 22 or 23, its functional equivalents, homologs or derivatives, the protein encoded by it (SEQ ID NO: 3, 6, 7 or 24), or the protein heteromer according to the invention, containing one of the proteins depicted in Table 1, and also reagents derived therefrom (oligonucleotides, antibodies and peptides), can be employed for the diagnosis and therapy of vascular or endothelial diseases as defined above.

Furthermore, it is possible to monitor the treatment of diseases. This relates, for example, to assessing the course of diseases, to assessing the success of therapies, and to grading a disease. Specifically, determining the quantity of L119 in cells or body fluids can be used for monitoring the course of vascular diseases, forms of hypertension or particular tumors.

In addition, the invention relates to a process for finding substances which bind specifically to a protein having an amino acid sequence as depicted in SEQ ID NO: 3, 6, 7 or 24 or to a nucleic acid sequence as depicted in SEQ ID NO: 1; 2, 4, 5, 22 or 23 and thereby induce inhibitory or activating functional effects on L119 signal transmission in vascular endothelial cells.

In situations in which the activity of the protein according to the invention having the sequence SEQ ID NO: 3, 6, 7 or 24, or of one of its functional equivalents, is deficient, several methods can be used for replacing this activity or increasing it. In principle, all the above-described methods which use a pro-L119 compound are suitable for this purpose. In the first place, the protein, which is natural or recombinant, can be administered either directly or, by taking suitable steps, in the form of its C; encoding nucleic acid (i.e. DNA or RNA). Both viral and nonviral vehicles can be employed for this purpose, as already described above. Another possible route is that of using suitable substances to stimulate the endogenous gene. Such substances can be found, for example, by determing their effect on the transcription elements of the L119 gene.

In situations in which the activity of a protein having the sequence SEQ ID NO: 3, 6, 7 or 24, or of one of its equivalents, is in excess, it is possible, in principle, to employ all the above-described methods which use an anti-L119 compound. In particular, it is possible to use specific, synthetic or natural, competitive or non-competitive, antagonists against the protein having the sequence SEQ ID NO: 3, 6, 7 or 24, or one of its functional equivalents, or else antibodies or antibody fragments which are directed against the protein having the sequence SEQ ID NO: 3, 6, 7 or 24, or against the protein heteromer or one of the functional equivalents thereof. It is furthermore possible to use antisense molecules or ribozymes or oligonucleotides or low molecular weight compounds to inhibit the L119 activity or the activity of the protein having the sequence SEQ ID NO: 3, 6, 7 or 24 or that of one of its functional equivalents.

Nucleic acids as depicted in SEQ ID NO: 1, 2, 4, 5, 22 or 23, or one of their functional equivalents, or complementary nucleic acid sequences which are derived therefrom, can be used for producing drugs. These drugs are preferably used for the therapy and prophylaxis of human and animal diseases, particularly preferably the therapy and prophylaxis of human diseases, very particularly preferably the therapy and prophylaxis of vascular or endothelial diseases which are defined above and which can be influenced positively by modulating or normalizing the expression of the L119 gene.

Proteins, protein fragments or peptides having the sequence SEQ ID NO: 3, 6, 7 or 24, or parts thereof, or one of their functional equivalents, can be used in just the same way. The invention also relates to the use of antibodies or antibody fragments or antibody mixtures which are directed against the protein having the sequence SEQ ID NO: 3, 6, 7 or 24, or against the protein heteromer, for producing drugs. These drugs are preferably used for the therapy and prophylaxis of human and animal diseases, particularly preferably the therapy and prophylaxis of human diseases, very particularly preferably the therapy and prophylaxis of vascular or endothelial diseases which are defined above and which can be positively influenced by modulating or normalizing the activity or quantity of L119 protein.

Compounds which bind specifically to a protein having an amino acid sequence as depicted in SEQ ID NO: 3, 6, 7 or 24, or one of its functional equivalents, or to a nucleic acid sequence as depicted in SEQ ID NO: 1, 2, 4, 5, 22 or 23, or one of its functional equivalents, or at least modulate or normalize an essential property, or the expression, of an L119 protein as depicted in SEQ ID NO: 3, 6, 7 or 24, or of one of its functional equivalents, can be used for producing drugs. These drugs are preferably used for the therapy and prophylaxis of human and animal diseases, particularly preferably the therapy and prophylaxis of human diseases, very particularly preferably the therapy and prophylaxis of vascular or endothelial diseases which are defined above and which can be positively influenced by modulating or normalizing the activity or quantity of L119 protein.

A modulation or normalization of L119, or a therapy and prophylaxis of diseases which can be positively influenced by modulating or normalizing the activity or quantity of L119 protein using one of the above-described approaches using the novel nucleic acids, nucleic acid constructs, proteins or compounds, can be usefully combined with other therapeutic approaches. Useful combinations comprise those with endothelin receptor antagonists, inhibitors of the renin-angiotensin system, such as renin inhibitors, angiotensin II antagonists and angiotensin converting enzyme (ACE) inhibitors, beta blockers, diuretics and VEGF antagonists.

Sequences

| | | |
|---|---|---|
| 1. | SEQ ID NO: 1 | Rattus norvegicus L119 cDNA |
| | | sequence clone 1 |
| 2. | SEQ ID NO: 2 | Rattus norvegicus L119 cDNA |
| | | sequence clone 2 |
| 3. | SEQ ID NO: 3 | Rattus norvegicus L119 protein |
| | | sequence |
| 4. | SEQ ID NO: 4 | Mus musculus L119 genomic sequence |
| 5. | SEQ ID NO: 5 | Homo sapiens L119 cDNA sequence |
| 6. | SEQ ID NO: 6 | Homo sapiens L119 protein sequence |
| | | (long form) |
| 7. | SEQ ID NO: 7 | Homo sapiens L119 protein sequence |
| | | (short form) |
| 8. | SEQ ID NO: 8 | rL119-4s oligonucleotide primer |
| | | 5′-TATCACTCAGCCCGGTCACCCTGG-3′ |
| 9. | SEQ ID NO: 9 | rL119-5as oligonucleotide primer |
| | | 5′-ACGCCTGGGGATGAGGAAGCCACG-3′ |
| 10. | SEQ ID NO: 10 | humL119-5′-myc (EcoRI) |
| | | oligonucleotide primer |
| | | 5′-CTATGAATTCACCATGATCCACTGGAAAC |
| | | AGA-3′ |
| 11. | SEQ ID NO: 11 | humL119-3′-myc (XbaI) |
| | | oligonucleotide primer |
| | | 5′-CACTAGTCTAGAGAAAAACAGCCCTGCA |
| | | CGC-3′ |
| 12. | SEQ ID NO: 12 | hL119-1s oligonucleotide primer |
| | | 5′-AGTTATGTCTTCTGGGTGACAGAC-3′ |
| 13. | SEQ ID NO: 13 | hL119-2s oligonucleotide primer |
| | | 5′-TTGCAAGCCTGATGTCCTATCAAG-3′ |
| 14. | SEQ ID NO: 14 | hL119-3s oligonucleotide primer |
| | | 5′-ATCGTGGGGCTCTCGCTCAG-3′ |
| 15. | SEQ ID NO: 15 | hL119-4s oligonucleotide primer |
| | | 5′-CGTCACCATCACGTCCGATCTC-3′ |
| 16. | SEQ ID NO: 16 | hL119-1as oligonucleotide primer |
| | | 5′-CAGTCTAGGAGATGACACCAGC-3′ |
| 17. | SEQ ID NO: 17 | hL119-2as oligonucleotide primer |
| | | 5′-AGGGTGCGGACAGATTGGGTAC-3′ |
| 18. | SEQ ID NO: 18 | hL119-3as oligonucleotide primer |
| | | 5′-GCTCTCGGCCAGTTTCTGAATC-3′ |
| 19. | SEQ ID NO: 19 | hL119-4as oligonucleotide primer |
| | | 5′-GCTCGCTGAGTTCGTCCAGAGC-3′ |
| 20. | SEQ ID NO: 20 | pHM2-7s oligonucleotide primer |
| | | 5′-GACCGCTATCAGGACATAGCGTTG-3′ |
| 21. | SEQ ID NO: 21 | mgL119-15as oligonucleotide primer |
| | | 5′-ACTATGTAGCCTGGGCTCAGGTAG-3′ |
| 22. | SEQ ID NO: 22 | Homo sapiens L119 genomic DNA |
| 23. | SEQ ID NO: 23 | Mus musculus L119 cDNA |
| 24. | SEQ ID NO: 24 | Mus musculus L119 protein |
| 25. | SEQ ID NO: 25 | rL119-5′-1-myc (EcoRI) |
| | | oligonucleotide primer |
| | | 5′-ACACCGGAATTCAGCATGGAGAAGTGGAC |
| | | GGC-3′ |
| 26. | SEQ ID NO: 26 | rL119-3′-738-myc (XbaI) |
| | | oligonucleotide primer |
| | | 5′-CCCTAGTCTAGAGAAAAACAACGCTGCATCC |
| | | AGA-3′ |
| 27. | SEQ ID NO: 27 | rL119-5′-2-flag (XbaI) |
| | | oligonucleotide primer |
| | | 5′-CCCTAGTCTAGAGAGAAGTGGACGGCC |
| | | TGG-3′ |
| 28. | SEQ ID NO: 28 | rL119-3′-741-flag (EcoRI) |
| | | oligonucleotide primer |
| | | 5′-ACACCGGAATTCTTAGAAAAACAACGCTGCA |
| | | TCC-3′ |
| 29. | SEQ ID NO: 29 | rL119-5′-ORF (SalI) |
| | | oligonucleotide primer |
| | | 5′-TGGTGGGTCGACATGGAGAGGTGGACG-3′ |
| 30. | SEQ ID NO: 30 | rL119-3′-ORF (NotI) |
| | | oligonucleotide primer |
| | | 5′-AGAAGAAGAGGCGGCCGCTTAGAAAAACAAC |
| | | GCTGC-3′ |
| 31. | SEQ ID NO: 31 | rL119-5′-pEGFPC1 (EcoRI) |
| | | oligonucleotide primer |
| | | 5′-ACACCGGAATTCTGAGAAGTGGACGGCCTGG |
| | | GAG-3′ |
| 32. | SEQ ID NO: 32 | rL119-3′-pEGFPC1 (BamHI) |
| | | oligonucleotide primer |
| | | 5′-CACGCGGATCCTTAGAAAAACAACGCTGCAT |
| | | CCAG-3′ |
| 33. | SEQ ID NO: 33 | rL119-5′-pEGFPN1 (EcoRI) |
| | | oligonucleotide primer |
| | | 5′-TCACTGGAATTCTGATGGAGAAGTGGACGGC |
| | | CTGG-3′ |
| 34. | SEQ ID NO: 34 | rL119-3′-pEGFPN1 (BamHI) |
| | | oligonucleotide primer |
| | | 5′-CACGCGGATCCGAGAAAAACAACGCTGCATC |
| | | CAGA-3′ |
| 35. | SEQ ID NO: 35 | 5′-L119-bait |
| | | oligonucleotide primer |
| | | 5′-GGTCGACGGAGAAGTGGACGGCCTGGGA |
| | | GC-3′ |
| 36. | SEQ ID NO: 36 | 3′-L119-bait |
| | | oligonucleotide primer |
| | | 5′-AGCGGCCGCTTAGAAAAACAACGCTGC |
| | | ATC-3′ |
| 37. | SEQ ID NO: 37 | rL119-5′-pGEX-4T2 (BamHI) |
| | | oligonucleotide primer |
| | | 5′-CACGCGGATCCAGGCGTGCGGAGGGGGC |
| | | CAC-3′ |
| 38. | SEQ ID NO: 38 | rL119-3′-pGEX-4T2 (SalI) |
| | | oligonucleotide primer |
| | | 5′-CCGACGTCGACTTAGAAAAACAACGCTGC |
| | | ATC-3′ |
| 39. | SEQ ID NO: 39 | GAPDHs oligonucleotide primer |
| | | 5′-CTACATGGTCTACATGTTCCAGTA-3′ |
| 40. | SEQ ID NO: 40 | GAPDHas oligonucleotide primer |
| | | 5′-TGATGGCATGGACTGTGGTCAT-3′ |
| 41. | SEQ ID NO: 41 | rS26-1s oligonucleotide primer |
| | | 5′-AAGTTTGTCATTCGGAACATTGT-3′ |
| 42. | SEQ ID NO: 42 | rS26-1as oligonucleotide primer |
| | | 5′-CACCTCTTTACATGGGCTTTG-3′, |
| 43. | SEQ ID NO: 43 | mgL119-3sNotI |
| | | oligonucleotide primer |
| | | 5′-AAATATGCGGCCGCAGTGTGCCCTTTCTGAG |
| | | ACC-3′ |
| 44. | SEQ ID NO: 44 | mgL119-4as oligonucleotide primer |
| | | 5′-CTCCATGCCCTGTGAGGGACACAG-3′ |
| 45. | SEQ ID NO: 45 | L119-17s oligonucleotide primer |
| | | 5′-GGGTCTGAATAGGAAGGGAGTCTG-3′ |
| 46. | SEQ ID NO: 46 | L119-19as oligonucleotide primer |
| | | 5′-ATAGGACATCAGGTTTCCAAGGTC-3′ |
| 47. | SEQ ID NO: 47 | Cyc5 (s) oligonucleotide primer |
| | | 5′-ACCCCACCGTGTTCTTCGAC-3′ |
| 48. | SEQ ID NO: 48 | acyc300 (as) |
| | | oligonucleotide primer |
| | | 5′-CATTTGCCATGGACAAGATG-3′ |
| 49. | SEQ ID NO: 49 | pHM2-8 oligonucleotide primer |
| | | 5′-GTGACCATGTCGTTTACTTTGACC-3′ |
| 50. | SEQ ID NO: 50 | pHM2-9 oligonucleotide primer |
| | | 5′-GGTTAACGCCTCGAATCAGCAACG-3′ |
| 51. | SEQ ID NO: 51 | L119-MG-F2 (s) |
| | | oligonucleotide primer: |
| | | 5′-CTCTAGCCTAGGGCAGCAAC-3′ |
| 52. | SEQ ID NO: 52 | L119-MG-R1 (as) |
| | | oligonucleotide primer: |
| | | 5′-GAGAGAGGTCGGACGTGATG-3′ |
| 53. | SEQ ID NO: 53 | L119-LacZ-R1 |
| | | oligonucleotide primer: |
| | | 5′-GGCGATTAAGTTGGGTAACG-3′ |

### FIGURES

FIG. 1: Diagrammatic depiction (not to scale) of the exon-intron structure of the L119 gene based on comparing the genomic sequence of the mouse with the two new cDNA splice variants in the rat. The exon limits in the mouse genomic sequence are given below the diagram of SEQ ID NO: 4 (A). The rat cDNAs are shown as gray quadrangles; the black part represents the open reading frame (ORF). The nucleotide positions which correspond to the exon limits are marked above the quadrangles. SEQ ID NO:1 is shown diagrammatically in (B) while SEQ ID NO:2 is shown diagrammaticaly in (C).

FIG. 2: Comparison of the sequence of the L119 protein (human; SEQ ID NO: 6) with those of the proteins ApoL-and CG12_{—}1.

FIG. 3: (A) Northern analysis which was originally intended to confirm induction in the hippocampus and cortex using MECS and cycloheximide. Following stimulation, total RNA was isolated from the rat hippocampus or cortex at the times indicated. The concentration and purity of the RNA were checked; for the analysis, 20 μg of RNA were fractionated on a denaturing gel. It should be noted that the same induction as in the control is obtained when cycloheximide is used on its own. GAPDH was used as the internal control.

(B) Northern analysis for investigating the induction of L119 by either MECS or cycloheximide on its own. Following stimulation, total RNA was isolated from the rat hippocampus or cortex at the times indicated. The concentration and purity of the RNA were checked; for the analysis, 20 μg of RNA were fractionated on a denaturing gel. The rapid and transient induction of L119 mRNA with MECS, and the superinduction with cycloheximide should be noted (mmecs=multiple massive electroconvulsive shock).

FIG. 4: Detecting a specific, inducible signal in rats by means of in situ hybridization using a digoxigenin-labeled antisense ribonucleotide probe. The upper left-hand half of each section represents a control rat brain while the lower right-hand half is the brain of a rat following 4 hour-long multiple administration of MECS and cycloheximide (CHX).

(A): The signal is generated in the stimulated animal.

(B) The signal can be destroyed by pretreating the slide with RNase A.

(C to F): The signal can be caused to disappear by adding increasing concentrations of unlabeled ribonucleotide probe (C: 2×, D: 5×, E: 20×, F: 50×).

FIG. 5: Induction of L119 mRNA in the rat brain. Gyrus dentatus (A) and cerebellum (B) of a control rat display nonspecific staining with an L119-specific probe in the in situ hybridization. A signal can be induced both in the gyrus dentatus (C) and in the cerebellum (D) by treating with cycloheximide (CHX). (E, F): Closer examination of the in situ hybridization in a rat brain shows that the staining pattern corresponds to expression in the blood vessels. (Magnification: A to D 50×; E, F 125×)

FIG. 6: in situ hybridization performed on a microvessel-enriched tissue preparation from rat brain. Preparations were obtained both from control rats (A, B) and from rats which had previously been treated with cycloheximide (CHX) (C to F). (Magnification: 125×)

FIG. 7: The expression of L119 is not restricted to blood vessel endothelium in the brain. The mRNA can also be detected by in situ hybridization in the adrenal (A), the kidney (B), the liver (C), the spleen (D), the lung (E) and the retina (F) of cycloheximide (CHX)-treated rats (right-hand side) whereas it was not possible to detect any signals in the corresponding tissues obtained from control rats (left-hand side) ([i: control rat; ii: cycloheximide-stimulated rat] (magnification: A to E 50×, F 125×).

FIG. 8: L119 is expressed at a basal level during ontogenesis.

C Brains of 10-day-old rats which had been stimulated with cycloheximide (CHX) displayed very strong signals in vascular endothelium (B). However, in contrast to adult animals, it was also possible to observe significant basal expression of L119 mRNA in these animals (A).

FIG. 9: Northern blot analyses carried out on rat brains of varying ages (day 9.5 embryo to adult) detected basal expression of L119 mRNA at all the stages analyzed. The strongest signals were obtained between postnatal days 8 and 21.

FIG. 10: Investigation of the pattern of expression of L119 mRNA in human organs in the basal state. A blot containing poly(A)+RNA from 12 different organs (Clontech) was hybridized with radioactive probes for L119 and S26 (small subunit ribosomal protein). Signals were obtained from all the organs investigated, including strong signals from the heart, the skeletal muscle, placenta, the lung and the kidney. The size of L119 mRNA which was detected was about 4.5 kb in all the organs. Additional bands of a different size (sizes of from about 5 to 6 kb and of 3 kb, respectively) could be observed in the lanes containing the strongest signals (skeletal muscle, heart and placenta). Loading of the lanes in blot 1: brain, 2: heart, 3: skeletal muscle, 4: colon, 5: thymus, 6: spleen, 7: kidney, 8: liver, 9: small intestine, 10: placenta, 11: lung, 12: peripheral blood leukocytes.

FIG. 11: The expression of L119-mRNA is upregulated in 9L glioblastoma tumors which are growing in the lower leg of the rat (A, C). The expression of L119 can be further induced in these tumors by pretreating the animal with cycloheximide (B, D). The expression of L119 in these tumors is evidently located in the blood vessel endothelium (arrows in E to H).

FIG. 12: The expression of L119 mRNA is upregulated in 9L-glioblastoma tumors which have been implanted into the brains of adult rats. Minute tumor masses were implanted into the left ventricle of the brains of adult rats and allowed to grow for 8 (A, C) or 18 (B, D) days before the animals were sacrificed. The sections were subjected either to a Nissl staining (A, B) or to an in situ hybridization (C, D) using an L119 antisense ribonucleotide probe. (Magnification: 50×)

FIG. 13: Subcellular fractionation of rL119 following expression in HEK 293 cells. 48 h after the transfection of HEK293 cells with L119-myc-His (A) or Flag-L119 (B) or the corresponding empty control plasmids pcDNA-mycHis and pRK5, the cells were disrupted, following hypotonic shock, by the cell suspension being drawn 25 times through a 23-gage needle and a cell nucleus-containing 1000 g precipitate, a 100000 g precipitate (mem) and a 100000 g supernatant (cyto) were prepared. A nuclear extract (NE) was obtained from the 1000 g precipitate by extracting with 0.42 M NaCl buffer (cell fractionation described in: Scheek S et al. (1998) Proc Natl Acad Sci USA 94, 11179-83). The content of L119 in the subcellular fractions was investigated by means of Western blot analysis. The antibodies used were an anti-myc antibody (invitrogen) and an anti-M2-flag antibody (SIGMA-Aldrich). An L119-specific band was detected exclusively in the 100000 g membrane fraction.

FIG. 14: COS-7 cells were transiently transfected either with an empty pRK5 vector (A) or with pRK-5-FL-L119 (B, C). The cells were cultured for a further 48 h and then fixed with 4% strength PFA and permeabilized with Triton X-100. The cells were stained with a rabbit anti-L119 primary antibody and then with an anti-rabbit FITC-secondary antibody.

FIG. 15: L119 interacts in vitro with myc-Notch 1. This interaction depends on cotransfecting the transgene constructs into the same cell population. HEK 293 cells were cotransfected with L119 and either myc-Notch 1 or empty vector. A coimmune precipitation was carried out using Notch1 antibody. The Western blot was probed with a rabbit anti-L119 antibody.

FIG. 16: L119 interacts in vitro with myc-neuropilin 1. An immunoprecipitation experiment was carried out on transiently transfected COS 7 cells. The cells were cotransfected with L119 and either the empty vector (A), myc-FL-Npn-1 (B), myc-ΔA-Npn-1 (C), myc-ΔB-Npn-1 (D) or myc-ΔC-Npn-1 (E). A coimmune staining was carried out using anti-myc monoclonal antibody. The Western blot was probed with a rabbit anti-L119 antibody. The neuropilin 1 diagram (F) at the bottom of the figure shows the nature of the deletions.

FIG. 17: The expression of L119-myc in HEK 293 cells. 48 h after been transfected with L119-myc-His, HEK293 cells were harvested and, after the cells had been disrupted, a 1000 g centrifugation was carried out. The resulting supernatant was fractionated in a denaturing protein gel. In each case three gel lanes were probed with preimmune serum (A), the immune serum which was obtained (7340) (B) and, as a control, with a rabbit anti-myc-antibody (Upstate Biotechnology) (C).

FIG. 18: Identification, by means of PCR and agarose gel electrophoresis, of ES cells which contain a mutated L119 allele following successful homologous recombination with an L119 knock-out construct. A band of the expected size was amplified from genomic DNA obtained from the ES cell lines #308 and #341 but not from #307. A negative control was analyzed in the first lane (PCR reaction without ES cell DNA). The MBI Fermentas 1 kb ladder was loaded as the marker. The desired homologous recombination took place in ES cell clones #308 and #341.

FIG. 19: Induction of L119 mRNA expression in primary microvascular endothelial cells (HMVEC-L; Clonetics) following a 1.5-hour treatment with cycloheximide (CHX) at the concentrations given in each case. 350000 cells were sown per 10 cm plate and cultured in EGM-2MV medium (Clonetics). The medium was changed after every 24 h until confluence had been reached. The cells were cultured for a further 24 h (A) or 48 h (B), without any change of medium, and in each case three plates were incubated for 90 min with the given concentrations of CHX. After the RNA had been prepared using the RNeasy kit (Qiagen), 10 μg of total RNA were in each case examined by Northern blot analysis in regard to the expression of L119 MRNA. A 2070 XhoI-HindIII cDNA fragment was used as the L119 probe. The blot was standardized with an S26 probe (data not shown).

FIG. 20: Induction of L119 mRNA expression in primary microvascular endothelial cells (HMVEC-L) following treatment with cycloheximide (CHX), TNF-α and IL-1β. A: 250 μg of CHX/ml, B: 25 nM TNFα, C: 10 ng of IL-1β/ml, D:100 ng of IL-1β/ml. The cells were sown in EGM-2-MV medium using 140000 cells per 6 cm plate, cultured for three days (medium change after every 24 h) and then incubated for a further 20 h in serum-free EGM basal medium (Clonetics). The cells were treated for 0, 30 and 90 min with CHX, TNF-α and IL-1β at the given concentrations and then examined by Northern blot analysis to determine L119 mRNA expression. A 2070 bp XhoI-HindIII cDNA fragment was used as the L119 probe. The blot was standardized with an S26 probe (data not shown).

FIG. 21: Stimulation of L119 mRNA expression in endothelial cells by hypoxia. A: L119 expression in HMVE cells. B: Expression in RBE4 cells. The cells were sown at a rate of 140000 cells per 6 cm plate and cultured for three days (medium change after every 24 h); they were then incubated for a further 20 h in serum-free basal medium. They were subsequently gassed with a gas mixture of 90% N₂, 5% CO₂ and 5% H₂, in the presence of a catalyst (BBL GasPak Replacement Charges; Becton Dickinson, Cat. No. 4370303), at 37° C. in a hypoxia chamber for between 0 and 180 min. After the RNA had been prepared using the RNeasy kit (Qiagen), in each case 10 μg of total RNA were examined by Northern blot analysis in order to determine the expression of L119 mRNA. A XhoI-HindIII cDNA fragment was used as the human L119 probe while a PCR fragment from the 3′-untranslated region of the L119 cDNA (pos. 2260 to 2920 in SEQ ID NO: 1) served as the rat probe. The blot was standardized with an S26 probe.

FIG. 22: Subcellular location of L119-myc in YPEN-1 cells following transient transfection.

A: Transfection with an L119-myc-His-expressing pEGFPΔEGFP vector.

B: Transfection with the pEGFPΔEGFP vector (vector control).

Following lipofection of the plasmids using Lipofectamine Plus (GibcoBRL), the cells were cultured in EGM-2-MV medium for a further 36 h and then fixed in 3% paraformaldehyde for 30 min. Following permeabilization with 0.15% Triton X-100, the immune staining was carried out using a polyclonal antibody directed against rat L119 (rL119) (2892), followed by an anti-rabbit IgG-FITC antibody (Jackson ImmunoResearch Laboratories Inc.).

FIG. 23: Subcellular location of EGFP-L119 (A) and L119-EGFP (B) in YPEN-1 cells following transient transfection. Following lipofection of the plasmids (A: pEGFPC1-L119 and B: pEGFPN1-L119) using Lipofectamine Plus (GibcoBRL), the cells were cultured for a further 36 h in complete medium and then fixed in 3% paraformaldehyde for 30 min. Following permeabilization with 0.15% Triton X-100, the immune staining was carried out using a polyclonal antibody directed against rL119 (2892), followed by an anti-rabbit IgG-FITC antibody (Jackson ImmunoResearch Laboratories Inc.).

FIG. 24: Subcellular location of EGFP-L119 (A) and L119-EGFP (B) in RBE4 cells following transient transfection. Following lipofection of the plasmids (A: pEGFPC1-L119 and B: pEGFPN1-L119) using Lipofectamine Plus (GibcoBRL), the cells were cultured for a further 36 h in complete medium and then fixed in 3% paraformaldehyde for 30 min. Following permeabilization with 0.15% Triton X-100, the immune staining was carried out using a polyclonal antibody directed against rL119 (2892), followed by an anti-rabbit IgG-FITC antibody (Jackson ImmunoResearch Laboratories Inc.).

FIG. 25: Western blot analysis using various polyclonal L119 antisera. The rabbit sera (peptide antibodies: 2892 to 2895; GST fusion proteins 3841 and 3843) were tested in Western blot experiments for a specific reaction with heterologously expressed L119 protein. For this, HEK293 cells were transiently transfected with rL119-myc-His and humL119-myc-His (long form) and, in parallel, with the corresponding control vector (pcDNA3.1-myc-His-A). After 48 hours, the cells were harvested and a 1000 g supernatant was prepared. This protein fraction was fractionated and blotted on a denaturing protein gel. The second antibody employed for the polyclonal L119 sera was an HRP-conjugated anti-rabbit IgG antibody (Jackson ImmunoResearch Laboratories Inc). A control hybridization with an anti-myc antibody (Biomol) was carried out in order to identify the L119-specific bands.

FIG. 26: Comparison of the L119 protein sequences in humans (human L119; SEQ ID NO: 7), in the mouse (mouse L119; SEQ ID NO: 24) and in the rat (rat L119; SEQ ID NO: 3).

FIG. 27: Panels 1 to 7 Comparison of the mouse L119 genomic DNA sequence (upper sequence; SEQ ID NO: 4) with the rat cDNA sequence (lower sequence; SEQ ID NO: 2).

FIG. 28: Comparison of the mouse L119 genomic DNA sequence (upper sequence; SEQ ID NO: 4) with parts of the rat cDNA sequence (lower sequence; SEQ ID NO: 1).

FIG. 29: Panels 1 to 18 Comparison of the human L119 genomic DNA sequence (upper sequence; SEQ ID NO: 22) with the mouse L119 genomic DNA sequence (lower sequence; SEQ ID NO: 4).

FIG. 30: L119 protein expression is induced after kainate treatment.

Rats were injected with either 12 mg/kg kainate or PBS only. 3 h after onset of seizures (4 h after injection) rats were anesthetized with sodium chloral hydrate and perfused with 75 ml PBS. The brain was removed, frozen on dry ice and sectioned in 20 μm cryosections. Immunohistochemistry was performed with polyclonal anti-L119 antibody 2892 and Vectastain Elite ABC immunoperoxidase system with DAB as peroxidase substrate.

FIG. 31: Induction of L119 gene expression by treatment with lipopolysaccharides (LPS).

Mice were injected with either 2.5 mg LPS/kg (i.p.) in PBS or with PBS only. After 3 h mice were anesthetized and perfused transcardially with Ringer solution. After decapitation the.brain was removed, frozen on dry ice and mRNA was prepared from brain tissue. First strand cDNA synthesis was performed and samples were analyzed by real time PCR. LPS treatment resulted in 4-5 fold increase of L119 mRNA levels normalized to cyclophilin A levels. Arrow bars represent SD.

FIG. 32: Strategy for generation of L119 ko mice (Replacement of entire ORF by LacZ/neo^{R} cassette with LacZ reporter under control of the endogenous L119 promoter)

A L119 gene targeting construct was generated by replacing exon 3, encoding the L119 ORF, by a LacZ/neoR cassette. The cassette consisted of a promotorless lacZ gene followed by pgk-neo driven by a thymidine kinase promotor. Homologous recombination resulted in L119 ko mice expressing the β-galactosidase reporter gene under control of the endogenous L119 promotor.

FIG. 33: Cycloheximide treatment of wt and L119 ko mice.

Northern blot analysis of total RNA derived from brain hemispheres from wt and L119 ko mice after 4 h cycloheximide (CHX) treatment. CHX induced L119 gene expression in wt animals (left and middle panel). In ko animals deficiency of L119 mRNA coding sequence (middle panel) and knock-in expression of β-galactosidase was demonstrated (right panel).

FIG. 34: Developmental expression of β-galactosidase in heterozygote E12.5 L119 ko embryos.

L119 promotor activity in heterozygote E12.5 embryos expressing β-galactosidase from the endogenous L119 was analyzed. Embryos were fixed in formaldehyde/glutaraldehyde solution and stained for 48 h at 30° C. with X-gal-staining solution. After incubation with increasing concentrations of glycerin in PBS (30-80%) embryos were embedded in gelatin/glutaraldehyde solution and sectioned at 50 μm. X-gal staining of brain (A,C), spinal cord (B) and heart sections (D,E) is shown.

FIG. 35: Increased infarct volume of L119 ko mice in a model of focal cerebral ischemia.

48 h after permanent occlusion of the left median cerebral artery coronal cryosections from wt (A) and L119 ko mice (B) were silverstained and the infarct volume was determined (in C (wild-type) and D (knockout) affected areas are colored in white for better visualization). L119 ko mice showed an increased infarct volume compared to wt littermates.

FIG. 36A: Determination of infarct volumes of wt and 1119 ko mice in a model of focal cerebral ischemia.

48 h after permanent occlusion of the left median cerebral artery coronal cryosections from wt and L119 ko mice were silverstained. The infarct volume was determined and corrected for brain edema. Data were obtained from 14 wt and 17 L119 ko mice, arrow bars represent SEM values. L119 ko mice showed a statistically significant increase in infarct volume compared to wt littermates.

FIG. 36B: Analysis of tail bleeding time of wt and L119 ko mice.

Mice were anesthetized with sodium pentobarbital and the tail immersed into a bath of PBS at 37° C. 5-8 mm of the tail was quickly cleaned and amputated using surgical scissors. Subaqueous bleeding time was defined by the time from the cut until blood flow had stopped for approximately 3-5 sec. Mean bleeding times for wt and ko mice are shown. Error bars represent standard errors. L119 ko mice showed significantly decreased tail bleeding times compared to wt littermates (p<0.0001; unpaired t-test).

FIG. 37: Whole blood aggregation assay.

Heparin blood was obtained from wt and L119 ko mice (total number of animals n=11). Blood cell counts were determined and aliquots of blood were placed into an aggregometer. After addition of agonists (collagen, A23187) aggregation was determined by measurement of increase in electrical resistance over a period of 5 minutes. Data are shown in arbitrary units and represent maximal resistance divided by platelet concentration. (Arrow bars represent SEM values.) Blood derived from L119 ko mice coagulated more vigorously than wt blood.

FIG. 38: Platelet aggregation of wt and L119 ko mice.

Platelet rich plasma (PRP) was prepared from Heparin blood derived from 2 wt and 2 L119 ko mice, respectively. Platelet counts were determined and aggregation of platelets was measured by increase of light transmission in an Bio-Data Aggregometer at 37° C. after addition of agonists (Agonists: 1 μM ADP (curve 1 (wild-type) and 2 (knockout)) or 0.5 μg/ml collagen (curve 3 (wild-type) and 4 (knockout))) for 6 minutes. Platelet poor plasma (PPP) was used as a control for definition of 100% light transmission. Platelets from L119 ko mice (curve 2 and 4) showed a more vigorous aggregation profile than platelets from wt littermates (curve 1 and 3).

FIG. 39: L119 mRNA expression in megakaryoctes. Bone marrow, derived from rat femur was embedded in OCT and sectioned at 10 μm. In situ hybridisations were performed using a digoxygenin-labeled L119 riboprobe followed by immunological detection with alkaline phosphatase. The tissue was counter-stained with nuclear fast red to visualize tissue morphology. Rats were either injected i.p. with 50 mg/kg cycloheximide (B-D) or with vehicle (PBS/Ethanol 1:1) (A) 4 h prior to decapitation. Megakaryocytes (marked by arrows) derived from CHX treated animals had induced L119 gene expression levels.

FIG. 40: L119 protein expression in white blood cells. Heparin blood obtained by cardiac puncture of wt and L119 KO mice was mixed with Hank's Balanced salt solution (2:1), layered on top of an equal volume of Histopaque-1119 (Sigma-Aldrich) and centrifuged at 400 g for 30 min. The plasma fraction and the white blood cells (WBC)/platelet fraction were combined in a fresh tube and centrifuged at 120 g for 8 min. The pellet represents white blood cells and the supernatant the platelet rich plasma (PRP). Platelets were collected by centrifugation of the PRP at 2000 g for 10 min. Cell pellets were lysed with 2× Laemmli-buffer and analyzed by western blotting (lanes 1 and 2). In parallel direct analysis of the WBC/platelet fraction was performed. After Histopaque-1119 centrifugation the upper plasma fraction was discarded and the layer consisting of WBC and platelets was transferred to a fresh tube, a 10 fold volume of HBSS was added and blood cells were collected by centrifugation at 2000 g for 10 min. Cell pellets were lysed with 2× Laemmli-buffer and analyzed by western blotting using an L119 specific antibody (protein A purified IgG 3843 at 1:500) (lanes 3 and 4).

### EXAMPLES

In the following implementation examples, the invention is clarified with reference to the enclosed figures.

General Methods

Oligonucleotides can be synthesized chemically in a known manner, for example using the phosphoamidite method (Voet, Voet, Biochemistry, 2nd edition, Wiley Press New York, pages 896-897). The cloning steps performed within the context of the present invention, such as restriction cleavages, agarose gel electrophoresis, purification of DNA fragments, transfer of nucleic acids to nitrocellulose and nylon membranes, the linking of DNA fragments, transformation of E. coli cells, culture of bacteria, replication of phages and sequence analysis of recombinant DNA, were carried out as described in Sambrook et al. (1989) Cold Spring Harbor Laboratory Press; ISBN 0-87969-309-6 or Ausubel FM et al., (1998) Current Protocols in Molecular Biology (New York: John Wiley & Sons). Recombinant DNA molecules were sequenced by the method of Sanger (Sanger et al., Proc. Natl. Acad. Sci. USA 74 (1977), 5463-5467) using an ABI laser fluorescence DNA sequencer.

Other methods which were routinely used were:

a) Polymerase Chain Reactions Polymerase chain reactions were carried out in a GeneAmp PCR system 9700 supplied by PE Applied Biosystems (Norwalk, Conn., USA). Taq-DNA polymerase (Cat. No. 10342-020) was obtained from Life Technologies GmbH (Eggenstein, Germany). The PCR conditions were selected in accordance with the recommendations of the Taq polymerase producer contained in the accompanying manual (Basic PCR Protocol) and using the ×10× PCR buffer minus Mg” which was supplied at the same time. The final concentration of the buffer was 1× while the concentration of the dNTP mixture (Cat. No. 1969064, Roche Diagnostics GmbH, Mannheim, Germany) was in each case 0.2 mM, the MgCl₂ concentration was 1.5 mM, and the concentrations of the two PCR primers were in each case 0.5 μM. The final volume of the PCR reaction was 50 μl.

b) Reverse Transcription

Reverse transcription for preparing first-strand cDNA was carried out using SUPERSCRIPT II RNase H Reverse Transcriptase (Cat. No. 18064-014; Life Technologies GmbH, Eggenstein, Germany) in accordance with the protocol given in the accompanying manual (“First strand CDNA synthesis using SUPERSCRIPT II for RT-PCR”). 5 μg of total RNA were used for the synthesis.

c) RNA Extraction

Total RNA was isolated from cells and tissues using the “Single step RNA isolation from cultured cells or tissues:

Basic Protocol” in Ausubel et al. (eds.), Current Protocols in Molecular Biology, Volume 1, Supplement 36 (1996), pp. 4.2.1.-4.2.2. and 4.2.6-4.2.7. (John Wiley and Sons).

d) Northern Blotting

10 g of total RNA were used for Northern blotting experiments. The RNA was fractionated electrophoretically on a denaturing agarose-formaldehyde gel and, after that, transferred to a nylon membrane (Hybond N+, Cat. No. RPN2222B, Amersham Pharmacia Biotech Europe GmbH, Freiburg, Germany). The procedure followed was that described in the protocols in Ausubel et al. (eds.), Current Protocols in Molecular Biology, Volume 1, Supplement 37 (1997), pages 4.9.2.-4.9.8. (John Wiley and Sons).

e) Southern Blotting

10 μg of DNA were used for Southern blotting experiments. The DNA was fractionated electrophoretically on an agarose gel and, after that, transferred to a nylon membrane (Hybond N+, Cat. No. RPN2222B, Amersham Pharmacia Biotech Europe GmbH, Freiburg, Germany). The procedure followed was that described in the protocols in Ausubel et al. (eds.), Current Protocols in Molecular Biology, Volume 1, Supplement 21 (1993), pp. 2.9.2.-2.9.6. (John Wiley and Sons).

f) Radioactive Labeling of DNA Fragments

Suitable DNA fragments (20 ng) were radioactively labeled by incorporating ³²β-adCTP (Amersham Pharmacia Biotech Europe GmbH, Freiburg, Germany) using the Rediprime II Kit (Cat. No. RPN 1633, Amersham Pharmacia Biotech Europe GmbH, Freiburg, Germany) in accordance with the protocol described in the manual accompanying the kit. The labeled DNA fragment was freed from unincorporated radioactivity by means of Biospin P6 (Cat. No. 732-6002; BioRad Laboratories GmbH, Munich) chromatography performed in accordance with the protocol in the accompanying manual.

g) Hybridization of Nylon Filters with Radioactively Labeled DNA Fragments

Nylon filters carrying immobilized DNA or RNA were hybridized as described in the protocols in Ausubel et al. (eds.), Current Protocols in Molecular Biology, Volume 1, Supplement 21 (1993), pp. 2.10.2.-2.10.3. (John Wiley and Sons) but with the following changes. The hybridization buffer used was: 7% SDS, 250 mM sodium phosphate (pH 7.2), 1 mM EDTA. The hybridization was carried out overnight at 68° C. in a hybridization oven (OV3, Biometra) in a rotating glass tube. The filters were washed in the tube using solutions which had been preheated to 68° C.: twice for 10 min with 5×SSC/0.1% SDS and twice for 10 min with 2×SSC/0.1% SDS. The washed filters were wrapped in SaranWrap film and exposed on imaging plates (Fujifilm, BAS-IP MS 2040), after which scanning took place in a phosphoimager (FLA 2000, Fuji), with the hybridization spots then being quantified.

h) Digoxigenin-Labeled Riboprobes

In order to prepare the antisense riboprobes, pBS(SK)-L119 was linearized with Not I; it was cut with Acc651I in order to prepare the sense probe. The linearized DNA fragments were extracted twice with phenol/chloroform/isoamyl alcohol and then extracted once with chloroform/isoamyl alcohol. The DNA was precipitated-with ethanol overnight then washed twice with 70% ethanol and resuspended to a concentration of 0.5 mg/ml in TE. The in vitro transcription was carried out using the Ambion Maxiscript T3/T7 kit (Cat. Nr. 1324) and the digoxigenin RNA labeling mix (Roche Diagnostics; Cat. No. 1277073). 1 μg of template DNA was employed for a 20 μl labeling reaction (in accordance with the manufacturer's istructions) and the reaction mixture was incubated at 37° C. for 2 h. The template DNA was subsequently degraded, at 37° C. for 20 min, by adding 2 μl of RNase-free DNase I (Roche Diagnostics; Cat. No. 776785). Unincorporated NTPs were separated off through a Quant G-50 spin column (Amersham Pharmacia Biotech Europe GmbH; Cat. No. 27-5335-01). For quality control, and for determining quantity, 1/10 of the riboprobes was loaded onto a 1% TBE agarose gel.

i) in situ Hybridizations

Tissue Preparation

The tissue was embedded in Tissue-Tek/OCT (Sakura; Cat. No. 4583) and shock-frozen in an ethanol/dry ice bath. 20 μm cryo sections were then prepared and mounted on Superfrost-Plus microscope slides. The sections were dried in air for 20 min and then stored at −20° C. for subsequent use. The sections were next fixed in 4% paraformaldehyde-PBS (PFA from Sigma-Aldrich; Cat. No. P6148) for 20 min and then washed, in-each case at RT for 5 min, three times with PBS and once with H₂O. For the acetylation, a 1% (v/v) solution of triethanolamine (TEA from Sigma-Aldrich; Cat. No. T1377) was prepared in water and the slides were immersed in the solution. Immediately after that, acetic anhydride (Sigma-Aldrich; Cat. No. A6404) was added dropwise to give a final concentration of 0.25%. After that, the slides were washed, in each case for 5 min, twice with PBS and twice with 2×SSC.

Prehybridization

The sections were firstly incubated, at RT for 2 hours, with in each case 100 μl of hybridization buffer (Amersham Pharmacia Biotech Europe GmbH, Cat. No. RPN3310) under a parafilm cover.

Hybridization

100 ng of the labeled riboprobe were added to 100 μl of hybridization buffer, denatured for 7 min at 85° C. and then cooled rapidly on ice for 10 min. The sections were incubated overnight at 55° C. in hybridization solution under parafilm. After that, they were washed for 10 min with 2×SSC at RT and once for 10 min at 55° C. They were subsequently washed for min with 0.2×SSC/50% formamide at 55° C. and then for 5 min with 0.2×SSC at room temperature.

Immunological Detection

The sections were equilibrated for 20 min at room temperature in buffer 1 (100 mM Tris-HCl, 250 mM NaCl, pH 7.5) and then blocked for 1 h with 0.5% casein (NEN, Cat. No. 734A) in buffer 1. After having been washed for 5 minutes in buffer 1, the sections were incubated for 2 h with an alkaline phosphatase-coupled anti-DIG antibody (Roche Diagnostics, Cat. No. 1093-274) diluted 1:5000. After having been washed twice for 15 minutes with buffer 1, the sections were equilibrated for 10 min in buffer 2 (100 mM Tris-HCl, 100 mM Nacl, 5 mM MgCl₂, pH 9.5). 10 ml of buffer 2 were mixed with 34 μl of NTB (Roche Diagnostics, Cat. No. 1383-213), 35 μl of BCIP (Roche Diagnostics, Cat. No. 1383-221) and 2.4 mg of Levamisole (Sigma-Aldrich, Cat. No. L9756), and the tissue sections were incubated with solution until the color had developed (overnight). For dehydration, the sections were washed in the following manner: 2 min with PBS, 2 min in H₂O, 2 min in 30% ethanol, 2 min in 70% ethanol, 2 min in 95% ethanol, 2×2 min in 100% ethanol and 2×2 min in xylene. After that, the sections were embedded with Permount (Fisher Scientific, Cat. No. SP15-10) under cover slips.

j) Nissl Staining

Tissue sections were incubated for 2 min in 0.5% aqueous cresyl violet solution (EM Sciences, Cat No. CX2065-1) and after that excess dye was washed out by immersing the slide 3 times in H₂O. The sections were then destained in 70% ethanol until the desired color intensity had been obtained. For dehydration, the sections were washed in the following manner: 2 min with PBS, 2 min in H₂O, 2 min in 30% ethanol, 2 min in 70% ethanol, 2 min in 95% ethanol, 2×2 min in 100% ethanol and 2×2 min in xylene. The sections were subsequently embedded with Permount (Fisher Scientific, Cat. No. SP15-10) under cover slips.

k) Transient Transfections

Calcium phosphate transfections of COS and HEK293 cells For the transfections, 10 μg of DNA were mixed with 50 μl of 2.5 M CaCl₂ and 450 μl of H₂O. The DNA solution was pipetted dropwise, while mixing, into 500 μl of 2×BBS (50 mM BES (Sigma-Aldrich; B9879 or B6266), 280 mM NaCl; 1.5 mM Na₂HPO₄, pH 7.03 at RT). The DNA solution was added dropwise to a 60 to 70% confluent 10 cm dish containing 10 ml of culture medium. The cells were cultured for from 8 to 20 h in a 3% CO₂ incubator and then cultured, until used, for a further 24 to 48 h in a 5% CO₂ incubator.

l) Western Blotting Analysis

Protein samples were fractionated in 12% denaturing SDS-polyacrylamide gels and blotted in transfer buffer (25 mM Tris-HCl, 150 mm glycine, 10% methanol, pH 8.3) onto a nitrocellulose membrane (Protran BA79, Schleicher and Schuell) for 60 to 90 min using a Semi-Dry Blotting Chamber (Biometra). As a control, the membrane was reversibly stained with Ponceau S solution and blocked for 60 min in PBS/0.02% Tween 20 containing 5% skim milk powder. Polyclonal L119 antibodies were used at a dilution of 1:1000 while the anti-myc antibody (Invitrogen, Cat. No. R950-25), and the anti-Flag-M2 antibody (Sigma-Aldrich, Cat. No. F3165) were used at a dilution of 1:2000. The incubations were carried out for 1 h in blocking buffer. After having been washed three times for in each case 10 min in PBS/0.02% Tween 20, the membranes were incubated for 20 min with HRP-conjugated anti-rabbit or anti-mouse IgG antibodies (Jackson ImmunoResearch Laboratories Inc.) which were diluted 1:4000 in blocking buffer. After having been washed three times for 15 to 20 min in PBS/0.02% Tween 20, the membranes were swiveled for 5 min in SuperSignal (Pierce, Cat. No. 34080). The chemiluminescence signals were detected using Hyperfilm-ECL (Amersham Pharmacia Biotech, Cat. No. RPN2103K).

### Example 1

### The Mouse L119 Genomic Sequence

Using two primers (SEQ ID NO: 8 and 9) obtained from the coding region of rat L119, a 329 bp-long fragment was amplified from mouse brain cDNA. In order to obtain the cDNA, total RNA was isolated from mouse brain in accordance with the “RNA extraction” protocol (see above) and 5 μg of this RNA were reverse-transcribed into cDNA in accordance with the “reverse transcription” protocol (see above). The primers used for amplifying the mouse probe were:

| | |
|---|---|
| rL119-4s: | |
| 5′-TATCACTCAGCCCGGTCACCCTGG-3′ | (SEQ ID NO: 8) |
| rL119-5as: | |
| 5′-ACGCCTGGGGATGAGGAAGCCACG-3′ | (SEQ ID NO: 9) |

The PCR was carried out in accordance with the “polymerase chain reaction” protocol but using the following modifications: 50 ng of cDNA, with an initial denaturation for 3 min at 96° C. and then 30 cycles with denaturation for 30 sec at 96° C., annealing for sec at 64° C. and elongation for 30 sec at 72° C.

The resulting fragment was labeled in accordance with the “radioactive labeling of DNA fragments” protocol (see above) and used for hybridizing a mouse genomic cosmid library (RZPD library No. 121; 129/ola mouse cosmid, RZPD Berlin; Germany). The hybridization took place in accordance with the “hybridization of nylon filters with radioactively labeled DNA fragments” protocol. One clone gave a strong positive signal with the probe employed. Cosmid DNA was isolated from one positive clone using the “large construct kit” (Cat.No. 12462; Qiagen GmbH, Hilden, Germany) and following the protocol in the manual accompanying the kit (Version 06/99). This clone was verified as being L119-positive by means of carrying out various restriction digestions and hybridizations with L119 probes (by comparing the band patterns which were obtained with those from mouse genomic DNA). Various fragments from the cosmid were subcloned into a plasmid vector and sequenced using a transposon insertion method (GPS-1, New England Biolabs, Beverly, Mass.; USA), and the sequences were then assembled using the SeqMan program (Lasergene, Madison, Wis., USA).

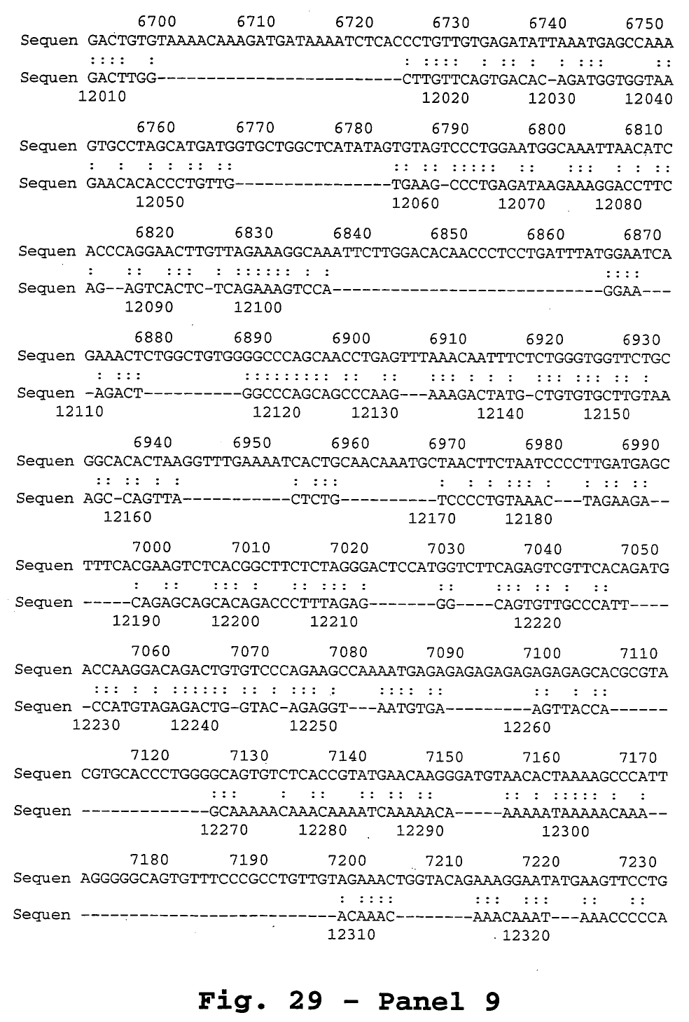

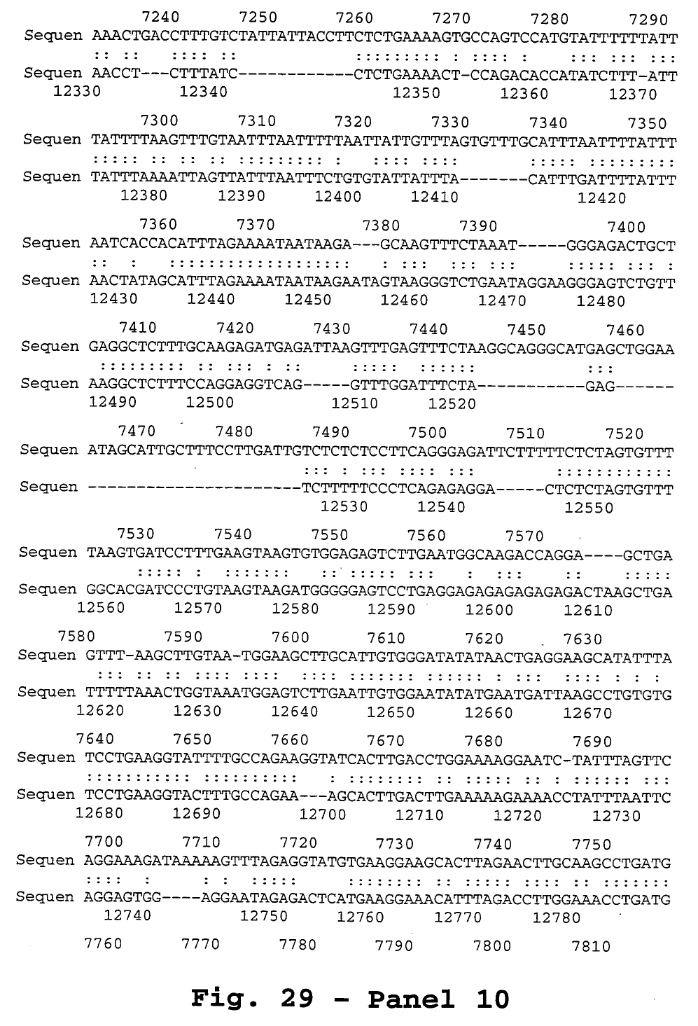

The mouse L119 genomic sequence is depicted in SEQ ID NO: 4. The mouse L119 protein sequence is depicted in SEQ ID NO: 24. Comparison of the mouse genomic sequence and the rat cDNA sequence shows that the region coding for the mouse L119 protein (longest open reading frame; from the start codon at position 10736 to 10738 to the stop codon at position 11474 to 11476 in SEQ ID NO:4) is not interrupted by introns. While the coding region is very strongly conserved between the mouse and the rat, very high sequence differences are found in some places in the 3′-flanking region (see FIG. 27).

For example when the sequences corresponding to position 11645 in sequence SEQ ID NO: 4 are compared, it is then seen that there is an insertion of 275 bp in the rat cDNA for which there is no correspondence in the mouse genomic sequence (SEQ ID NO: 4). The region from position 12004 to position 12076 is likewise not conserved. Interestingly, however, 7 of the 8 AUUUA motifs (see above) which are found in the rat cDNA are also conserved in the mouse.

### Example 2

### New Splice Variants of the Rat L119

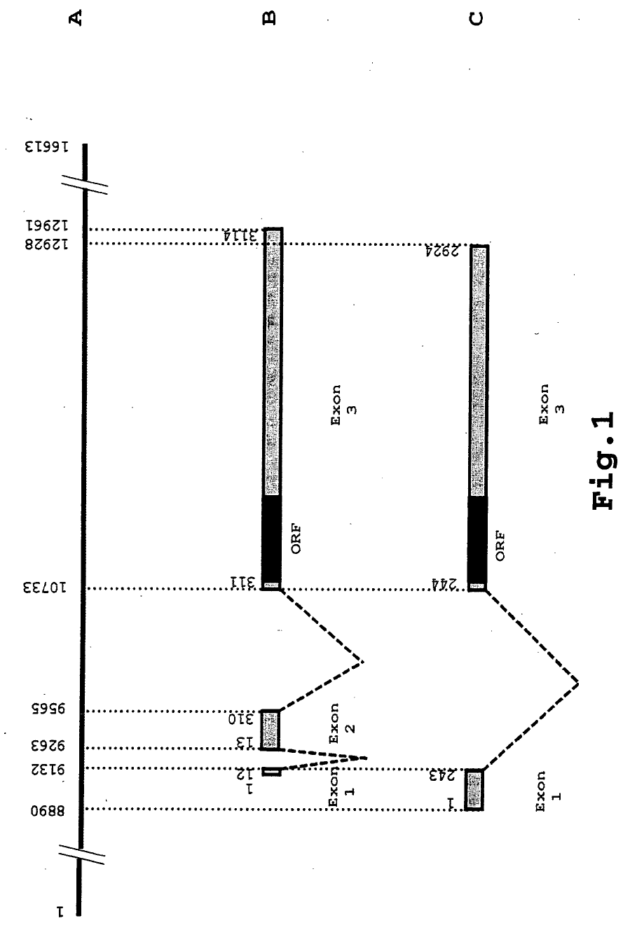

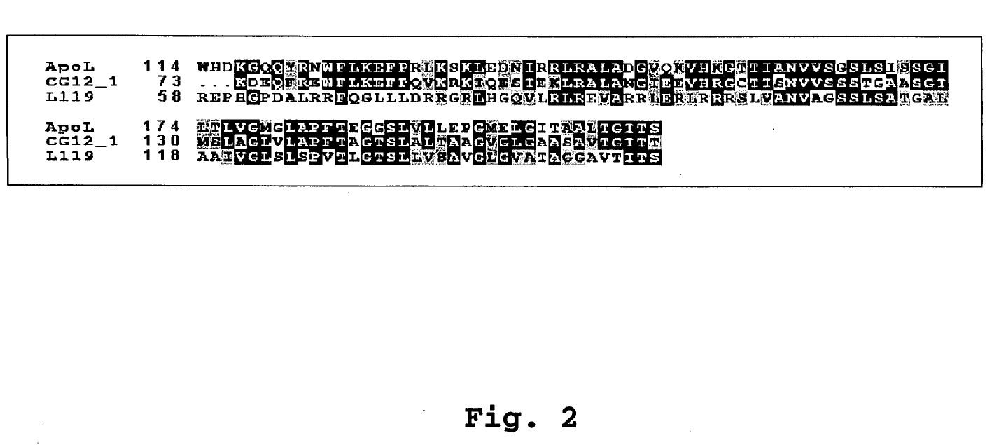

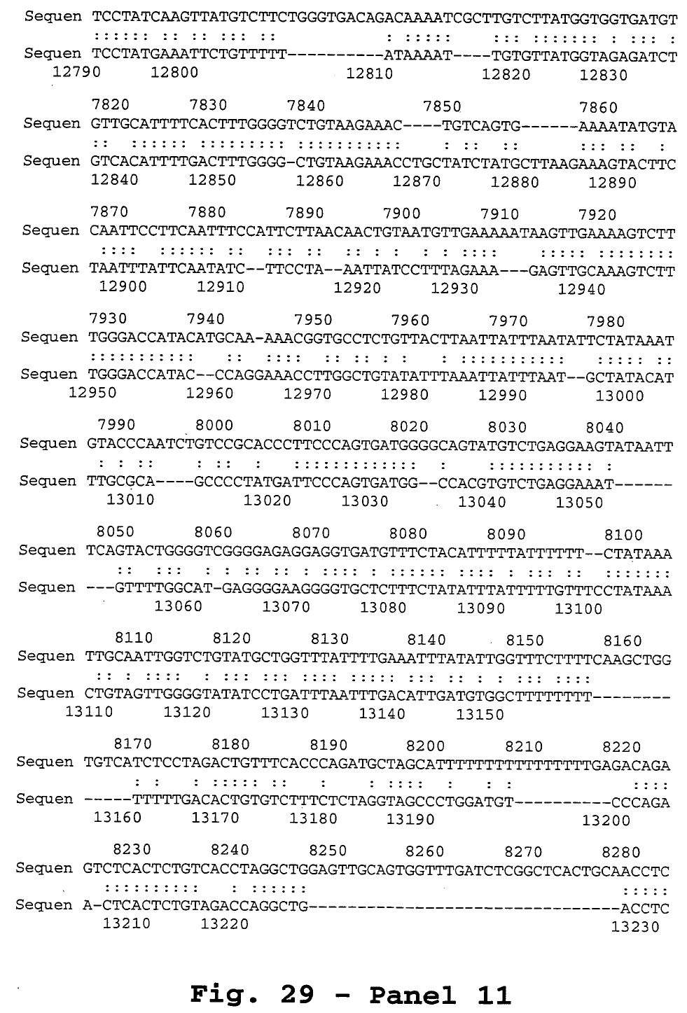

In order to search for new splice variants of L119, a rat hippocampus λ phage library (in the vector Lambda ZAP II; from Stratagene), which was prepared from the animals following stimulation with MECS and the administration of cycloheximide, was screened with the abovementioned 329 bp long fragment from the L119 coding region as described in Worley et al. (WO 99/40225). Approximately 500000 clones from the phage library were transferred to nylon membranes using the “Plating and transferring bacteriophage libraries: Basic protocol” in Ausubel et al. (eds.), Current Protocols in Molecular Biology, Volume 1, Supplement 34 (1996), pp. 6.1.1.-6.1.4. (John Wiley and Sons) and hybridized with the L119-specific probe (preparation and labeling, see above) in accordance with the “hybridization of nylon filters with radioactively labeled DNA fragments” protocol (see above). Because the animals were stimulated with cycloheximide, the library contains a large number of L119 clones. The clones which were positive in the hybridization were isolated using the “Purification of bacteriphage clones: Basic protocol” in Ausubel et al. (eds.), Current Protocols in Molecular Biology, Volume 1, Supplement 13 (1987), pages 6.5.1.-6.5.2. (John Wiley and Sons). The phagemids containing the cDNA were excized in vivo using a rapid excision kit (Cat. No. 45 211204; Stratagene, CB Amsterdam Zuidoost) following the protocol in the accompanying manual. The inserts were sequenced using a transposon-insertion method (GPS-1, New England Biolabs, Beverly, Mass.; USA) and the sequences were assembled using the SeqMan program (Lasergene, Madison, Wis., USA) and subjected to sequence analysis. Sequence comparisons carried out using the rat L119 cDNA showed that two clones had significantly longer 5′ ends (which were both different from each other; SEQ ID NO: 1 and SEQ ID NO: 2). Interestingly, the two 5′ ends exhibited a very high degree of sequence homology with two regions of the mouse genomic sequence (SEQ ID NO: 4) which are located further 5′ upstream of the coding sequence (FIG. 27 and FIG. 28; compare also FIG. 1). In this connection, perfect splice donor and acceptor sequences (compare Alberts B et al. (1994) Molecular Biology of the Cell, Garland Publishing, New York) were located at the points at which the sequence homologies between the mouse genomic sequence and the rat cDNA clones ceased. Consequently, the two isolated cDNA sequence represent two alternative splice variants of the rat L119. An insertion of 76 bp in length (positions 2427 to 2502), which was not present in the other rat cDNA clones, was found in the 3′-untranslated region of the cDNA clone SEQ ID NO: 1. The following sequence differences were found in SEQ ID NO: 1 as compared with the sequence given in WO 99/40225: position 1443: A instead of G, position 1557: A instead of G, position 1882; C instead of T, position 2884: C instead of T, position 2973: A instead of G, position 3090: C instead of T and also an insertion of 8 nucleotides at position 2531 (the position numbers relate to SEQ ID NO: 1).

FIG. 1 shows a diagrammatic depiction (not to scale) of the exon/intron structure of the L119 gene, with this diagram being obtained on the basis of comparing the sequence of the mouse genomic sequence (FIG. 1A; SEQ ID NO: 4) with those of the two new rat cDNA splice variants (SEQ ID NO: 1 in FIG. 1B and SEQ ID NO: 2 in FIG. 1C). The exons were numbered 1 to 3 in accordance with their positions in the mouse genomic L119 sequence (SEQ ID NO: 4). As can be seen from the figure, exon 2 is used in the SEQ ID NO: 1 cDNA but not in the SEQ ID NO: 2 cDNA. The exon limits in the mouse genomic sequence are given above the diagram of SEQ ID NO: 4. The rat cDNAs are depicted as gray quadrangles; the black part represents the open reading frame (ORF). The nucleotide positions which correspond to the exon limits are marked above the quadrangles.

The translated sequence of the longest open reading frame in the two new splice variants of the rat L119 (which is identical in both the splice variants SEQ ID NO: 1 and SEQ ID NO: 2) is depicted in SEQ ID NO: 3. Profile database searches showed significant identities (26%), over a region of 97 amino acids, with human apolipoprotein L, a new HDL lipoprotein (ApoL,

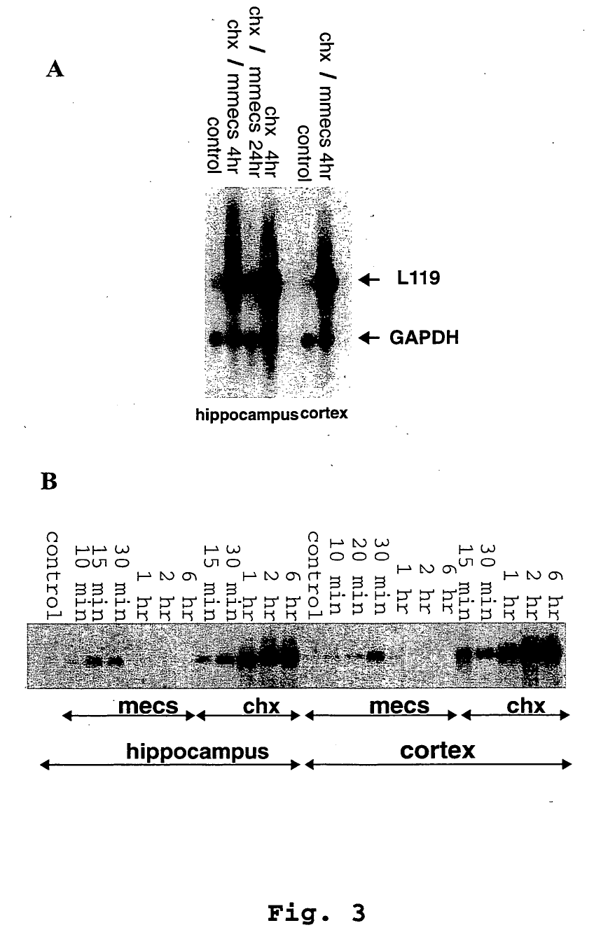

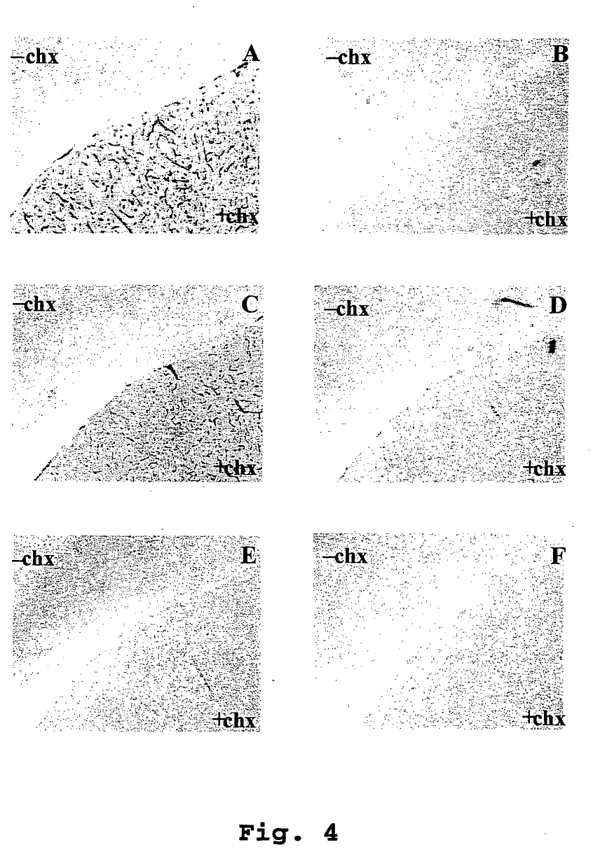

Duchateau PN et al. (1997) J Biol Chem 272, 25576-25582; EMBL database entry AF019225) and 28% identities with a TNFalpha-inducible gene (CG12_{—}1; Horrevoets AJ et al. (1999) Blood 93, 3418-3431; EMBL database entry AF070675). FIG. 2 depicts a multiple alignment of the regions extending from amino acid 114 to amino acid 210 in ApoL with amino acids 73 to 166 in CG12_{—}1 and amino acids 58 to 154 in SEQ ID NO: 6. The conserved amino acids are bordered in black while the amino acids having similar properties are bordered in gray. Despite the fact that the degrees of identity are not very high, the alignment which is shown is of relevance since the conserved amino acids are located in a group of previously undescribed human proteins. The alignment was generated using the Clustal method in the MegAlign program in DNASTAR, version 4.0.1., and depicted using Boxshade from Dr. K. Hofmann (Cologne). Programs for predicting transmembrane regions detected three significant hits having different scores: amino acids 77 to 93 (PSORTII program; WWW version 1.12.1998 from Dr. K. Nakai) and amino acids 77-110 (Hofmann K and Stoffel W (1993) Biol. Chem. Hoppe-Seyler 347, 166) and also amino acids 55 to 85 and 157 to 173 (Hofmann K and Stoffel W (1993) Biol. Chem. Hoppe-Seyler 347, 166). Furthermore, there are significant scores for a leucine zipper motif (amino acids 15 to 36 and 22 to 43 respectively). Coiled coil structures were predicted for amino acids 37 to 50 and 189 to 220.

### Example 3

### The Human L119 Coding Sequence

A homology search of the EMBL nucleotide database was carried out using the sequence depicted in SEQ ID NO: 4. In this connection, a very high degree of similarity was established with an entry (AC007215; Release 62, last updated, Version 21; dated 21 Feb. 2000) of 131 unordered sequence segments of the human BAC clone RPCI11-59H1. The question then arose as to whether the sequence which had been found was indeed the homologous gene in humans. It is not possible to answer this question unambiguously simply using the sequence information contained in database entry AC007215. When this sequence is compared with the mouse L119 genomic sequence (SEQ ID NO: 4), it is observed that, aside from the coding sequence, regions which in part have a very high degree of similarity with the mouse sequence are present, in particular, in the 5′-flanking region. Another pointer to identifying the human sequence as human L119 is obtained when the rat L119 splice variants (SEQ ID NO: 1 and SEQ ID NO: 2) are included in the sequence comparison. This comparison shows that exon 1 is strongly conserved (including the splice donor and acceptor sequences) to about 78%, between mouse and human and that the human exon 1 is located at about the same distance from the coding exon as in the mouse genomic sequence. If the sequence in AC007215 were a pseudogene, this exon in the AC007215 sequence ought not then be separated from the coding sequence by an intron. However, it is not possible to identify exon 1 simply from the sequence information in AC007215. Translating the sequence segment in AC007215 which was homologous to the mouse coding sequence resulted in an open reading frame of 297 amino acids in length and consequently 51 amino acids longer, in the aminoterminal direction, than the mouse and rat proteins. Examination of the sequences obtained from the mouse and rat failed to identify any such open reading frame, which was extended at the aminoterminus, in these species. In order to check whether the L119-like sequence in database entry AC007215 is in fact expressed, PCR primers were designed which span the entire coding region. The primers which were used for amplifying the human L119 cDNA were:

| | |
|---|---|
| humL119-5′-myc (EcoRI): | |
| 5′-CTATGAATTCACCATGATCCACTGGAAACA | (SEQ ID NO: 10) |
| GA-3′ | |
| humL119-3′-myc (XbaI): | |
| 5′-CACTAGTCTAGAGAAAAACAGCCCTGCA | (SEQ ID NO: 11) |
| CGC-3′ | |

These primers were used to carry out an RT-PCR proceeding from human placental cDNA (obtained from mRNA provided by Clontech). The Clontech SMART-RACE cDNA amplification kit (Cat. No. K1811-1) was used, in accordance with the manufacturer's instructions (protocol No. PT3269-1; Version PR88571), for synthesizing the first cDNA strand. 1 μg of the human placental total RNA contained in the kit was used as the RNA template for doing this. The 3′-RACEcDNA synthesis primer (3° C.DS: 5′-AAGCAGTGGTAACAACGCAGAGTAC(T) 30N-1N-3′) was used as the primer. The reaction product from the first-strand synthesis was diluted 1:10 with tricine-EDTA buffer (SMART-RACE kit) and then used as the template for the subsequent PCR reaction. For this, a 50 μl mixture was prepared from 5 μl of 10× cloned Pfu buffer (Stratagene); 2 μl of dNTP mixture (5 mM; Cat. No. 1969064, Roche Diagnostics GmbH, Mannheim, Germany); in each case 1 μl of the primers SEQ ID NO: 10 and 11 (stock conc. 5 μM); 5 μl of first-strand cDNA template, 35 μl of H₂O and 1 μl of Pfu-turbo DNA polymerase (Stratagene) and, after the mixture had been incubated at 96° C. for 3 minutes, 30 PCR cycles corresponding to the temperature program: 30 sec of primer annealing at 58° C., 70 sec of strand extension at 72° C and 30 sec of DNA double-strand melting at 96° C., together with a concluding extension step of 7 min at 72° C., were then carried out. After that, 1 μl of the PCR reaction mixture was used as the template for a subsequent PCR reaction.

The reaction was carried out in an analogous manner to that of the first PCR reaction. The resulting PCR product was gel-purified, cleaved with the restriction enzymes EcoRI and XbaI, subcloned into the expression vector pcDNA3.1-myc/His-A (Invitrogen), and then sequenced. The sequence in fact contained an open reading frame of 297 amino acids (SEQ ID NO: 6). The human protein containing 246 amino acids, corresponding to the mouse and rat proteins, is depicted in SEQ ID NO: 7. When compared to the AC007215 sequence, there is a base exchange of T instead of C at position 132, and a base exchange of C instead of T at position 171, in SEQ ID NO: 5, these base exchanges not, however, leading to any change in the protein sequence.

In order to verify that the investigated human sequences in the 3′-flanking region were expressed, various primer pairs from this region were used in RT-PCR reactions which were carried out with human hippocampus cDNA (obtained from mRNA supplied by Clontech). All the cases which were investigated resulted in the amplification of bands which demonstrated that the underlying DNA sequences were expressed in the human hippocampus. For the RT-PCR, human brain total RNA (Cat. No. 64020-1; from Clontech Heidelberg, Germany) was transcribed into cDNA (“Reverse transcription” protocol). All the PCR reactions were carried dut in accordance with the “polymerase chain reactions” protocol (see above) under the following conditions: 0.2 μl of the cDNA in a reaction volume of 15 μl, with 3 min at 96° C. for initial denaturation and then 35 cycles of 30 sec at 96° C. for denaturation, 30 sec at 62° C. for annealing and 30 sec at 72° C. for elongation. Advantage cDNA Polymerase Mix (Cat. No. 8417-1; from Clontech Heidelberg, Germany) was used as the enzyme while employing the reaction buffer (which already contained MgCl₂) which was supplied with it (no additional MgCl₂ was added).

The primer combinations employed, and the fragment sizes which were correspondingly obtained, were:

| | | |
|---|---|---|
| a) | hL119-1s and hL119-1as | 416 bp |
| b) | hL119-1s and hL119-2as | 243 bp |
| c) | hL119-2s and hL119-1as | 438 bp |
| d) | hL119-2s and hL119-2as | 265 bp |
| e) | hL119-3s and hL119-3as | 405 bp |
| f) | hL119-3s and hL119-4as | 445 bp |
| g) | hL119-4s and hL119-3as | 316 bp |
| h) | hL119-4s and hL119-4as | 356 bp. |

The primers which were used for amplifying the human cDNA were:

| | |
|---|---|
| hL119-1s: | |
| 5′-AGTTATGTCTTCTGGGTGACAGAC-3′ | (SEQ ID NO: 12) |
| hL119-2s: | |
| 5′-TTGCAAGCCTGATGTCCTATCAAG-3′ | (SEQ ID NO: 13) |
| hL119-3s: | |
| 5′-ATCGTGGGGCTCTCGCTCAG-3′ | (SEQ ID NO: 14) |
| hL119-4s: | |
| 5′-CGTCACCATCACGTCCGATCTC-3′ | (SEQ ID NO: 15) |
| hL119-1as: | |
| 5′-CAGTCTAGGAGATGACACCAGC-3′ | (SEQ ID NO: 16) |
| hL119-2as: | |
| 5′-AGGGTGCGGACAGATTGGGTAC-3′ | (SEQ ID NO: 17) |
| hL119-3as: | |
| 5′-GCTCTCGGCCAGTTTCTGAATC-3′ | (SEQ ID NO: 18) |
| hL119-4as: | |
| 5′-GCTCGCTGAGTTCGTCCAGAGC-3′ | (SEQ ID NO: 19) |

### Example 4

### Flanking Genomic Sequences Exhibiting a High Degree of Conservation During Evolution

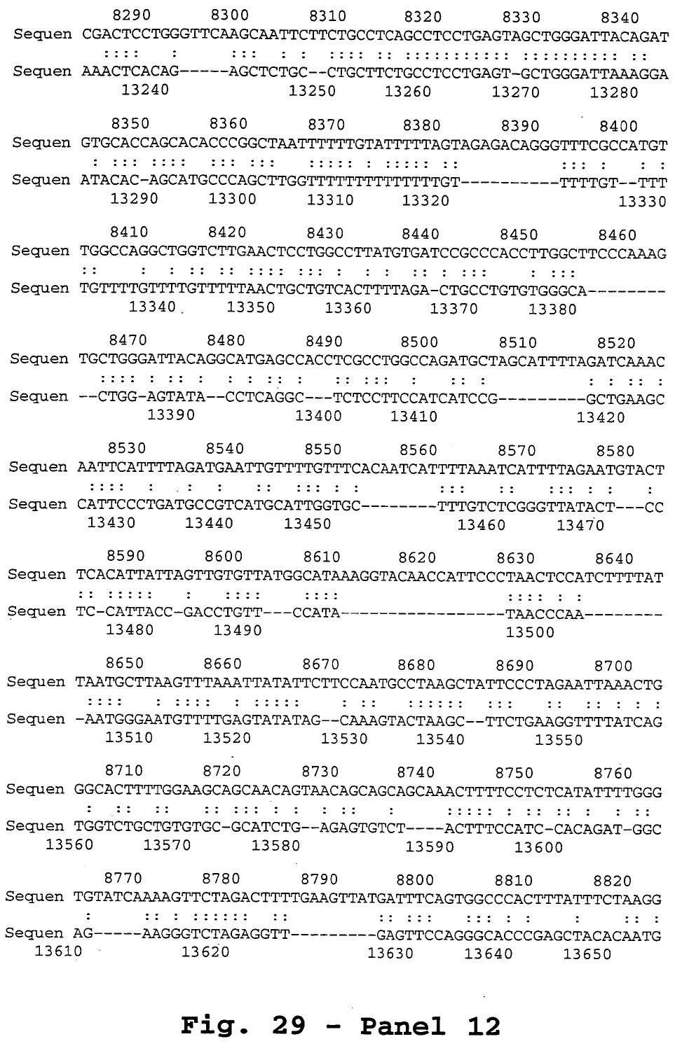

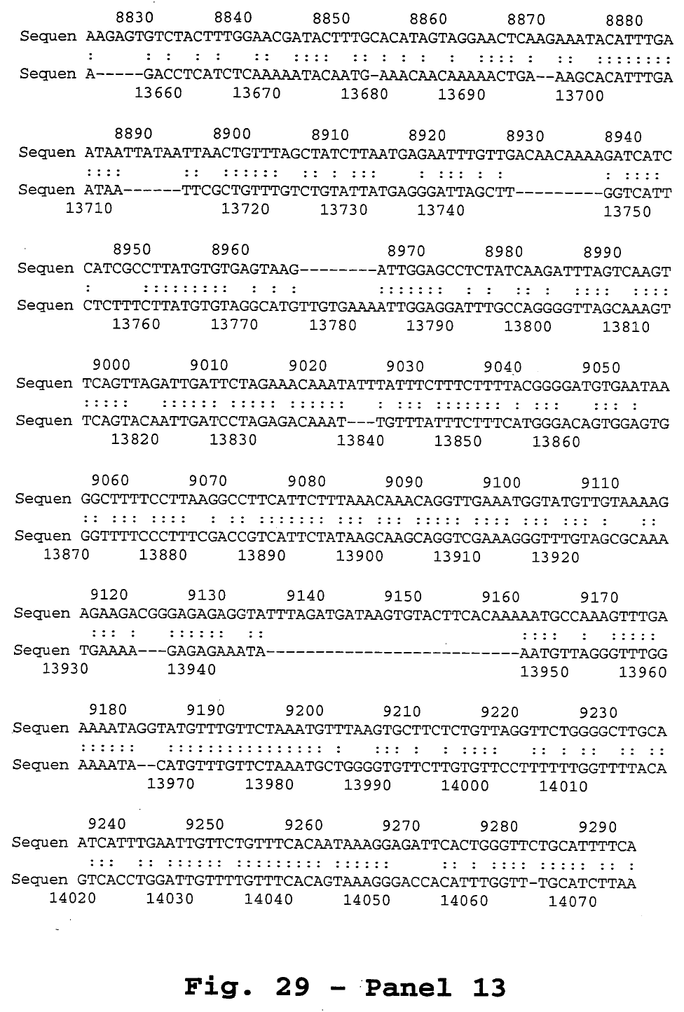

During evolution, certain gene sequences undergo a lower rate of mutation than do other segments of the genome. It can be assumed that these gene sequences are sequences which are particularly important from the functional point of view and that there is a particularly high degree of selection pressure which is militating against the sequences being mutated. If the sequences at a genomic locus are compared in two different species, it is then possible to find the regions which are more strongly conserved. In these regions, the sequences in the noncoding and flanking moieties will, inter alia, be important regulatory sequences (see, for example, Gottgens B et al. (2000) Nat Biotechnol 18, 181-186). These regulatory sequences include, inter alia: elements which influence the stability of the transcript and/or the translation; intron regulatory-elements (splicing regulators, enhancers and silencers); flanking enhancers, silencers, locus control regions and matrix attachment regions. Comparison of the mouse L119 genomic sequence (SEQ ID NO: 4) with the sequences of parts of the EMBL sequence entry AC007215, which contains the human L119 locus (SEQ ID NO: 22), showed the presence of extended conserved regions directly 5′ upstream of exon 1 (FIG. 29). It can be assumed that these regions constitute promoter elements and important cis-regulatory regions. It was also possible to find conserved sequences in the 3′ flanking region.

### Example 5

### Expression of L119 Following MECS and the Administration of Cycloheximide

After it had been discovered that it was possible to induce expression of the L119 gene in the wake of convulsion-triggering stimuli, such as multiple MECS, accompanied by the administration of cycloheximide (see the Worley et al. patent application WO 99/40225), the intention was then to investigate the influence which cycloheximide had on the induction of the expression of L119 mRNA. For this, the ability to induce L119 was compared in multiple MECS/cycloheximide-treated rats and in rats which had been treated either with cycloheximide (50 mg/kg of body weight i.p.) or with MECS. After the stimulation protocol had been performed on rats using MECS (massive electroconvulsive shock) (Worley PF et al. (1993) J Neurosci 13, 4776-4786) in combination with cycloheximide (Cole AJ et al. (1990) J Neurochem 55, 1920-1927; Lanahan A and Worley P (1998) Neurobiol Learn Mem 70, 37-43) (or using only one of the two stimuli) mRNA was then isolated from the hippocampus and cortex of these rats, and also from control animals, using the “RNA extraction” protocol (see above). Northern blot analyses (carried out in accordance with the “Northern blot′ and “radioactive labeling of DNA fragments” and “hybridization of nylon filters with radioactively labeled DNA fragments' protocols; see above) using probes for L119 and GAPDH showed that only scarcely detectable quantities of L119 mRNA were present in the control animals. A PCR fragment of 329 bp in length (description, see above) was used as the L119 probe. A GAPDH probe for the hybridization was prepared from rat brain total RNA by RT-PCR using the “RNA extraction”, “reverse transcription” and “polymerase chain reactions” protocols (see above). The following primers were used in the polymerase chain reaction:

| | |
|---|---|
| GAPDHs | |
| 5′-CTACATGGTCTACATGTTCCAGTA-3′ | (SEQ ID NO: 39) |
| and | |
| GAPDHas | |
| 5′-TGATGGCATGGACTGTGGTCAT-3′ | (SEQ ID NO: 40) |

In addition, the following conditions applied: 50 ng of cDNA with 3 min at 96° C. for the initial denaturation and then with 30 cycles of 30 sec at 96° C. for denaturation, 30 sec at 56° C. for annealing and 30 sec at 72° C. for elongation.

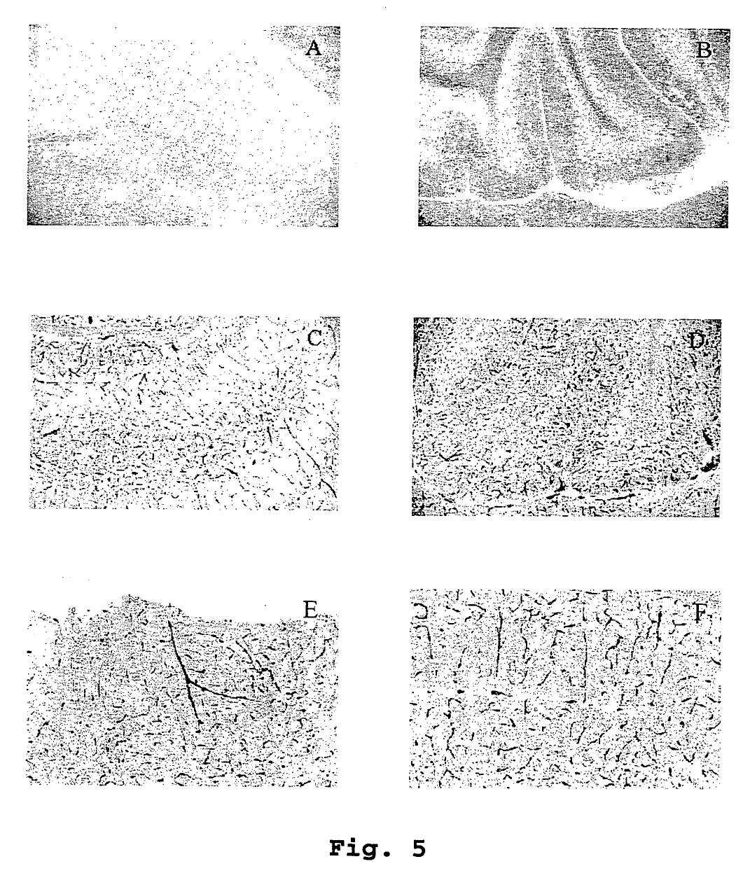

Surprisingly, the induction which was obtained in the two tissues examined was comparable, 4 hours after cycloheximide administration, with the stimulation which was obtained after using the combination of cycloheximide and multiple MECS (FIG. 3A). FIG. 3B shows a Northern blot which compares the expression of L119 mRNA after stimulation with MECS on its own and after administration of cycloheximide. RNA was isolated from rat hippocampus (left) and rat cortex (right) at the given times after stimulation. Analysis showed that each stimulus was able, on its own, to induce L119 mRNA expression in the given organs. The L119 expression which was induced by MECS was very rapid, with a transient peak at about 20 to 30 minutes after the convulsion and with a return to basal expression after about 1 hour. While the induction following cycloheximide administration was just as rapid, it continued to rise until the longest time to be analyzed, i.e. 6 hours after administration, and, taken overall, reached a substantially higher signal strength.

The cellular resolution of the expression of L119 following systemic administration of cycloheximide was qualitatively identical to the induction which occurred following endogenous stimuli (e.g. following the triggering of convulsions), and this expression was only detectable in blood vessels. In agreement with this observation, no induction of L119 gene expression was seen, following cycloheximide administration, in cell cultures which were of nonendothelial origin. In this way, L119 inducibility can be used as a marker staining for vascular endothelial cells. Another use of L119 inducibility is for being able to find, for example, suitable (endogenous) stimuli for inducing L119 expression in these cells.

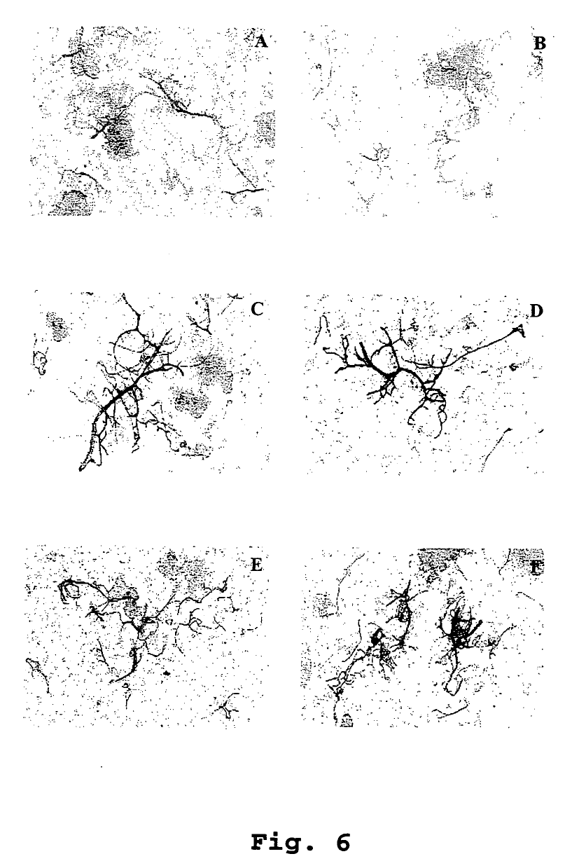

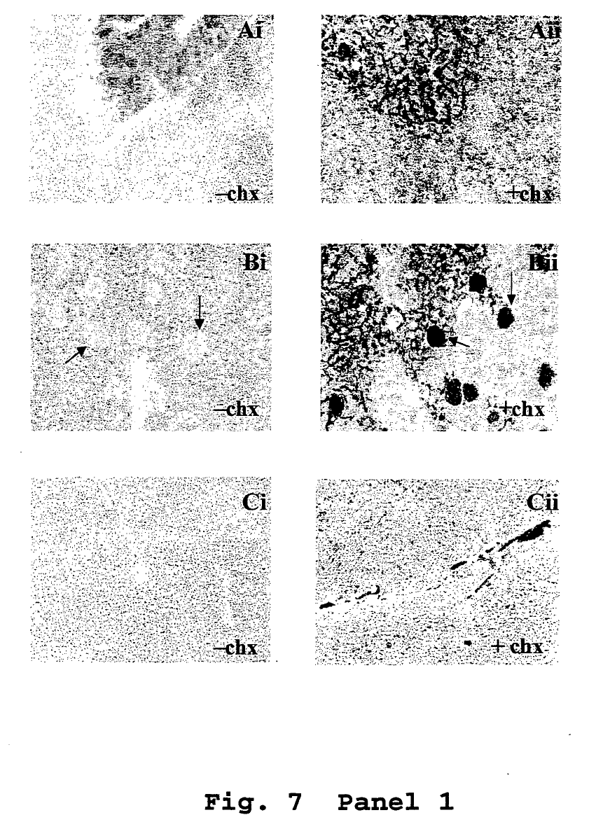

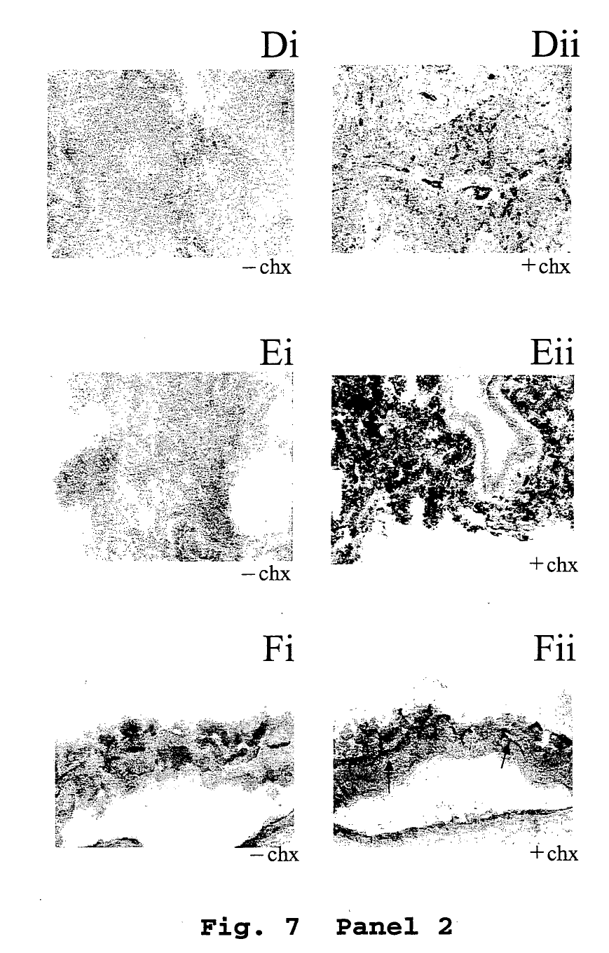

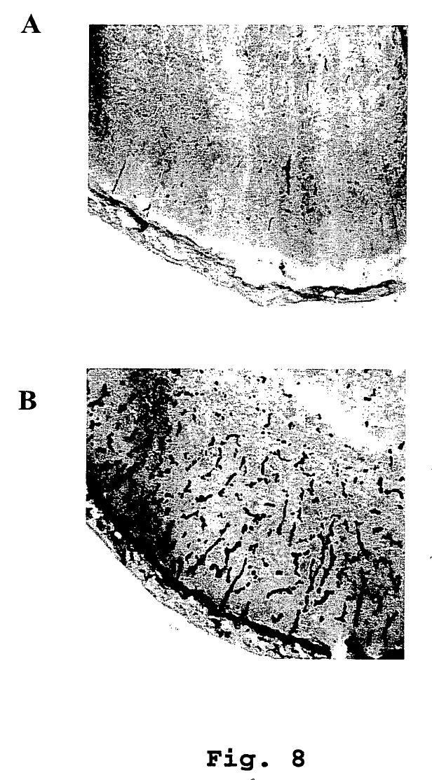

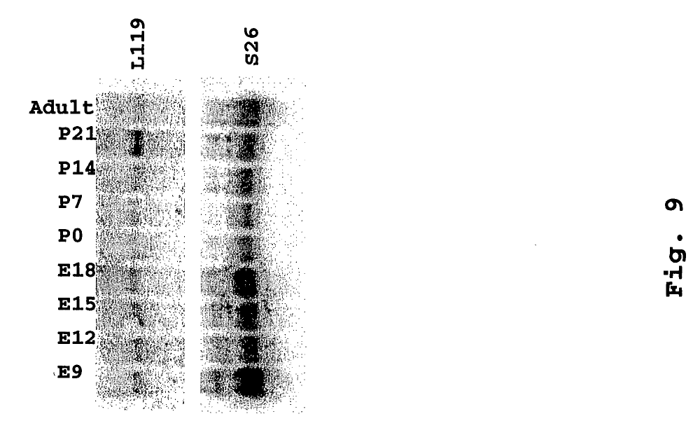

A digoxigenin-labeled L119 antisense riboprobe, which was prepared in accordance with the directions given in the “digoxigenin-labeled riboprobes” protocol (see above), gave a strong, specific and cycloheximide-inducible signal in rat brain (FIG. 4A, lower, right-hand half). The in situ hybridizations for L119 were carried out in accordance with “in situ hybridizations” protocol (see above). The induction of L119 in the brain following cycloheximide administration can be detected in all the areas of the brain. FIG. 5 shows examples of stainings which were obtained using sections of the gyrus dentatus (C, E) and cerebellum (D, F). All the capillaries located on these sections were stained, as were all the vessels of larger diameter (see FIG. 5E). This finding was confirmed by carrying out L119 in situ hybridizations on preparations of brain microvessels which were obtained from cycloheximide-treated rats and from control rats. Rat brains were carefully homogenized in medium (containing 5 mg of BSA/ml) in a glass-Teflon douncer and centrifuged in the presence of 13% dextran. The pellet was carefully resuspended and filtered through fine meshes (183 μm pore size). Vessels which were retained by a filter having a pore size of 53 μm were concentrated and freeze-dried on microscope slides; they were then subjected to an in situ hybridization in accordance with the “in situ hybridizations” protocol (see above). While preparations from control animals did not exhibit any L119 signals (FIGS. 6A and B), vessel preparations from cycloheximide-treated rats exhibited very strong signals in all the vessels investigated, i.e. both in relatively large vessels and in end-flow vessels (FIGS. 6C and D). It was shown that, by administering cycloheximide and subsequently detecting L119 mRNA, it was possible to identify cells in which there was the potential for inducing L119.

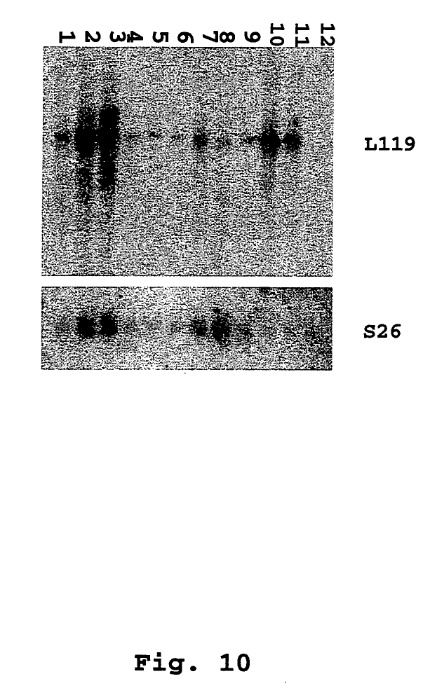

As the next step, an investigation was carried out to determine whether cycloheximide is also able to induce L119 mRNA in other organs apart from the brain. For this, non-radioactive in situ hybridizations were carried out, in accordance with the “in situ hybridizations” protocol (see above), on the organs of cycloheximide-treated rats and control rats. L119 signals are specific for the induced state and for vascular endothelium in all the organs investigated (adrenal gland, kidney, liver, spleen, lung and retina; FIG. 7). In addition to the capillaries, vessels of larger diameter are also stained in all the tissues investigated (see, in particular, FIGS. 7 Cii and Dii). The vas afferens, the vas efferens, the capillaries within the Bowman's capsule, and also the capillaries which run along the Henle's loop, inter alia, but not the epithelial cells themselves, are stained in kidney section. By being expressed in these kidney blood vessels, and in the endothelial cells of the lung (see FIG. 7 Eii) and of the adrenal cortex (FIG. 7 Aii), L119 is thus expressed in all the important organs of the renin-angiotensin-aldosterone system, which is an important regulator for the blood pressure.

L119 is expressed at a basal level during ontogenesis. Brains of 10-day-old rats which had been stimulated with cycloheximide exhibited very strong signals in the vascular endothelium. However, in contrast to adult animals, it was possible to observe a significant basal expression of L119 mRNa in these animals (FIG. 8). Systematic Northern blot analyses carried out on rat brains of varying age (embryo-day 9.5 to adult) detected expression at all stages. The strongest signals were obtained between postnatal days 8 and 21 (FIG. 9).

The intention was to investigate the appearance of the pattern of L119 mRNA expression in human organs in the basal state. For this, a blot carrying poly(A)⁺ RNA from 12 different organs (Human 12 lane MTN Blot, Cat. No. 7780-1; from Clontech GmbH Heidelberg, Germany) was hybridized with radioactive probes for L119 and S26, a small subunit ribosomal protein. The hybridization was carried out in accordance with the “radioactive labeling of DNA fragments” and “hybridization of nylon filters with radioactively labeled DNA fragments” protocols (see above). A 329 bp long PCR fragment (description, see above) was used as the probe for L119. The S26 probe for the hybridization was prepared by RT-PCR from rat brain total RNA (“RNA extraction”, “reverse transcription” and “polymerase chain reactions” protocols; see above). The following primers were employed:

| | |
|---|---|
| rS26-1s | |
| 5′-AAGTTTGTCATTCGGAACATTGT-3′ | (SEQ ID NO:41) |
| and | |
| rS26-1as | |
| 5′-CACCTCTTTACATGGGCTTTG-3′. | (SEQ ID NO:42) |

The following conditions applied in the polymerase chain reaction: 50 ng of cDNA, with 3 min at 96° C. for the initial denaturation and then 30 cycles of 30 sec at 96° C. for denaturation, 30 sec at 56° C. for annealing and 30 sec at 72° C. for elongation.

Signals were obtained from all the organs investigated, including strong signals from the heart, the skeletal muscles, the placenta, the lung and the kidneys (FIG. 10). The size of the detected L119 mRNA was about 4.5 kb in all the organs. Additional bands of different sizes (sizes from about 5 to 6 kb and of 3 kb) could be seen in the lanes containing the strongest signals (skeletal muscle, heart and placenta).

A number of stimuli can stimulate the expression of L119 mRNA in the hippocampus. These stimuli include acute convulsions which are induced by the systemic administration of kainat (10 mg/kg of body weight, injected intraperitoneally into male Sprague-Dawley rats weighing from 300 to 350 g) or pentylenetetrazole (50 mg/kg of body weight, injected intraperitoneally into male Sprague-Dawley rats weighing from 300 to 350 g), and also by global ischemia (which is elicited by 15-minute bilateral occlusion of the carotid artery together with additional hypotension of 35 mm Hg arterial blood pressure) (Worley patent application, WO 99/40225). The expression of L119 mRNA is also induced in an animal model of focal cerebral ischemia (a valid model for human ischemic stroke). In order to produce the focal cerebral ischemia, use was made of what is termed the thread model, in which a coated nylon thread is advanced through the internal carotid artery to the departure of the middle cerebral artery and induces an ischemic stroke (Clark WM et al. (1997) Neurol. Res. 19, 641-648). In cerebral ischemia, the regulation of gene expression plays a role which is crucial for determining the development and extent of the neuronal damage (Koistinaho J and Hokfelt T (1997) Neuroreport 8, i-viii; Schneider A et al. (1999) Nat Med 5, 554-559). In particular, immediate early genes, such as cox-2 (Yamagata K et al., (1993) Neuron 11, 371-386; Nogawa S et al. (1997) J. Neurosci. 17, 2746-2755) are of importance in this context (Atkins PT et al. (1996) Stroke 27, 1682-1687).

In situ hybridizations which were carried out, in accordance with the “in situ hybridizations” protocol (see above), on brains following 60 min of ischemia and 23 h of reperfusion showed L119 mRNA signals, both in the infarct region and in the periinfarct region (penumbra), which were strong compared with those on the control side in the same animal.

### Example 6

### The Expression of L119 mRNA in the Vascular Endothelium of Tumors

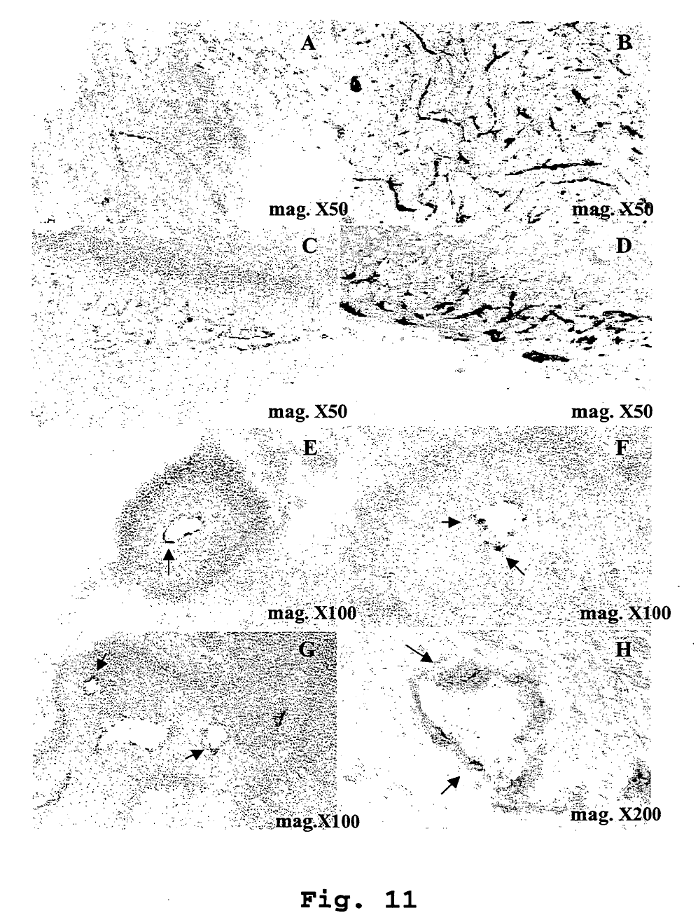

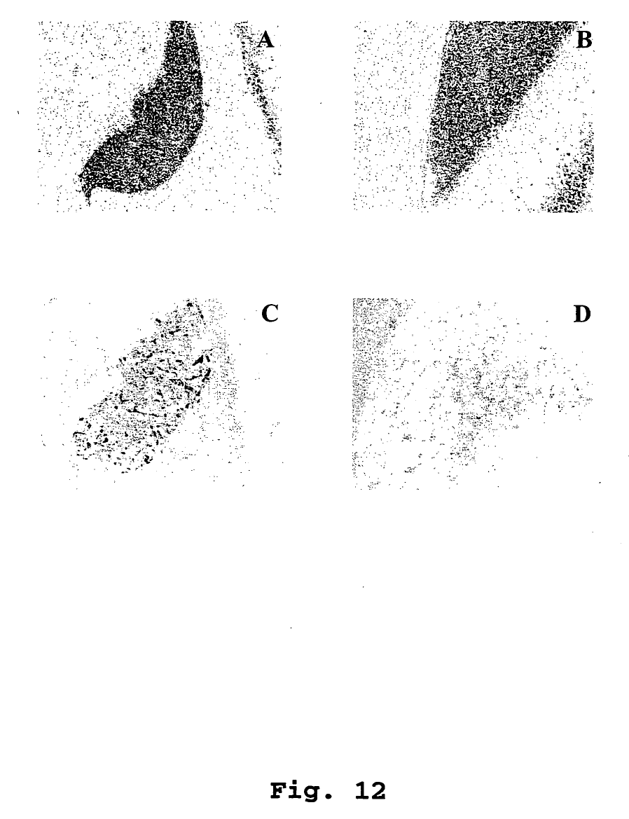

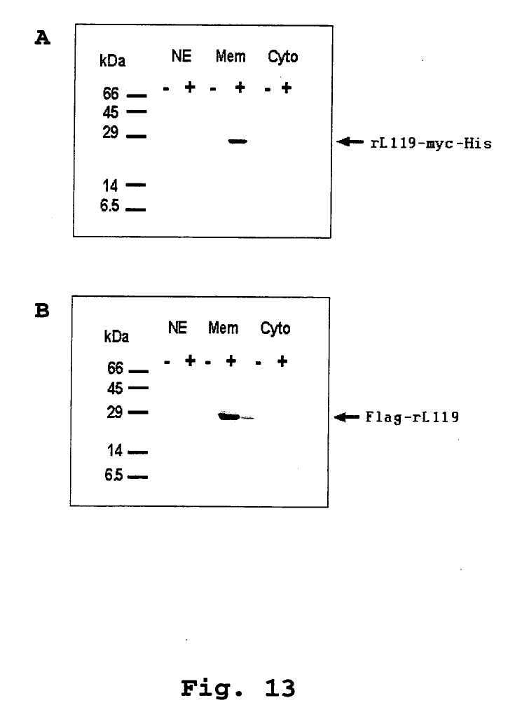

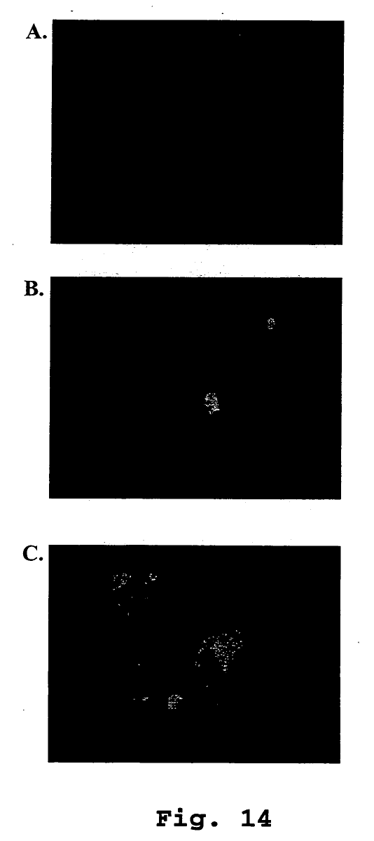

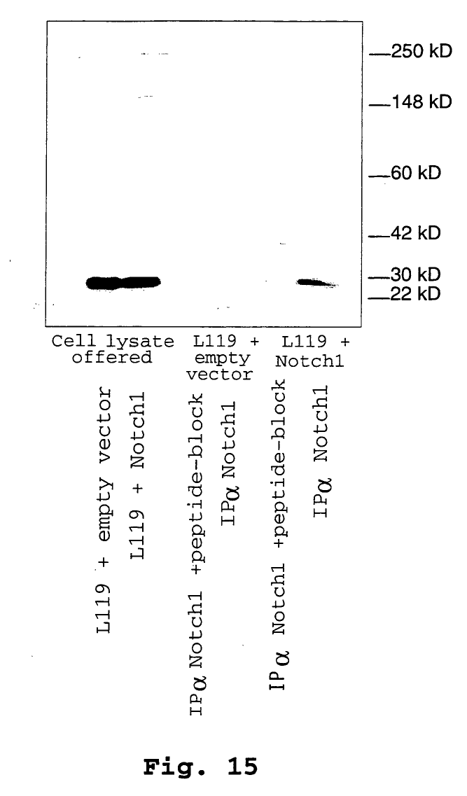

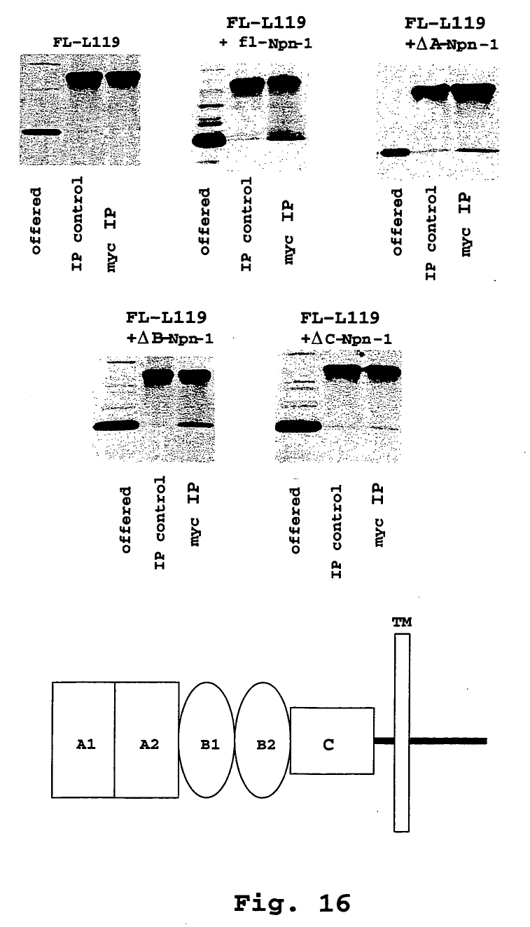

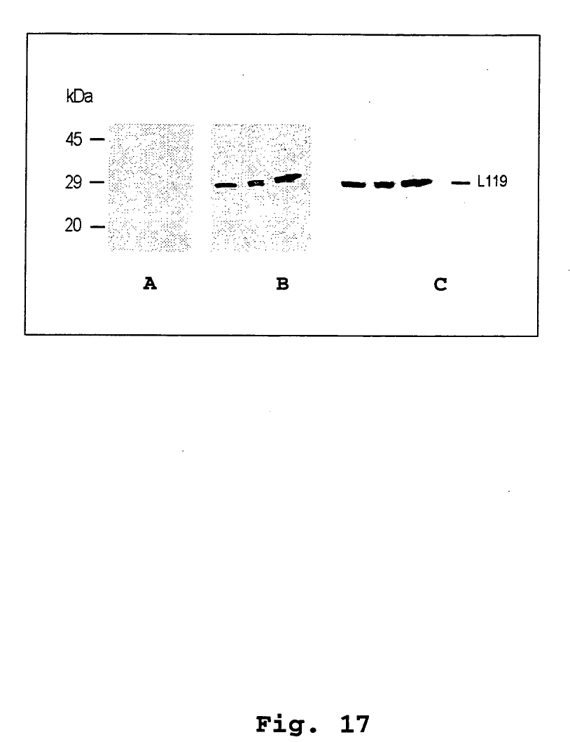

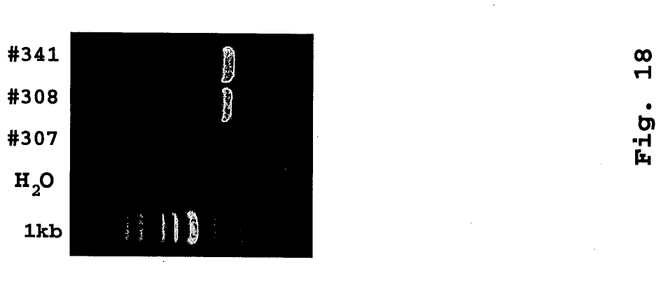

L119 mRNA expression was detected in endothelial cells and could be detected during the development of the organism in phases involving active angiogenesis (see above). The intention was to investigate whether L119 is also expressed in tissues in which pathological angiogenesis is occurring. For this, about 100000 tumor cells from a 9L glioblastoma were injected subcutaneously into the flanks of rats. The growth of the tumor cells was monitored amd the tumors were removed after their size had increased to about 1 g. Sections were prepared and hybridized, in accordance with the “in situ hybridization′ protocol (see above), with probes for L119 (described under “digoxigenin-labeled riboprobes”). Very strong L119 expression was detected in capillaries (FIGS. 11A and C) and in larger vessels (E to H). It can clearly be seen that it is only the endothelial layers which are L119-positive in the larger vessels (arrows in E to H). It was possible to augment the expression of L119 still further if the rats had been administered cycloheximide systemically before the tumors were removed (cf. above; FIGS. 11B and D).

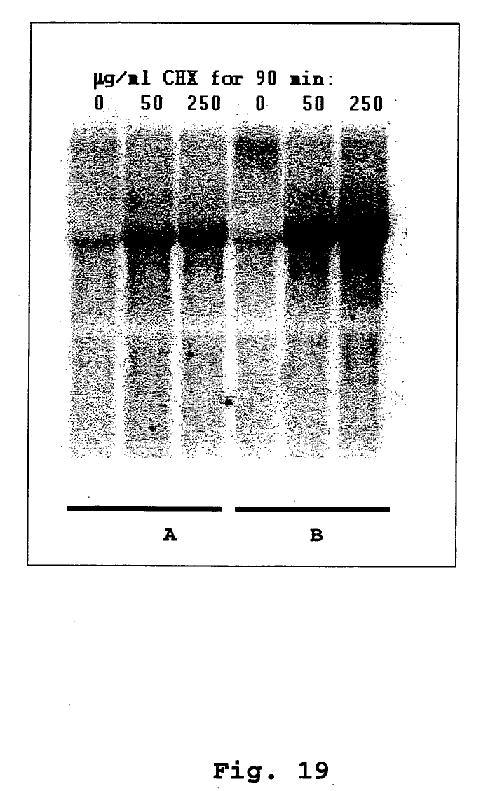

It was possible to observe very strong L119 mRNA expression in tumor blood vessels during tumor angiogenesis. For this, small quantities of tumor (9L glioblastoma; approximately 1 mm in diameter) were transplanted unilaterally, in a stereotactic operation, into the lateral ventricle (method described in: Guerin C. et al. (1992) Am. J. Pathol. 140:417-425). Further growth in the size of the tumor was dependent on the formation of new blood vessels. After 8 and 18 days, respectively, with the tumors having grown correspondingly, in situ hybridizations were carried out on the brains containing the implanted tumors; the procedure for this corresponded to the “in situ hybridizations” protocol (see above). FIG. 12 shows very strong L119 signals in the vascular endothelium of the tumor after 8 days (C) and 18 days (D), respectively, whereas it is not possible to detecet any L119 expression in the adjoining, healthy brain tisuse (on the left and on the right alongside the tumor tissue in the figure). (A) and (B) depict control Nissl staining of adjacent sections (carried out in accordance with the “Nissl staining” protocol; see above). The next thing to be investigated was whether L119 mRNA expression during tumor angiogenesis is specific for glioblastomas. For this, total RNA was extracted from various human tumors and metastases of the head and investigated for the expression of L119 by means of Northern blot analysis (carried out in accordance with the “RNA extraction”, “Northern blot”, “radioactive labeling of DNA fragments” and “hybridization of nylon filters with radioactively labeled DNA fragments” protocols). A 329 bp long PCR fragment (description, see above), was used as the probe for L119. When the ratio of the signal strength of L119 to that of ubiquitin was used for the comparison, it was possible to detect L119 mRNA expression, albeit to different extents, in all the tumors analyzed. For to example, a particularly high ratio for the expression of L119 relative to that of ubiquitin was found in a rhabdomyosarcoma metastasis in a 5-year-old boy.

### Example 7

### Expression of L119 mRNA in Cultured Endothelial Cells

Northern blot analyses carried out on primary human microvascular endothelial cells obtained from lung tissue (HMVEC-L; Clonetics/BioWhittaker) were used to examine where the above-described stimuli can induce L119 mRNA expression in cultured cells. HMVE cells were plated out in EGM-2-MV medium (Clonetics/BioWhittaker) at a rate of 350000 cells/10 cm plate. Fresh medium was added to the cells after every 24 h. After 48 h, the cells were confluent and were cultured further, without any change of medium, for 24 h or 48 h and then stimulated by adding from 0 to 250 μg of cycloheximide (CHX)/ml for 90 min. The total

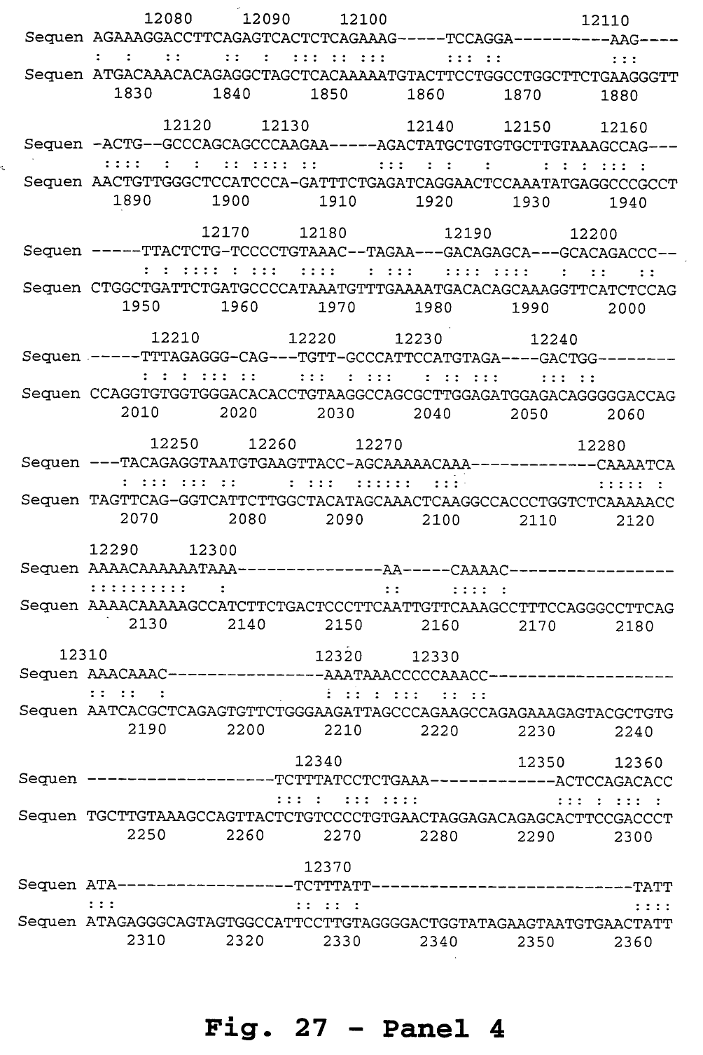

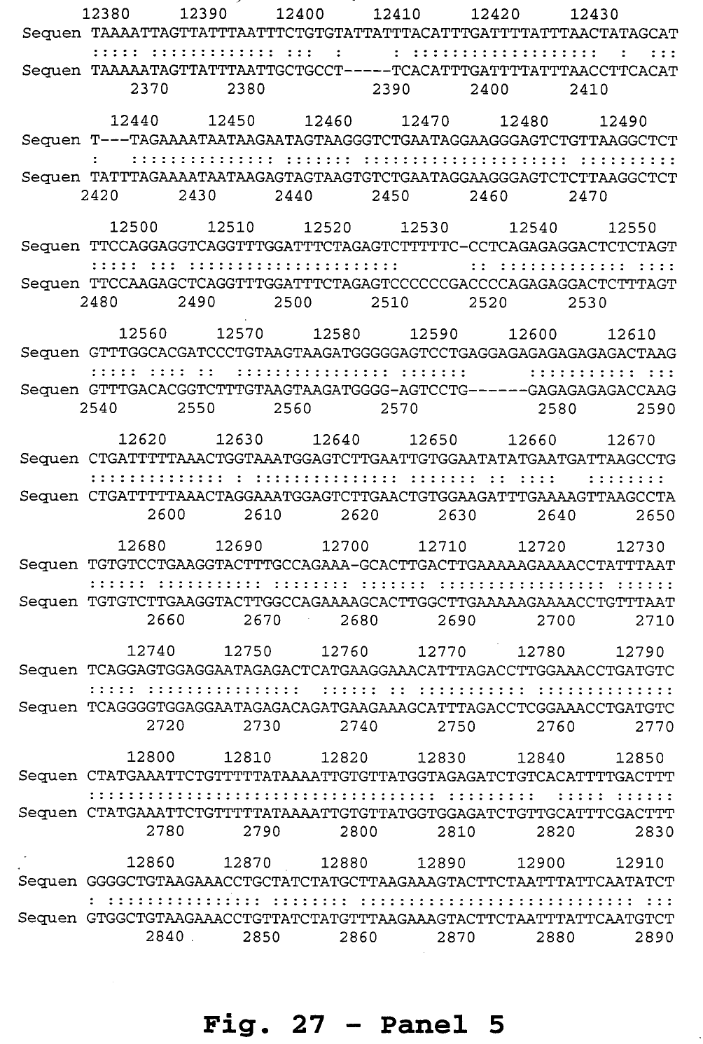

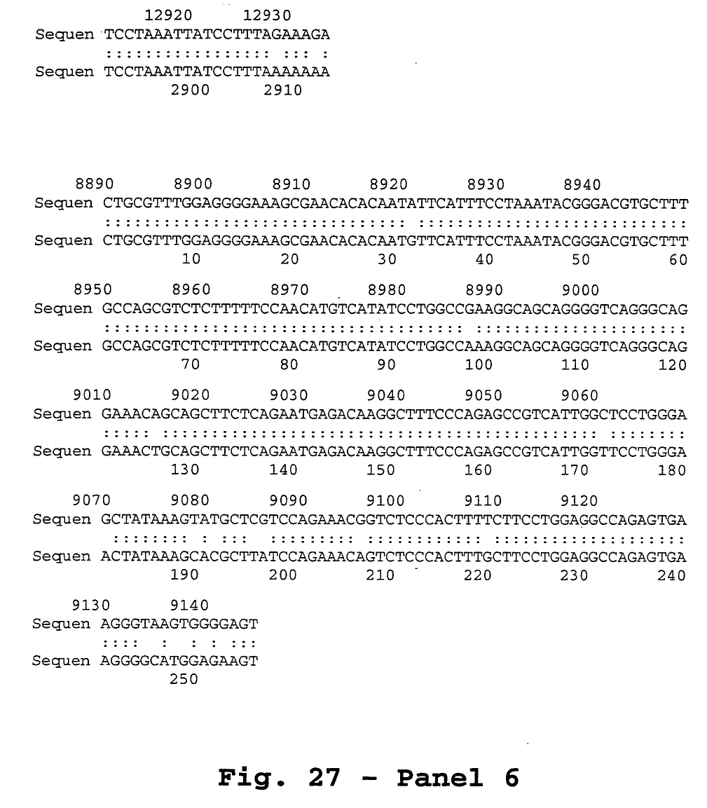

RNA was prepared using the RNeasy RNA preparation kit (Cat. No. 74104, from Qiagen; Hilden, Germany) in accordance with the protocol given in the manual accompanying the kit). In each case, gg of RNA were loaded onto a Northern gel per lane and the blotted membrane was analyzed by hybridizing it with a human L119 probe (XhoI/HindIII 2070 bp fragment) (“Northern blot”, “radioactive labeling of DNA fragments” and “hybridization of nylon filters with radioactively labeled DNA fragments” protocols; see above) (FIG. 19). An analogous procedure was followed for stimulating the cells with TNF-α (25 nM) and interleukin 1-β (10 to 100 ng/ml), with the cells being cultured for 24 h, while confluent, in serum-free basal medium (EBM-2-MV; Clonetics) before the reagents were added. A 2- to 5-fold induction of L119 mRNA expression was observed after incubating with cycloheximide (250 μg/ml) and after administering IL-1β (100 ng/ml). By contrast, the addition of TNF-α had no effect on the expression of L119 mRNA (FIG. 20).

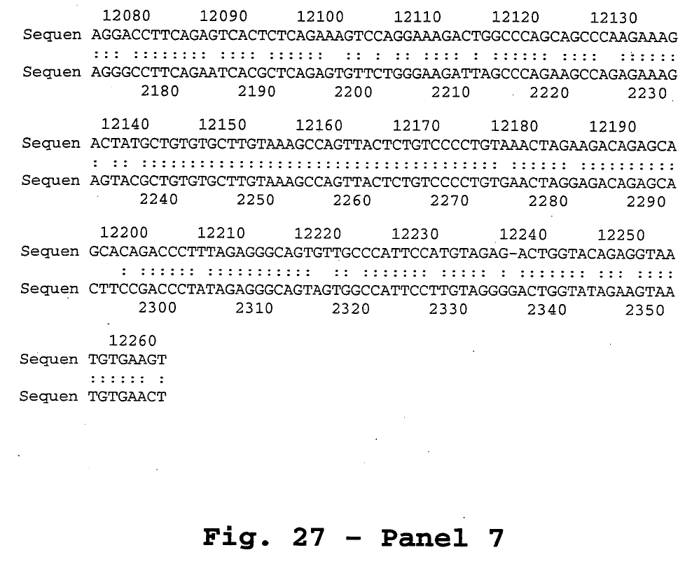

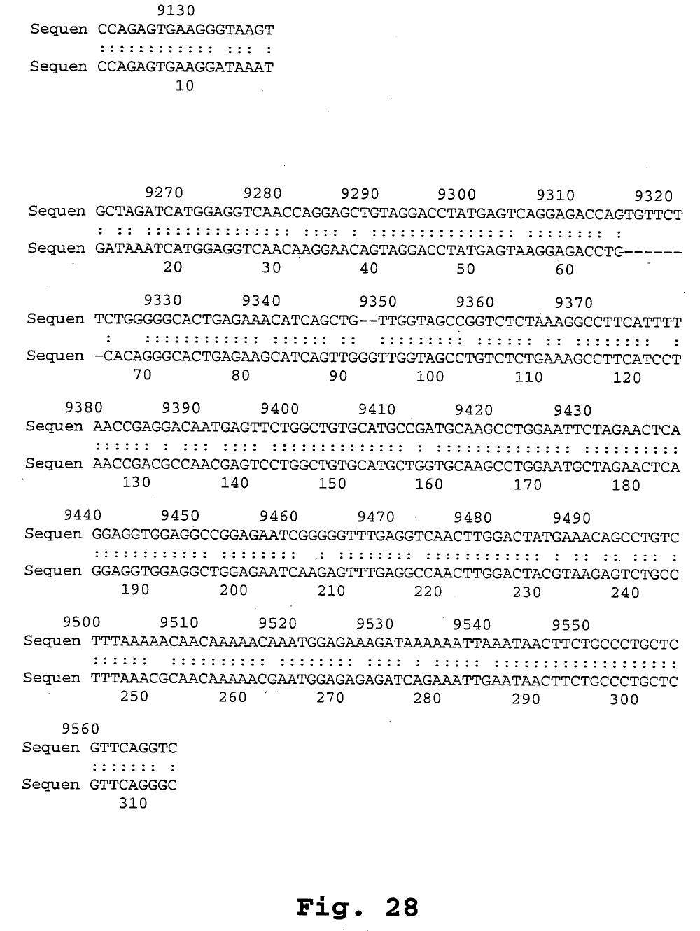

Since it was possible to demonstrate that cerebral ischemia strongly induces L119 mRNA expression both in the infarct region and in the peri infarct region in the animal model, an investigation was carried out, on cultured endothelial cells, to determine whether the expression of L119 mRNA can be influenced by hypoxic culture conditions. For this, subconfluent (approx. 80 to 90% confluent; 4 ml of EGM-2-MV medium/10 cm plate) HMVE cells and RBE4 cells (immortalized microvascular endothelial cells from rat brain; Roux F. et al., (1994) J Cell. Physiol. 159:101-113) were gassed for 3 h, in an hypoxia chamber at 37° C., with a mixture consisting of 90% N₂, 5% CO₂ and 5% H₂ in the presence of a palladium catalyst (reduces the free O₂ to H₂O; BBL GasPak Replacement Charges; Becton Dickinson, Cat. No. 4370303). An RNA preparation was then carried out (RNeasy Kit; Qiagen) and in each case 10 μg of total RNA were analyzed, per lane, by means of Northern blotting. For this, the RNA from HMVE cells was hybridized with a human L119 probe (XhoI/HindIII 2070 bp fragment, see below), and the RNA which had been isolated from RBE4 cells was hybridized with a probe from the 3′-untranslated region of the rat L119 cDNA (pos. 2260 to 2920 of SEQ ID No: 1) (FIGS. 21a and b). In both cell types, the hypoxic culture conditions induced L119 mRNA expression approximately 2- to 3-fold following normalization with the ribosomal factor S26. In order to obtain the human probe, filters containing a human BAC library (high density CITB human BAC colony DNA membranes; Cat. No. 96055; from Research Genetics) were hybridized with an L119-specific probe (“radioactive labeling of DNA fragments” and “hybridization of nylon filters with radioactively labeled DNA fragments” protocols). The L119 probe for the hybridization was prepared by RT-PCR from human brain total RNA (Cat. No. 64020-1; from Clontech Heidelberg, Germany) (“reverse transcription” and “polymerase chain reactions” protocols; see above) . The primers hL119-4s (SEQ ID NO: 15) and hL119-4 as (SEQ ID NO: 19) were used in this context (sequences, see above). In addition, the following conditions applied: 50 ng of cDNA, with 3 min at 96° C. for the initial denaturation and 35 cycles of 30 sec at 96° C. for denaturation, 30 sec at 62° C. for annealing and 30 sec at 72° C. for elongation. Two clones gave strong positive signals with the probe employed. BAC-DNA (large construct kit; Cat.No. 12462; Qiagen GmbH, Hilden, Germany) was isolated from one positive clone using the protocol given in the manual accompanying the kit (version 06/99). This clone was verified as being L119-positive by means of a variety of restriction digestions and hybridizations with L119 probes. Various EcoRI fragments from the BAC were subcloned into a plasmid vector. A XhoI/HindIII fragment of approx. 2070 bp in length was subcloned from an L119-positive EcoRI plasmid clone into a plasmid vector. The XhoI/HindIII insert in this clone was isolated by gel electrophoresis and subsequent purification of the DNA (using QiaexII; Cat. No. 20021; Qiagen, Hilden, Germany).

### Example 8

### Protein Expression Studies

Intracellular Location of L119

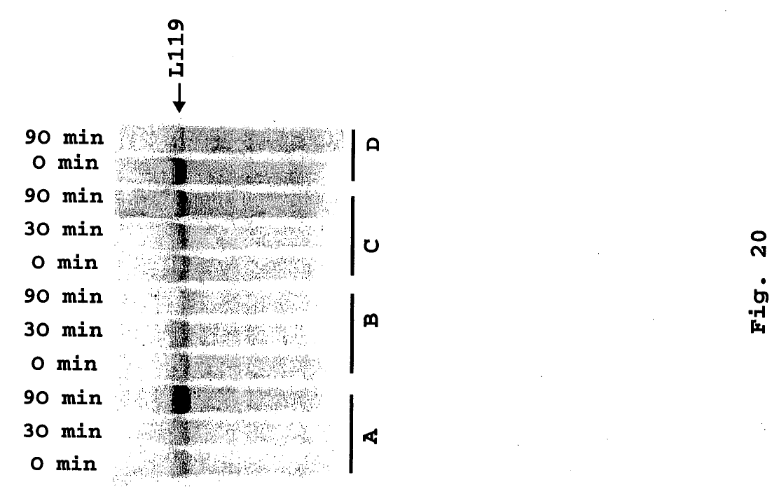

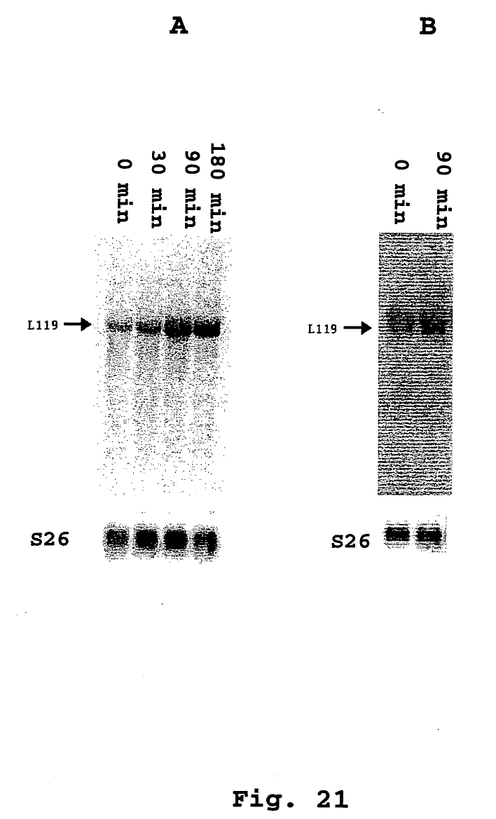

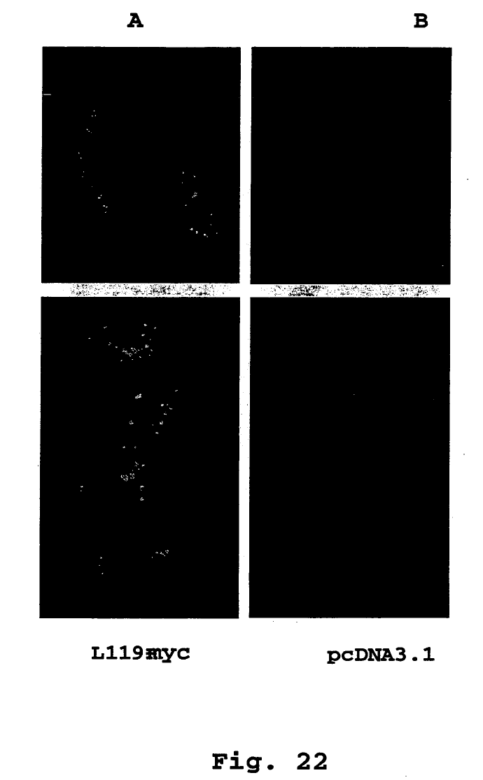

The coding region of rL119 cDNA was fused to a carboxy-terminal Myc-histidine tag in the vector pcDNA3.1-myc-His (Invitrogen), and provided with an aminoterminal flag tag in the vector pRK5. For this, the L119 ORF was amplified by PCR using the primer pairs SEQ ID NO: 25 and 26 or SEQ ID NO:27 and 28. A 50 μl mixture was prepared from 5 μl of 10× cloned Pfu buffer (Stratagene); 2 μl of dNTP mixture (5 mM; Cat. No. 1969064, Roche Diagnostics GmbH, Mannheim, Germany); in each case 2 μl of said primer pairs (stock conc. 10 μM); 100 ng of rL119 cDNA template, 35 μl of H₂O and 1 μl of Pfu turbo DNA polymerase (Stratagene) and, after the mixture had been incubated at 94° C. for 3 minutes, 28 PCR cycles were performed in accordance with the following temperature program: 30 sec of primer annealing at 60° C., 70 sec of strand extension at 72° C., and 30 sec of DNA double-strand melting at 94° C., together with a concluding extension step of 7 min at 72° C. The resulting PCR products were gel-purified, cleaved with the restriction enzymes EcoRI and XbaI and subcloned into the expression vectors pcDNA3.1-myc/His-A (Invitrogen) and pRK5-flag, which had likewise been cut with EcoRI and XbaI; they were then sequenced. Both constructs were transiently transfected into HEK293 cells (in accordance with the “transient transfection” protocol; see above), and the cells were harvested after 48 hours. The proteins from the cells were separated, in fractionation experiments into a nuclear fraction, a membrane-located fraction and a cytosolic fraction (Scheek S et al. (1998) Proc Natl Acad Sci USA 94, 11179-83) and then subjected to Western blot analysis. The filters were hybridized, in accordance with the “Western blot analysis” protocol (see above), with antibodies directed against the respective tags in the L119 constructs (monoclonal anti-myc antibody, Invitrogen; monoclonal anti-flag M2 antibody, Sigma-Aldrich). In both cases, signals were obtained in the 100000 g membrane fraction (FIG. 13). Immunohistochemical analyses were carried out on COS7 cells in parallel. For this, the cells were transfected with a pRK5-L119 expression construct (coding region of the L119 cDNA in vector pRK5). In order to prepare the construct, a PCR was carried out, as described above and using the primers SEQ ID NO: 29 and 30, under the following conditions: after 3 minutes of denaturation at 94° C., 25 PCR cycles were carried out in accordance with the following temperature program: 1 min of primer annealing at 56° C., 1 min of strand extension at 72° C. and 1 min of DNA double-strand melting at 94° C., together with a concluding extension step of 7 min at 72° C. The resulting PCR product was gel-purified, cut with the restriction enzymes SalI and NotI and subcloned into the expression pRK5, which had likewise been cut with SalI and NotI; for verification, the PCR product was then sequenced. For the immunohistochemical analysis, L119 and the control vector pRK5 were transfected into COS 7 cells in accordance with the “transient transfection” protocol. 48 hours after the transfection, the cells were fixed for 2×15 min in 4% paraformaldehyde, after which they were permeabilized with 0.25% Triton X-100 for 15 min and then blocked for 1.5 h at RT with 10% (NGS/PBS (Normal Goat Serum, Jackson ImmunoResearch Laboratories Inc., Cat. No. 005-000-121). The antibody reactions were carried out, in each case at RT for 1.5 h in 3% NGS/PBS, using a polyclonal antibody directed against rat rL119, followed by an anti-rabbit IgG-FITC antibody. After each antibody incubation, the cells were washed in each case 3× for 10 min with PBS. The cover slips were melted with Permaflout (Immunon/Shandon, Cat. No. 434990) on microscope slides. The majority of the overexpressed L119 protein was detected in vesicular structures. By means of double staining, it was possible to demonstrate that these vesicular structres were constituents of the secretory pathway, in particular of the Golgi apparatus (FIGS. 14B and C).

The empty pRK5 control plasmid did not give any specific signals (FIG. 14A). Cell-surface biotinylationi studies carried out on pRK5-L119-transiently transfected COS 7 cells showed that L119 protein could also be detected on the cell surface.

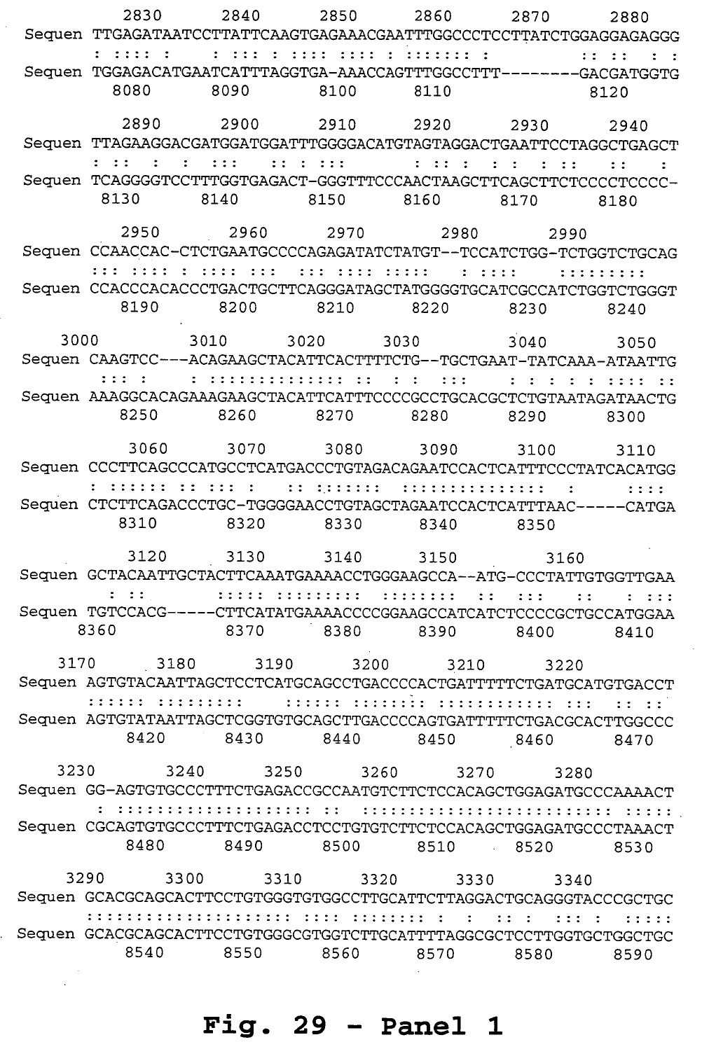

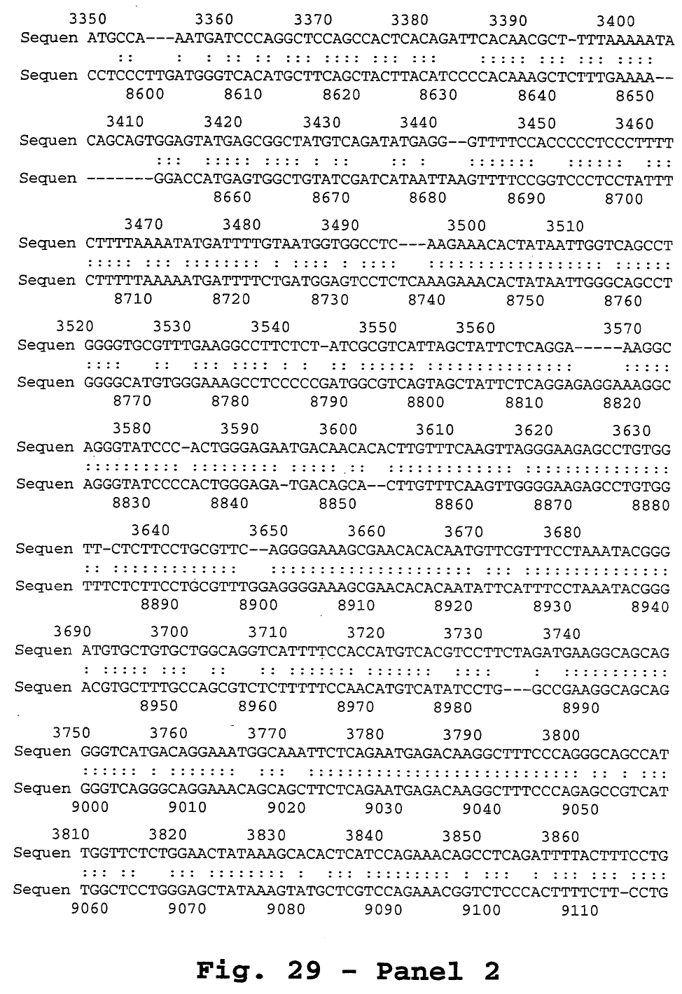

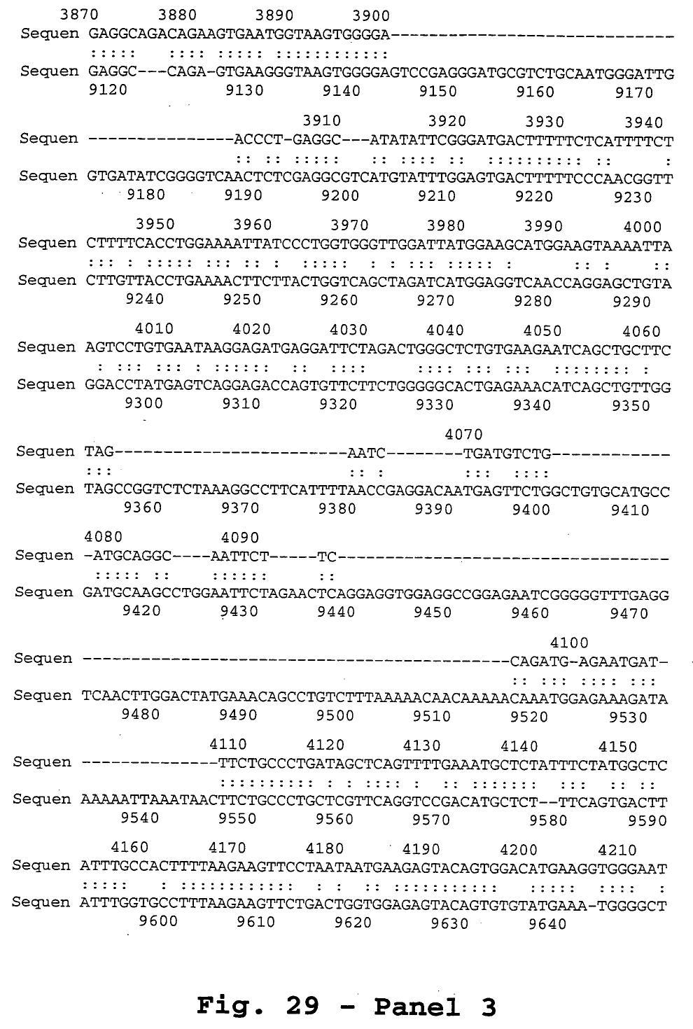

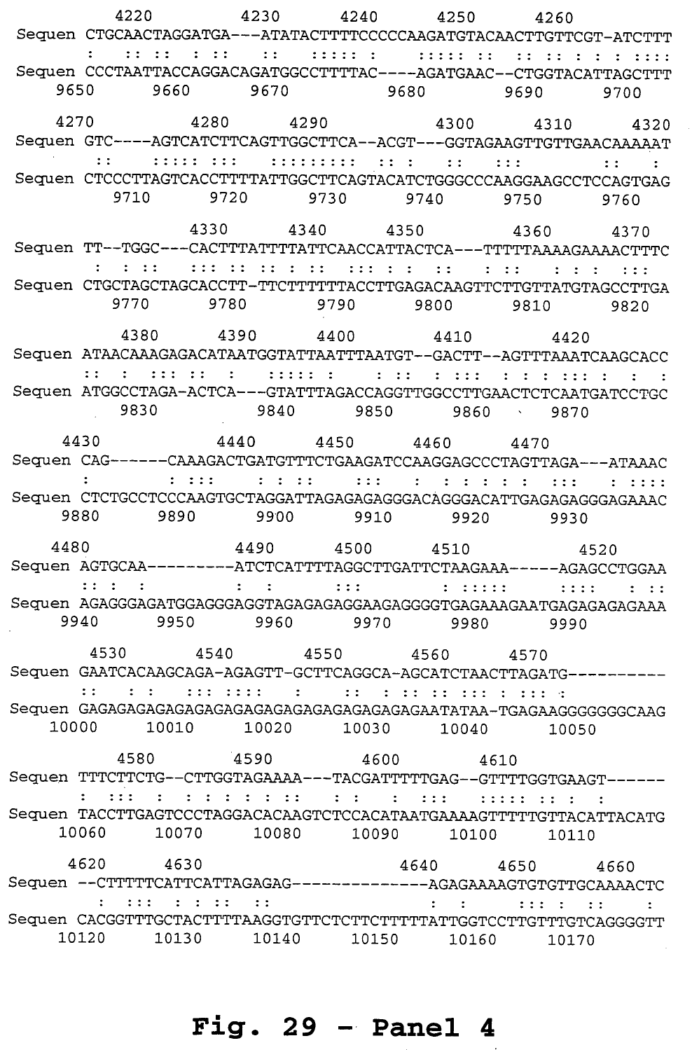

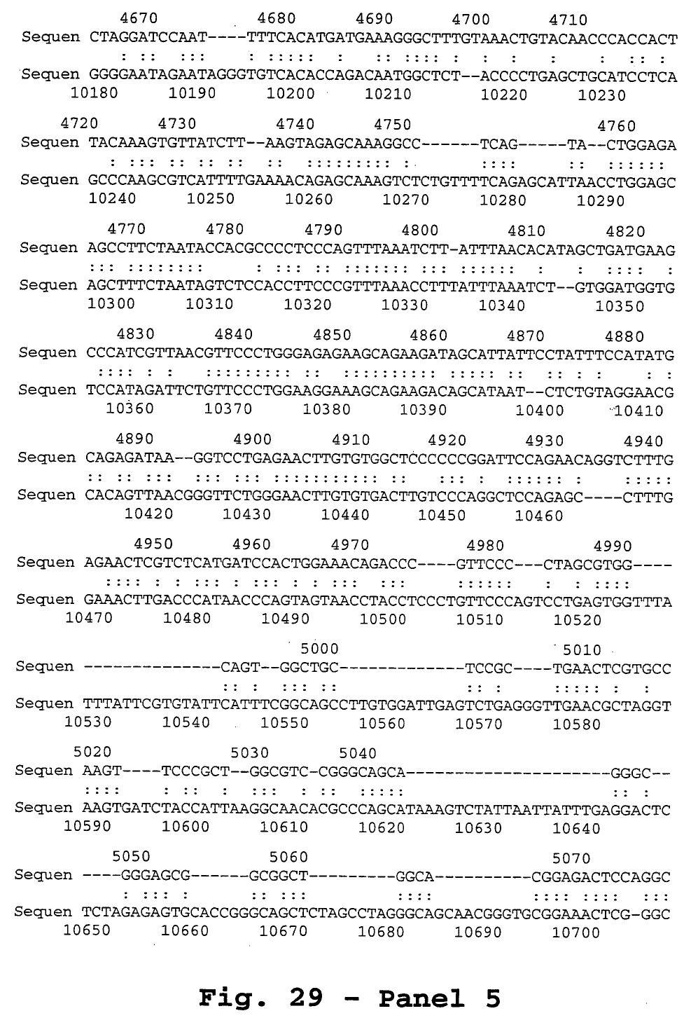

In addition, the subcellular location of L119 was investigated in transiently transfected RBE4 and YPEN-1 cells. The RBE4 cell line is derived from immortalizing microvascular endothelial cells obtained from rat brain (Roux F et al. (1994) J. Cell. Physiol. 159, 101-13), while YPEN-1 cells were obtained by immortalizing rat prostate endothelial cells using an adenovirus-12SV40 hybrid virus (Yamazaki K et al. (1995) In Vivo 9, 421-6). For this, the cells were sown on fibronectin-coated cover slips at the rate of 30000 to 40000 cells per well of a 24-well plate in EGM-2-MV medium. On the following day, the lipofection of L119 constructs was carried out using Lipofectamine Plus (GibcoBRL) (per well of a 24-well plate: 400 ng of DNA, 4 μl of Plus reagent and 1 μl of lipofectamine in in each case 50 μl of serum-free EBM-2). After 3 h of incubation in 500 μl of serum-free EBM-2-MV medium, the cells were incubated for a further 36 to 48 h in complete medium and then fixed for 30 min in 3% paraformaldehyde/PBS. After having been washed several times in 10 mM Tris-HCL, pH 8.0, EGFP-L119- and L119-EGFP-transfected cells were mounted, together with the corresponding vector control (pEGFPAEGFP), on microscope slides using AquaPolyMount (Polysciences Inc., Cat. No. 18606). After permeabilizing with 0.15% Trition X-100, immunocytochemical analyses were carried out using a polyclonal antibody directed against rL119 (2892), followed by an anti-rabbit IgG FITC antibody (Jackson ImmunoResearch Laboratories, Inc.). The following expression constructs were transfected for overexpressing L119 in endotheliel cells: pRK5-L119, pRK5-FlagL119, pEGFPN1ΔEGFP-L119 myc, pEGFPN1-L119 and pEGFPC1-L119. The fusions of L119 with the enhanced green fluorescent protein (EGFP) were obtained by means of PCR using the oligonucleotide primers SEQ ID NO: 31 and 32 and SEQ ID NO: 33 and 34, respectively. For this, a 50 μl mixture was in each case prepared from 5 μl of 10× cloned Pfu buffer (Stratagene); 2 μl of dNTP mixture (5 mM; Cat. No. 1969064, Roche Diagnostics GmbH, Mannheim, Germany); in each case 2 μl of the abovementioned primer pairs (stock conc. 10 μM); 100 ng of rL119 cDNA template, 35 μl of H₂O and 1 μl of Pfu turbo DNA polymerase (Stratagene) and, after the mixture had been.incubated at 94° C. for 3 minutes, 28 PCR cycles were then carried out in accordance with the following temperature program: 30 sec of primer annealing at 62° C., 70 sec of strand extension at 72° C. and 30 sec of DNA double-strand melting at 94° C., together with a concluding extension step of 7 min at 72° C. The resulting PCR products were gel-purified, cut with the restriction enzymes EcoRI and BamHI and then cloned into the corresponding restriction cleavage sites of the vectors pEGFP-N1 and pEGFP-C1. An L119-specific immune staining was detected in vesicular structures independently of the cell type and the expression construct (FIGS. 22 to 24). By means of double staining, it was possible to demonstrate that, in contrast to the L119 staining carried out on cells which were not of endothelial origin, these structures do not represent any Golgi elements. Consequently, these structures should be organelles which belong to the post-Golgi compartments of the secretory pathway.

### Example 9

### Identification of Proteins which Interact with L119 in vitro

A yeast two hybrid screen was carried out in order to identify A proteins which interact with L119. The entire coding region of the L119 cDNA was amplified in a polymerase chain reaction (PCR) and cloned into vector pPC86. The oligonucleotide primers having the sequences SEQ ID NO: 35 and 36 were used to do this. A 50 μl PCR mixture was prepared from 5 μl of 10× cloned Pfu buffer (Stratagene); 2 μl of DNTP mixture (5 mM; Cat. No. 1969064, Roche Diagnostics GmbH, Mannheim, Germany); in each case 2 μl of the abovementioned primer pairs (stock conc. 10 μM); 100 ng of rL119 cDNA template, 35 μl of H₂O and 1 μl of Pfu turbo DNA polymerase (Stratagene), and, after it had been incubated at 94° C. for 3 minutes, 28 PCR cycles were then carried out in accordance with the following temperature programm: 1 min of primer annealing at 56° C., 1 min of strand extension at 72° C. and 1 min of DNA double-strand melting at 94° C., together with a concluding extension step of 7 min at 72° C. The resulting PCR products were gel-purified, cut with the restriction enzymes SalI and NotI and cloned into the corresponding restriction cleavage sites of pPC86. The DNA construct obtained in this way encodes a protein in which the Gal4 DNA-binding domain is fused to the L119 protein. The yeast strain Y190 (Flick JS and Johnston M (1990) Mol. Cell. Biol. 10, 4757-4769; Harper J et al. (1993) Cell 75, 805-816) (from Life Technologies) was transformed with this construct. The resulting yeast strain was transformed with a rat brain cDNA library (obtained from cortex and hippocampus RNA, following maximal electroconvulsive shock (MECS) (Antony Lanahan and Paul Worley)) in the vector pPC86 (from Life Technologies), and 3×10⁶ transformants were plated out. After 3 to 5 days of growth at 30° C., colonies having a diameter of more than 2 mm were isolated and subjected to X-Gal staining (protocol: ProQuest™ Two-Hybrid System, Cat. Series 10835, Life Technologies) . In all, 14 colonies proved to be His3 and lacZ positive. The respective cDNA from these colonies was amplified using vector-specific primers and the-amplicon was sequenced (protocol: ProQuest™ Two-Hybrid System, Cat. Series 10835, Life Technologies). The sequence analysis gave 8 different putative interacting proteins (Table 1):

TABLE 1
L119 interactors fished from the yeast two-hybrid system.

| Yeast two hybrid | # | |
|---|---|---|
| Interactor | Frequency | Desription |
| Nel | 5 | PXC-binding protein, enriched |
| | | in the brain |
| Notch 4 | 3 | Endothelial transmembrane |
| | | protein; determination of the |
| | | fate of the cell |
| Notch 3 | 1 | Endothelial transmembrane |
| | | protein; determination of the |
| | | fate of the cell |
| Notch 2 | 1 | Neuronal transmembrane protein; |
| | | determination of the fate of the cell |
| Matrilin-2 | 1 | Oligomeric protein in the |
| | | extracellular matrix |
| TIED | 1 | Ten beta integrin EGF-like |
| | | repeat domains |
| Laminin alpha-4 chain | 1 | Protein in the extracellular matrix |
| Ten-m3 | 1 | Dimeric type II transmembrane |
| | | protein |

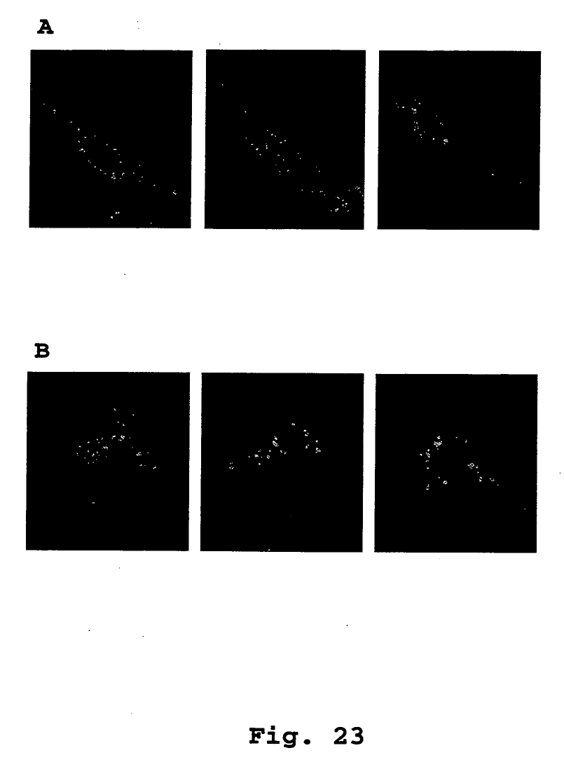

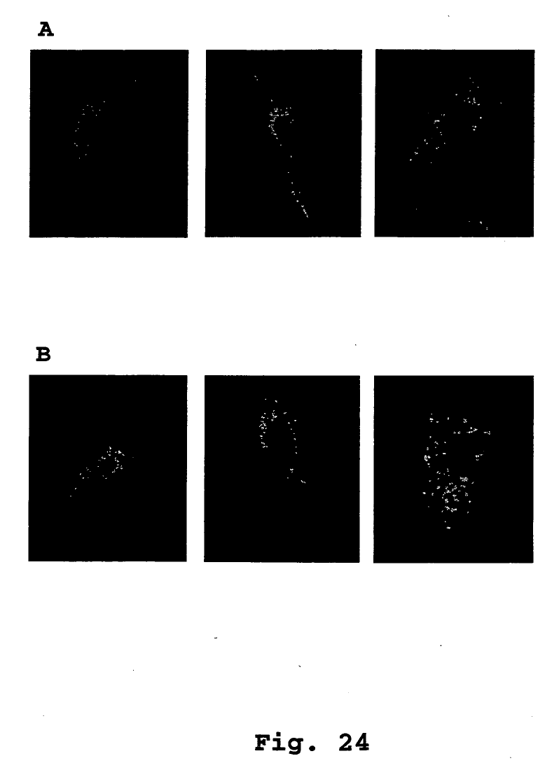

Coimmunoprecipitation was used, by way of example, to investigate whether the interactions which were identified in the yeast two-hybrid screen were physiologically relevant. A construct for expressing the transmembrane receptor Notch 1 (provided with a myc tag; provided by J. Nye, Northwestern University; described in Nye JS et al. (1994) Development 120, 2421-30), or an empty vector control, was cotransfected, together with a pRK5-L119 expression construct, into HEK293 cells in accordance with the “transient transfection” protocol (see above). The expression of the two proteins was confirmed by Western blot analysis (data not shown). 48 hours after the transfection, the cells were lysed by sonication in PBS/1% Triton X-100 and protease inhibitors. Following a centrifugation at 16000 g for 20 min and at 4° C., the supernatant was then used for coimmunoprecipitations. A monoclonal mouse anti-myc antibody (Calbiochem, Cat. No. OP10) was employed as the immunoprecipitating antibody. For this, 0.5 μg of the antibody was pipetted into 300 μl of lysate and the mixture was incubated at 4° C. for 2 h. 40 μl of protein A agarose (Pierce, Cat. No. 20333) were then added and the mixture was incubated at 4° C. for 30 min. The solid material was then washed 3× with PBS/1% Triton X-100/protease inhibitors and 2× with PBS/1% Triton X-100/protease inhibitors/500 mM NaCl. The agarose beads were eluted with Laemmli sample buffer and the eluate was fractionated on a denaturing SDS gel. The coimmunoprecipitation was detected by means of a Western blot experiment (see “Western blot analysis” protocol; see above) using an anti-L119 antibody (2894). FIG. 15 shows that the anti-Notch 1 antibody (Santa Cruz Biotechnology, Cat. No. sc-6015), which is directed against the C terminus of Notch 1, was only able to coprecipitate L119 protein when Notch 1 protein was present. In a control experiment, a peptide which blocked the Notch 1 antibody (Santa Cruz Biotechnology, Cat. No. sc-6015p), was also added, resulting in the disappearance of the specific immunoprecipitated band (“Ip-blocked Notch 1 AB” lanes in FIG. 15). This thereby demonstrated that the L119 and Notch 1 proteins interacted in heterologously transfected cells. It is readily possible to use the above-described method to verify the interactions with the other proteins which were found in the yeast two-hybrid assay.

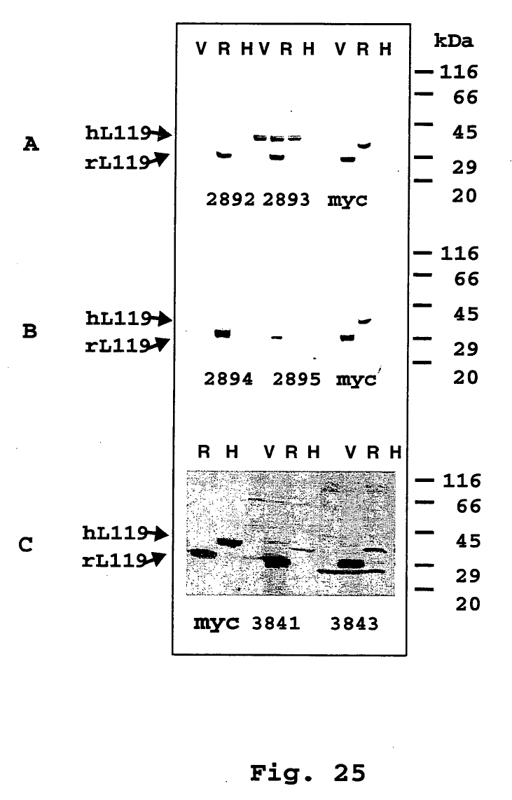

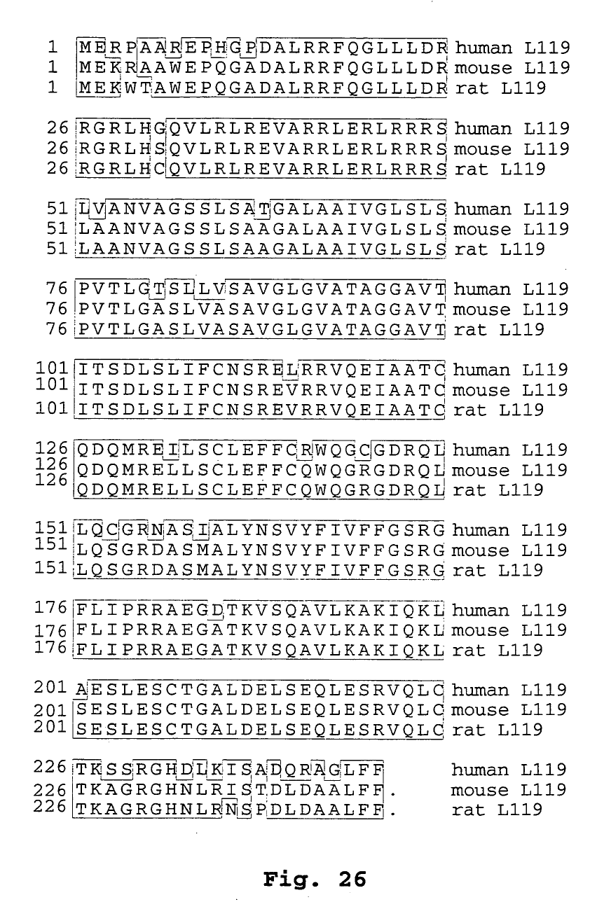

Coimmunoprecipitation was used to investigate whether L119 protein is able to interact with membrane receptors-which are expressed in vascular endothelial cells. Neuropilin-1 (Npn-1) was identified as being an isoform-specific (165-) VEGF receptor in endothelial cells. In this connection, Npn-1 appears to act as a coreceptor for the VEGF receptor KDR and transmits mitogenicity and migration signals in VEGF-165-stimulated endothelial cells (Soker S et al., (1998) Cell 92, 735-745). Npn-1 has also been described as being a cell-surface receptor for secreting semaphorin SemaIII (He Z and Tessier-Lavigne M (1997) Cell 90, 739-751; Kolodkin AL et al.(1997) Cell 90, 753-762). A construct for expressing the transmembrane receptor Npn-1 (provided with a myc tag; FL-Npn-1; provided by D. Ginty, Johns Hopkins University, Baltimore; described in Giger RJ et al. (1998) Neuron 21, 1079-92), or an empty vector control, was cotransfected, together with a pRK5 L119 expression construct, into COS 7 cells (transfection carried out in accordance with the “transient transfection” protocol). Expression of the two proteins was confirmed by Western blot analysis (data not shown). 48 hours after transfection, the cells were lysed by sonication in PBS/1% Triton X-100/protease inhibitors and the immunoprecipitation was performed in an analogous manner to the Notch 1 coimmunoprecipitation (cf. above). A monoclonal mouse anti-myc antibody (Calbiochem, Cat. No. OP10) was used as the immunoprecipitating antibody. The coimmunoprecipitation was detected by means of a Western blot analysis using an L119 antibody (2894) (carried out in accordance with the “Western blot analysis” protocol). FIG. 16 (upper row) shows that the anti-myc antibody was only able to coprecipitate L119 protein when myc-Npn-1 protein was present. In a controlled experiment, a peptide which blocked the myc antibody was also added, resulting in the disappearnace of the specific immunoprecipitated band (“IP control” lanes in the figure). This thereby demonstrated an interaction of L119 and Npn-1 proteins in heterologously transfected cells.

Npn-1 is a type I transmembrane protein having a large extracellular region and a short cytoplasmic tail (see, for example, Fujisawa H et al. (1997) Cell Tissue Res 290, 465-470). The extracellular region comprised 5 domains: two complement-binding domains (termed a1 and a2; see FIG. 16), two coagulation factor (V/VIII) domains (b1 and b2) and what is termed a MAM domain (c) (see bottom of FIG. 16 for a diagram). The domains al, a2, b1 and b2 are essential for binding SemaIII, while the domains b1 and b2 are essential for binding VEGF-165 (Giger RJ et al. (1998) Neuron 21, 1079-1092). It has been speculated that the MAM domain could be responsible for dimerizing or multimerizing Npn-1.

In order to identify the Npn-1 domains which interact with L119 protein, various deletion constructs of Npn-1 (as myc-tag fusion protein; provided by D. Ginty, Johns Hopkins University, Baltimore; described in Giger RJ et al. (1998) Neuron 21, 1079-92) were tested in a coimmunoprecipitations with L119. The following extracellular domains of Npn-1 were deleted: a1 and a2 in ΔA-Npn-1; b1 and b2 in AB-Npn-1; and c in AC-Npn-1 (compare bottom of FIG. 16). Coimmunoprecipitations using these expression constructs were carried out in COS 7 cells as described above for Notch 1. The middle row in FIG. 16 shows that, while deletion of the a and b domains did not have any influence on the interaction of Npn-1 with L119 protein, deletion of the c domain from the Npn-1 expression construct prevented this interaction.

### Example 10

### Antibodies Directed against the L119 Protein

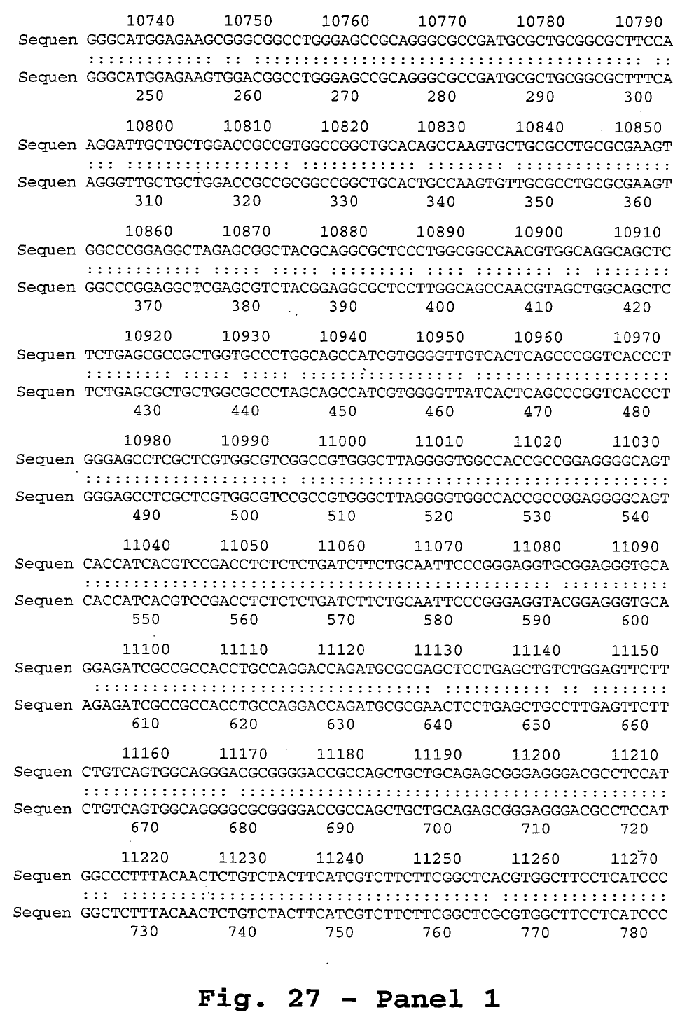

In order to prepare polyclonal antibodies which were directed against the L119 protein (rat), a peptide consisting of amino acids 8 to 20 (corresponding to the sequence in SEQ ID NO: 3) was synthesized, coupled by way of an additional terminal cysteine keyhole limpet hemacyanin (KLH), and injected into rabbits in order to produce antibodies (performed by the company Eurogentec). The antigen injections took place in accordance with the “standard immunization scheme” in Freud's adjuvant on days 0, 14, 28 and 56; blood was withdrawn from the animals on days 0 (preimmune serum), 38, 66 and 80. The sera of the rabbits were tested in Western Blot experiments for a specific reaction with heterologously expressed L119 protein. HEK293 cells were transiently transfected with an expression construct containing a fusion consisting of a myc tag and the entire open reading frame of L119 (pcDNA3.1-rL119-myc-His). After 48 hours, the cells were harvested and lysed and the protein extract was fractionated in triplicate on a denaturing protein gel and then blotted. While the Western blot analysis using the preimmune serum did not give any signals, the L119 antiserum 7340 gave a specific signal of the expected size (FIGS. 17, A and B). A control hybridization with an anti-myc antibody (Invitrogen) (FIG. 17C) labeled a band of the same size, thereby underlining the specificity of the 7340 antibody for the L119 protein.

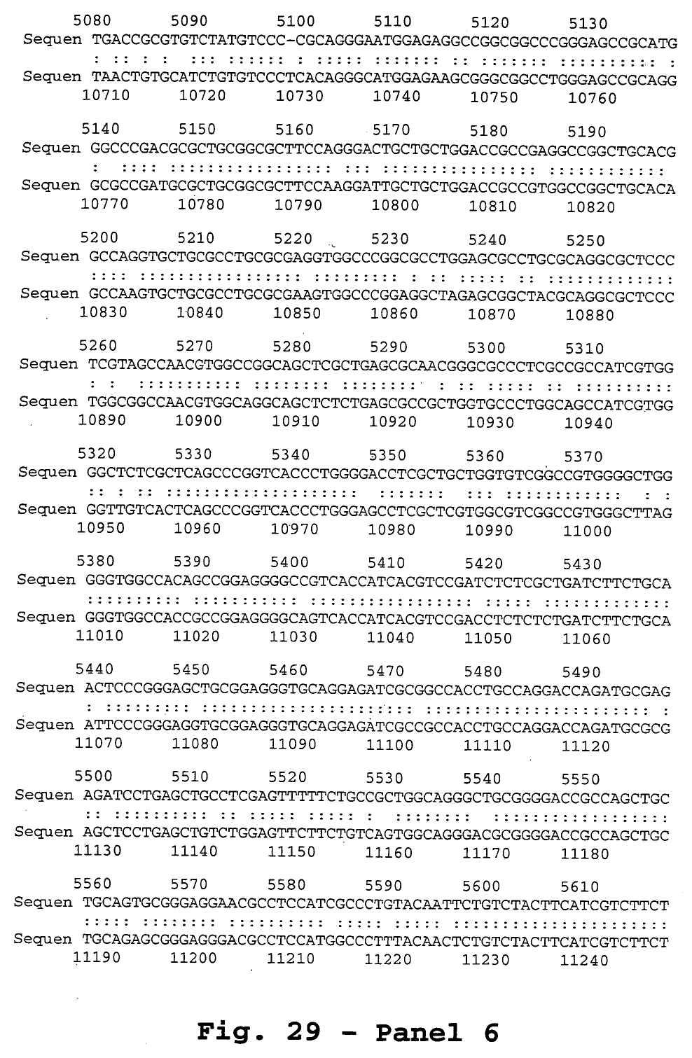

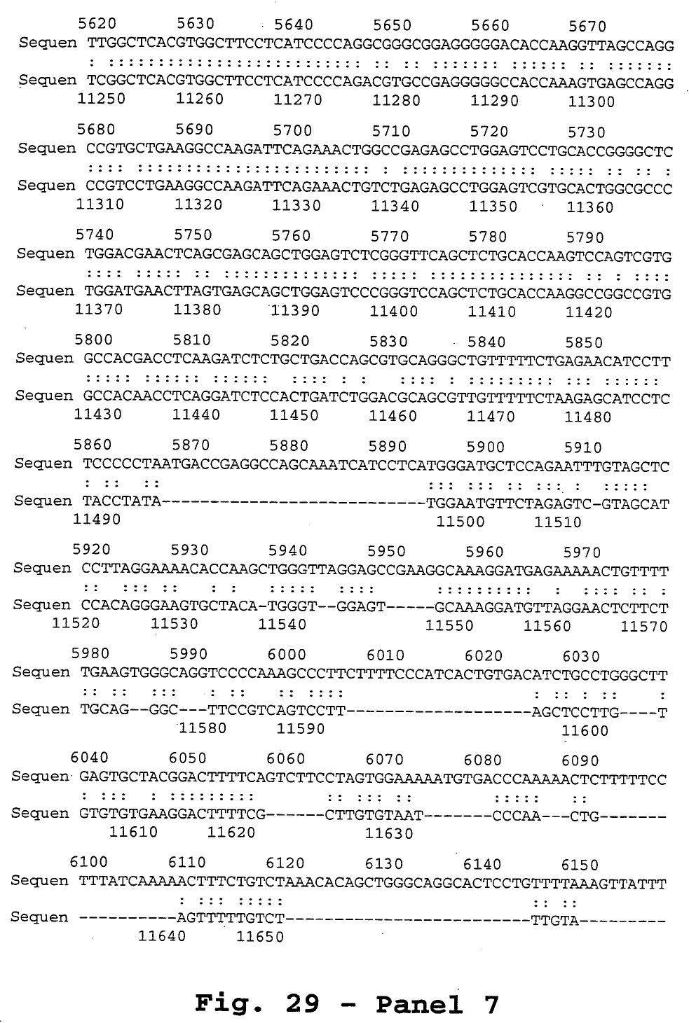

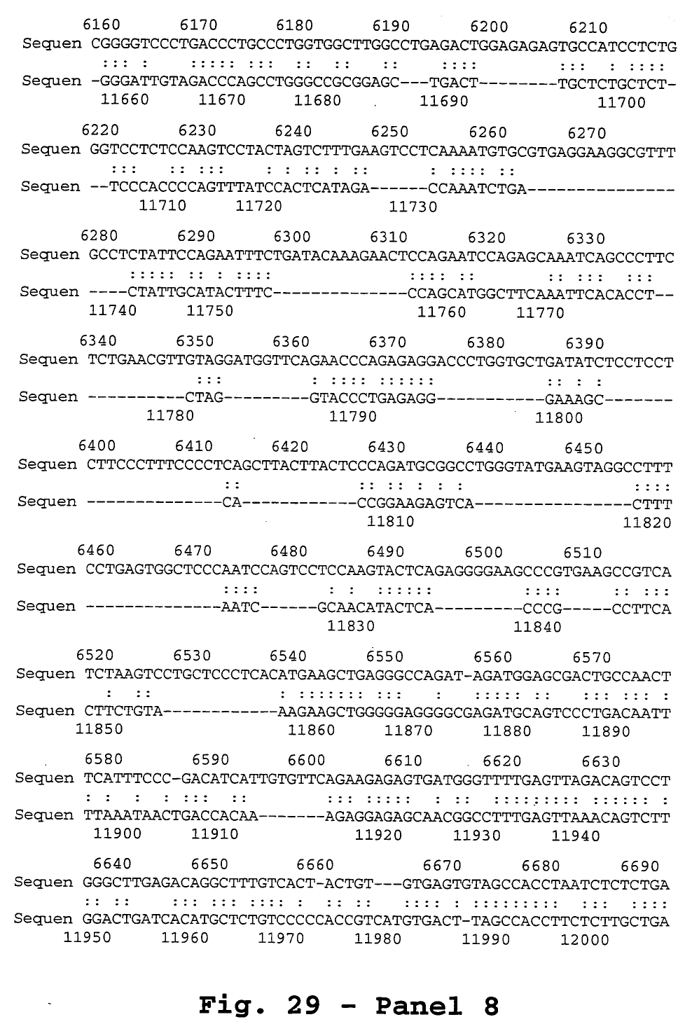

In order to prepare two further peptide antibodies, peptides consisting of the 19 N-terminal amino acids (MEKWTAWEPQGADALRRFQC) and the 29 C-terminal amino acids (CTKAGRGHNLRNSPDLDAALFF) of the L119 rat sequence (corresponding to the sequence in SEQ ID NO: 3) were coupled, by way of an additional terminal cysteine, to thyroglobulin (Sigma-Aldrich, Cat. No. T1001). For this, 10 mg of thyroglobulin were dissolved in 0.5 ml of 0.1 M phosphate buffer pH 6.8, while 2.5 mg of MBS (Pierce, Cat No. 22311ZZ) were dissolved in 0.1 ml of dimethylformamide (Sigma-Aldrich, Cat. No. P4254). 50 μl of the MBS solution were added dropwise to the thyroglobulin solution while agitating and the mixture was agitated at room temperature for a further 30 min. The MBS-thyroglobulin was purified on a PD-10 column (Amersham Pharmacia Biotech, Cat. No. 17-0851-01) in accordance with the manufacturer's instructions. The MBS-thyroglobulin-containing fractions were detected by measuring the absorption at 280 nm and then combined. 1 mg/ml solutions of the N- and C-terminal peptides were prepared in 0.1 M phosphate buffer, pH 6.8/20 mM EDTA, and 3 ml of the MBS-thyroglobulin solution were mixed with 3 ml of peptide solution under a protective gas (N₂) and the whole was stirred at room temperature for 4 h. The coupling products were dialyzed against PBS overnight and then used as immunogen. In addition to this, a GST-L119 fusion protein was also used for the immunization. In order to prepare the fusion protein, a PCR fragment was prepared which consisted of the 67 C-terminal amino acids of the rat L119 (corresponding to the sequence in SEQ ID NO: 3). The oligonucleotide primers having the sequences SEQ ID NO: 37 and 38 were used for this purpose. A 50 μl PCR mixture was prepared from 5 μl of 10× cloned Pfu buffer (Stratagene); 2 μl of dNTP mixture (5 mM; Cat. No. 1969064, Roche Diagnostics GmbH, Mannheim, Germany); in each case 2 μl of the abovementioned primer pairs (stock conc. 10 KM); 100 ng of rL119 cDNA template, 35 μl of H₂O and 1 μl of Pfu turbo DNA polymerase (Stratagene), and, after the mixture had been incubated at 94° C. for 3 minutes, 28 PCR cycles were then performed in accordance with the following temperature program: 1 min of primer annealing at 62° C., 1 min of strand extension at 72° C., and 1 min of DNA double-strand melting at 94° C., together with a concluding extension step of 7 min at 72° C. The resulting PCR products were gel-purified and cloned into the BamHI and SalI cloning sites of the vector pGEX-4T2 (Amersham Pharmacia Biotech, Cat. No. 27-4581-01). The GST-fusion proteins were sequenced and then expressed, in accordance with the manufacturer's standard protocol, in E. coli BL21 cells (cell growth at up to an OD₆₀₀ of 0.8; induction with IPTG (Amersham Pharmacia Biotech, Cat. No. US17884-5g) for 2 h); the bacterial pellet was then lysed by sonicating in PBS/1% Triton-X100 and centrifuged at 25000 g (4° C.) for 30 min; the supernatant which was obtained was then incubated with glutathione-agarose beads at 4° C. for 2 h. The beads were washed 4× with PBS/Triton-X100 and the fusion protein was eluted with 2 ml of 10 mM glutathione/50 mM Tris-HCl, pH 8.0 (by incubating at 4° C. for 1 h) and then dialyzed against PBS. The dialyzed protein solution was used as the antigen. The immunization of in each case two rabbits was carried out by Covance Research Products Inc. (Antigen injections took place, in Freud's adjuvant, in accordance with the “Master Schedule list”, on days 0, 14, 35 and 56, 77 and 98, the blood being withdrawn from the animals on days 0 (preimmune serum), 25, 46, 67, 88 and 109). The sera from the rabbits (peptide antibody: 2892-2895; GST-fusion proteins 3841 and 3843) were tested in Western blot experiments for a specific reaction with heterologously expressed L119 protein. For this, HEK293 cells were transiently transfected with expression constructs containing a fusion consisting of a myc tag and the entire open reading frame of the rat or human 119, and also transiently transfected in parallel with the corresponding vector construct. After 48 hours, the cells were harvested and, after 15 min on ice, disrupted in a hypotonic buffer (10 mM HEPES pH 7.6, 1.5 mM MgCl₂, 10 MM KCR, 1 mM EDTA) by being drawn 30 times through a 22 gage needle, after which they were centrifuged at 1000 g for 10 min (4° C.). The 1000.g supernatant was fractionated on a denaturing protein gel and then blotted. The Western blot was carried out in accordance with the “Western blot analysis” protocol. A control hybridization with an anti-myc antibody (Biomol) was carried out in order to identify the L119-specific bands. The sera 2892 to 2895 displayed a specific reaction with the rat L119 protein whereas it was not possible to detect any immune reaction with the human L119 protein (FIGS. 25a and b). The sera 3841 and 3843 were tested for an immune reaction in an analogous manner while incubation with an anti-myc antibody once again served as the control. In this case, both the L119 antibodies were found to react strongly with the rat L119 protein and to give a weak immune reaction with the human L119 (FIG. 25c).

### Example 11

### Preparation of L119-Transgenic Animals

Important additional information about the (patho)physiological mechanisms in which the L119 gene is involved can be obtained by specifically mutating the L119 gene in the mouse germ line and analyzing the resulting phenotype. In order to prepare what is termed a knock-out mouse, i.e. a mouse lacking any functional L119 protein, a targeting construct was first of all used. For this, two genomic fragments which flanked the L119 coding region, and which corresponded to positions 2820 to 10736 and 13536 to 14986 in the sequence according to the invention SEQ ID NO: 4, were cloned, as homology arms for the homologous recombination in embryonic stem cells (ES), into the vector pHM2 (Kaestner KH et al. (1994) Gene 148, 67-70; EMBL Database Accession No. X76683) This vector carries a neomycin resistance cassette and enables a reporter gene to be inserted into the allele which is to be mutated. For this, the lacZ reporter gene of the vector was fused to the 5′-untranslated region of L119 and was consequently under the control of the endogenous L119 promoter. In detail, an approx. 1400 bp-long mouse genomic HindIII/EcoRI fragment from the 3′-untranslated region of L119 (corresponding to positions 13536 to 14986 in the sequence according to the invention SEQ ID NO: 4) was cloned into the correspondingly cleaved vector pBluescriptIIKS-Minus (from Stratagene). The insert was isolated once again from the construct with SalI/SpeI and cloned into the vector pHM2, which had been cut with SalI and XbaI. This thereby cloned the 3′ homology arm for the homologous recombination. The 5′ homology arm was cloned in 2 constituent steps. As the first step, the construct was digested, for the subsequent cloning, with NotI and PmlI. In order to generate the insert which was to be cloned in, a PCR was carried out on 10 ng. of mouse L119 cosmid DNA using the primers

| | |
|---|---|
| mgL119-3sNotI | |
| 5′-AAATATGCGGCCGCAGTGTGCCCTTTCTGAG | (SEQ ID NO: 43) |
| ACC-3′ | |
| mgL119-4as | |
| 5′-CTCCATGCCCTGTGAGGGACACAG-3′ | (SEQ ID NO: 44) |

and employing the following conditions: 3 min at 96° C. for the initial denaturation and then 25 cycles of 30 sec at 96° C. for denaturation, 30 sec at 65° C. for annealing and 4 min at 72° C. for elongation. The PCR product was digested with NotI and cloned into the previously prepared construct (cut with NotI and PmlI). The resulting plasmid was digested with NotI and XhoI and the intervening fragment of about 730 bp in length (originating from the 5′ region of the previously cloned PCR product including the NotI cleavage site of the primer used for the PCR) was replaced with a NotI/XhoI fragment of about 6600 bp in length. The NotI/XhoI fragment which was inserted was obtained from a plasmid containing an L119 genomic EcoRI fragment into which, following transposon insertion (GPS-1, New England Biolabs, Beverly, Mass., USA; carried out in accordance with the protocol in the manual accompanying the kit) an additional NotI cleavage site had been introduced. Inserting the NotI/XhoI fragment restored the genomic context of the mouse L119 (corresponding to positions 2820 to 10736 in the sequence SEQ ID NO: 4 according to the invention) in the knock-out construct. After linearizing the targeting construct with SmaI, the DNA was electroporated into embryonic stem cells and G418-resistant clones were selected (performed by EUROGENTEC). Genomic DNA was isolated from these clones (in accordance with the “preparation of genomic DNA from mammalian tissue: Basic Protocol” in Ausubel et al. (eds.), Current Protocols in Molecular Biology, Volume 1, Supplement 42 (1998), pages 2.2.1.-2.2.3. (John Wiley and Sons)) and examined by PCR for homologous recombination between the targeting construct and the endogenous L119 allele. For the PCR, a pair of primers was selected, one of which primers (pHM2-7s) bound to specific sequences in the targeting construct (corresponding to positions 7727 to 7750 in pHM2; EMBL Accession Number X76683), while the second primer (mgL119-15as) was selected from the 3′ flanking sequence of the genomic L119 allele (corresponding to positions 15116 to 15093 in SEQ ID NO: 4):

The primers employed for the PCR were:

| | |
|---|---|
| pHM2-7s: | |
| 5′-GACCGCTATCAGGACATAGCGTTG-3′ | (SEQ ID NO: 20) |
| mgL119-15as: | |
| 5′-ACTATGTAGCCTGGGCTCAGGTAG-3′ | (SEQ ID NO: 21) |

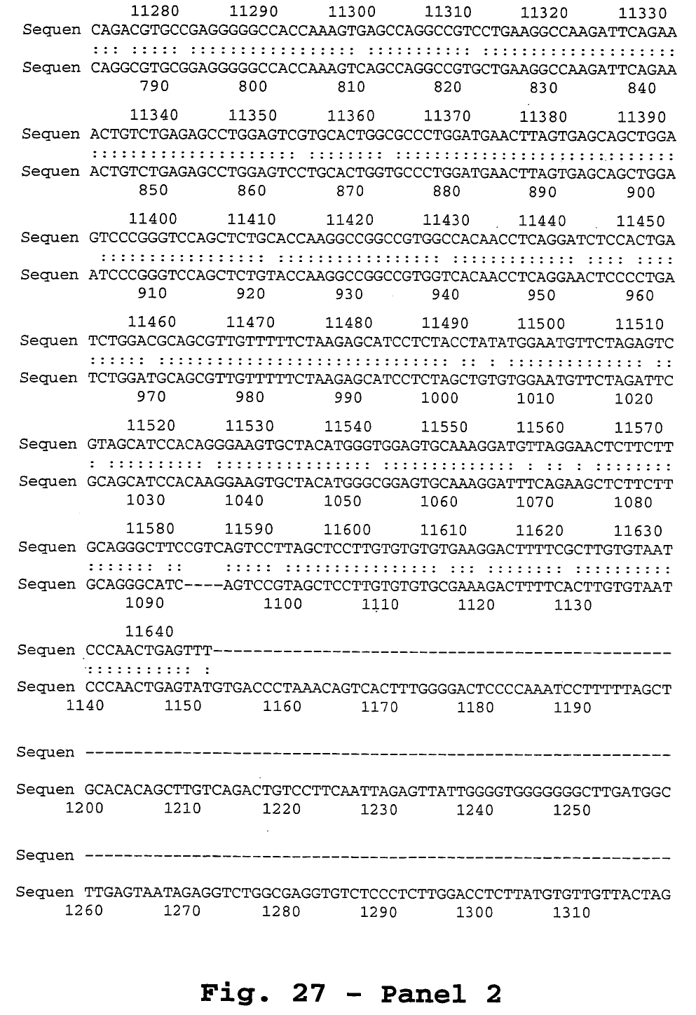

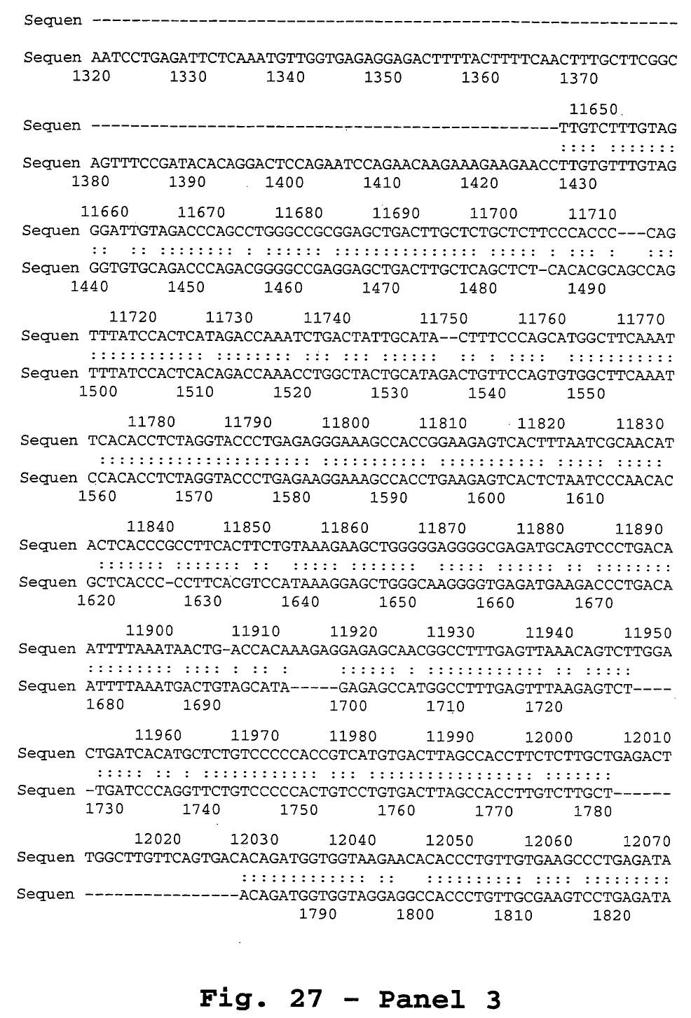

The PCR was carried out in accordance with the “polymerase chain reactions” protocol under the following conditions: 50 ng of genomic DNA with 4 min at 96° C. for initial denaturation and then 40 cycles of 15 sec at 96° C. for denaturation, 30 sec at 60° C. for annealing and 3 min at 72° C. for elongation. The two primers are only able to amplify a PCR product (2217 bp) after the L119 targeting construct has successfully recombined homologously with the endogenous L119 allele. FIG. 18 shows a photograph of an agarose gel of such a PCR amplification. A band of the expected size was amplified from the genomic DNA in the ES cells #308 and #341 but not from the genomic DNA in #307. A negative control (PCR reaction without ES cell DNA) was analyzed in the first lane. The 1 kb ladder supplied by MBI Fermentas was loaded as the marker. In summary, it was possible to demonstrate that the desired homologous recombination took place in the ES cell clones #308 and #341.

ES cell clone #341 was injected into blastocysts of C57Bl/6 mice which were implanted into pseudopregnant foster females (according to standard protocols in “Manipulating the Mouse Embryo: A Laboratory Manual” by B. Hogan, R. Beddington, F. Costantini, E. Lacy (Cold Spring Harbor Laboratory, 2^{nd} edition 1994). Chimeric males capable of germline transmission of the L119 ko gene were identified. Heterozygous progeny was propagated for studies by backcrossing to C57Bl/6 mice. For experiments, heterozygotes were interbred and wildtype and mutant mice subjected to analysis.

### Example 12

### L119 Protein Expression is Induced after Kainate Treatment

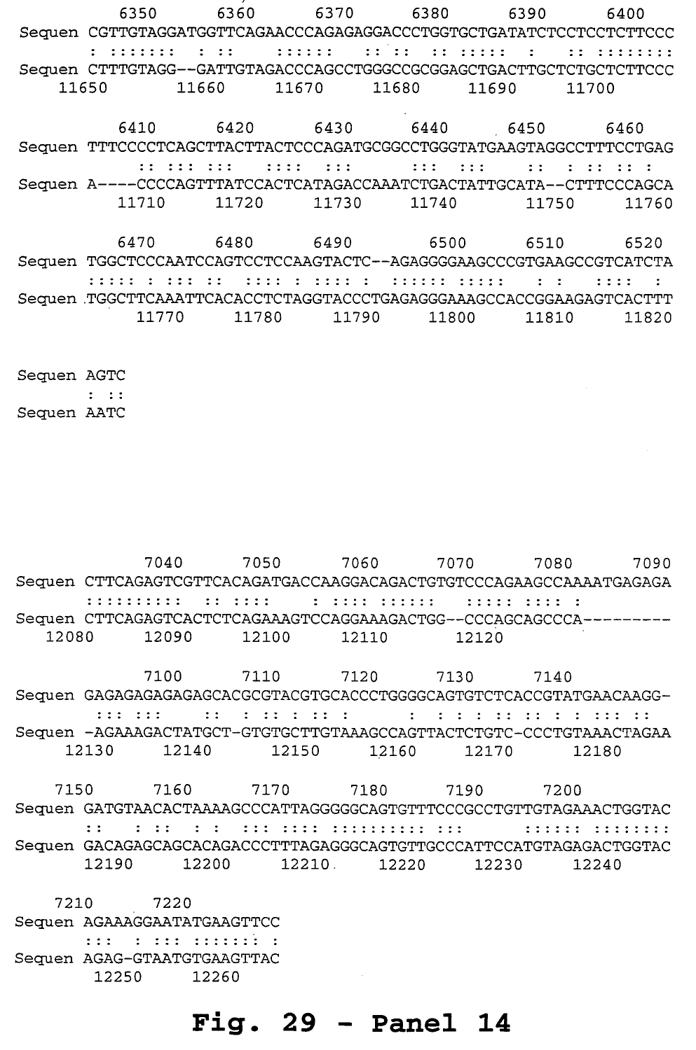

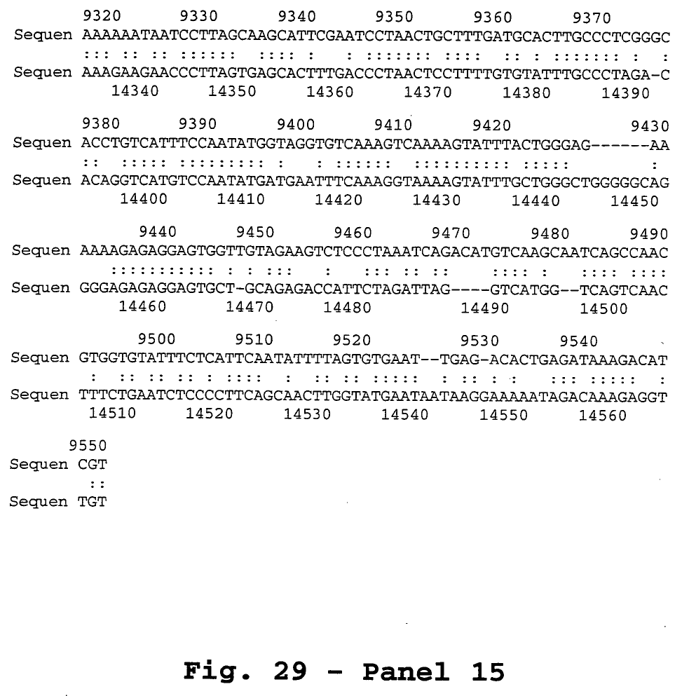

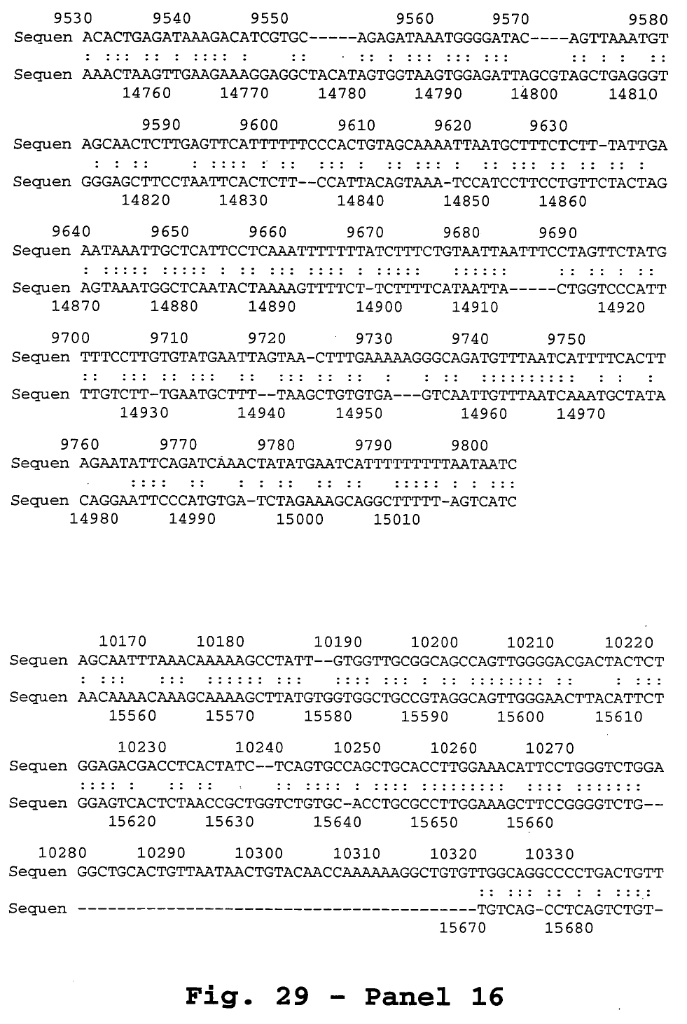

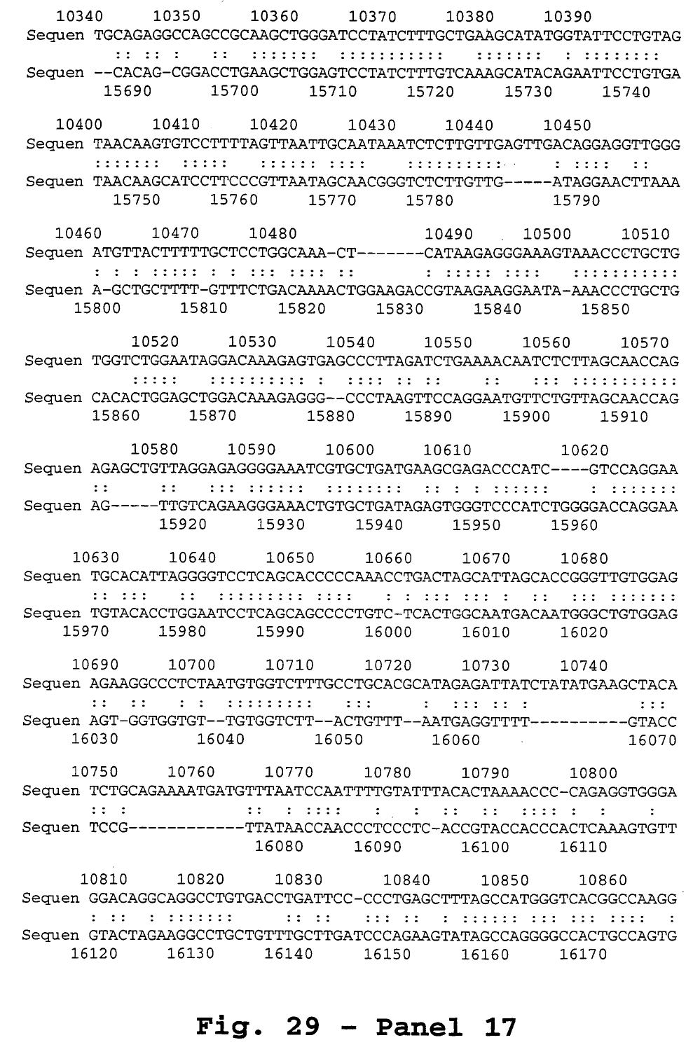

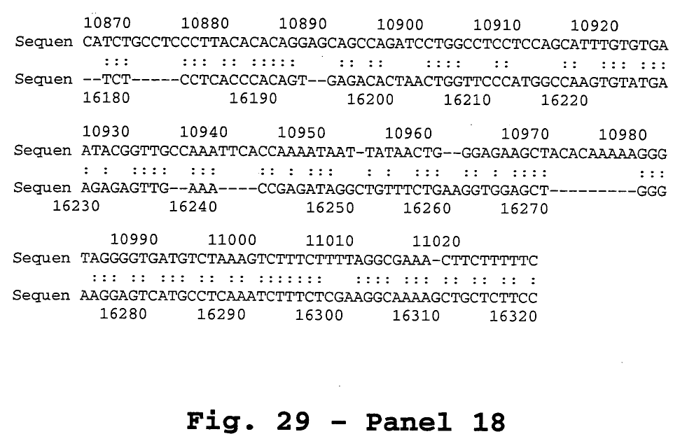

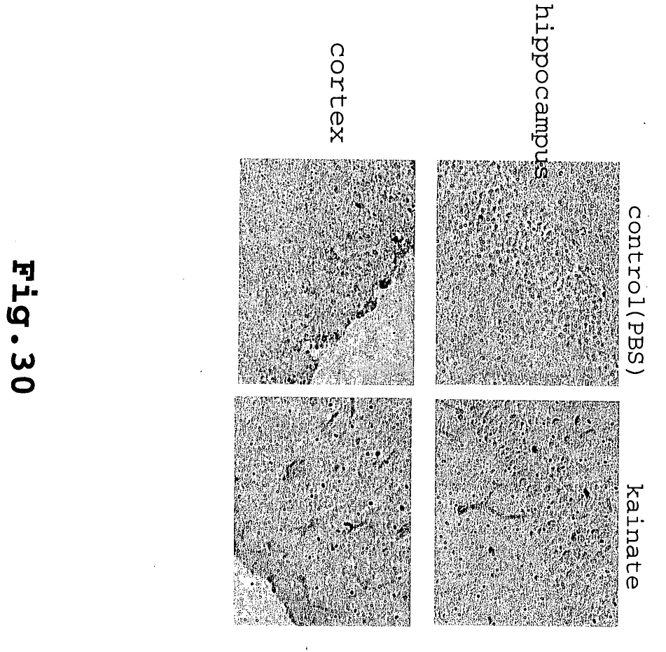

Male wistar rats (5 weeks old) were injected intraperitoneally with either 12 mg/kg kainate or PBS only. 3 h after onset of seizures (4 h after injection) rats were anesthetized with chloral hydrate (Sigma, Cat. No. C-8383) (3.6%, 1 ml/100 g body weight, i. p.) and perfused with 75 ml PBS. The brain was removed, frozen on dry ice and sectioned in 20 μm cryosections. For immunohistochemical analysis sections from wt and ko animals were thawed at room temperature and fixed for 20 min in PBS with 2% paraformaldeyde (pH 7.0) (Merck, Cat. No. 1.04005.1000) with gentle shaking. The following procedure was carried out at room temperature and incubation and washing steps were performed with gentle shaking. Sections were washed twice for 5 min with PBS and then incubated for 30 min with 1% hydrogen peroxide in PBS/methanol (1:1) for quenching of endogenous peroxidase activity. Permeabilization was performed with PBS/0.2% Triton-X100 (PBST) for 2×15 min. Sections were blocked in PBS/5% normal goat serum /0.2% Triton-X100 (normal goat serum (NGS) from Jackson ImmunoResearch Laboratories, Cat. No. 005-000-121) for 30 min followed by over night incubation with the L119 specific polyclonal antibody 2892 (1:200) in PBS/4% NGS/0.1% Triton-X100 at 4° C. The sections were washed 3× for 5 min with PBST. Secondary antibody incubation was done with anti-rabbit Vectastain Elite ABC immunoperoxidase system (Vector Laboratories, Inc.). 10 ml PBST were mixed with 2 drops of goat serum and 1 drop of biotinylated secondary anti-rabbit antibody from the anti-rabbit staining kit. Sections were incubated for 30 min with the reaction mix and then washed 3× for 15 min with PBST. To 10 ml PBST 2 drops of reagent A (avidin) and 2 drops of reagent B (biotinylated peroxidase) were added and incubated with gentle shaking for 30 min at room temperature. Sections were incubated with the A plus B reagent solution for 30 min. After 3 washing steps for 15 min with PBST followed by 3×15 min washing steps with PBS the DAB (3,3′-diaminobenzidene) staining was performed according to the manufacturers' instructions (Vector Laboratories, Inc., Peroxidase Substrate Kit DAB, Cat. No. SK-4100) whereas only half of the amount of DAB was used. DAB staining reagent was prepared by mixing of 5 ml of water with 2 drops of buffer stock solution, 2 drops of DAB stock solution and 2 drops of peroxidase solution. For staining slides were immersed for 2-4 min in a coplin jar with DAB staining reagent. Color development was stopped by transferring the slides to a coplin jar with PBS. Sections were washed 3×2 min with 10 mM Tris-HCl pH 7.6 and mounted with Aqua PolyMount (Polysciences, Inc., Cat. No. 18606). In kainate treated animals an endothelial specific L119 staining could be detected with the strongest signal intensity in the hippocampus and in the cortex of stimulated animals. (FIG. 30)

### Example 13

### Induction of L119 Gene Expression by Treatment with Lipopolysaccharides (LPS)

C57Bl/6 mice were injected with lipopolysaccharides (Sigma L-2630, 2.5 mg/kg, i.p.) (n=6) or PBS (n=6). After 3 h mice were anesthetized and perfused transcardially with 20 ml of Ringer solution. They were decapitated, the brain was carefully removed and frozen on dry ice. The brains were stored at −80° C. RNA was extracted with the RNA clean kit (AGS, Heidelberg, Germany) and RNA was reverse transcribed using random hexamer primers and MMLV (Promega, Mannheim, Germany) according to the manufacturers instructions. L119 cDNA levels were determined by real time PCR (LightCycler, Roche Diagnostics). The PCR was performed with L119 specific primers resulting in a 330 bp L119 PCR-fragment.

| | |
|---|---|
| L119-17s: | |
| 5′-GGGTCTGAATAGGAAGGGAGTCTG-3′ | (SEQ ID NO: 45) |
| L119-19as: | |
| 5′-ATAGGACATCAGGTTTCCAAGGTC-3′ | (SEQ ID NO: 46) |

As an internal standard a 300 bp fragment of cyclophilin A was amplified in parallel using the primers:

| | |
|---|---|
| Cyc5 (s): | |
| 5′-ACCCCACCGTGTTCTTCGAC-3′ | (SEQ ID NO: 47) |
| acyc300 (as): | |
| 5′-CATTTGCCATGGACAAGATG-3′ | (SEQ ID NO: 48) |

50 PCR cycles were performed using the DNA Master SYBR Green I kit (Roche Diagnosics, Mannheim, Germany) with an annealing temperature of 60° C. in a volume of 20 μl. 0.5 μM of each primer, 4 mM MgCl₂ (final concentration) and 0.16 μl TaqStart AB (Clontech; Heidelberg, Germany) was used per reaction.

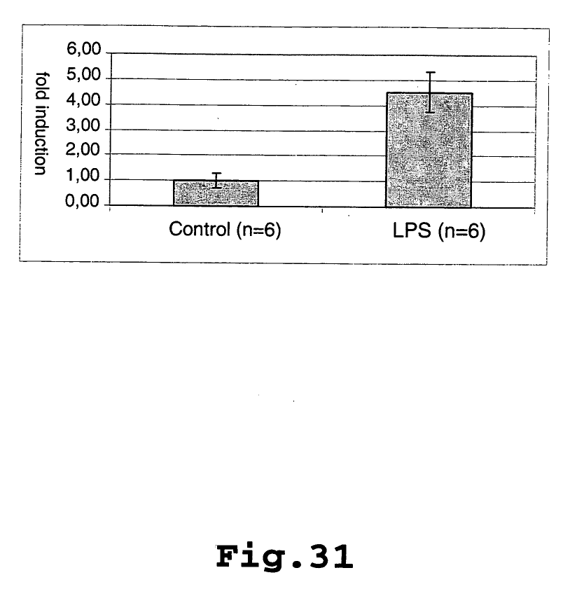

LPS treatment represents a common model for septic shock and caused a 4-5 fold increase of L119 mRNA levels (FIG. 31; normalized to cyclophilin A levels; arrow bars represent SD). These results suggest that the immediate early gene L119 might be involved in acute or chronic inflammatory processes and defense mechanisms.

### Example 14

### Cycloheximide Treatment of L119 wt and ko Mice

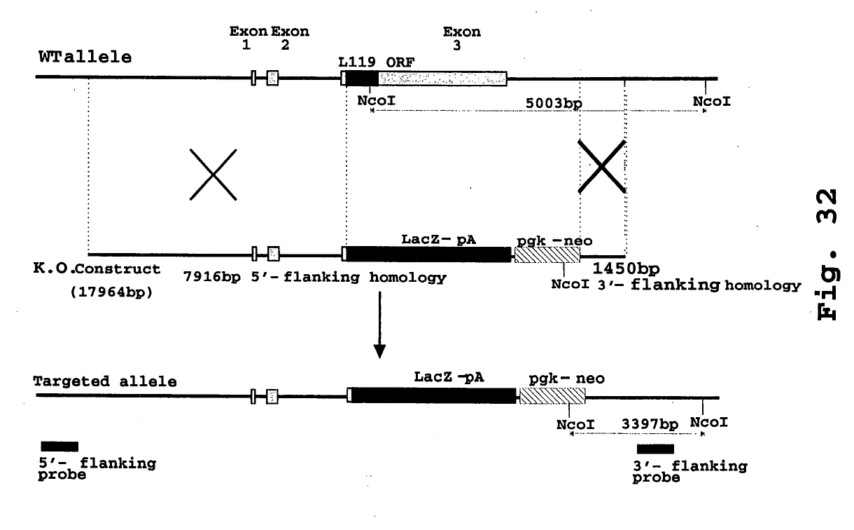

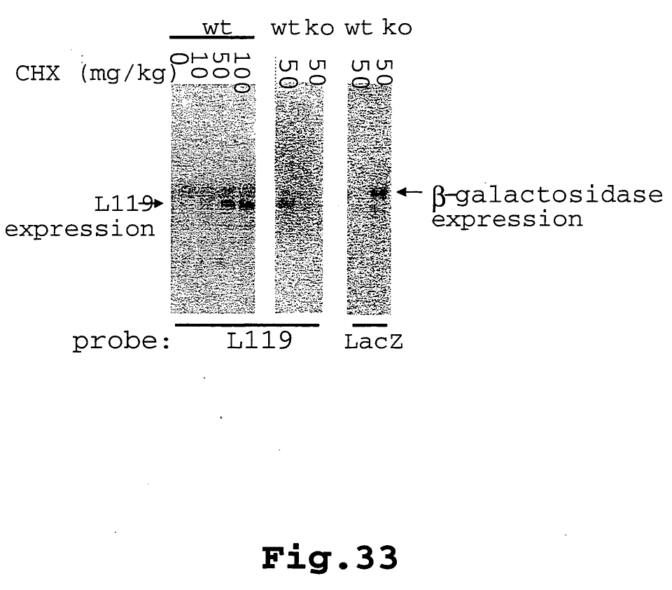

To verify deficiency of L119 gene expression in L119 ko mice northern blot analysis was performed after cycloheximide (CHX) treatment of wt and ko mice (FIG. 33). Four male wt mice (six month old) were injected with either PBS/Ethanol (1:1) or 10, 50 or 100 mg CHX/kg (i. p.) dissolved in PBS/Ethanol (1:1), respectively and two male ko littermates received either PBS/EtOH or 50 mg CHX/kg. Four hours after injection, mice were decapitated, the brain carefully removed and the right half of each brain was frozen on dry ice. From the left half total RNA was prepared as described under Methods (section c). 10 μg of total RNA was used for northern blot analysis (as described in Method section d). Pre-treatment with 50 and 100 mg CHX/kg body weight induced L119 gene expression in wt animals (FIG. 33, left and middle panel). In contrast, no L119 specific signal could be detected in CHX treated ko animals (50 mg CHX/kg body weight) by northern blotting with the identical L119 probe verifying the absence of L119 coding sequence. Instead, a probe for β-galactosidase gave a specific northern signal in CHX treated ko animals, which was absent in wt mice and untreated ko animals. Both probes were generated according to the protocol “Radioactive labeling of PCR fragments” (section f). As L119 specific probe a 329 bp PCR fragment was used (described in Example 1 and 5) and for generation of the β-galactosidase probe a 1120 bp fragment was generated by PCR using the following primers:

| | |
|---|---|
| pHM2-8: | |
| 5′-GTGACCATGTCGTTTACTTTGACC-3′ | (SEQ ID NO: 49) |
| pHM2-9: | |
| 5′-GGTTAACGCCTCGAATCAGCAACG-3′ | (SEQ ID NO: 50) |

The fragment was amplified using 25 ng vector DNA of pHM2 (EMBL accession number X76683) as a template with standard PCR conditions (methods section a).

In conclusion, it could be demonstrated that in L119 ko mice the coding sequence of L119 had been successfully deleted and substituted by a functional β-galactosidase reporter gene (FIG. 33).

### Example 15

### Developmental β-Galactosidase Expression in Heterozygote E12.5 L119 ko Mice

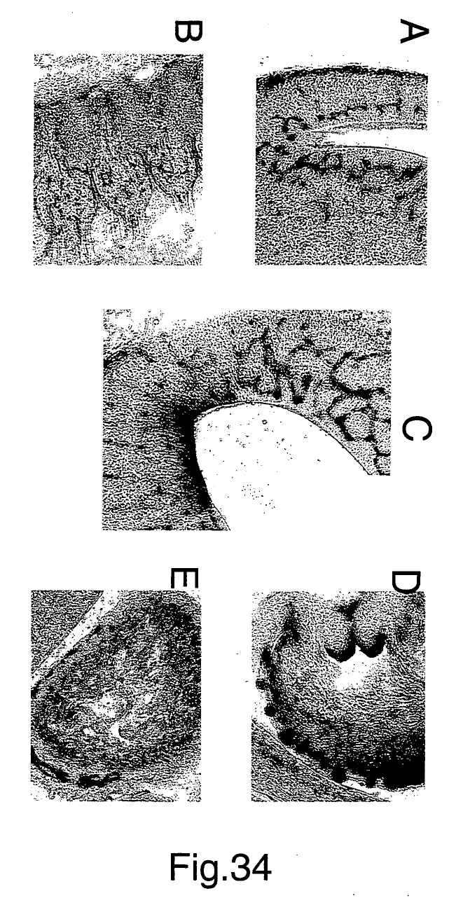

L119 is upregulated in endothelial cells during embryogenesis (FIGS. 8 and 9). L119 promotor activity in heterozygote E12.5 embryos expressing β-galactosidase from the endogenous L119 promotor was analyzed. Pregnant mice were killed and embryos removed from the uterus. They were separated from placenta and yolk sac and transferred to a well of a 12-well plate containing PBS. The placenta was recovered for genotyping and frozen in liquid nitrogen. Embryos were fixed for 30 min at 4° C. in fixation solution (PBS/1% formaldehyde/0.2% glutaraldehyde/0.02% NP-40). They were washed 3× for 20 min at room temperature with PBS. PBS was removed and embryos were stained for 48 h at 30° C. with X-gal-staining-solution (PBS/5 mM K₃Fe(CN)₆/5 mM K₄Fe(CN)₆/2 mM MgCl₂ and 1 mg/ml 5-bromo-4-chloro-3-indolyl-beta-D-galactoside (X-gal)). After the staining embryos were washed 3× for 20 min in PBS at 4° C. and then transferred every 3 days to fresh PBS with increasing concentrations of glycerol (30%, 50%, 80%). For embedding of embryos 0.5 g gelatin (Sigma-Aldrich, Cat. No. G-1393) was dissolved in 100 ml PBS under constant stirring and heating. After the solution had cooled down to room temperature 30 g bovine albumin (Sigma-Aldrich, Cat. No. A-7906) followed by 20 g sucrose (Sigma-Aldrich, Cat. No. S-7903) was dissolved in the gelatin solution. 0.2 ml of a 25% glutaraldehyde-solution (Sigma-Aldrich, Cat. No. G-6257) were added and the mixture quickly transferred to 6 cm petridishes. The embryos were placed on top of the embedding mixture before it completely solidified. Embryos were then quickly covered with a layer of embedding mixture. After solidification of embedding material a block containing the embryo was cut out and placed for 15 min in water. The block was then sectioned at 50 μm using a Leica VT 1000 S vibratom. X-gal staining of brain (A,C), spinal cordm (B) and heart was analyzed (D,E). (FIG. 34)

For genotyping of embryos total RNA was prepared from placenta and first strand cDNA synthesis was performed according to method sections b and c. The mouse genotype was determined by multiplex PCR analysis using the primer set:

| | |
|---|---|
| L119-MG-F2 (s): | |
| 5′-CTCTAGCCTAGGGCAGCAAC-3′ | (SEQ ID NO: 51) |
| L119-MG-R1 (as): | |
| 5′-GAGAGAGGTCGGACGTGATG-3′ | (SEQ ID NO: 52) |
| L119-LacZ-R1: | |
| 5′-GGCGATTAAGTTGGGTAACG-3′ | (SEQ ID NO: 53) |

35 PCR cycles were performed according to methods (section a). All three primers were used at a concentration of 1 μM and annealing steps were done at 59° C. resulting in a 400 bp PCR product (wt allele) and/or a 200 bp PCR product (ko allele). The L119 knock-out/β-galactosidase knock-in mice are a suitable .model system for studying gene regulation of this locus. In contrast, due to the short half-life of the L119 MRNA and protein, it is difficult to study the low abundant L119 gene product directly.

### Example 16

### General Physiology of L119 ko Mice

L119 ko mice develop normal, are fertile and appear healthy. Moreover, they show no obvious behavioral deficits. For further analysis of their health condition standard laboratory parameters were determined. 6 wt and 6 L119 ko mice (8 month old) were kept for 24 h in a metabolic cage and excretion within the 24 h period was monitored. Urine was collected for 24 h and urea (uurea), creatinin (ucrea), salt and protein concentrations were analyzed on a Hitachi Automatic Analyzer and determined as excretion within 24 h per g of body weight. L119 ko mice showed a mild decrease in urea and creatinin excretion over a 24 h time period (urea: 2.418 g/24 h for ko mice, 3.215 g/24 h for wild-type mice; creatinine: 0,011 g/24 h for ko mice, 0,0129 g/24 h for wild-type mice).

On the next day blood was taken from ether anesthetized animals and Li-EDTA plasma samples and urine samples were analyzed on a Hitachi Automatic Analyzer. No significant differences were observed between ko and wt animals. Three days later blood pressure and heart frequency of both groups were determined. Values for systolic pressure and for mean arterial pressure were similar for both groups of animals. In summary, no significant differences in standard laboratory parameters were determined for L119 ko mice compared to wt littermates.

### Example 17

### Increased Infarct Volume in L119 ko Mice in a Model of Focal Cerebral Ischemia

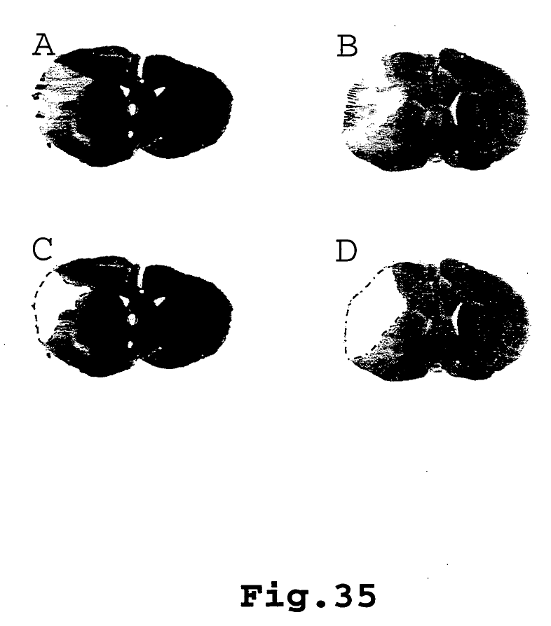

The occlusion of the left median cerebral artery (MCA) was 5 performed according to Backhaui and colleagues (Backhau: et al. (1992) A mouse model of focal cerebral ischemia for screening neuroprotective drug effects, J Pharmacol Meth 27: 27-32). Mice were anesthetized with avertin (15 μl 2.5% avertin/g, i.p.). A skin incision was made on the left temporoparietal region of the head between the ear and the orbit. The parotid gland and the temporalis muscle were removed by electrical coagulation (ICC 300, Erbe, Tubingen, Germany). A small borehole was drilled, and the left MCA was occluded at three sites by microbipolar coagulation. Body temperature was maintained at 37° C. by placing the mice on a heating pad that was controlled by a rectal temperature probe. After surgery the mice were placed under a heating lamp for 1 hour. Two days after the surgery mice were anesthetized once more with avertin and were perfused transcardially with 20 ml of Ringer solution. They were decapitated, the brain was carefully removed and frozen in isopentane. Brains were stored at −80° C. until sectioning. Coronal cryosections (20 μm in thickness) were cut every 400 μm, starting rostrally. Sections from wt (FIG. 35 A) and L119 ko mice (FIG. 35 B) were silverstained according to Vogel et al. (1999, Early delineation of ischemic tissue in rat brain cryosections by high contrast staining, Stroke 30: 1134-1141). Stained sections were directly scanned at 600 dpi and the infarct area was measured (NIH Image). The total infact volume was obtained from integrating infarcted areas and correcting for brain edema by subtracting the difference in the volumes of left and right hemisphere. For better visualization affected areas were colored in white in FIGS. 35 C (wt) and D (ko).

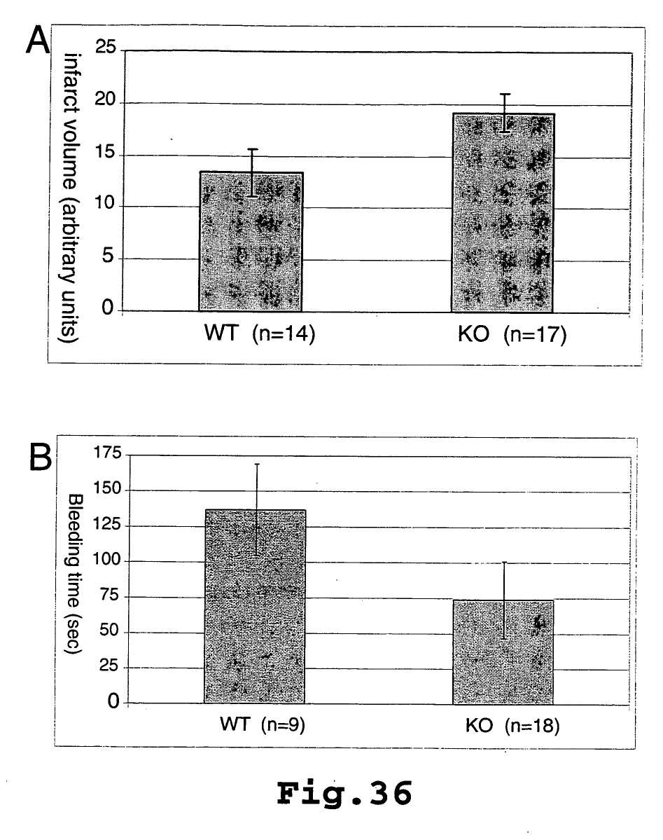

L119 ko mice showed an increased infarct volume compared to wt littermates. Infarct volumes were determined for 14 wt and 17 ko mice in total and corrected for brain edema. L119 ko mice showed a significant increase in infarct volume (19,2±1,8) compared to wt littermates (13,4±2,3; SEM) (FIG. 36A), p=0,047 for the nonparametric Mann-Whitney test. From these data it can be concluded that L119 has an positive effect on the outcome of an ischemic event and might act as a protective factor.

### Example 18

### Analysis of Tail Bleeding Time of wt and L119 ko Mice

In a blinded experiment L119 ko and wildtype mice (8-12 weeks old) were anesthetized by intraperitoneal injection of sodium pentobarbital (60 mg/kg) and their ear tag number was noted. The tail was immersed into a bath of PBS at 37° C. 5-8 mm of the tail was quickly cleaned and amputated using surgical scissors. Subaqueous bleeding time was defined by the time from the cut until blood flow had stopped for approximately 3-5 sec. At the end of trial, the tag number and the bleeding time were matched to the genotype. L119 ko mice (n=18) showed significantly decreased bleeding times compared to wt littermates (n=9). Mean bleeding times for wt (137±32.4 s) and ko mice (74±27.2 s) are shown in FIG. 36B, error bars represent standard errors. Statistical significance was determined using an unpaired student's t-test (p<0.0001). For further characterization of the L119 phenotype whole blood aggregation and platelet aggregation assays were performed.

### Example 19

### Whole blood aggregation assay of wt and L119 ko mice

Blood Cell Counts

Heparin blood (1000 units/ml Heparin in 137 mM NaCl, 1:9) was drawn from wt and ko mice and peripheral blood counts were determined (Beckman Coulter Counter). Although a variability was observed between animals within one group, there were no significant differences in red blood cell (RBC), white blood cell (WBC) or platelet counts between both groups of animals.

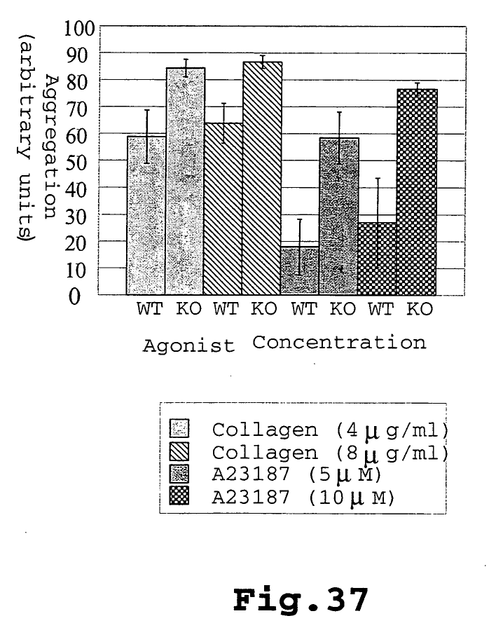

Heparin blood (1000 units/ml Heparin in 137 mM NaCl, 1:9) was obtained from wt and L119 ko mice (n=11). Blood cell counts were determined and aliquots of blood were placed into an aggregometer. After addition of agonists (collagen (4 or 8 μg/μl; n=6), calcium ionophore A23187 (5 or 10 μM; n=5)) aggregation of platelets was determined by measurement of the increase in electrical resistance over a period of 5 min. Data are shown in arbitrary units and represent maximal resistance divided by platelet concentration (FIG. 37; arrow bars represent SEM values). Blood derived from L119 ko mice aggregated more vigorously than wt blood in response to the same concentration of agonist suggesting that the L119 gene product might have anti-thrombotic effects.

### Example 20

### Platelet Aggregation of wt and L119 ko Mice

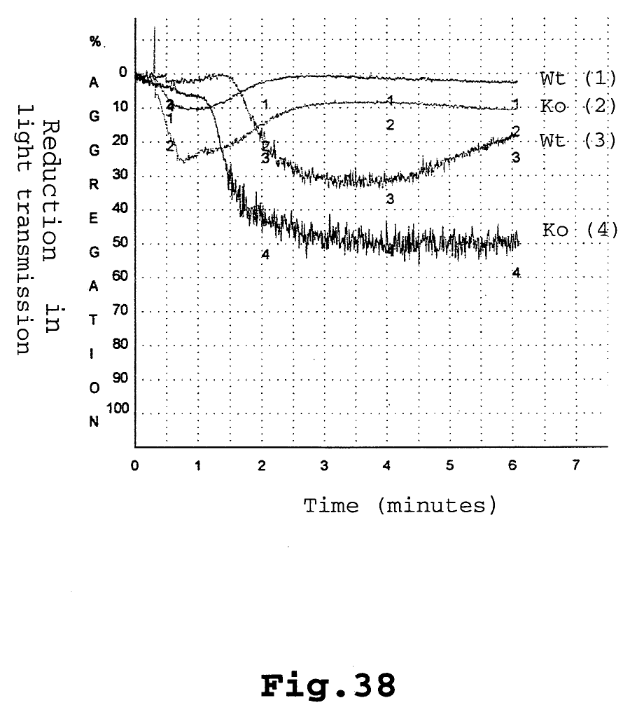

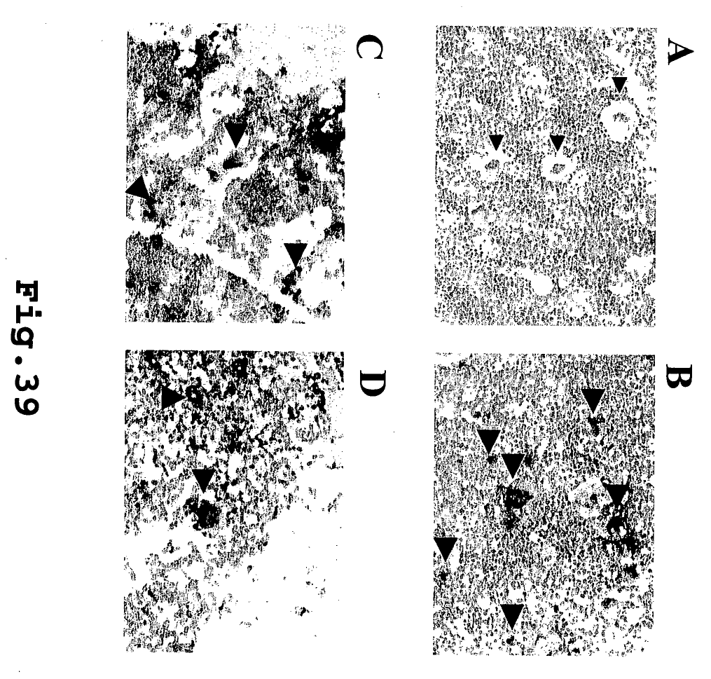

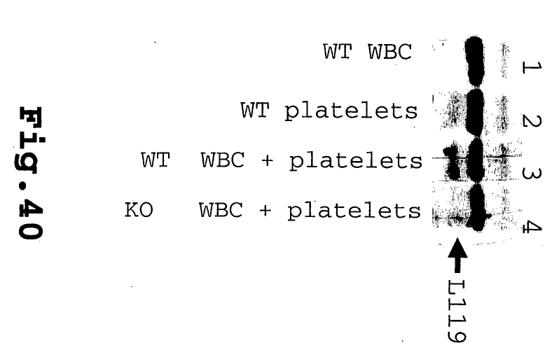

Wt and L119 ko mice were anesthetized using 200 mg/kg sodium pentobarbital. The chest cavity was opened and 900 gl blood was drawn into a syringe containing 100 μl Heparin (1000 units/ml in 137 mM NaCl) by direct cardiac puncture into the right ventricle. 2 mL of Heparin blood derived from 2 wt and 2 L119 ko mice respectively was layered each on top of 3 ml Histopaque 1077. After centrifugation at 250 g for 10 min at RT 900 μl of platelet-rich plasma (PRP) were removed and diluted 1:1 with RT Tyrodes buffer (5 mM HEPES pH 7.35, 135 mM NaCl, 2.7 mM KCl, 2 mM MgCl₂, 11.8 mM NaHCO₃, 0.42 mM NaH₂PO₄, 0.1% Dextrose, 0.35% BSA) and the platelet count was determined (Beckman Coulter Counter). Aggregation of platelet rich plasma (PRP) with 200000 platelets/μl was measured by analysis of light transmission in an aggregometer (Bio-Data Aggregometer Platelet Aggregation Profiler PAP4) at 37° C. Platelet poor plasma (PPP) was used as a control for definition of 100% light transmission. It was obtained by centrifugation of PRP at 10000 g for 2 min. The supernatant was used for measurements. Platelet suspensions (PRP) were constantly stirred and after addition of agonists (Agonists: ADP (1 μm) or Collagen (0.5 μg/ml)) increase in light transmission during the aggregation process was monitored for 6 min. Platelets from L119 ko mice (FIG. 38, curve 2 and 4) showed a more vigorous aggregation profile than platelets from wt littermates (FIG. 38, curve 1 and 3). The experiments reveal that L119 ko mice exert a stronger, more intense pro-thrombotic response to injuries. This effect was seen with two different agonists in a dose dependent manner, supporting the hypothesis that the L119 null phenotype is related to a hyper-activation of platelet function.

### Example 21

### L119 mRNA Expression in Megakaryoctes

For induction of L119 gene expression male Wistar rats were injected intraperitoneal with 50 mg/kg cycloheximide (Sigma-Aldrich) in PBS/EtOH (1:1), controls obtained vehicle (PBS/Ethanol, 1:1). 4 h later rats were decapitated and the femur was taken out, muscle and connective tissue was excised, and both ends of the bone were removed using a bone cutter. A 10 ml syringe with PBS was placed at one end of the bone and by pressure the bone marrow was released. Bone marrow was embedded in Tissue-Tek/OCT (Sakura; Cat. No. 4583) cryosectioned at 10 μm and collected on glass slides. In situ hybridizations (FIG. 39) were performed using a digoxygenin-labeled L119 riboprobe followed by immunological detection with alkaline phosphatase as described under Methods. The tissue was counter-stained using nuclear fast red (Vector Laboratories, Cat. No. H-3404) according to the manufacturers' instructions. In situ hybridizations of bone marrow derived from cycloheximide treated animals showed a L119 specific staining of megacaryocytes (FIG. 39 B-D) whereas L119 mRNA levels in unstimulated controls were below the detection limit (FIG. 39A). Megakaryocytes are marked by arrows.

### Example 22

### L119 Protein Expression in Blood Cells

Wt and L119 ko mice were anesthetized using 100 mg/kg sodium pentobarbital. The chest cavity was opened and 900 μl blood was drawn into a syringe containing 100 μl of Heparin (1000 units/ml in 137 mM NaCl) by direct cardiac puncture into the right ventricle. Heparin blood of wt and.L119 ko mice was mixed by inversion 2:1 (vol:vol) with Hank's Balanced salt solution (Invitrogen, Cat. No. 14170-112). The blood/HBSS mixture was gently layered on top of an equal volume of Histopaque-1119 (Sigma-Aldrich, Cat. No. 1119-1) and centrifuged at 400 g for 30 min) in a 15 ml conical tube. Histopalue-1119 had been pre-warmed to room temperature before use.

For preparation of the white blood cell (WBC)/platelet fraction the clear upper plasma layer was removed and discarded. The (WBC)/platelet layer was then transferred to a fresh 15 ml conical tube, a 10-fold volume of HBSS was added and blood cells were collected by centrifugation at 2000 g for 10 min. The supernatant was discarded and the cell pellet was resuspended in 500 μl 2× Laemmli buffer. After sonication, cell lysates were boiled for 5 min and centrifuged for 15 min at 12000 g at room temperature. 12 μl of each lysate was subjected to western blot analysis (FIG. 40 lane 3 and 4). Protein A-purified IgG from rabbit 3843 (at 1:500) was used as primary antibody and HRP-conjugated donkey anti-rabbit (Jackson ImmunoResearch Laboratories, Inc.) as secondary antibody. Western blot analysis was performed as.described under Methods. A L119 immunoreactive band could be detected in the WBC/platelet fraction derived from wt mice but it was absent in ko mice. For further analysis WBC and platelets from wt animals were prepared separately and analyzed by western blotting. For preparation of WBC and platelets Heparin/HBSS blood was centrifuged on top of a Histopaque-119 layer as described above. After centrifugation the plasma fraction and the white blood cells (WBC)/platelet fraction were combined in a fresh tube and centrifuged at 120 g for 8 min. The pellet represents white blood cells and the supernatant the platelet rich plasma (PRP). Platelets were collected by centrifugation of the PRP at 2000 g for 10 min. Cell pellets were lysed in 2× Laemmli-buffer and analyzed by western blotting as described above (FIG. 40 lanes 1 and 2). A L119 specific immunoreactive band could be detected in of WBC/platelet preparations of wt animals (FIG. 40 lane 3) which was absent in ko mice (lane 4). The L119 specific band segregated with the platelet fraction (lane 2) and was not found in the WBC fraction of wt animals.

We claim:

1. A protein, which comprises
a) one of the amino acid sequences depicted in SEQ ID NO: 3, 6, 7 or 24, or
b) a sequence which can be obtained by the substitution, insertion or deletion of one or more amino acid residues in one of the amino acid sequences depicted in SEQ ID NO: 3, 6, 7 or 24, with at least one of the essential properties of the proteins depicted in SEQ ID NO: 3, 6, 7 or 24 being retained, or
c) a functional equivalent or functionally equivalent part of one of the proteins depicted in SEQ ID NO: 3, 6, 7 or 24.

2. An isolated protein as claimed in claim 1, which comprises at least one of the following sequence motifs:

| | |
|---|---|
| a) | DALRRFQGLLLDRRGRLH |
| b) | QVLRLREVARRLERLRRRSL |
| c) | GALAAIVGLSLSPVTLG |
| d) | SAVGLGVATAGGAVTITSDLSLIFCNSRE |
| e) | RRVQEIAATCQDQMRE |
| f) | ALYNSVYFIVFFGSRGFLIPRRAEG |
| g) | TKVSQAVLKAKIQKL |
| h) | ESLESCTGALDELSEQLESRVQLCTK |

3. A nucleic acid sequence which encodes a protein as claimed in claim 1 or 2.

4. A nucleic acid sequence as claimed in claim 3, which
a) encodes a protein which has at least 60% identity with the sequence depicted in SEQ ID NO: 3, 6, 7 or 24, or
b) has an identity of at least 60% with one of the nucleic acid sequences depicted in SEQ ID NO: 1, 2, 4, 5, 22 or 23.

5. A nucleic acid sequence as claimed in claim 3 or 4, which comprises the sequence depicted in SEQ ID NO: 1, 2, 4, 5, 22 or 23.

6. A nucleic acid construct which comprises a nucleic acid sequence as claimed in one of claims 3 to 5 linked to at least one genetic regulatory element.

7. A transgenic, nonhuman organism which is transformed with a functional or non-functional transgenic nucleic acid sequence as claimed in one of claims 3 to 5 or with a functional or non-functional transgenic nucleic acid construct as claimed in claim 6.

8. A transgenic, nonhuman organism as claimed in claim 7, which is an animal organism.

9. A transgenic, nonhuman animal in whose germ cells, or the entirety or a part of the somatic cells, or in whose germ cells and the entirety or a part of the somatic cells, the nucleic acid sequence as claimed in one of claims 3 to 5 has been transgenically altered by recombinant methods or interrupted by inserting DNA elements.

10. A process for finding compounds having specific binding affinity for a protein as claimed in claim 1 or 2, which comprises the following steps:
a) incubating the protein as claimed in claim 1 or 2 with the compound to be tested,
b) detecting the binding of the compound to be tested to the protein.

11. A process for finding compounds having specific binding affinity for a nucleic acid as claimed in one of claims 3 to 5, which comprises the following steps:
a) incubating a nucleic acid as claimed in one of claims 3 to 5 with the compound to be tested,
b) detecting the binding of the compound to be tested to the nucleic acid.

12. A process for finding compounds which modulate or normalize at least one essential property, or the expression, of a protein as claimed in claim 1 or 2, which comprises the following steps:
a) incubating a protein as claimed in claim 1 or 2, or a nucleic acid sequence as claimed in one of claims 3 to 5, or a nucleic acid construct as claimed in claim 6, or a transgenic organism as claimed in claim 7 or 8, or a transgenic animal as claimed in claim 9, with the compound to be tested,
b) determining the modulation or normalization of an essential property, or of the expression, of a protein as claimed in claim 1 or 2.

13. A process as claimed in claim 12, wherein the modulation or normalization of an essential property is determined by direct binding of the compound to be tested to said protein, nucleic acid sequence or nucleic acid construct.

14. A process as claimed in one of claims 10 to 13, wherein the following are used
a) an immunoprecipitation, or
b) an N-hybrid system, or
c) a phage display system, or
d) a library of low molecular weight compounds, or
e) a reporter system, or
f) antibody selection techniques, or
g) immunoassays such as ELISA or Western blotting, or
h) molecular modeling using the structural information for a protein as claimed in claim 1 or 2 or for a nucleic acid sequence as claimed in one of claims 3 to 5, or
i) affinity chromatography, or
j) microphysiometer.

15. A compound which can be obtained by a process as claimed in one of claims 10 to 14.

16. A compound as claimed in claim 15, which is
a) a protein, or
b) a nucleic acid, or
c) a low molecular weight compound having a molecular weight of less than 1000 g/mol.

17. A compound as claimed in claim 15 or 16, which is selected from the group consisting of polyclonal or monoclonal antibodies, antibody mixtures, single-chain antibodies or antibody fragments, aptamers, natural or artificial transcription factors, antisense nucleic acids, double-stranded RNA molecules, α-anomeric nucleic acids, low molecular weight compounds and ribozymes.

18. The use of a nucleic acid sequence as claimed in one of claims 3 to 5, of a nucleic acid construct as claimed in claim 6 or of a protein as claimed in claim 1 or 2 for identifying proteins which possess specific binding affinities for a protein as claimed in claim 1 or 2, or for identifying nucleic acids which encode proteins which possess specific binding affinities for a protein as claimed in claim 1 or 2.

19. The use of a nucleic acid sequence as claimed in one of claims 3 to 5, or of a fragment thereof, for isolating a genomic sequence by means of screening for homology.

20. The use of a nucleic acid sequence as claimed in one of claims 3 to 5 as
a) a marker for human hereditary diseases, or
b) for detecting sequence polymorphisms which correlate with predispositions to diseases.

21. The use of a nucleic acid sequence as claimed in one of claims 3 to 5, of a nucleic acid sequence which is complementary to a nucleic acid sequence as claimed in one of claims 3 to 5, of a nucleic acid construct as claimed in claim 6, or of a transgenic organism as claimed in claim 7 or 8, or parts thereof, for the treatment of human diseases by gene therapy.

22. A process for qualitatively or quantitatively detecting the presence, the absence, the incorrectly regulated expression, or an incorrect function, of a protein as claimed in claim 1 or 2, or of a nucleic acid sequence as claimed in one of claims 3 to 5, in a biological sample, which comprises one or more of the following steps:
a) isolating a biological sample from a test subject
b) incubating the biological sample with a reagent which is suitable for detecting a protein as claimed in claim 1 or 2 or a nucleic acid sequence as claimed in one of claims 3 to 5, in a manner such that the presence, the absence, the incorrectly regulated expression or an incorrect function, of a protein as claimed in claim 1 or 2, or of a nucleic acid sequence as claimed in one of claims 3 to 5, can be detected.

23. A process for qualitatively or quantitatively detecting a nucleic acid as claimed in one of claims 3 to 5 in a biological sample, which comprises one or more of the following steps:
a) incubating a biological sample with a known quantity of nucleic acid as claimed in one of claims 3 to 5 or a known quantity of oligonucleotides which are suitable for use as primers for amplifying the nucleic acid as claimed in one of claims 3 to 5, or mixtures thereof,
b) detecting the nucleic acid as claimed in one of claims 3 to 5 by means of specific hybridization or PCR amplification,
c) comparing the quantity of hybridizing nucleic acid as claimed in one of claims 3 to 5, or of nucleic acid obtained by PCR amplification as claimed in one of claims 3 to 5, with a standard.

24. A process for qualitatively or quantitatively detecting a protein as claimed in claim 1 or 2 in a biological sample, which comprises one or more of the following steps:
a) incubating a biological sample with an antibody which is specifically directed against proteins as claimed in claim 1 or 2,
b) detecting the antibody/antigen complex,
c) comparing the quantities of the antibody/antigen complex with a quantity standard.

25. The use of proteins as claimed in claim 1 or 2, or of protein fragments or peptides which are derived therefrom, of nucleic acids as claimed in one of claims 3 to 5, or of complementary nucleic acid sequences, or parts thereof, which are derived therefrom, of nucleic acid constructs as obtained in claim 6, or of compounds as claimed in one of claims 15 to 17, for producing drugs.

38. A protein, which comprises
a) the amino acid sequence depicted in SEQ ID NO: 24, or
b) a sequence which can be obtained by the substitution, insertion or deletion of one or more amino acid residues in the amino acid sequence depicted in SEQ ID NO: 24, with at least one of the essential properties of the proteins depicted in SEQ ID NO: 24 being retained, or
c) a functional equivalent or functionally equivalent part of the protein depicted in SEQ ID NO: 24.

39. An isolated protein as claimed in claim 38, which comprises at least one of the following sequence motifs:

| | |
|---|---|
| a) | DALRRFQGLLLDRRGRLH |
| b) | QVLRLREVARRLERLRRRSL |
| c) | GALAAIVGLSLSPVTLG |
| d) | SAVGLGVATAGGAVTITSDLSLIFCNSRE |
| e) | RRVQEIAATCQDQMRE |
| f) | ALYNSVYFIVFFGSRGFLIPRRAEG |
| g) | TKVSQAVLKAKIQKL |
| h) | ESLESCTGALDELSEQLESRVQLCTK |

40. A nucleic acid sequence which encodes a protein as claimed in claim 38.

41. A nucleic acid sequence as claimed in claim 40, which
a) encodes a protein which has at least 60% identity with the sequence depicted in SEQ ID NO: 24, or
b) has an identity of at least 60% with the nucleic acid sequence depicted in SEQ ID NO: 23.

42. A nucleic acid sequence as claimed in claim 40, which comprises the sequence depicted in SEQ ID NO: 4 or 23.

43. A nucleic acid construct which comprises a nucleic acid sequence as claimed in claim 40 linked to at least one genetic regulatory element.

44. A transgenic, nonhuman organism which is transformed with a functional or nonfunctional transgenic nucleic acid sequence as claimed in claim 40.

45. A transgenic, nonhuman organism which is transformed with a functional or nonfunctional transgenic nucleic acid construct as claimed in claim 43.

46. A transgenic, nonhuman organism as claimed in claim 44, which is an animal organism.

47. A transgenic, nonhuman organism as claimed in claim 45, which is an animal organism.

48. A transgenic, nonhuman animal in whose germ cells, or the entirety or a part of the somatic cells, or in whose germ cells and the entirety or a part of the somatic cells, the nucleic acid sequence as claimed in claim 40 has been transgenically altered by recombinant methods or interrupted by inserting DNA elements.

49. A process for finding compounds having specific binding affinity for a protein as claimed in claim 38, which comprises the following steps:
a) incubating the protein as claimed in claim 38 with the compound to be tested,
b) detecting the binding of the compound to be tested to the protein.

50. A process for finding compounds having specific binding affinity for a nucleic acid as claimed in claim 40, which comprises the following steps:
a) incubating a nucleic acid as claimed in claim 40 with the compound to be tested,
b) detecting the binding of the compound to be tested to the nucleic acid.

51. A process for finding compounds which modulate or normalize at least one essential property, or the expression, of a protein as claimed in claim 38, which comprises the following steps:
a) incubating a protein as claimed in claim 38 with the compound to be tested,
b) determining the modulation or normalization of an essential property, or of the expression, of a protein as claimed in claim 38.

52. A process for finding compounds which modulate or normalize at least one essential property, or the expression, of a protein as claimed in claim 38, which comprises the following steps:
a) incubating a nucleic acid sequence which encodes a protein as claimed in claim 1, with the compound to be tested,
b) determining the modulation or normalization of an essential property, or of the expression, of a protein as claimed in claim 38.

53. A process for finding compounds which modulate or normalize at least one essential property, or the expression, of a protein as claimed in claim 38, which comprises the following steps:
a) incubating a nucleic acid construct which comprises a nucleic acid sequence which encodes a protein as claimed in claim 38, with the compound to be tested,
b) determining the modulation or normalization of an essential property, or of the expression, of a protein as claimed in claim 38.

54. A process for finding compounds which modulate or normalize at least one essential property, or the expression, of a protein as claimed in claim 38, which comprises the following steps:
a) incubating a transgenic, non-human organism which is transformed with a functional or non-functional transgenic nucleic acid construct, which encodes a protein as claimed in claim 38, with the compound to be tested,
b) determining the modulation or normalization of an essential property, or of the expression, of a protein as claimed in claim 38.

55. A process for finding compounds which modulate or normalize at least one essential property, or the expression, of a protein as claimed in claim 38, which comprises the following steps:
a) incubating a transgenic, non-human organism which is transformed with a functional or non-functional transgenic nucleic acid construct which comprises a nucleic acid sequence which encodes a protein as claimed in claim 38, with the compound to be tested,
b) determining the modulation or normalization of an essential property, or of the expression, of a protein as claimed in claim 38.

56. A process for finding compounds which modulate or normalize at least one essential property, or the expression, of a protein as claimed in claim 38, which comprises the following steps:
a) incubating a transgenic, non-human animal in whose germ cells, or the entirety or a part of the somatic cells, or in whose germ cells and the entirety or a part of the somatic cells, the nucleic acid which encodes a protein as claimed in claim 38 has been transgenically altered by recombinant methods or interrupted by inserting DNA elements, with the compound to be tested,
b) determining the modulation or normalization of an essential property, or of the expression, of a protein as claimed in claim 38.

57. A process as claimed in claim 51, wherein the modulation or normalization of an essential property is determined by direct binding of the compound to be tested to said protein, nucleic acid sequence or nucleic acid construct.

58. A process as claimed in claim 52, wherein the modulation or normalization of an essential property is determined by direct binding of the compound to be tested to said protein, nucleic acid sequence or nucleic acid construct.

59. A process as claimed in claim 53, wherein the modulation or normalization of an essential property is determined by direct binding of the compound to be tested to said protein, nucleic acid sequence or nucleic acid construct.

60. A process as claimed in claim 54, wherein the modulation or normalization of an essential property is determined by direct binding of the compound to be tested to said protein, nucleic acid sequence or nucleic acid construct.

61. A process as claimed in claim 55, wherein the modulation or normalization of an essential property is determined by direct binding of the compound to be tested to said protein, nucleic acid sequence or nucleic acid construct.

62. A process as claimed in claim 49, wherein the following are used
a) an immunoprecipitation, or
b) an N-hybrid system, or
c) a phage display system, or
d) a library of low molecular weight compounds, or
e) a reporter system, or
f) antibody selection techniques, or
g) immunoassays such as ELISA or Western blotting, or
h) molecular modelling using the structural information for a protein which comprises
a) the amino acid sequence depicted in SEQ ID NO: 24, or
b) a sequence which can be obtained by the substitution, insertion or deletion of one or more amino acid residues in the amino acid sequence depicted in SEQ ID NO: 24, with at least one of the essential properties of the proteins depicted in SEQ ID NO: 24 being retained, or
c) a functional equivalent or functionally equivalent part of the protein depicted in SEQ ID NO: 24, or
i) affinity chromatography, or
j) microphysiometer.

63. A process as claimed in claim 49, wherein the following are used
a) an immunoprecipitation, or
b) an N-hybrid system, or
c) a phage display system, or
d) a library of low molecular weight compounds, or
e) a reporter system, or
f) antibody selection techniques, or
g) immunoassays such as ELISA or Western blotting, or
h) molecular modelling using the structural information for a nucleic acid sequence which encodes a protein, which comprises
a) the amino acid sequence depicted in SEQ ID NO: 24, or
b) a sequence which can be obtained by the substitution, insertion or deletion of one or more amino acid residues in the amino acid sequence depicted in SEQ ID NO: 24, with at least one of the essential properties of the proteins depicted in SEQ ID NO: 24 being retained, or
c) a functional equivalent or functionally equivalent part of the protein depicted in SEQ ID NO: 24, or
i) affinity chromatography, or
j) microphysiometer.

64. A compound which can be obtained by a process as claimed in claim 49.

65. A compound as claimed in claim 64, which is
a) a protein, or
b) a nucleic acid, or
c) a low molecular weight compound having a molecular weight of less than 1000 g/mol.

66. A compound as claimed in claim 64, which is selected from the group consisting of polyclonal or monoclonal antibodies, antibody mixtures, single-chain antibodies or antibody fragments, apatamers, natural or artificial transcription factors, antisense nucleic acids, double-stranded RNA molecules, a-anomeric nucleic acids, low molecular weight compounds and ribozymes.

67. The method of using a nucleic acid sequence which encodes a protein as claimed in claim 38, for identifying proteins which possess specific binding affinities for a protein as claimed in claim 38.

68. The method of using a nucleic acid construct which comprises a nucleic acid sequence which encodes a protein as claimed in claim 38, for identifying proteins which possess specific binding affinities for a protein as claimed in claim 38.

69. The method of using a protein as claimed in claim 38, for identifying proteins which possess specific binding affinities for a protein as claimed in claim 38.

70. The method of using a nucleic acid sequence which encodes a protein as claimed in claim 38, for identifying nucleic acids which encode proteins which possess specific binding affinities for a protein as claimed in claim 38.

71. The method of using a nucleic acid construct which comprises a nucleic acid sequence which encodes a protein as claimed in claim 38, for identifying nucleic acids which encode proteins which possess specific binding affinities for a protein as claimed in claim 38.

72. The method of using a protein as claimed in claim 38, for identifying nucleic acids which encode proteins which possess specific binding affinities for a protein as claimed in claim 38.

73. The method of using a nucleic acid sequence as claimed in claim 40, or a fragment thereof, for isolating a genomic sequence by means of screening for homology.

74. The method of using a nucleic acid sequence as claimed in claim 40 as
a) a marker for hereditary diseases, or
b) for detecting sequence polymorphisms which correlate with predispositions to diseases.

75. The method of using a nucleic acid sequence as claimed in claim 40, or a nucleic acid sequence which is complementary to a nucleic acid sequence as claimed in claim 40, or parts thereof, for the treatment of human diseases by gene therapy.

76. The method of using a nucleic acid construct as claimed in claim 43, or parts thereof, for the treatment of human diseases by gene therapy.

77. The method of using a transgenic organism as claimed in claim 44, or parts thereof, for the treatment of human diseases by gene therapy.

78. The method of using a transgenic organism as claimed in claim 45, or parts thereof, for the treatment of human diseases by gene therapy.

79. The method of using a protein as claimed in claim 38, or which comprises a sequence selected from the group of SEQ ID NO 3, 6 and 7, or of protein fragments or peptides which are derived therefrom, for producing drugs for the treatment and prophylaxis of diseases, which can be influenced by the modulation of the activity or the amount of an L119 protein, except epilepsy or ischemic diseases.

80. The method of using a nucleic acid sequence as claimed in claim 40 or which is selected from the group of SEQ ID NO 1, 2, 5, 22 and 23, or of complementary sequences or parts thereof for producing drugs for the treatment and prophylaxis of diseases, which can be influenced by the modulation of genexpression of an L119 protein, except epilepsy or ischemic diseases.

81. The method of using compounds as claimed in claim 64 for producing drugs for the treatment and prophylaxis of diseases, which can be influenced by the modulation of the activity or the amount of an L119 protein, except epilepsy or ischemic diseases.

82. The method of using compounds which posses specific binding affinities for proteins comprising a sequence selected from the group of SEQ ID NO 3, 6 and 7, for producing drugs for the treatment and prophylaxis of diseases, which can be influenced by the modulation of the activity or the amount of an L119 protein, except epilepsy or ischemic diseases.

83. The method of using compounds which modulate or normalize at least one essential property or the expression of a protein comprising a sequence selected from the group of SEQ ID NO 3, 6 and 7, for producing drugs for the treatment and prophylaxis of diseases, which can be influenced by the modulation of the activity or the amount of an L119 protein, except epilepsy or ischemic diseases.

84. The method of using as claimed in claim 79 for the treatment of vascular or endothelial diseases, wherein the vascular or endothelial disease is selected from the group of vascular homeostasis disease, endothelial disease, coagulation disease, thrombotic disease and platelet disease.

85. The method of using as claimed in claim 80 for the treatment of vascular or endothelial diseases, wherein the vascular or endothelial disease is selected from the group of vascular homeostasis disease, endothelial disease, coagulation disease, thrombotic disease and platelet disease.

86. The method of using as claimed in claim 81 for the treatment of vascular or endothelial diseases, wherein the vascular or endothelial disease is selected from the group of vascular homeostasis disease, endothelial disease, coagulation disease, thrombotic disease and platelet disease.

87. The method of using as claimed in claim 82 for the treatment of vascular or endothelial diseases, wherein the vascular or endothelial disease is selected from the group of vascular homeostasis disease, endothelial disease, coagulation disease, thrombotic disease and platelet disease.

88. The method of using as claimed in claim 83 for the treatment of vascular or endothelial diseases, wherein the vascular or endothelial disease is selected from the group of vascular homeostasis disease, endothelial disease, coagulation disease, thrombotic disease and platelet disease.

89. The method of using as claimed in claim 79 wherein the disease is cancer.

90. The method of using as claimed in claim 80 wherein the disease is cancer.

91. The method of using as claimed in claim 81 wherein the disease is cancer.

92. The method of using as claimed in claim 82 wherein the disease is cancer.

93. The method of using as claimed in claim 82 wherein the disease is cancer.

94. The method of using a protein as claimed in claim 38, or which comprises a sequence selected from the group of SEQ ID NO 3, 6 and 7, or of protein fragments or peptides which are derived therefrom, for producing drugs for the treatment and prophylaxis of epilepsy and ischemic diseases, which can be influenced by the modulation of the activity or the amount of an L119 protein, and results in an alteration of the endothelium.

95. The method of using a nucleic acid sequence as claimed in claim 40 or which is selected from the group of SEQ ID NO 1, 2, 5, 22 and 23, or of complementary sequences or parts thereof for producing drugs for the treatment and prophylaxis of epilepsy or ischemic diseases, which can be influenced by the modulation of genexpression of an L119 protein, and results in an alteration of the endothelium.

96. The method of using compounds as claimed in claim 64 for producing drugs for the treatment and prophylaxis of epilepsy or ischemic diseases, which can be influenced by the modulation of the activity or the amount of an L119 protein, and results in an alteration of the endothelium.

97. The method of using compounds which posses specific binding affinities for proteins comprising a sequence selected from the group of SEQ ID NO 3, 6 and 7, for producing drugs for the treatment and prophylaxis of epilepsy or ischemic diseases, which can be influenced by the modulation of the activity or the amount of an L119 protein, and results in an alteration of the endothelium.

98. The method of using compounds which modulate or normalize at least one essential property or the expression of a protein comprising a sequence selected from the group of SEQ ID NO 3, 6 and 7, for producing drugs for the treatment and prophylaxis of epilepsy or ischemic diseases, which can be influenced by the modulation of the activity or the amount of an L119 protein, and results in an alteration of the endothelium.

99. A process for qualitatively or quantitatively detecting the presence, the absence, the incorrectly regulated expression, or an incorrect fumction, of a protein as claimed in claim 38, in a biological sample, which comprises one or more of the following steps:
a) isolating a biological sample from a test subject
b) incubating the biological sample with a reagent which is suitable for detecting a protein as claimed in claim 38 or a nucleic acid sequence which encodes a protein as claimed in claim 38, in a manner such that the presence, the absence, the incorrectly regulated expression or an incorrect function, of a protein as claimed in claim 38, or of a nucleic acid sequence which encodes a protein as claimed in claim 38, can be detected.

100. A process for qualitatively or quantitatively detecting the presence, the absence, the incorrectly regulated expression, or an incorrect function, of a nucleic acid sequence which encodes a protein as claimed in claim 38, in a biological sample, which comprises one or more of the following steps:
a) isolating a biological sample from a test subject
b) incubating the biological sample with a reagent which is suitable for detecting a protein as claimed in claim 38 or a nucleic acid sequence which encodes a protein as claimed in claim 38, in a manner such that the presence, the absence, the incorrectly regulated expression or an incorrect function, of a protein as claimed in claim 38, or of a nucleic acid sequence which encodes a protein as claimed in claim 38, can be detected.

101. A process for qualitatively or quantitatively detecting a nucleic acid as claimed in claim 40 in a biological sample, which comprises one or more of the following steps:
a) incubating a biological sample with a known quantity of nucleic acid as claimed in claim 40 or a known quantity of oligonucleotides which are suitable for use as primers for amplifying the nucleic acid as claimed in claim 40, or mixtures thereof,
b) detecting the nucleic acid as claimed in claim 40 by means of specific hybridization or PCR amplification,
c) comparing the quantity of hybridizing nucleic acid as claimed in claim 40, or of nucleic acid obtained by PCR amplification as claimed in claim 40, with a standard.

102. A process for qualitatively or quantitatively detecting a protein as claimed in claim 38 in a biological sample, which comprises one or more of the following steps:
a) incubating a biological sample with an antibody which is specifically directed against proteins as claimed in claim 38,
b) detecting the antibody/antigen complex,
c) comparing the quantities of the antibody/antigen complex with a quantity standard.
